(19) United States
(12) Patent Application Publication
(10) Pub. No.: US 2022/0249779 A1
(43) Pub. Date: Aug. 11, 2022

(54) SYSTEMS, DEVICES, AND METHODS RELATING TO MEDICATION DOSE GUIDANCE

(71) Applicant: ABBOTT DIABETES CARE INC., Alameda, CA (US)

(72) Inventors: Gary A. Hayter, Oakland, CA (US); Aparajita Bhattacharya, Dublin, CA (US); Erwin S. Budiman, Fremont, CA (US); Matthew T. Novak, Oakland, CA (US); Taihao Jin, Richmond, CA (US); Marc B. Taub, Mountain View, CA (US); Jonathan M. Fern, Alameda, CA (US); Yongjin Xu, San Ramon, CA (US); Kaiyuan Zhu, Hangzhou City (CN); Kendall Covington, Oakland, CA (US); Kelly Cook, Alameda, CA (US); Jessica Rose Floeh, Baltimore, MD (US); Rosina Bosco, Boulder, CO (US); Taylor M. Charlesworth, Danville, CA (US); Vyshnnavi Parthasarathy, Fremont, CA (US)

(21) Appl. No.: 17/591,229

(22) Filed: Feb. 2, 2022

Related U.S. Application Data
(63) Continuation-in-part of application No. 29/817,852, filed on Dec. 3, 2021.
Application No. 29/817,852 is a continuation-in-part of application No. 29/817,851, filed on Dec. 3, 2021.
Application No. 29/817,851 is a continuation-in-part of application No. 29/814,001, filed on Nov. 2, 2021.
Application No. 29/814,001 is a continuation-in-part of application No. 29/813,998, filed on Nov. 2, 2021.
(60) Provisional application No. 63/237,769, filed on Aug. 27, 2021.
(60) Provisional application No. 63/225,140, filed on Jul. 23, 2021.
(60) Provisional application No. 63/145,131, filed on Feb. 3, 2021.

(51) Int. Cl. A61M 5/315

(52) U.S. Cl. CPC A61M 5/31535; A61M 2005/3126; A61M 2205/3553; A61M 2205/52

(57) ABSTRACT

Systems, devices and methods are provided for determining a medication dose for a patient or user. The dose determination can account for recent and/or historical analyte levels of the patient or user. The dose determination can also take into account other information about the patient or user, such as physiological information, dietary information, activity, and/or behavior. Many different dose determination embodiments are set forth, pertaining to a wide array of different aspects of the system or environment in which the embodiments can be implemented. Systems, devices and methods are provided for displaying information related to glucose levels, including a time in range display and a graph of analyte levels containing an identification of a pattern type of a segment of the day.

### CROSS-REFERENCE TO RELATED APPLICATIONS

This application claims priority to U.S. Application Ser. No. 63/237,769, filed Aug. 27, 2021, U.S. Application Ser. No. 63/225,140, filed Jul. 23, 2021, and U.S. Application Ser. No. 63/145,131, filed Feb. 3, 2021, all of which are herein expressly incorporated by reference in their entirety for all purposes. This application is also a continuation-in-part of U.S. application Ser. No. 29/817,852, filed Dec. 3, 2021, a continuation-in-part of U.S. application Ser. No. 29/817,851, filed Dec. 3, 2021, a continuation-in-part of U.S. application Ser. No. 29/814,001, filed Nov. 2, 2021, and a continuation-in-part of U.S. application Ser. No. 29/813,998, filed Nov. 2, 2021, all of which are herein expressly incorporated by reference in their entirety for all purposes. This application is also related to U.S. application Ser. No. 16/944,736, filed Jul. 31, 2020, which claims priority to, and the benefit of, U.S. Provisional Application No. 62/882,249, filed Aug. 2, 2019, U.S. Provisional Application No. 62/979,578, filed Feb. 21, 2020, U.S. Provisional Application No. 62/979,594, filed Feb. 21, 2020, U.S. Provisional Application No. 62/979,618, filed Feb. 21, 2020, and U.S. Provisional Application Ser. No. 63/058,799, filed Jul. 30, 2020, all of which are herein expressly incorporated by reference in their entirety for all purposes.

### FIELD

The subject matter described herein relates generally to systems, devices, and methods relating to medication dose guidance such as, for example, the determination of an insulin dose for the treatment of elevated glucose levels resulting from diabetes.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors including inconvenience, testing discretion, pain associated with glucose testing, and cost.

For patients that rely on the administration of medications (e.g., insulin) to treat or manage diabetes, it is desirable to have systems, devices, or methods that can automatically utilize glucose information collected by an analyte monitoring system to provide medication dose guidance in a readily accessible manner on an as-needed basis. It is further desirable for such systems, devices, or methods to take into account the physiology, diet, activity, and/or behavior of a user or patient to be treated in providing such medication dose guidance, as such may improve accuracy and reliability. Further, in some circumstances, it is also desirable for such systems, devices, or methods to be capable of automatically delivering a selected medication dose.

For these and other reasons, needs exist for improved systems, methods, and devices relating to medication dose guidance.

### SUMMARY

Provided herein are example embodiments of systems, devices and methods relating to the provision of medication dose guidance and, in some embodiments, medication delivery. According to one aspect, many of the embodiments described herein comprise a dose guidance system (DGS) that includes a display device, a sensor control device, and a medication delivery device. The dose guidance system can include a dose guidance application (e.g., software) that can determine and output dose guidance (e.g., recommendations regarding dose amounts, corrections, and titrations) to a patient. Furthermore, according to some embodiments, the dose guidance system can learn a patient's dosing strategy during a learning period in which the dose guidance system can estimate key dosing parameters. According to some embodiments, the dose guidance system can also provide guidance for titrations and corrections once the system is configured with a patient's current dosing strategies. The dose guidance system can also provide guidance for different meal dosing scenarios. For example, in some embodiments, the dose guidance system can provide dose guidance at or before a start of a meal or after a meal has started. The dose guidance system can also provide dose guidance for compounded meals (e.g., dessert) or for “touch up” doses to address high post-prandial glucose levels. Exemplary system and safety features of the dose guidance system are also described.

Many of the embodiments provided herein comprise improved software features or graphical user interfaces for use with analyte monitoring systems that are highly intuitive, user-friendly, and provide for rapid access to physiological information of a user. More specifically, these embodiments allow a user (or an HCP) to rapidly determine an appropriate medication therapy based on information relating to the user's physiological conditions, historic dosing patterns, and other factors, without requiring the user (or an HCP) to go through the arduous task of examining large volumes of analyte data. Furthermore, some of the GUIs and GUI features, allow for users (and their caregivers) to better understand and improve a user's dosing patterns and subsequent hypo and hyperglycemic episodes. Likewise, many other embodiments provided herein comprise improved software features for dose guidance systems that improve upon: dose guidances provided to users by allowing for safe titration strategies that minimize hypoglycemic episodes, methods for altering dose guidances depending on when the dose is to be administered relative to a meal start time (e.g., before, at the start, or after the start of a meal), consideration of real-world occurrences that effect dosing strategies, post-prandial alarms based on a predicted occurrence probability rather than a threshold, to name only a few.

Moreover, many embodiments described herein are improvements over traditional bolus calculators in which the HCP needs to configure numerous settings, which is a very time-consuming process. Alternatively, the system may require the patient to enter numerous settings, which are often confusing for the patient. The embodiments described herein significantly reduce the manual input required by both the patient and the HCP by requiring no or minimal input (e.g., typical meal dose amounts) from the patient, and the system can learn the other necessary parameters related to the patient's insulin dosing practice. Additionally, many patients have poor adherence to a suggested dosing regimen. During a learning period, the system may collect data regarding both glucose levels and insulin administration and can assess the level of the patient's adherence. Thus, the system's features can solve problems related to the HCP's time investment and the sophistication level of the patient. Another advantage of the system is that, whereas traditional bolus calculators use fixed parameters that need to be configured, the system may automatically titrate (i.e., optimize) dose regimen parameters over time; that is, optimize their dose regimen to reduce patterns of low glucose or high glucose. This relieves the HCP of the time burden of having to perform the task of periodically reviewing the patient's glucose data and updating their dose parameters to address glycemic problems. Another advantage is that, where many patients frequently miss their insulin doses, the system may detect and alert the patient that they missed a dose, and may provide a means for the patient to dose late safely, which will help reduce their overall glucose levels. Current standard practice is for the patient to wait over two hours after they started eating and take a correction dose or just wait until their next meal dose to correct for high glucose; the ability to safely dose after the start of a meal but within 2 hours, will help reduce overall glucose levels with minimal additional risk of hypoglycemia. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are block diagrams of example embodiments of a dose guidance system.

FIG. 2A is a schematic diagram depicting an example embodiment of a sensor control device.

FIG. 2B is a block diagram depicting an example embodiment of a sensor control device.

FIG. 3A is a schematic diagram depicting an example embodiment of a medication delivery device.

FIG. 3B is a block diagram depicting an example embodiment of a medication delivery device.

FIG. 4A is a schematic diagram depicting an example embodiment of a display device.

FIG. 4B is a block diagram depicting an example embodiment of a display device.

FIG. 5 is a block diagram depicting an example embodiment of a user interface device.

FIGS. 6A-1-6A-4 are exemplary layouts of glucose patterns reports.

FIGS. 6B-1-6B-2, 6C-1-6C-2, 6D-1-6D-2, 6E-1-6E-2, 6F-1-6F-2, and 6G-1-6G-2 are exemplary glucose concentration profiles.

FIG. 7A is a flow diagram depicting an example embodiment of a process flow for a portion of a dose guidance application directed to a learning method for estimating an insulin dosing practice by a patient.

FIG. 7B is a flow diagram depicting an example embodiment of a method for parameterizing a patient's medication dosing practice for configuring dose guidance settings.

FIGS. 7C-7E are flow diagrams depicting additional, optional elements for the method shown in FIG. 7B.

FIG. 8A is a flow diagram depicting an example embodiment of a process flow for operations by a dose guidance application for assessing a meal bolus titration for a multiple daily injection (MDI) dosing therapy.

FIG. 8B is a flow diagram depicting an example embodiment of a process flow for operations by a dose guidance application for a glucose pattern analysis (GPA).

FIG. 8C is an example embodiment of a graph depicting information for determining a hypoglycemia risk and other metrics for a GPA.

FIGS. 8D-8H are flow diagrams showing aspects of various example embodiments of algorithms for assessing meal bolus titrations for MDI insulin dosing therapies.

FIGS. 9A-1 and 9A-2 depict exemplary reports for review by an HCP.

FIG. 9B is an exemplary adherence report.

FIG. 9C is an exemplary plot of associated with a clustering analysis of meal periods.

FIG. 9D is an exemplary plot of meal dose clustering and dose amounts.

FIG. 9E is an exemplary plot associated with premeal correction factor determination.

FIG. 9F is a flow diagram depicting an example embodiment of a process for facilitating access by an HCP to an electronic medical record.

FIG. 9G is an exemplary summary report for an HCP.

FIG. 9H is an exemplary plot demonstrating patient adherence to a recommended dosing regimen.

FIG. 9I is an exemplary summary report of a patient's therapy.

FIG. 10 is a state transition diagram governing when insulin dosing algorithms can be called during the Guidance Period.

FIG. 11 is a flow diagram depicting an example embodiment of methods for displaying dose guidance related to meal doses and correction doses.

FIG. 12 is a flow diagram depicting an example embodiment of a method for displaying a dose guidance screen.

FIG. 13 is a flow diagram depicting an example embodiment of a method for displaying a plurality of meal icons.

FIG. 14 is a flow diagram depicting an example embodiment of a method for displaying a dose calculation.

FIG. 15 is a flow diagram depicting an example embodiment of a method for asserting a missed meal dose alert.

FIG. 16A-D are flow diagrams depicting example embodiments of methods for rescinding missed meal dose alerts.

FIG. 17 is a flow diagram depicting an example embodiment of a method for asserting a correction dose alert.

FIGS. 18A-C are flow diagrams depicting example embodiments of methods for rescinding correction dose alerts.

FIG. 19 is IOB remaining from a previous injection as a function of DIA for rapid acting insulin.

FIGS. 20A-C are flow diagrams depicting example embodiments of methods for classifying doses.

FIG. 21A is a flow diagram depicting an example embodiment of a method for providing dose guidance in response to analyte data.

FIG. 21B is a flow diagram depicting an example embodiment of a method for determining a glucose pattern indicator.

FIGS. 21C and 21D are flow diagrams illustrating certain additional operations that may be performed in conjunction with one or more of the methods illustrated by FIGS. 21A and 21B.

FIG. 21E is a flow diagram illustrating supplemental or alternative operations for glucose pattern indication.

FIGS. 22A and 22B are exemplary data flow diagrams.

FIG. 22C is a flow diagram depicting an example embodiment of a method for a delivery device to determine if stored dose data is complete.

FIGS. 22D and 22E are flow diagrams depicting example embodiments of methods for an application to determine if received dose data is complete.

FIGS. 23A and 23B are flow diagrams depicting example embodiments of methods for tagging meals for a recommended dose.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

Generally, embodiments of the present disclosure include systems, devices, and methods related to medication dose guidance. The dose guidance can be based on a broad array of information and categories of information specific to a user, such as the user's current and prior analyte levels, the user's current and prior diet, the user's current and prior physical activities, the user's current and prior medication history, and other physiological information about the user. According to one aspect of the embodiments, the dose guidance provided by the systems, devices, and methods of the present disclosure can be based—not only on individual categories of information—but also on the predicted impact of such categories of information on the user's future analyte levels.

The dose guidance functionality can be implemented as a dose guidance application (DGA) that includes software and/or firmware instructions stored in a memory of a computing device for execution by at least one processor or processing circuitry thereof. The computing device can be in the possession of a user or healthcare professional (HCP), and the user or HCP can interface with the computing device through a user interface. According to some embodiments, the computing device can be a server or trusted computer system that is accessible through a network, and the dose guidance software can be presented to the user in the form of an interactive web page by way of a browser executed on a local display device (having the user interface) in communication with the server or trusted computer system through the network. In this and other embodiments, the dose guidance software can be executed across multiple devices, or executed, in part, on processing circuitry of a local display device and, in part, on processing circuitry of a server or trusted computer system. It will be understood by those of skill in the art that when the DGA is described as performing an action, such action is performed according to instructions stored in a computer memory (including instructions hardcoded in read only memory) that, when executed by at least one processor of at least one computing device, causes the DGA to perform the described action. In all cases the action can alternatively be performed by hardware that is hardwired to implement the action (e.g., dedicated circuitry) as opposed to performance by way of instructions stored in memory.

Furthermore, as used herein, a system on which the DGA is implemented can be referred to as a dose guidance system. The dose guidance system can be configured for the sole purpose of providing dose guidance or can be a multifunctional system of which dose guidance is only one aspect. For example, in some embodiments the dose guidance system can also be capable of monitoring analyte levels of a user. In some embodiments the dose guidance system can also be capable of delivering medication to the user, such as with an injection or infusion device. In some embodiments, the dose guidance system is capable of both monitoring analytes and delivering medication.

These embodiments and others described herein represent improvements in the field of computer-based dose determination, analyte monitoring, and medication delivery systems. The specific features and potential advantages of the disclosed embodiments are further discussed below.

Before describing the dose guidance embodiments in detail, it is first desirable to describe examples of dose guidance systems on or through which the dose guidance application can be implemented.

### Example Embodiments of Dose Guidance Systems

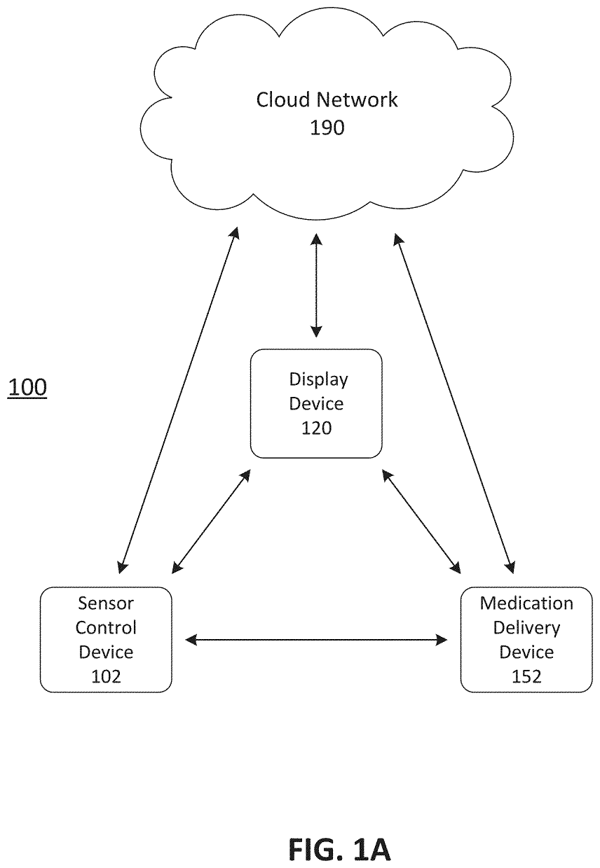

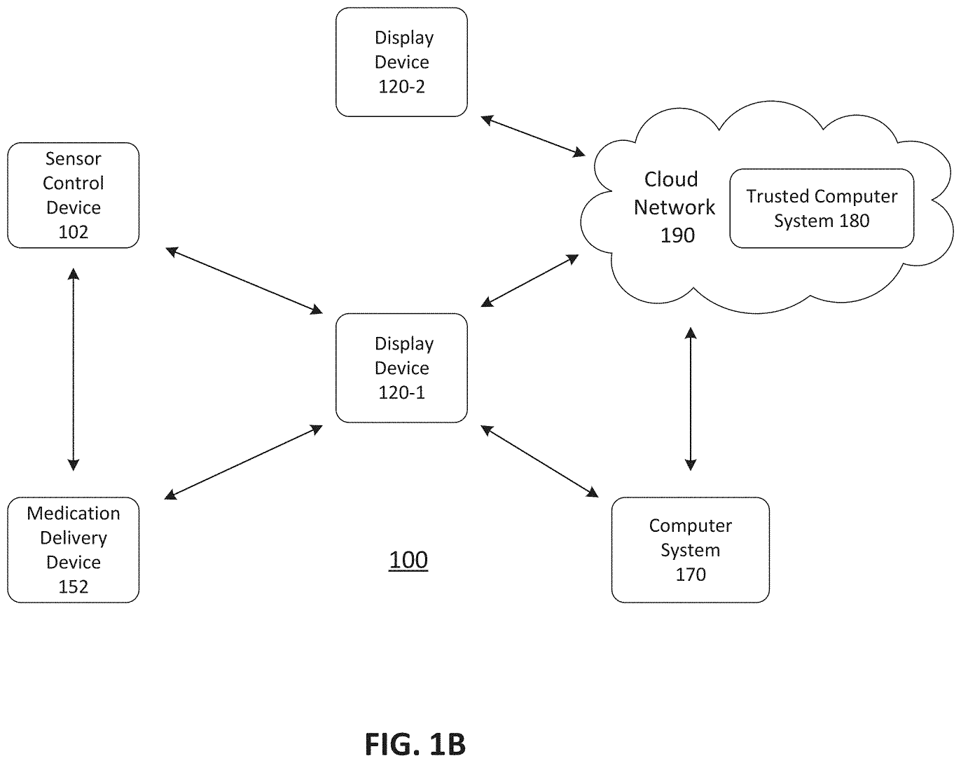

FIG. 1A is a block diagram depicting an example embodiment of dose guidance system 100. In this embodiment, dose guidance system 100 is capable of providing dose guidance, monitoring one or more analytes, and delivering one or more medications. This multifunctional example is used to illustrate the high degree of interconnectivity and performance obtainable by system 100. However, in the embodiments described herein, the analyte monitoring components, the medication delivery components, or both can be omitted if desired.

Here, system 100 includes a sensor control device (SCD) 102 configured to collect analyte level information from a user, a medication delivery device (MDD) 152 configured to deliver medication to the user, and a display device 120 configured to present information to the user and receive input or information from the user. The structure and function of each device will be described in detail herein.

System 100 is configured for highly interconnected and highly flexible communication between devices. Each of the three devices 102, 120, and 152, can communicate directly with each other (without passing through an intermediate electronic device) or indirectly with each other (such as through cloud network 190, or through another device and then through network 190). Bidirectional communication capability between devices, as well as between devices and network 190, is shown in FIG. 1A with a double-sided arrow. However, those of skill in the art will appreciate that any of the one or more devices (e.g., SCD) can be capable of unidirectional communication such as, for example, broadcasting, multicasting, or advertising communications. In each instance, whether bidirectional or unidirectional, the communication can be wired or wireless. The protocols that govern communication over each path can be the same or different, and can be either proprietary or standardized. For example, wireless communication between devices 102, 120, and 152 can be performed according to a Bluetooth (including Bluetooth Low Energy) standard, a Near Field Communication (NFC) standard, a Wi-Fi (802.11x) standard, a mobile telephony standard, or others. All communications over the various paths can be encrypted, and each device of FIG. 1A can be configured to encrypt and decrypt those communications sent and received. In each instance the communication pathways of FIG. 1A can be direct (e.g., Bluetooth or NFC) or indirect (e.g., Wi-Fi, mobile telephony, or other internet protocol). Embodiments of system 100 do not need to have the capability to communicate across all of the pathways indicated in FIG. 1A.

In addition, although FIG. 1A depicts a single display device 120, a single SCD 102, and a single MDD 152, those of skill in the art will appreciate that system 100 can comprise a plurality of any of the aforementioned devices. By way of example only, system 100 can comprise a single SCD 102 in communication with multiple (e.g., two, three, four, etc.) display devices 120 and/or multiple MDDs 152. Alternatively, system 100 can comprise a plurality of SCDs 102 in communication with a single display device 120 and/or a single MDD 152. Furthermore, each of the plurality of devices can be of the same or different device types. For example, system 100 can comprise multiple display devices 120, including a smart phone, a handheld receiver, and/or a smart watch, each of which can be in communication with SCD 102 and/or MDD 152, as well in communication with each other.

Analyte data can be transferred between each device within system 100 in an autonomous fashion (e.g., transmitting automatically according to a schedule), or in response to a request for analyte data (e.g., sending a request from a first device to a second device for analyte data, followed by transmission of the analyte data from the second device to the first device). Other techniques for communicating data can also be employed to accommodate more complex systems like cloud network 190.

FIG. 1B is a block diagram depicting another example embodiment of dose guidance system 100. Here, system 100 includes SCD 102, MDD 152, a first display device 120-1, a second display device 120-2, local computer system 170, and trusted computer system 180 that is accessible by cloud network 190. SCD 102 and MDD 152 are capable of communication with each other and with display device 120-1, which can act as a communication hub for aggregating information from SCD 102 and MDD 152, processing and displaying that information where desired, and transferring some or all of the information to cloud network 190 and/or computer system 170. Conversely, display device 120-1 can receive information from cloud network 190 and/or computer system 170 and communicate some or all of the received information to SCD 102, MDD 152, or both. Computer system 170 may be a personal computer, a server terminal, a laptop computer, a tablet, or other suitable data processing device. Computer system 170 can include or present software for data management and analysis and communication with the components in system 100. Computer system 170 can be used by the user or a medical professional to display and/or analyze analyte data measured by SCD 102. Furthermore, although FIG. 1B depicts a single SCD 102, a single MDD 152, and two display devices 120-1 and 120-2, those of skill in the art will appreciate that system 100 can include a plurality of any of the aforementioned devices, wherein each plurality of devices can comprise the same or different types of devices.

Referring still to FIG. 1B, according to some embodiments, trusted computer system 180 can be within the possession of a manufacturer or distributor of a component of system 100, either physically or virtually through a secured connection, and can be used to perform authentication of the devices of system 100 (e.g., devices 102, 120-n, 152), for secure storage of the user's data, and/or as a server that serves a data analytics program (e.g., accessible via a web browser) for performing analysis on the user's measured analyte data and medication history. Trusted computer system 180 can also act as a data hub for routing and exchanging data between all devices in communication with system 180 through cloud network 190. In other words, all devices of system 100 that are capable of communicating with cloud network 190 (e.g., either directly with an internet connection or indirectly via another device), are also capable of communicating with all of the other devices of system 100 that are capable of communicating with cloud network 190, either directly or indirectly.

Display device 120-2 is depicted in communication with cloud network 190. In this example, device 120-2 can be in the possession of another user that is granted access to the analyte and medication data of the person wearing SCD 102. For example, the person in possession of display device 120-2 can be a parent of a child wearing SCD 102, as one example, or a caregiver of an elderly patient wearing SCD 102, as another example. System 100 can be configured to communicate analyte and medication data about the wearer through cloud network 190 (e.g., via trusted computer system 180) to another user with granted access to the data.

### Example Embodiments of Analyte Monitoring Devices

The analyte monitoring functionality of dose guidance system 100 can be realized through inclusion of one or more devices capable of collecting, processing, and displaying analyte data of the user. Example embodiments of such devices and their methods of use are described in Int'l Publ. No. WO 2018/152241 and U.S. Patent Publ. No. 2011/0213225, both of which are incorporated by reference herein in their entireties for all purposes.

Analyte monitoring can be performed in numerous different ways. “Continuous Analyte Monitoring” devices (e.g., “Continuous Glucose Monitoring” devices), for example, can transmit data from a sensor control device to a display device continuously or repeatedly with or without prompting, e.g., automatically according to a schedule. “Flash Analyte Monitoring” devices (e.g., “Flash Glucose Monitoring” devices or simply “Flash” devices), as another example, can transfer data from a sensor control device in response to a user-initiated request for data by a display device (e.g., a scan), such as with a Near Field Communication (NFC) or Radio Frequency Identification (RFID) protocol.

Analyte monitoring devices that utilize a sensor configured to be placed partially or wholly within a user's body can be referred to as in vivo analyte monitoring devices. For example, an in vivo sensor can be placed in the user's body such that at least a portion of the sensor is in contact with a bodily fluid (e.g., interstitial (ISF) fluid such as dermal fluid in the dermal layer or subcutaneous fluid beneath the dermal layer, blood, or others) and can measure an analyte concentration in that bodily fluid. In vivo sensors can use various types of sensing techniques (e.g., chemical, electrochemical, or optical). Some systems utilizing in vivo analyte sensors can also operate without the need for finger stick calibration.

“In vitro” devices are those where a sensor is brought into contact with a biological sample outside of the body (or rather “ex vivo”). These devices typically include a port for receiving an analyte test strip carrying bodily fluid of the user, which can be analyzed to determine the user's blood glucose level. Other ex vivo devices have been proposed that attempt to measure the user's internal analyte level non-invasively, such as by using an optical technique that can measure an internal body analyte level without mechanically penetrating the user's body or skin. In vivo and ex vivo devices often include in vitro capability (e.g., an in vivo display device that also includes a test strip port).

The present subject matter will be described with respect to sensors capable of measuring a glucose concentration, although detection and measurement of concentrations of other analytes are within the scope of the present disclosure. These other analytes can include, for example, ketones, lactate, oxygen, hemoglobin A1C, acetyl choline, amylase, bilirubin, cholesterol, chorionic gonadotropin, creatine kinase (e.g., CK-MB), creatine, DNA, fructosamine, glutamine, growth hormones, hormones, peroxide, prostate-specific antigen, prothrombin, RNA, thyroid stimulating hormone, troponin and others. The concentration of drugs, such as, for example, antibiotics (e.g., gentamicin, vancomycin, and the like), digitoxin, digoxin, drugs of abuse, theophylline, and warfarin, may also be monitored. The sensor can be configured to measure two or more different analytes at the same or different times. In some embodiments, the sensor control device can be coupled with two or more sensors, where one sensor is configured to measure a first analyte (e.g., glucose) and the other one or more sensors are configured to measure one or more different analytes (e.g., any of those described herein). In other embodiments, a user can wear two or more sensor control devices, each of which is capable of measuring a different analyte.

The embodiments described herein can be used with all types of in vivo, in vitro, and ex vivo devices capable of monitoring the aforementioned analytes and others.

In many embodiments, the sensor operation can be controlled by SCD 102. The sensor can be mechanically and communicatively coupled with SCD 102, or can be just communicatively coupled with SCD 102 using a wireless communication technique. SCD 102 can include the electronics and power supply that enable and control analyte sensing performed by the sensor. In some embodiments the sensor or SCD 102 can be self-powered such that a battery is not required. SCD 102 can also include communication circuitry for communicating with another device that may or may not be local to the user's body (e.g., a display device). SCD 102 can reside on the body of the user (e.g., attached to or otherwise placed on the user's skin, or carried in the user's clothes, etc.). SCD 102 can also be implanted within the body of the user along with the sensor. Functionality of SCD 102 can be divided between a first component implanted within the body (e.g., a component that controls the sensor) and a second component that resides on or otherwise outside the body (e.g., a relay component that communicates with the first component and also with an external device like a computer or smartphone). In other embodiments, SCD 102 can be external to the body and configured to non-invasively measure the user's analyte levels. The sensor control device, depending on the actual implementation or embodiment, can also be referred to as a “sensor control unit,” an “on-body electronics” device or unit, an “on-body” device or unit, an “in body electronics” device or unit, an “in-body” device or unit, or a “sensor data communication” device or unit, to name a few.

In some embodiments, SCD 102 may include a user interface (e.g., a touchscreen) and be capable of processing the analyte data and displaying the resultant calculated analyte levels to the user. In such cases, the dose guidance embodiments described herein can be implemented directly by SCD 102, in whole or in part. In many embodiments, the physical form factor of SCD 102 is minimized (e.g., to minimize the appearance on the user's body) or the sensor control device may be inaccessible to the user (e.g., if wholly implanted), or other factors may make it desirable to have a display device usable by the user to read analyte levels and interface with the sensor control device.

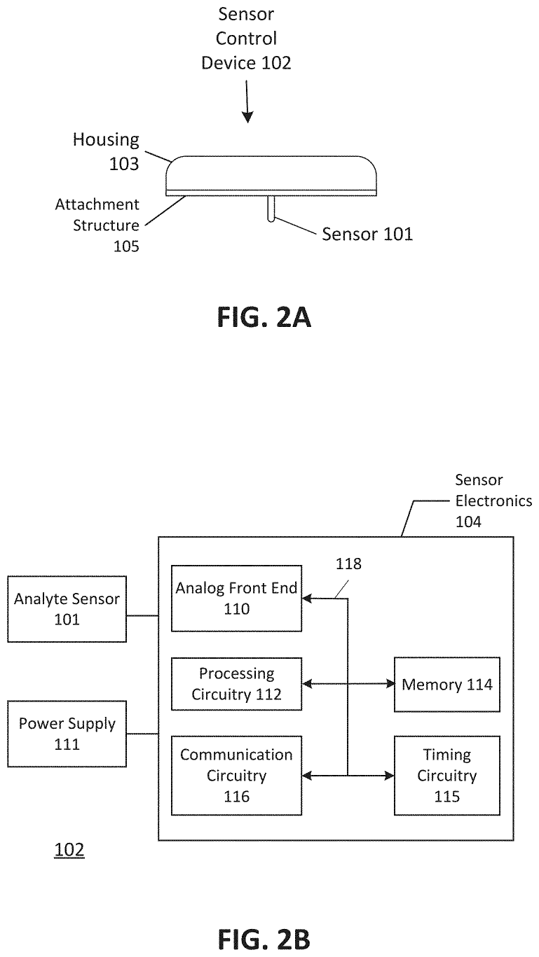

FIG. 2A is a side view of an example embodiment of SCD 102. SCD 102 can include a housing or mount 103 for sensor electronics (FIG. 2B), which can be electrically coupled with an analyte sensor 101, which is configured here as an electrochemical sensor. According to some embodiments, sensor 101 can be configured to reside partially within a user's body (e.g., through an exterior-most surface of the skin) where it can make fluid contact with a user's bodily fluid and be used, along with the sensor electronics, to measure analyte-related data of the user. A structure for attachment 105, such as an adhesive patch, can be used to secure housing 103 to a user's skin. Sensor 101 can extend through attachment structure 105 and project away from housing 103. Those of skill in the art will appreciate that other forms of attachment to the body and/or housing 103 may be used, in addition to or instead of adhesive, and are fully within the scope of the present disclosure.

SCD 102 can be applied to the body in any desired manner. For example, an insertion device (not shown), sometimes referred to as an applicator, can be used to position all or a portion of analyte sensor 101 through an external surface of the user's skin and into contact with the user's bodily fluid. In doing so, the insertion device can also position SCD 102 onto the skin. In other embodiments, the insertion device can position sensor 101 first, and then accompanying electronics (e.g., wireless transmission circuitry and/or data processing circuitry, and the like) can be coupled with sensor 101 afterwards (e.g., inserted into a mount), either manually or with the aid of a mechanical device. Examples of insertion devices are described in U.S. Patent Publ. Nos. 2008/0009692, 2011/0319729, 2015/0018639, 2015/0025345, and 2015/0173661, 2018/0235520, all which are incorporated by reference herein in their entireties for all purposes.

FIG. 2B is a block diagram depicting an example embodiment of SCD 102 having analyte sensor 101 and sensor electronics 104. Sensor electronics 104 can be implemented in one or more semiconductor chips (e.g., an application specific integrated circuit (ASIC), processor or controller, memory, programmable gate array, and others). In the embodiment of FIG. 1B, sensor electronics 104 includes high-level functional units, including an analog front end (AFE) 110 configured to interface in an analog manner with sensor 101 and convert analog signals to and/or from digital form (e.g., with an A/D converter), a power supply 111 configured to supply power to the components of SCD 102, processing circuitry 112, memory 114, timing circuitry 115 (e.g., such as an oscillator and phase locked loop for providing a clock or other timing to components of SCD 102), and communication circuitry 116 configured to communicate in wired and/or wireless fashion with one or more devices external to SCD 102, such as display device 120 and/or MDD 152.

SCD 102 can be implemented in a highly interconnected fashion, where power supply 111 is coupled with each component shown in FIG. 2B and where those components that communicate or receive data, information, or commands (e.g., AFE 110, processing circuitry 112, memory 114, timing circuitry 115, and communication circuitry 116), can be communicatively coupled with every other such component over, for example, one or more communication connections or buses 118.

Processing circuitry 112 can include one or more processors, microprocessors, controllers, and/or microcontrollers, each of which can be a discrete chip or distributed amongst (and a portion of) a number of different chips. Processing circuitry 112 can include on-board memory. Processing circuitry 112 can interface with communication circuitry 116 and perform analog-to-digital conversions, encoding and decoding, digital signal processing and other functions that facilitate the conversion of data signals into a format (e.g., in-phase and quadrature) suitable for wireless or wired transmission. Processing circuitry 112 can also interface with communication circuitry 116 to perform the reverse functions necessary to receive a wireless transmission and convert it into digital data or information.

Processing circuitry 112 can execute instructions stored in memory 114. These instructions can cause processing circuitry 112 to process raw analyte data (or pre-processed analyte data) and arrive at a final calculated analyte level. In some embodiments, instructions stored in memory 114, when executed, can cause processing circuitry 112 to process raw analyte data to determine one or more of: a calculated analyte level, an average calculated analyte level within a predetermined time window, a calculated rate-of-change of an analyte level within a predetermined time window, and/or whether a calculated analyte metric exceeds a predetermined threshold condition. These instructions can also cause processing circuitry 112 to read and act on received transmissions, to adjust the timing of timing circuitry 115, to process data or information received from other devices (e.g., calibration information, encryption or authentication information received from display device 120, and others), to perform tasks to establish and maintain communication with display device 120, to interpret voice commands from a user, to cause communication circuitry 116 to transmit, and others. In embodiments where SCD 102 includes a user interface, then the instructions can cause processing circuitry 112 to control the user interface, read user input from the user interface, cause the display of information on the user interface, format data for display, and others. The functions described here that are coded in the instructions can instead be implemented by SCD 102 with the use of a hardware or firmware design that does not rely on the execution of stored software instructions to accomplish the functions.

Memory 114 can be shared by one or more of the various functional units present within SCD 102, or can be distributed amongst two or more of them (e.g., as separate memories present within different chips). Memory 114 can also be a separate chip of its own. Memory 114 is non-transitory, and can be volatile (e.g., RAM, etc.) and/or non-volatile memory (e.g., ROM, flash memory, F-RAM, etc.).

Communication circuitry 116 can be implemented as one or more components (e.g., transmitter, receiver, transceiver, passive circuit, encoder, decoder, and/or other communication circuitry) that perform the functions for communications over the respective communications paths or links. Communication circuitry 116 can include or be coupled to one or more antenna for wireless communication.

Power supply 111 can include one or more batteries, which can be rechargeable or single-use disposable batteries. Power management circuitry can also be included to regulate battery charging and monitor usage of power supply 111, boost power, perform DC conversions, and the like.

Additionally, an on-skin or sensor temperature reading or measurement can be collected by an optional temperature sensor (not shown). Those readings or measurements can be communicated (either individually or as an aggregated measurement over time) from SCD 102 to another device (e.g., display device 120). The temperature reading or measurement, however, can be used in conjunction with a software routine executed by SCD 102 or display device 120 to correct or compensate the analyte measurement output to the user, instead of or in addition to, actually outputting the temperature measurement to the user.

### Example Embodiments of Medication Delivery Devices

The medication delivery functionality of dose guidance system 100 can be realized through inclusion of one or more medication delivery devices (MDDs) 152. MDD 152 can be any device configured to deliver a specific dose of medication. The MDD 152 can also include devices that transmit data regarding doses to the DGA, e.g., pen caps, even though the device itself may not deliver the medication. The MDD 152 can be configured as a portable injection device (PID) that can deliver a single dose per one injection, such as a bolus. The PID can be a basic manually-operated syringe, where the medication is either preloaded in the syringe or must be drawn into the syringe from a container prior to injection. In most embodiments, however, the PID includes electronics for interfacing with the user and performing the delivery of the medication. PIDs are often referred to as medication pens, although a pen-like appearance is not required. PIDs having user interface electronics are often referred to as smart pens. PIDs can be used to deliver one dose and then disposed of, or can be durable and used repeatedly to deliver many doses over the course of a day, week, or month. PIDs are often relied upon by users that practice a multiple daily injection (MDI) therapy regimen.

The MDD can also comprise a pump and infusion set. The infusion set includes a tubular cannula that resides at least partially within the recipient's body. The tubular cannula is in fluid communication with a pump, which can deliver medication through the cannula and into the recipient's body in small increments repeatedly over time. The infusion set can be applied to the recipient's body using an infusion set applicator, and the infusion set often stays implanted for 2 to 3 days or longer. A pump device includes electronics for interfacing with the user and for controlling the slow infusion of the medication. Both a PID and a pump can store the medication in a medication reservoir.

MDD 152 can function as part of a closed-loop system (e.g., an artificial pancreas system requiring no user intervention to operate), semi-closed loop system (e.g., an insulin loop system requiring seldom user intervention to operate, such as to confirm changes in dose), or an open loop system. For example, the diabetic's analyte level can be monitored in a repeated automatic fashion by SCD 102, and that information can be used by the dose guidance embodiments described herein to automatically calculate or otherwise determine the appropriate drug dosage to control the diabetic's analyte level and subsequently deliver that dose to the diabetic's body. This calculation can occur within MDD 152 or any other device of system 100 and the resulting determined dosage can then be communicated to MCD 152.

In many embodiments, the dose guidance provided by the embodiments described herein will be for a type of insulin (e.g., rapid-acting (RA), short-acting insulin, intermediate-acting insulin (e.g., NPH insulin), long-acting (LA), ultra long-acting insulin, and mixed insulin), and will be the same medication delivered by MDD 152. The type of insulin includes human insulin and synthetic insulin analogs. The insulin can also include premixed formulations. However, the dose guidance embodiments set forth herein and the medication delivery capabilities of MDD 152 can be applied to other non-insulin medications. Such medications can include, but are not limited to exenatide, exenatide extended release, liraglutide, lixisenatide, semaglutide, pramlintide, metformin, SLGT1-i inhibitors, SLGT2-i inhibitors, and DPP4 inhibitors. The dose guidance embodiments can also include combination therapies. Combination therapies can include, but are not limited to, insulin and glucagon-like peptide-1 receptor agonists (GLP-1 RA), insulin and pramlintide.

For ease of description of the dose guidance embodiments herein, MDD 152 will often be described in the form of a PID, specifically a smart pen. However, those of skill in the art will readily understand that MDD 152 can alternatively be configured as a pen cap, a pump, or any other type of medication delivery device.

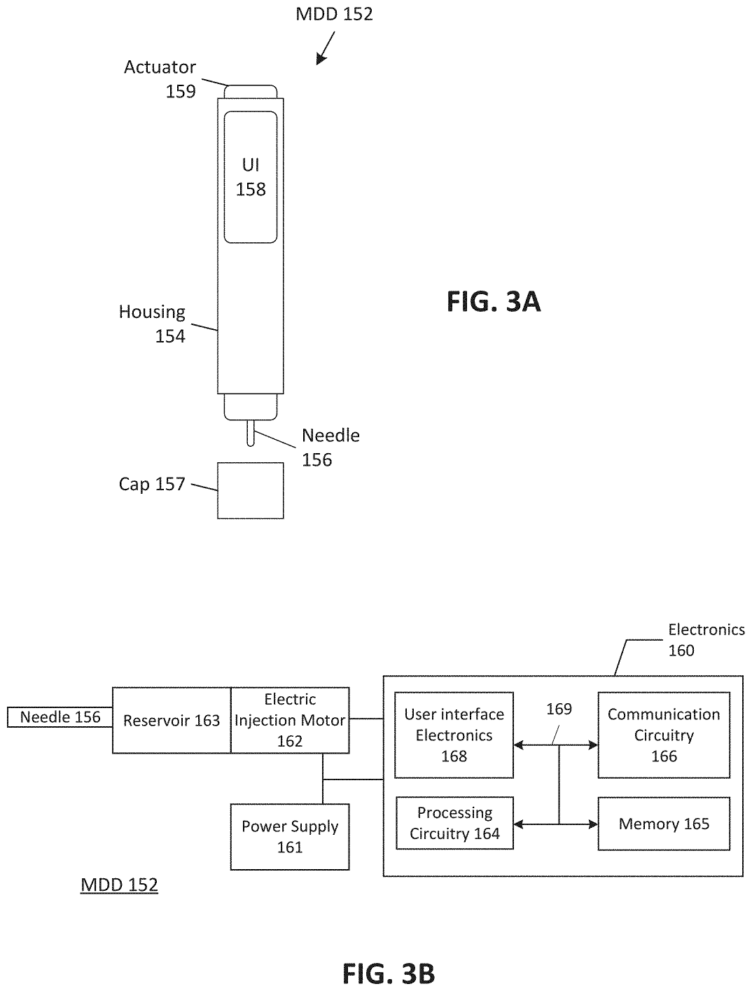

FIG. 3A is schematic diagram depicting an example embodiment of an MDD 152 configured as a PID, specifically a smart pen. MDD 152 can include a housing 154 for electronics, an injection motor, and a medication reservoir (see FIG. 3B), from which medication can be delivered through needle 156. Housing 154 can include a removable or detachable cap or cover 157 that, when attached, can shield needle 156 when not in use, and then be detached for injection. MDD 152 can also include a user interface 158 which can be implemented as a single component (e.g., a touchscreen for outputting information to the user and receiving input from the user) or as multiple components (e.g., a touchscreen or display in combination with one or more buttons, switches, or the like). MDD 152 can also include an actuator 159 that can be moved, depressed, touched or otherwise activated to initiate delivery of the medication from an internal reservoir through needle 156 and into the recipient's body. According to some embodiments, cap 157 and actuator 159 can also include one or more safety mechanisms to prevent removal and/or actuation to mitigate risk of a harmful medication injection. Details of these safety mechanisms and others are described in U.S. Patent Publ. No. 2019/0343385 (the '385 publication), which is hereby incorporated in its entirety for all purposes.

FIG. 3B is a block diagram depicting an example embodiment of MDD 152 having electronics 160, coupled with a power supply 161 and an electric injection motor 162, which in turn is coupled with power supply 161 and a medication reservoir 163. Needle 156 is shown in fluid communication with reservoir 163, and a valve (not shown) may be present between reservoir 163 and needle 156. Reservoir 163 can be permanent or can be removable and replaced with another reservoir containing the same or different medication. Electronics 160 can be implemented in one or more semiconductor chips (e.g., an application specific integrated circuit (ASIC), processor or controller, memory, programmable gate array, and others). In the embodiment of FIG. 3B, electronics 160 can include high-level functional units, including processing circuitry 164, memory 165, communication circuitry 166 configured to communicate in wired and/or wireless fashion with one or more devices external to MDD 152 (such as display device 120), and user interface electronics 168.

MDD 152 can be implemented in a highly interconnected fashion, where power supply 161 is coupled with each component shown in FIG. 3B and where those components that communicate or receive data, information, or commands (e.g., processing circuitry 164, memory 165, and communication circuitry 166), can be communicatively coupled with every other such component over, for example, one or more communication connections or buses 169.

Processing circuitry 164 can include one or more processors, microprocessors, controllers, and/or microcontrollers, each of which can be a discrete chip or distributed amongst (and a portion of) a number of different chips. Processing circuitry 164 can include on-board memory. Processing circuitry 164 can interface with communication circuitry 166 and perform analog-to-digital conversions, encoding and decoding, digital signal processing and other functions that facilitate the conversion of data signals into a format (e.g., in-phase and quadrature) suitable for wireless or wired transmission. Processing circuitry 164 can also interface with communication circuitry 166 to perform the reverse functions necessary to receive a wireless transmission and convert it into digital data or information.

Processing circuitry 164 can execute software instructions stored in memory 165. These instructions can cause processing circuitry 164 to receive a selection or provision of a specified dose from a user (e.g., entered via user interface 158 or received from another device), process a command to deliver a specified dose (such as a signal from actuator 159), and control motor 162 to cause delivery of the specified dose. These instructions can also cause processing circuitry 164 to read and act on received transmissions, to process data or information received from other devices (e.g., calibration information, encryption or authentication information received from display device 120, and others), to perform tasks to establish and maintain communication with display device 120, to interpret voice commands from a user, to cause communication circuitry 166 to transmit, and others. In embodiments where MDD 152 includes user interface 158, then the instructions can cause processing circuitry 164 to control the user interface, read user input from the user interface (e.g., entry of a medication dose for administration or entry of confirmation of a recommended medication dose), cause the display of information on the user interface, format data for display, and others. The functions described here that are coded in the instructions can instead be implemented by MDD 152 with the use of a hardware or firmware design that does not rely on the execution of stored software instructions to accomplish the functions.

Memory 165 can be shared by one or more of the various functional units present within MDD 152, or can be distributed amongst two or more of them (e.g., as separate memories present within different chips). Memory 165 can also be a separate chip of its own. Memory 165 is non-transitory, and can be volatile (e.g., RAM, etc.) and/or non-volatile memory (e.g., ROM, flash memory, F-RAM, etc.).

Communication circuitry 166 can be implemented as one or more components (e.g., transmitter, receiver, transceiver, passive circuit, encoder, decoder, and/or other communication circuitry) that perform the functions for communications over the respective communications paths or links. Communication circuitry 166 can include or be coupled to one or more antenna for wireless communication. Details of exemplary antenna can be found in the '385 publication, which is hereby incorporated in its entirety for all purposes.

Power supply 161 can include one or more batteries, which can be rechargeable or single-use disposable batteries. Power management circuitry can also be included to regulate battery charging and monitor usage of power supply 161, boost power, perform DC conversions, and the like.

MDD 152 may also include an integrated or attachable in vitro glucose meter, including an in vitro test strip port (not shown) to receive an in vitro glucose test strip for performing in vitro blood glucose measurements.

Communication Functionality

Connected insulin pens and pen caps devices are types of MDDs 152 that measure the amount of insulin a patient injects and then transmit that data to a display device 120, e.g., a smartphone. With connected pens, the electronics and mechanism needed to transmit the data are built into the insulin pen. With connected pen caps, the electronics and mechanism are contained in a “cap” that is attached to an insulin pen.

Connected MDDs 152 are an important part of a DGS 100. Traditionally, bolus calculator applications have required patients to enter their dosing information manually, which limits the application's usability. Having a connected MDD 152 to transmit insulin delivery data automatically to the DGA substantially improves the usability of the DGS 100.

Functionality related to how and what the information is communicated between the DGA and the connected MDD 152 may have a large impact on the degree of usability of the DGS 100.

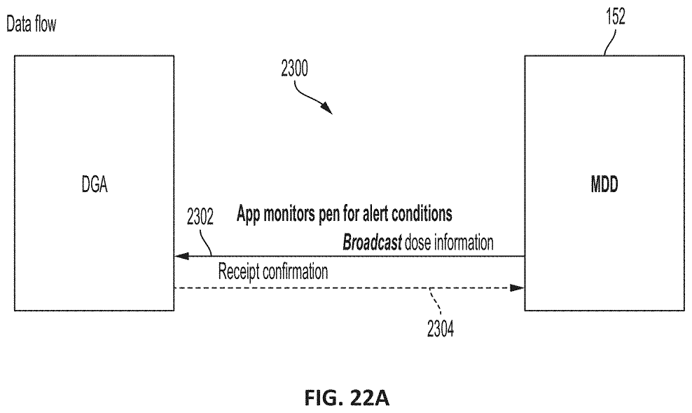

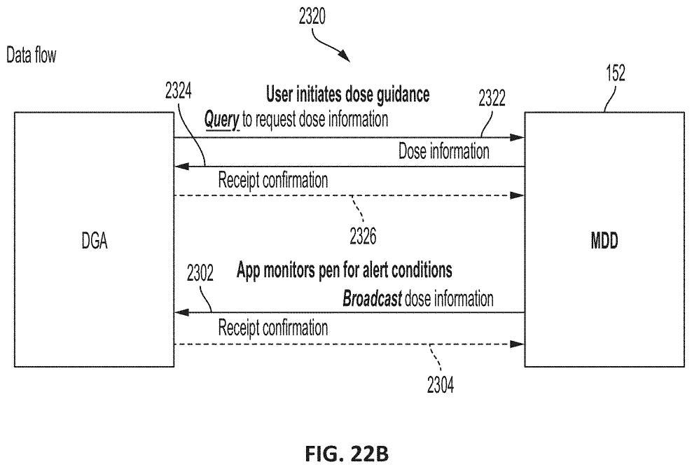

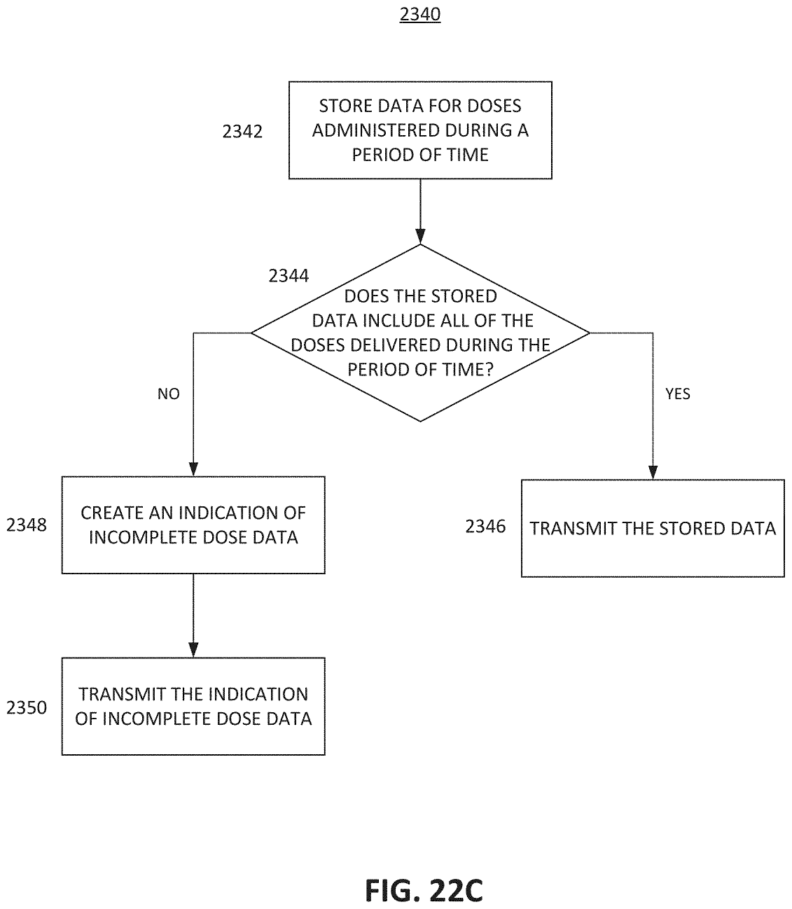

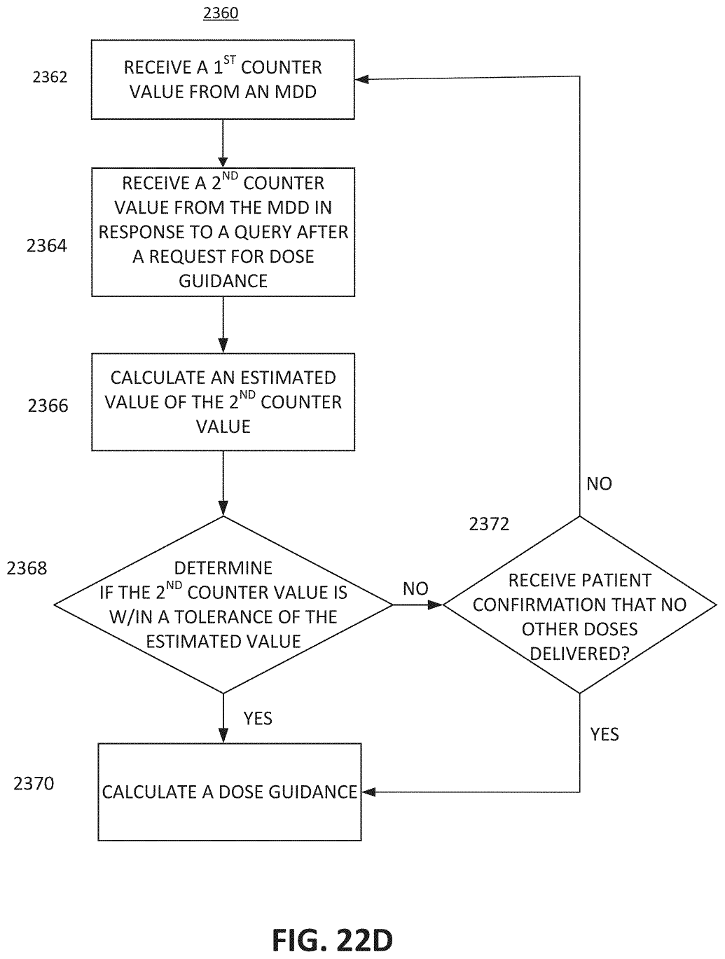

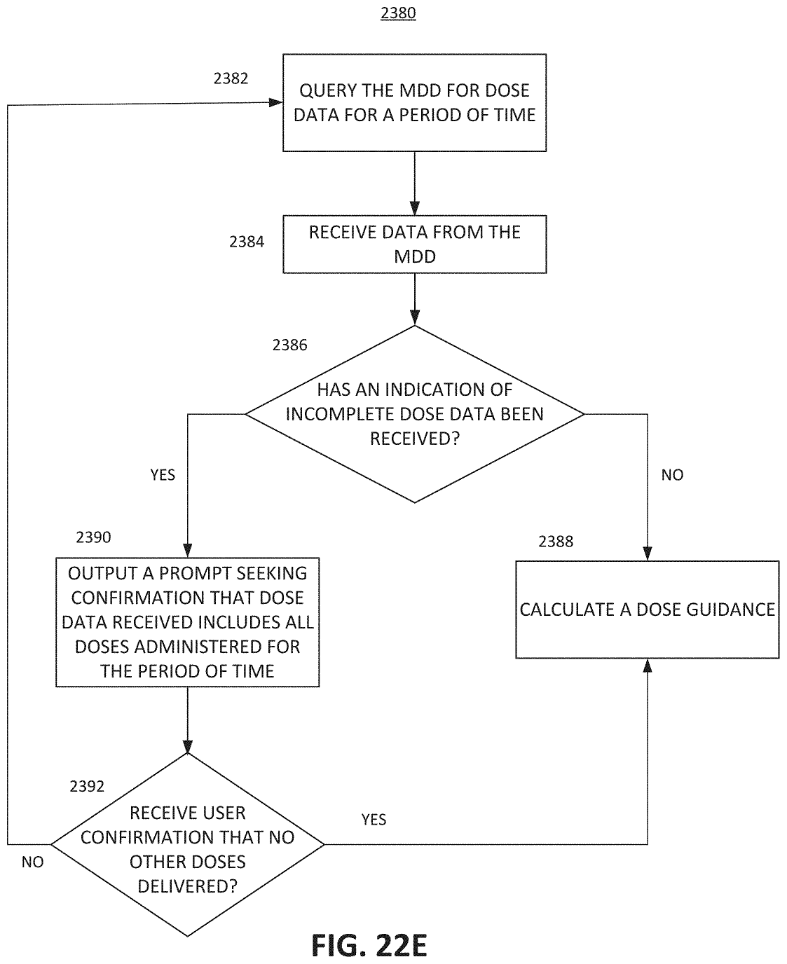

Current connected MDDs 152 may include circuitry that can broadcast the record of an insulin dose once the dose has been administered. In addition, many designs may rebroadcast the record until the receiving application has confirmed that it has received the dose record. As seen in FIG. 22A, in data flow design 2300, MDD 152 may broadcast dose information 2302 to an application, and the application may send a receipt confirmation to the MDD 152. This data flow design may work well for applications that use the dose information for certain functions, e.g., dose logging function.

This data flow design, however, may not adequately address the need of a software application that is intended to provide insulin dosing guidance. In particular, dose calculations typically require knowledge of prior insulin doses, within the time frame of the insulin's action time (e.g., typically about 4.5 hours for most rapid-acting insulins). Dose calculators may keep track of the metric referred to commonly as IOB (insulin-on-board). The IOB is typically subtracted from the calculated dose before being displayed to the user. When the user requests dose guidance, then the application needs to calculate an insulin dose, and subsequently needs to calculate the patient's JOB. The problem with data flow design 2300 is that if the MDD 152 is non-functional or the communication path is interrupted, the application may not receive information about a recent dose and then would incorrectly calculate the user's JOB, potentially resulting in the user overdosing.

In one embodiment, this hazard may be mitigated by having the DGA display a prompt that requires the user to confirm that no doses other than the ones received by the DGA have occurred. In order to proceed with the dose guidance calculation, when the DGA is missing a recent dose record, the DGA may provide a means for the patient to enter the dose amount and time manually. In another embodiment, the DGA may provide instructions for the user to correct the communication interruption and a means to retry the dose guidance calculation. This method, however, may be cumbersome and add additional user steps in the process of requesting dose guidance that may severely degrade the usability of the DGA.

In another embodiment, as seen in FIG. 22B, the MDD 152 may be designed to provide a way for the DGA to query the pen for the latest dose records. In addition to or in the place of data flow 2300 in which the DGA monitors the MDD 152 for alert conditions, in data flow 2320, after the user initiates (e.g., requests) dose guidance, the DGA may send a query to the MDD 152 to request dose information 2322. The query may be for recent dose information or for dose information from a specific period of time, e.g., a period of time since the last received insulin dose data, as specified in a communication protocol. The MDD 152 may transmit the requested dose information back to the DGA 2324 in response to the query, and the DGA may transmit a receipt confirmation 2326 back to the MDD 152. When the DGA has received all of the recent dose records, the DGA may calculate the JOB and dose guidance amount, as described elsewhere in this application, to display to the user.

The data flow design 2320 may address situations where the communication channel has been interrupted. To ensure that the IOB is accurate, however, the DGA may require confirmation from the MDD 152 that the DGA has received all of the recent dose information, including confirmation that no other doses were provided by the MDD 152 recently or during a specific period of time. Some scenarios where this may occur include: (a) the battery died in the MDD 152, or some other temporary malfunction prevented the MDD 152 properly recording doses that were delivered, or (b) for a pen cap delivery device, the pen cap may not have been engaged with the insulin pen.

In one exemplary embodiment, in method 2340 as seen in FIG. 22C, in a first step 2342, the MDD 152 may store data for doses administered during a period of time. The data may include the dose amounts and times administered. The data may also include the remaining amount of medication, e.g., insulin, remaining in the medication delivery device. In step 2344, the MDD 152 may determine if the stored data includes all of the doses delivered during the period of time. In order to make this determination, the MDD 152 may include self-test circuitry that could periodically ensure proper function and battery power. This self-test circuitry may maintain a counter that increments after every self-test cycle, with a fixed periodicity, e.g., every minute. When the MDD 152 is queried, the MDD 152 may check the self-test counter to confirm that the counter value is equal to the estimated counter value based on the current elapsed time, which may be provided by a separate circuit in the MDD 152 electronics. In another embodiment, the MDD 152 may transmit the counter value to the DGA as part of the query, and the DGA may perform the counter value check, comparing the MDD's 152 counter value to the estimated counter value based on an elapsed time of a clock in the DGA. The DGS 100 may include some level of timing tolerance between the DGA clock and the MDD clock.

In step 2346, if it is determined that the stored data contains all of the doses delivered during the period of time, then the MDD may transmit the stored data to an application that has sent the query.

If it is determined that the stored data does not contain all of the doses delivered during the period of time, in step 2348, the MDD 152 may create an indication of incomplete dose data. In step 2350, the MDD 152 may transmit the indication of incomplete dose data to the application that has sent the query.

In an alternative embodiment, the DGA may include the circuitry to determine if the data transmitted from the MDD 152 contains all of the doses administered during the period fo time. In exemplary method 2360, as depicted in FIG. 22D, in step 2362, the DGA may query the MDD 152, e.g., an insulin pen, and receive a first self-test counter value. This first counter value may be the current counter value at the time of the query. At a later time, in step 2364, the DGA may send an additional query the MDD 152 and receive a second self-test counter value. The additional query may be in response a request for dose guidance from a user. The second self-test counter value may be the current counter value at the time of the additional query. In step 2366, the DGA may calculate an estimated value of the second self-test counter value. The estimated value may be calculated based on the first counter value+(elapsed time between the query and the additional query/period of self tests). The period of self tests may be the time period in which the MDD 152 self-test circuitry is configured to increment the counter value by “1.”

In step 2368, the DGA may compare the second counter value and the estimated counter value to determine if the values are within a tolerance. If the comparison (e.g., difference) of the values is within a tolerance, then in step 2370, the DGA may calculate an insulin dose guidance that may be displayed to the user. If the comparison is not within the tolerance, in step 2372, the DGA may request that the user confirm that no other doses were delivered aside from the doses that were recorded (e.g., received in the data transfer) by the DGA. If the user confirms that no other doses were delivered, then in step 2370, the DGA may calculate an insulin dose guidance that may be displayed to the user. If the user does not confirm that no additional doses were delivered, then the DGA may not calculate and display a dose guidance and the system can recheck the counter values.

In another alternative embodiment, in method 2380 as seen in FIG. 22E, the DGA may query the MDD 152 for dose data for a period of time in step 2382. In step 2384, the DGA may receive data from the MDD 152. The data received may include dose data and may also include an indication of incomplete dose data. In step 2386, the DGA may determine if an indication of incomplete dose has been received from the MDD 152. If no indication of incomplete dose has been received, in step 2388, the DGA may calculate a dose guidance based on data transmitted from the MDD 152. If the DGA has received an indication of incomplete dose has been received, in step 2390, the DGA may output a prompt seeking confirmation from the user that dose data received includes all of the doses administered over the period of time. In step 2392, if it is determined that the DGA has received confirmation from the user that no other doses were delivered during the period of time, then the DGA may calculate a dose guidance in step 2388. If it is determined that the DGA has not received confirmation from the user that no other doses were delivered during the period of time, then the DGA may query the MDD 152 for dose data again, as in step 2382.

For a DGS 100 that includes a pen cap system as the MDD 152, when the cap has been removed from the insulin pen for a time, and then reattached, the system may be able to detect that doses have been delivered and may also be able to detect the accumulated dose amount (if more than one dose delivery occurred). The actual timing of these one or more doses, however, may not be known. In one embodiment, the pen cap may include a mechanism that detects when it is attached or detached from the insulin pen, in addition to a mechanism that detects the current insulin remaining in the pen. The pen cap controller system may store the date-time of the last instance when the pen cap is reattached AND the insulin level is different from when the pen cap was last detached. The date-time of this error indication may be sent to the DGA in response to a query. The pen cap controller system may exclude from storing this error indicator for a special case when the pen cap is detached when the pen is empty (or almost empty) and the pen is reattached with a full pen. The DGA may deal with this indicator similarly as described for the self-test indicator.

### Example Embodiments of Display Devices

Display device 120 can be configured to display information pertaining to system 100 to the user and accept or receive input from the user also pertaining to system 100. Display device 120 can display recent measured analyte levels, in any number of forms, to the user. The display device can display historical analyte levels of the user as well as other metrics that describe the user's analyte information (e.g., time in range, ambulatory glucose profile (AGP), hypoglycemia risk levels, etc.). Display device 120 can display medication delivery information, such as historical dose information and the times and dates of administration. Display device 120 can display alarms, alerts, or other notifications pertaining to analyte levels and/or medication delivery.

Display device 120 can be dedicated for use with system 100 (e.g., an electronic device designed and manufactured for the primary purpose of interfacing with an analyte sensor and/or a medication delivery device), as well as devices that are multifunctional, general purpose computing devices such as a handheld or portable mobile communication device (e.g., a smartphone or tablet), or a laptop, personal computer, or other computing device. Display device 120 can be configured as a mobile smart wearable electronics assembly, such as a smart glass or smart glasses, or a smart watch or wristband. Display devices, and variations thereof, can be referred to as “reader devices,” “readers,” “handheld electronics” (or handhelds), “portable data processing” devices or units, “information receivers,” “receiver” devices or units (or simply receivers), “relay” devices or units, or “remote” devices or units, to name a few.

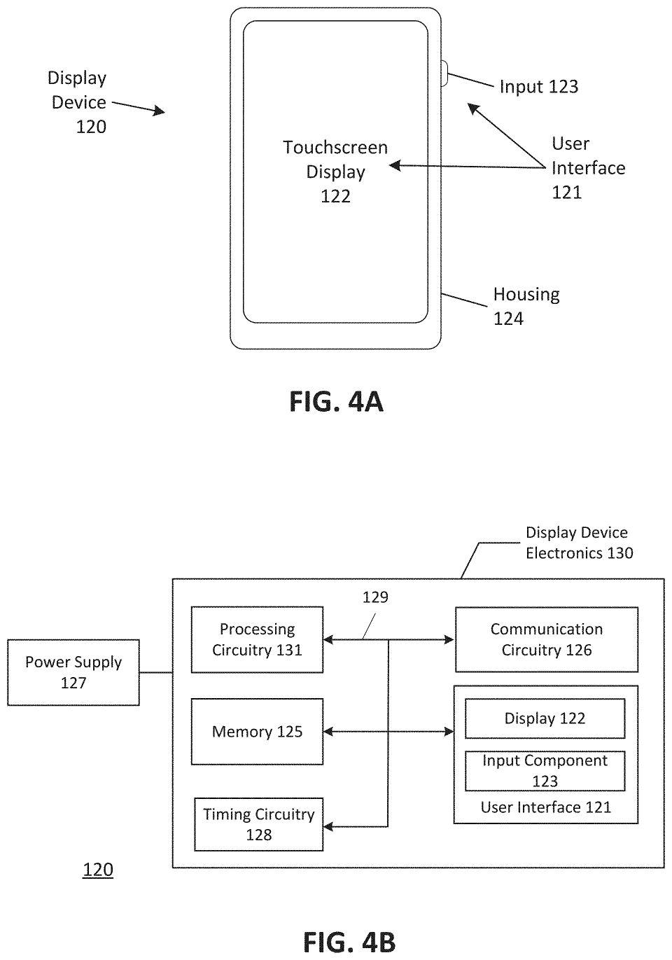

FIG. 4A is a schematic view depicting an example embodiment of display device 120. Here, display device 120 includes a user interface 121 and a housing 124 in which display device electronics 130 (FIG. 4B) are held. User interface 121 can be implemented as a single component (e.g., a touchscreen capable of input and output) or multiple components (e.g., a display and one or more devices configured to receive user input). In this embodiment, user interface 121 includes a touchscreen display 122 (configured to display information and graphics and accept user input by touch) and an input button 123, both of which are coupled with housing 124.

Display device 120 can have software stored thereon (e.g., by the manufacturer or downloaded by the user in the form of one or more “apps” or other software packages) that interface with SCD 102, MDD 152, and/or the user. In addition, or alternatively, the user interface can be affected by a web page displayed on a browser or other internet interfacing software executable on display device 120.

FIG. 4B is a block diagram of an example embodiment of a display device 120 with display device electronics 130. Here, display device 120 includes user interface 121 including display 122 and an input component 123 (e.g., a button, actuator, touch sensitive switch, capacitive switch, pressure sensitive switch, jog wheel, microphone, speaker, or the like), processing circuitry 131, memory 125, communication circuitry 126 configured to communicate to and/or from one or more other devices external to display device 120), a power supply 127, and timing circuitry 128 (e.g., such as an oscillator and phase locked loop for providing a clock or other timing to components of SCD 102). Each of the aforementioned components can be implemented as one or more different devices or can be combined into a multifunctional device (e.g., integration of processing circuitry 131, memory 125, and communication circuitry 126 on a single semiconductor chip). Display device 120 can be implemented in a highly interconnected fashion, where power supply 127 is coupled with each component shown in FIG. 4B and where those components that communicate or receive data, information, or commands (e.g., user interface 121, processing circuitry 131, memory 125, communication circuitry 126, and timing circuitry 128), can be communicatively coupled with every other such component over, for example, one or more communication connections or buses 129. FIG. 4B is an abbreviated representation of the typical hardware and functionality that resides within a display device and those of ordinary skill in the art will readily recognize that other hardware and functionality (e.g., codecs, drivers, glue logic) can also be included.

Processing circuitry 131 can include one or more processors, microprocessors, controllers, and/or microcontrollers, each of which can be a discrete chip or distributed amongst (and a portion of) a number of different chips. Processing circuitry 131 can include on-board memory. Processing circuitry 131 can interface with communication circuitry 126 and perform analog-to-digital conversions, encoding and decoding, digital signal processing and other functions that facilitate the conversion of data signals into a format (e.g., in-phase and quadrature) suitable for wireless or wired transmission. Processing circuitry 131 can also interface with communication circuitry 126 to perform the reverse functions necessary to receive a wireless transmission and convert it into digital data or information.

Processing circuitry 131 can execute software instructions stored in memory 125. These instructions can cause processing circuitry 131 to process raw analyte data (or pre-processed analyte data) and arrive at a corresponding analyte level suitable for display to the user. These instructions can cause processing circuitry 131 to read, process, and/or store a dose instruction from the user, and because the dose instruction to be communicated to MDD 152. These instructions can cause processing circuitry 131 to execute user interface software adapted to present an interactive group of graphical user interface screens to the user for the purposes of configuring system parameters (e.g., alarm thresholds, notification settings, display preferences, and the like), presenting current and historical analyte level information to the user, presenting current and historical medication delivery information to the user, collecting other non-analyte information from the user (e.g., information about meals consumed, activities performed, medication administered, and the like), and presenting notifications and alarms to the user. These instructions can also cause processing circuitry 131 to cause communication circuitry 126 to transmit, can cause processing circuitry 131 to read and act on received transmissions, to read input from user interface 121 (e.g., entry of a medication dose to be administered or confirmation of a recommended medication dose), to display data or information on user interface 121, to adjust the timing of timing circuitry 128, to process data or information received from other devices (e.g., analyte data, calibration information, encryption or authentication information received from SCD 102, and others), to perform tasks to establish and maintain communication with SCD 102, to interpret voice commands from a user, and others. The functions described here that are coded in the instructions can instead be implemented by display device 120 with the use of a hardware or firmware design that does not rely on the execution of stored software instructions to accomplish the functions.

Memory 125 can be shared by one or more of the various functional units present within display device 120, or can be distributed amongst two or more of them (e.g., as separate memories present within different chips). Memory 125 can also be a separate chip of its own. Memory 125 is non-transitory, and can be volatile (e.g., RAM, etc.) and/or non-volatile memory (e.g., ROM, flash memory, F-RAM, etc.).

Communication circuitry 126 can be implemented as one or more components (e.g., transmitter, receiver, transceiver, passive circuit, encoder, decoder, and/or other communication circuitry) that perform the functions for communications over the respective communications paths or links. Communication circuitry 126 can include or be coupled to one or more antenna for wireless communication.

Power supply 127 can include one or more batteries, which can be rechargeable or single-use disposable batteries. Power management circuitry can also be included to regulate battery charging and monitor usage of power supply 127, boost power, perform DC conversions, and the like.

Display device 120 can also include one or more data communication ports (not shown) for wired data communication with external devices such as computer system 170, SCD 102, or MDD 152. Display device 120 may also include an integrated or attachable in vitro glucose meter, including an in vitro test strip port (not shown) to receive an in vitro glucose test strip for performing in vitro blood glucose measurements.

Display device 120 can display the measured analyte data received from SCD 102 and can also be configured to output alarms, alert notifications, glucose values, etc., which may be visual, audible, tactile, or any combination thereof. In some embodiments, SCD 102 and/or MDD 152 can also be configured to output alarms, or alert notifications in visible, audible, tactile forms or combination thereof. Further details and other display embodiments can be found in, e.g., U.S. Patent Publ. No. 2011/0193704, which is incorporated herein by reference in its entirety for all purposes.

### Example Embodiments Related to Dose Guidance

The following example embodiments relate to dose guidance functionality provided by dose guidance system 100. The dose guidance functionality will, in many embodiments, be implemented as a set of software instructions stored and/or executed on one or more electronic devices. This dose guidance functionality will be referred to herein as a dose guidance application (DGA). In some embodiments, the DGA is stored, executed, and presented to the user on the same single electronic device. In other embodiments, the DGA can be stored and executed on one device, and presented to the user on a different electronic device. For example, the DGA can be stored and executed on trusted computer system 180 and presented to the user by way of a webpage displayed through an internet browser executed on display device 120. The DGA may be a stand-alone application or may be incorporated in whole or in part into another software application.

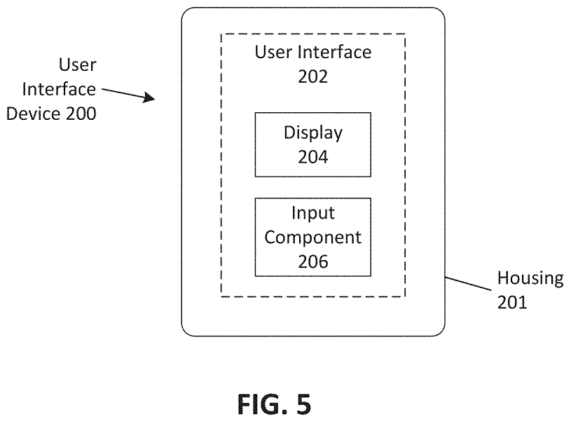

Thus, there are many different embodiments pertaining to the number and type of electronic devices that are used in storing, executing, and presenting the DGA to a user. With respect to presentation to the user, the device that is configured to implement this capability will be referred to herein as a user interface device (UID) 200. FIG. 5 is a block diagram depicting an example embodiment of UID 200. In this embodiment, UID 200 includes a housing 201 that is coupled with a user interface 202. The user interface 202 is capable of outputting information to the user and receiving input or information from the user. In some embodiments, the user interface 202 is a touchscreen. As shown here, the user interface 202 includes a display 204, that may be a touchscreen, and an input component 206 (e.g., a button, actuator, touch sensitive switch, capacitive switch, pressure sensitive switch, jog wheel, microphone, touch pad, soft keys, keyboard, or the like).

Many of the devices described herein can be implemented as UID 200. For example, display device 120 will, in many embodiments, be used as UID 200. In some embodiments, MDD 152 can be implemented as UID 200. In embodiments where SCD 102 includes a user interface, then SCD 102 can be implemented as UID 200. Computer system 170 can also be implemented as UID 200.

### Purpose

The Dose Guidance System (DGS 100) leverages glucose and insulin data to learn, provide, and titrate insulin doses. The DGS includes an application, e.g., a mobile application based on a smart-phone, integrated with a connected insulin pen and continuous glucose sensor to improve therapy management for insulin-intensive people with diabetes (PWDs) on multiple daily injections (MDI).

The DGS 100 may perform three major tasks. First, the DGS 100 may learn the patient's insulin dose regimen (i.e., how frequently insulin doses are administered and at what dosages) during a “Learning Period” of the DGS. Second, the DGS 100 may provide dose recommendations to the patient for mealtime dosing and postmeal corrections. Third, the DGS 100 may titrate current dose settings in order to maximize glycemic control. These second and third tasks may occur in parallel during a “Guidance Period” of the DGS.

Continuous glucose data in various forms (scan, historic and streaming) as well as insulin data, may serve as inputs to perform all three functions above. The DGS 100 may receive the glucose data by different means and in various forms, including scan, historic, and streaming. Scanned data, including latest glucose values and trend values, may have been retrieved by the user on demand. Historic glucose data may be generated by components of the DGS, which may generate and record glucose and trend values at a regular interval, e.g., every 15 minutes. Past historic data may be retrieved by the user with a scan. Streaming data may include glucose and trend values that are generated and recorded at a regular interval, e.g., every minute, and automatically sent to the DGA. Similarly, the insulin data may come from multiple sources. Insulin data may be manually logged or transferred from an MDD 152, e.g., insulin pen. The glucose data and insulin data may be transferred through any known means, e.g., wireless communication technology such as Bluetooth or NFC.

During the “Learning Period,” the DGA may determine a user's insulin dose regimen through clustering of insulin data by time of day and subsequent curve fitting when combined with glucose data. This learning portion of the DGA may require glucose and insulin data from the user for a period of time, e.g., about 14 days.

The Guidance Period, which includes providing dose recommendations and titrating dose settings, may be initiated after the Learning Period is complete and an initial insulin dose regimen has been determined. During the Guidance Period, the DGS may supply mealtime dose recommendations upon user request. Users may request dose recommendations for meals that the DGS has determined that the user currently treats with insulin. Dose calculations may utilize a bolus calculator formalism to provide dose recommendations that modify a learned fixed dose to account for high pre-meal glucose and residual insulin remaining in the bloodstream from previous injections.

The DGS may also notify users if a mealtime dose was missed and recommend a modified dose. Rapid acting insulin analogs for prandial dosing are recommended to be taken either at or just before mealtime. Missed mealtime insulin doses are common and therapy concordance is known to be a factor impacting glycemic management. To accommodate these behavioral tendencies, the DGS may leverage streaming data, e.g., data sent at regular intervals such as every minute, to detect periods of considerable glucose excursion without an associated insulin dose. Such a period may be indicative of an instance where the user ate a meal but without taking their prescribed insulin dose. In this instance, a prompt may appear to notify the user. Once the missed meal event has been confirmed by the user, a modified mealtime insulin dose may be recommended to account for the timing mismatch between the meal start and insulin injection.

The DGS may also notify users when it is appropriate to take a correction dose and recommend a dose amount to correct for high glucose between meals. Every minute, the DGS may process the streaming data to identify periods where users are presenting with high glucose in between meal doses. In this instance, the DGS may display a prompt to notify the user of the occurrence. The user may then request a dose recommendation from the DGS to correct for high glucose. Alternatively, the DGS may provide a dose amount recommendation within the notification, and eliminate the need for the user to request it.

The DGS may also titrate the user's insulin dose regimen to lower the user's glucose levels while avoiding excessive time below a low threshold, e.g., 70 mg/dL, and thus maximize glycemia in a target range, e.g., 70 to 180 mg/dL. Once the user has transitioned to the “Guidance Period,” the DGS may periodically analyze insulin and glucose data to titrate previously learned or titrated insulin dose regimen parameters.

### Detecting MDI Dose Strategy

Turning now to the aspects of the DGA and more particularly, the DGA can use knowledge of the patient's dosing strategy and analyte levels in order to provide accurate dose guidance. Example embodiments for automatic detection of the patient dosing strategy that can ease and speed the setup of the DGA is described herein. The detection of the dosing strategy can be based on the numerous characteristics of monitored drug (e.g., insulin) doses. For example, the embodiments can identify a dose as basal or bolus based on the MDD 152 used to administer the dose. Some patients may have more than one MDD 152. For example, a patient may have one MDD to administer long-acting insulin (e.g., basal doses) and another MDD to administer rapid-acting insulin (e.g., meal doses). The count (e.g., number of doses) and timing of basal doses per administration can also be used to categorize the basal strategy as a ‘single’ or ‘split’ basal dosing strategy. For example, in a ‘split’ basal dosing strategy, a daily basal dose of 20 U can be split into two 10 U doses, where one dose can be administered before bed and another dose can be administered upon awakening.

When successive bolus doses are administered in close succession, the system can attempt to distinguish between the original meal dose, an augmentation to the original meal dose, or a correction dose for high glucose between meals. When the DGA detects a small dose quickly followed by a larger dose, both occurring close to the start of a meal, the DGA can group the doses together as a single meal dose, even if the first dose may have been a priming dose that was not injected into the patient. Afterwards, if a dose occurs far after a known meal and/or a dose (group) tagged as a meal dose, the DGA can tag the later dose as a correction dose for high post-meal glucose following the meal, or an augmentation to the previous meal dose to account for extra food consumed. When a meal event is recognized, either based upon a meal detector algorithm or a user-entered meal event, the DGA can use the amount of the previous dose event and timing relative to the current detected meal to help to delineate if the previous dose was the first of multiple meal doses versus a correction for high glucose between meals. It is assumed that correction doses are smaller in size to mealtime doses. Moreover, if the time elapsed between the previous dose and the current meal event is sufficiently long, it would be reasonable to assume that those two events would not be related as treating the same glucose excursion event, removing the possibility that the previous dose was the first of multiple doses for a given meal. Therefore, if the earlier dose is sufficiently smaller than logged meal doses within this window on previous days, and is sufficiently far away from the current meal, the previous dose could be classified as a correction dose event.

The DGA can be configured to use a real time meal-detection algorithm and the time of the dose to identify the doses additional to basal as breakfast, lunch, and/or dinner bolus doses, and/or correction doses. The DGA can also be configured to use the number of bolus doses each day to identify the dosing strategy as basal only, basal plus one, basal plus two, etc.

These different scenarios and aspects of the DGA are discussed elsewhere in the specification in more detail.

### On-Boarding

To increase the safety profile of the DGA, HCPs can approve learned insulin dosing parameters and subsequent titrations calculated by the DGA. The DGA embodiments include numerous methods of interaction between the HCP and the DGA, so that the HCP is provided with relevant evidence to approve suggested dose learning and titration in a concise informative way that improves workflow.

For patients with diabetes that are already on an insulin dosing regimen, HCPs can leverage existing reports that give insights into patients' glucose patterns to identify users who may benefit from dose guidance. Embodiments of the DGA provide for a learning period that can classify a patient's dosing strategies and tendencies (e.g., while using DGS 100). If the combined insulin and glucose data further confirm that the user is a good candidate for the DGA, e.g., a candidate for whom the DGA can learn their particular dosing strategy, insulin dose parameters learned during the learning period can serve as initial conditions for dose guidance that can be titrated as needed by the DGA. An HCP notification method for dose parameter initialization and titration for the DGA can also be presented. This process can aid both HCPs and users by streamlining the DGA onboarding and titration, while also helping to ensure that the DGA is used only by those for whom it is indicated. When the DGA is not able to learn a patient's dosing parameters, the DGA could indicate patient dosing inconsistencies, which could be used by the HCP to address the dosing inconsistencies with the patient.

A first step of identifying potential DGA users can involve an introductory analysis of a patient's glycemic control via their glucose concentration profile. To promote access of the DGA to as many users as possible, the process can be agnostic to a user's current methods for glucose monitoring.

For a person with diabetes currently using an SCD 102, a glucose patterns report, as discussed in further detail below with respect to FIGS. 6A-1-6G-2, may be available that includes key metrics, a glucose concentration profile (e.g., an ambulatory glucose profile (AGP)), patterns identified for different times of the day, and titration and lifestyle suggestions to ameliorate instances where glucose is consistently outside of target range. The patterns can be identified using a GPA algorithm, as described in more detail elsewhere.

For a person not currently using a device or system (e.g., SCD 102) that is associated with an application that can generate a glucose patterns report 250, as described above, the HCP can suggest that, the patient be monitored by a different device or system, such that a report 250, or similar, can be generated. For example, a patient could wear an SCD 102 to collect glucose data over a multi-day or multi-week period, wherein SCD 102 is configured in a masked or blinded mode where the user does not have access to the measured glucose levels, and thus cannot modify his or her behavior during this time. From these data, a glucose patterns report could be generated. If suggested insulin titration is included in the glucose patterns report, then the glucose patterns report 250 can also include a suggestion that the patient is a good candidate for the DGS 100 and a learning period for the drug dosing strategy could be suggested.

During the learning period, an MDD 152 can be incorporated with the glucose sensing system used for the initial screening to provide a more complete portrait of insulin intensive diabetes management. The learning period can utilize algorithms, such as those described elsewhere herein, to detect a user's insulin dosing strategy. During the learning period, the DGA can be configured to determine the manner in which the user determines a meal time dose. For example, the DGA can determine if the user is determining a meal time dose based on a carbohydrate counting technique, an experiential technique such as one where the user learns proper dosing based on past experience with the meal or a meal similar thereto, if the user doses a fixed insulin amount for meals, if the user modifies insulin meal dose amounts (determined from fixed dosing, carbohydrate counting, or experiential dosing) based upon premeal glucose values, if the user accounts for residual insulin from prior injections (IOB) when determining a dose amount (determined from fixed dosing, carbohydrate counting, or experiential dosing), or another technique. The DGAA can also determine if the user's mealtime doses are fixed according to meal type (e.g., breakfast, lunch, and dinner) or if the mealtime doses vary. A determination that mealtime doses are varying could be an indication that the user is basing the mealtime doses on a carbohydrate counting technique. The DGA can also determine if the user is adjusting a mealtime dose to account for high pre-prandial glucose. In some embodiments, the DGA may also determine a target glucose level, where the user is adjusting or correcting the mealtime dose when their level is above or predicted to be above the target glucose level. The DGA can also determine which meals are associated with insulin doses. The DGA can also determine a pattern of missed meal doses. For example, the DGA may detect if a user did not administer an insulin dose associated with a meal or time period at least two times, alternatively at least three times, in a period of time (e.g., one week or two weeks).

In some embodiments, the DGS may also prompt the user to enter typical meal dose amounts and typical time periods during which the meal dose is typically administered. In some embodiments, the DGS may prompt the user to enter the amount of rapid-acting insulin that they typically take for each meal when their glucose level is at a certain level. For example, the DGS may prompt the user to enter the amount of rapid-acting insulin that they typically take for each meal when their glucose level is at about 120 mg/dL. For rapid-acting insulin, the DGA may prompt the user for start and end time periods for each of breakfast, lunch, and dinner. Thereafter, a rapid-acting dose in the time period designated for each of the various meals may be logged as the dose for that meal. In some embodiments, the rapid-acting dose may be logged as the dose for that meal with no additional input from the user, e.g., the user may not be prompted after the administration of the dose to verify that the dose was for a particular meal. For example, a bolus time period for breakfast may be from about 2:00 am to about 11:00 am, A bolus time period for lunch may be about 11:30 am to about 2:00 pm. A bolus time period for dinner may be about 5:00 μm to about 8:00 μm. In this example embodiment, a rapid-acting insulin dose administered at 12:30 pm may be automatically logged as a lunch dose without any additional confirmation from the user.

In some embodiments, the DGS may also prompt the user for an amount of a typical basal dose that is administered and a time or time period that the typical basal dose is administered. The DGS may also prompt the user to review and verify all of the dosage amounts and administration times and time periods before finalizing.

The learning period can last any time period sufficient to achieve the requisite information. In many embodiments, this period is at least two days, more preferable a week or longer (e.g., 14 days), and can vary depending on how well the DGA can learn the trends. Results can be compiled into a summary report for both the user and physician,

### Glucose Patterns Report

As seen in FIGS. 6A-1-6G-2, a glucose patterns report 250 may include various elements that can be arranged in different layouts. Those of skill in the art will understand that the glucose patterns report 250 can be a graphical user interface outputted to a display of a computing device. The elements may include an identification of a most important glucose pattern 278, medication considerations 260, a variability statement 286, lifestyle considerations 284, and an excursion statement 288. The glucose patterns report 250 may also include an identification of the time period 264 that the report covers, an identification of the amount of time that the CGM sensor is active 266, the average number of scans or view per day 268, a glucose metrics or statistics section 270, a time-in-range (TIR) section 272, a considerations for clinician 276 section, and a glucose patterns 282 section. The considerations for clinician 276 section may include the medication considerations 260, variability statement 286, lifestyle considerations 284, and excursion statement 288.

The time period 264 that the report covers may be included in the glucose patterns report 250. The time period 264 may be about 7 days, about 14 days, about 1 month, about 2 months, or alternatively about 3 months. The time period 264 may be reported as a start and end date, a total number of days, and/or both the start and end date and the total number of days (e.g., “May 31-Jun. 13, 2018 (14 days)”). The time period 264 may be listed at the top of the report, e.g., under the report name, or alternatively, at the bottom of the report, in a header or footer, or elsewhere in the layout of the report.

The amount of time that the CGM sensor is active 266 may also be reported in the glucose patterns report 250, as, e.g., a percentage. The amount of time that the CGM sensor is active 266 may be listed at the top of the report, e.g., near the time period 264. Alternatively, the amount of time that the CGM sensor is active 266 may be listed at the top of the report, e.g., under the report name, or alternatively, at the bottom of the report, in a header or footer, or elsewhere in the layout of the report.

The average number of scans or view per day 268 during the time period 264 may also be included in the glucose patterns report 250. The average number of scans or view per day 268 may be listed at the top of the report, e.g., near the amount of time that the CGM sensor is active 266. Alternatively, the average number of scans or view per day 268 may be listed at the top of the report, e.g., under the report name, or alternatively, at the bottom of the report, in a header or footer, or elsewhere in the layout of the report.

The glucose metrics section 270 may also be included in the glucose patterns report 250. The glucose metrics section 270 may include an average glucose over the time period 264. The glucose metrics section 270 may also include the glucose management indicator (GMT) for the time period 264. A goal for each of the average glucose and GMI may optionally be listed next to the average glucose and GMI values to enable the user to quickly see how close or far they were to meeting their goals for the time period 264. The goals may be displayed in a different color (e.g., gray lettering vs. black for the actual calculate average glucose and GMI values) and may also be displayed in a smaller font size.

The time-in-range MR) section 272 may include a TIR graphical display 252 and a text component 274, which describes the amount of time in each different range. The TIR graphical display 252 may be a bar graph, a pie graph, a histogram graph, or any other graphical representation that show the relative amounts of time in a plurality of different concentration ranges. The TIR graphical display 252 may include at least 3, alternatively at least 4, alternatively at least 5, alternatively at least 6 different concentration ranges. The graphical display for each of the concentration ranges may reflect the time in range for that concentration range. For example, a relative area or a relative height of the graphical display for each concentration range may be proportional to or relate to the percentage of time determined for that concentration range during the time period 264.

The ranges may include below a very low threshold 290 (e.g., below about 54 mg/dL), between a very low threshold 290 and a low threshold 291 (e.g., between about 54 mg/dL and about 69 mg/dL), between a low threshold 291 and a high threshold 292 (e.g., between about 70 mg/dL and 180 mg/dL), between a high threshold 292 and a very high threshold 293 (e.g., between about 181 mg/dL and about 250 mg/dL), and above a high threshold 293 (e.g., above about 250 mg/dL).

The very low threshold 290 may be between about 50 mg/dL and about 65 mg/dL, alternatively between about 50 mg/dL, and about 60 mg/dL, alternatively about 53 mg/dL, alternatively about 54 mg/dL, alternatively about 55 mg/dL, alternatively about 56 mg/dL, alternatively about 57 mg/dL, alternatively about 58 mg/dL, alternatively about 59 mg/dL, alternatively about 60 mg/dL, alternatively about 61 mg/dL, alternatively about 62 mg/dL, alternatively about 63 mg/dL, alternatively about 64 mg/dL, alternatively about 65 mg/dL. The low threshold may be between about 60 mg/dL, and about 75 mg/dL, alternatively between about 65 mg/dL and about 75 mg/dL, alternatively between about 67 mg/dL and about 72 mg/dL, alternatively about 67 mg/dL, alternatively about 68 mg/dL, alternatively about 69 mg/dL, alternatively about 70 mg/dL, alternatively about 71 mg/dL, alternatively about 72 mg/dL, alternatively about 73 mg/dL, alternatively about 74 mg/dL, alternatively about 75 mg/dL, The high threshold may be between about 170 mg/dL and about 190 mg/dL, alternatively between about 175 mg/dL and about 185 mg/dL, alternatively about 175 mg/dL, alternatively about 176 mg/dL, alternatively about 177 mg/dL, alternatively about 178 mg/dL, alternatively about 179 mg/dL, alternatively about 180 mg/dL, alternatively about 181 mg/dL, alternatively about 182 mg/dL, alternatively about 183 mg/dL, alternatively about 184 mg/dL, alternatively about 185 mg/dL. The very high threshold may be between about 230 mg/dL and about 270 mg/dL, alternatively between about 240 mg/dL and about 260 mg/dL, alternatively about 245 mg/dL, alternatively about 246 mg/dL, alternatively about 247 mg/dL, alternatively about 248 mg/dL alternatively about 249 mg/dL, alternatively about 250 mg/dL, alternatively about 251 mg/dL, alternatively about 252 mg/dL, alternatively about 253 mg/dL, alternatively about 254 mg/dL, alternatively about 255 mg/dL. The thresholds and limits for the various ranges may be customizable by the user. Alternatively, the thresholds and limits for the various ranges may not be able to be customized by the user.

The different concentration ranges in the TIR graphical display may each have a different color. For example, a graphical display for the below the very low threshold graphical display may be colored dark red or maroon, a graphical display for the range between the very low threshold and the low threshold may be colored a lighter red, a graphical display for the range between the low threshold and the high threshold may be colored green, a graphical display for the range between the high threshold and the very high threshold may be colored yellow, and a graphical display for the range above the very high threshold may be colored orange.

The text component 274 of the TIR display 272 may include a label for each range. The range below the very low threshold may be labeled “very low”, the range between the very low threshold and the low threshold may be labeled “low”, the range between the low threshold and the high threshold may be labeled “target”, the range between the high threshold and the very high threshold may be labeled “high”, and the range above the very high threshold may be labeled “very high.” The text component 274 of the TIR display 272 may also include, or in the alternative, explanations as to the numerical limits of the concentration ranges of the plurality of ranges. The different concentration ranges may be listed next to or in close proximity to the corresponding graphical element and/or the label for that range. For example, for the graphical element for the time below the very low threshold 290, the text may read “Very Low <54” or “below 54 mg/dL”, “Low 54-69” or “54-69 mg/dL′, “Target 70-180” or Target 70-180 mg/dL”, “High 181-250” or “181-250 mg/dL”, and/or “Very High >250” or “above 250 mg/dL.” The text component 274 may also contain a numerical value, e.g., a percentage, for the time spent in each range of the concentration ranges. Alternatively or in addition to the individual numerical values for each concentration range, the text component 274 may include a combined numerical value, e.g., a combined percentage, for two or more ranges. For example, the numerical values for the time below the very low threshold 290 and the time between the low threshold 291 and the very low threshold 290 may be reported as a single combined numerical value. Moreover, the numerical values for the time above the very high threshold 293 and the time between the high threshold 292 and the very high threshold 293 may be reported as a single combined numerical value. The numerical or combined numerical values may be located next to or in close proximity with the graphical element(s) and or explanatory text for each respective concentration range(s). Where both the individual numerical values and combined numerical values are reported, the combined numerical values may be visibly different than the individual numerical values. For example, the combined numerical values may be bolded, italicized, or a different color. The sum of the reported numerical or combined numerical values may equal 100 or may equal a number other than 100. The text component 274 may also contain a goal 275 (see, e.g., FIGS. 6A-2 and 6A-3), e.g., percentage, for each concentration range. Alternatively, the text component 274 may also contain a combined goal for two or more concentration ranges. For example, the goals for the time below the very low threshold and the time between the low threshold and the very low threshold may be reported as a single goal. Moreover, the goal for the time above the above the very high threshold and the time between the high threshold and the very high threshold may be reported as a single goal. The goals for each of the concentration ranges and/or the combined goals may be listed next to or in close proximity with the determined numerical value for the time period 264 for each respective concentration range.

A section detailing considerations for the clinician, HCP, or patient 276 may also be included in the glucose patterns report 250. The considerations for the clinician 276 section may include a most important patterns section 278, a medication considerations section 260, and a lifestyle considerations section 284.

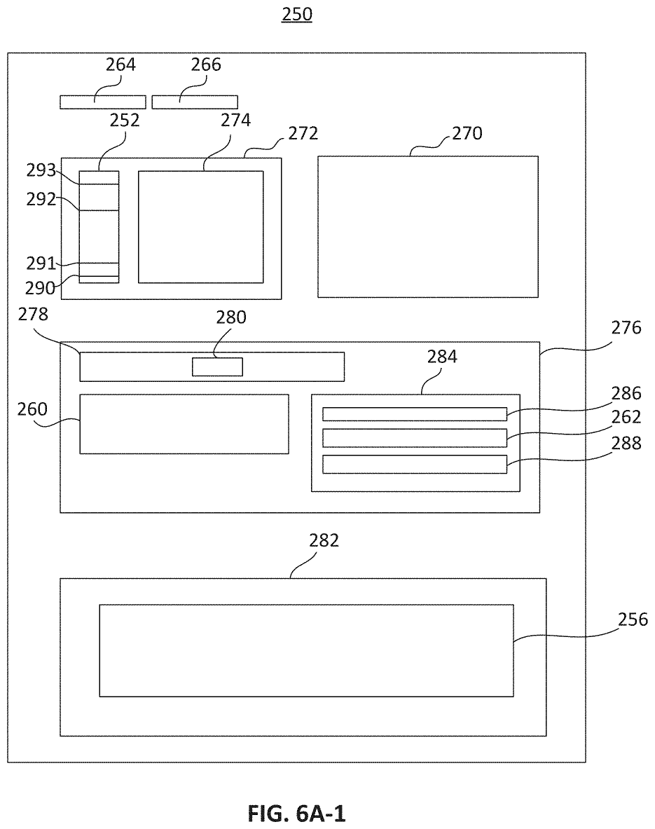

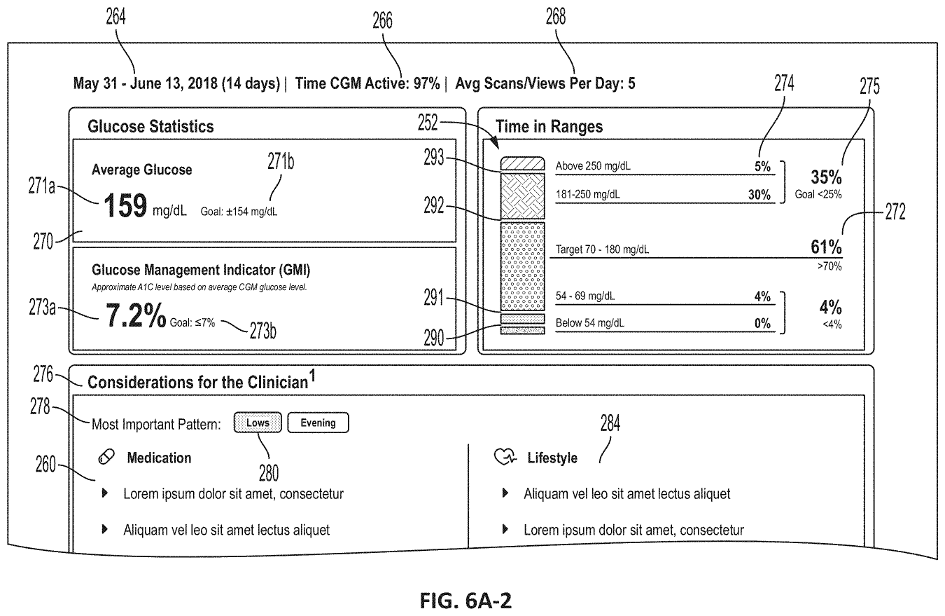

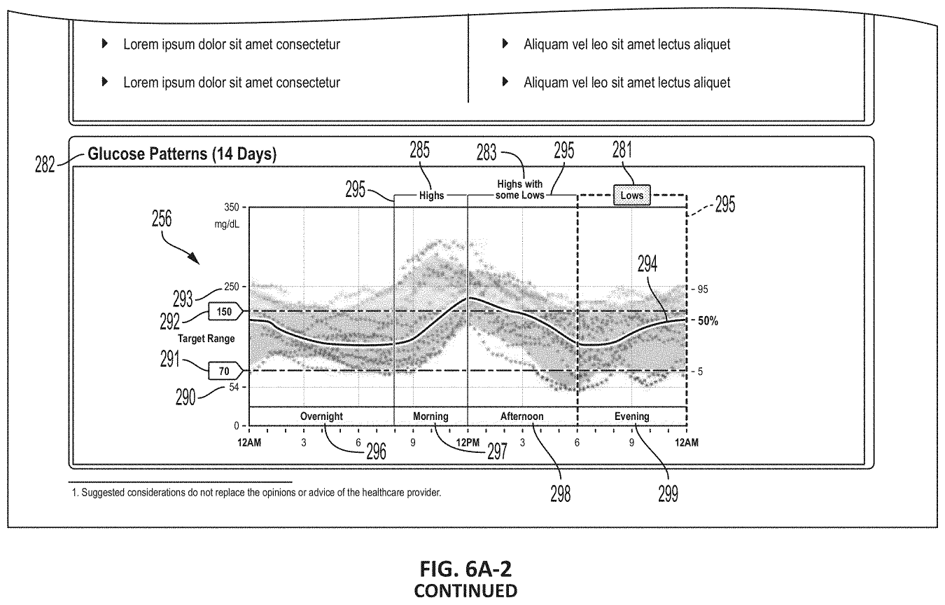

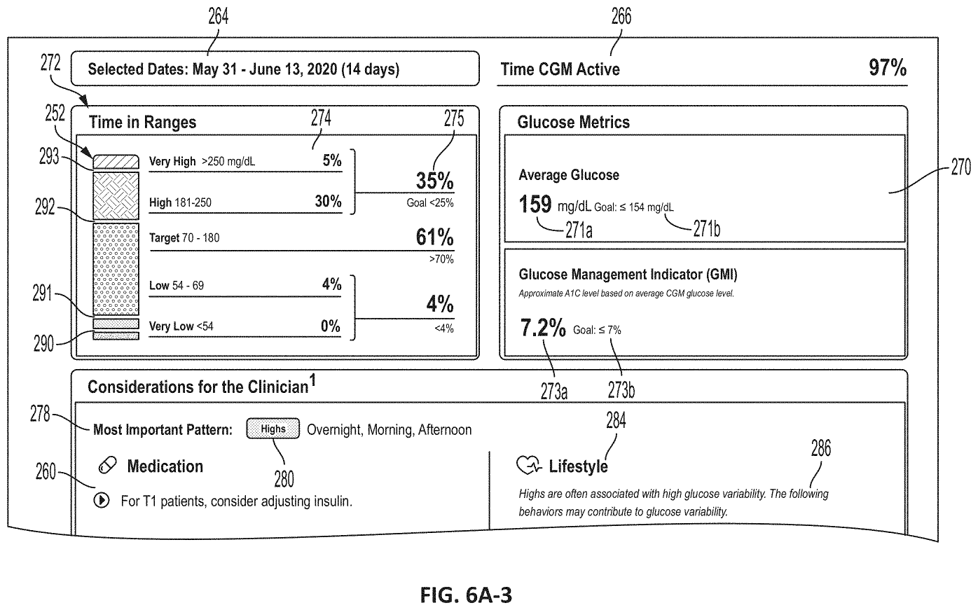

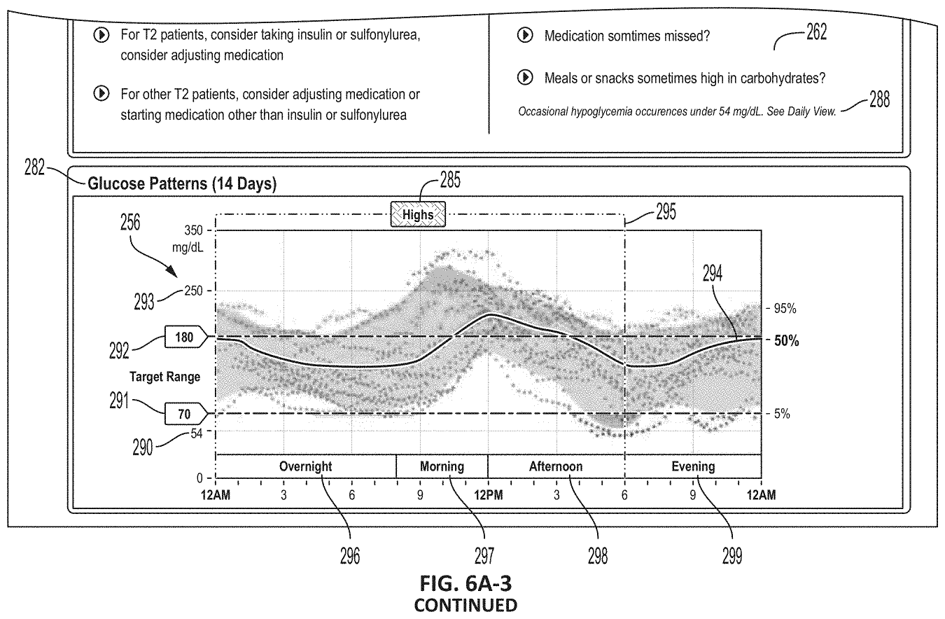

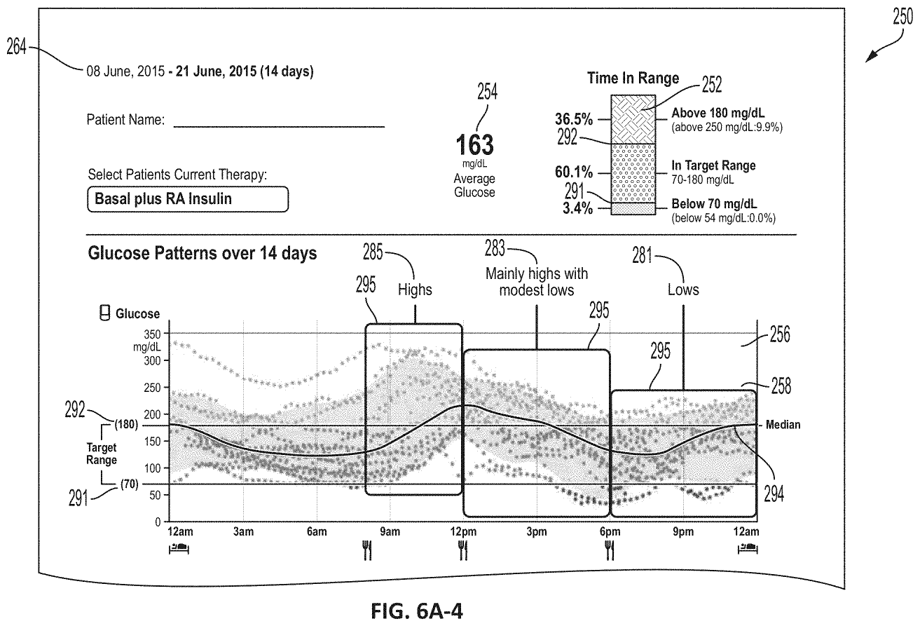

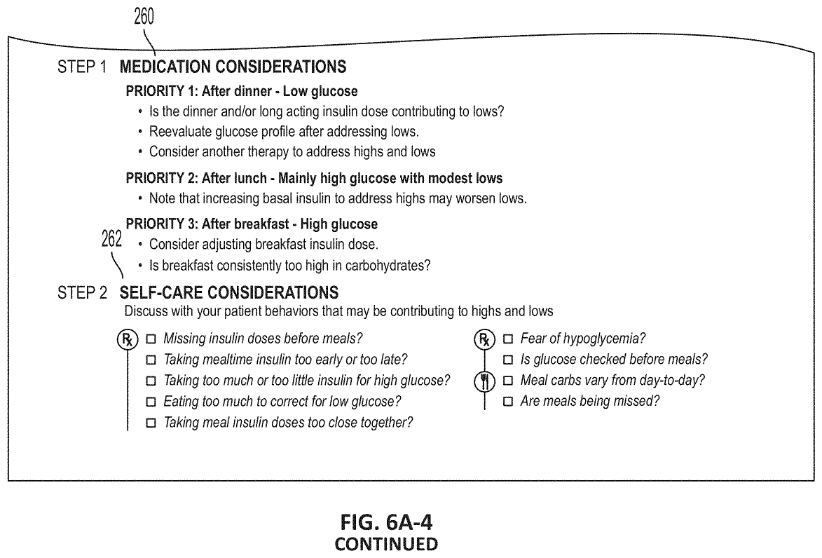

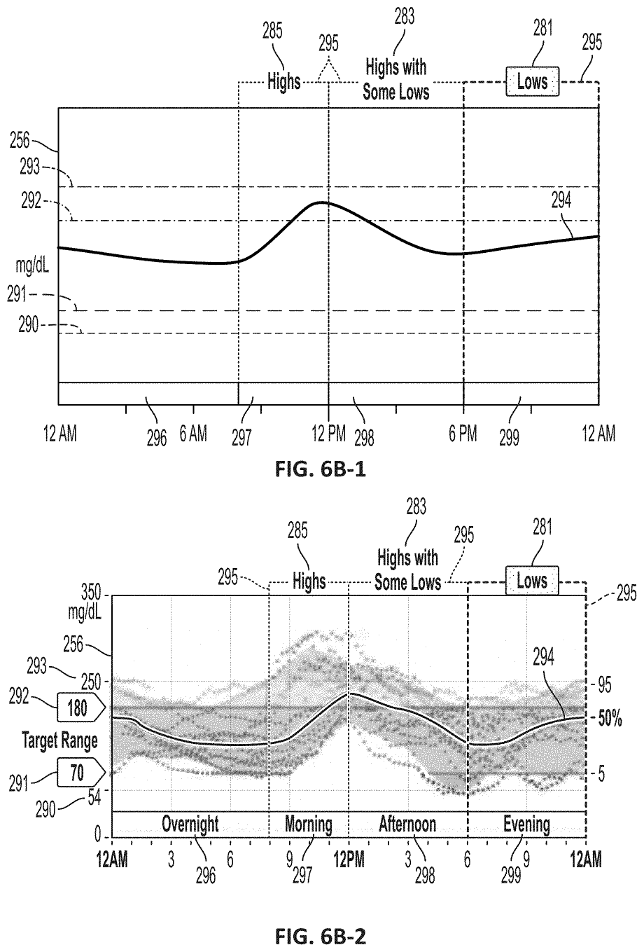

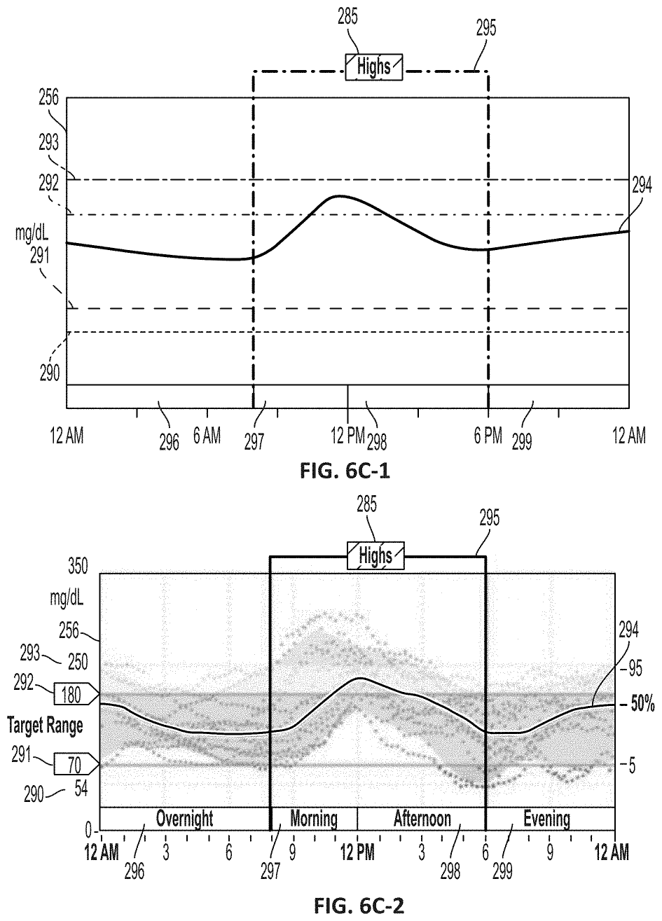

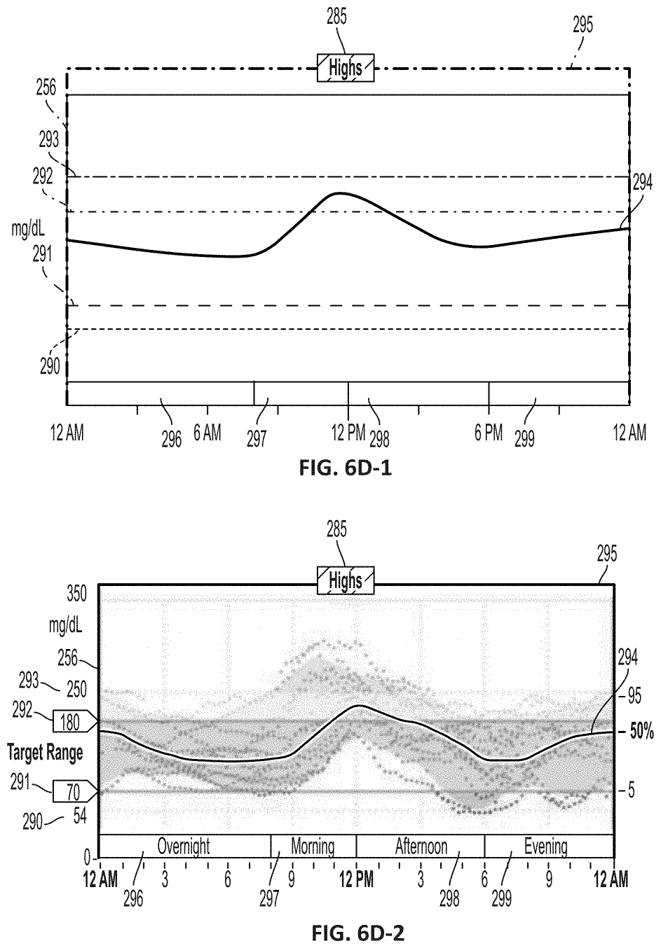

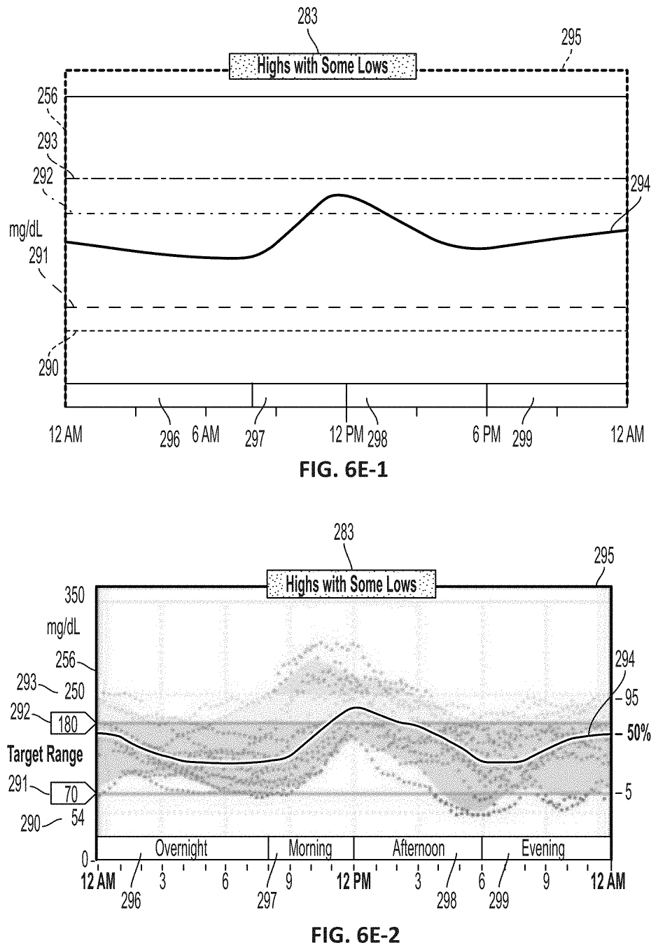

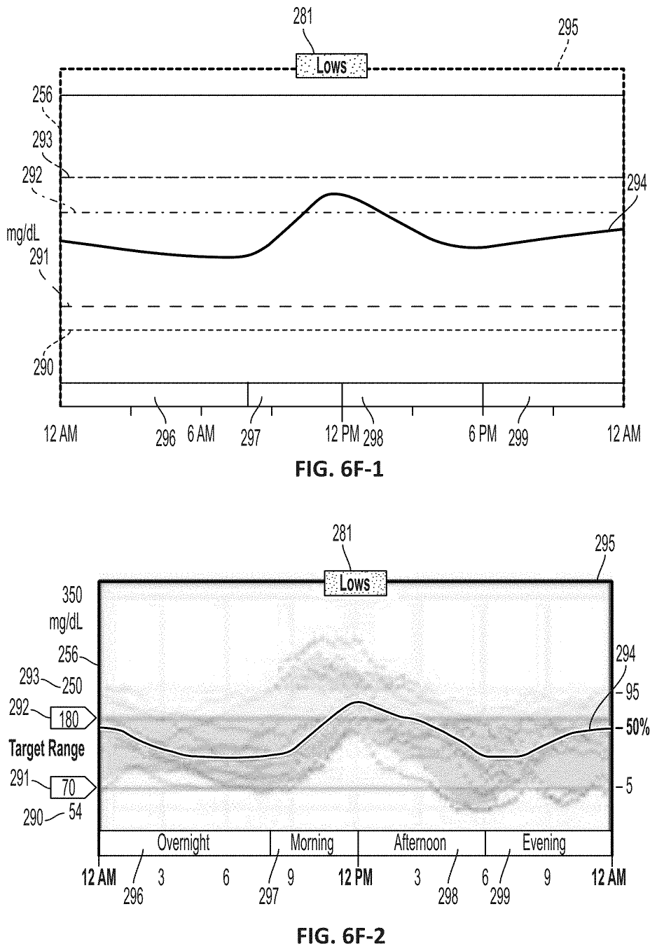

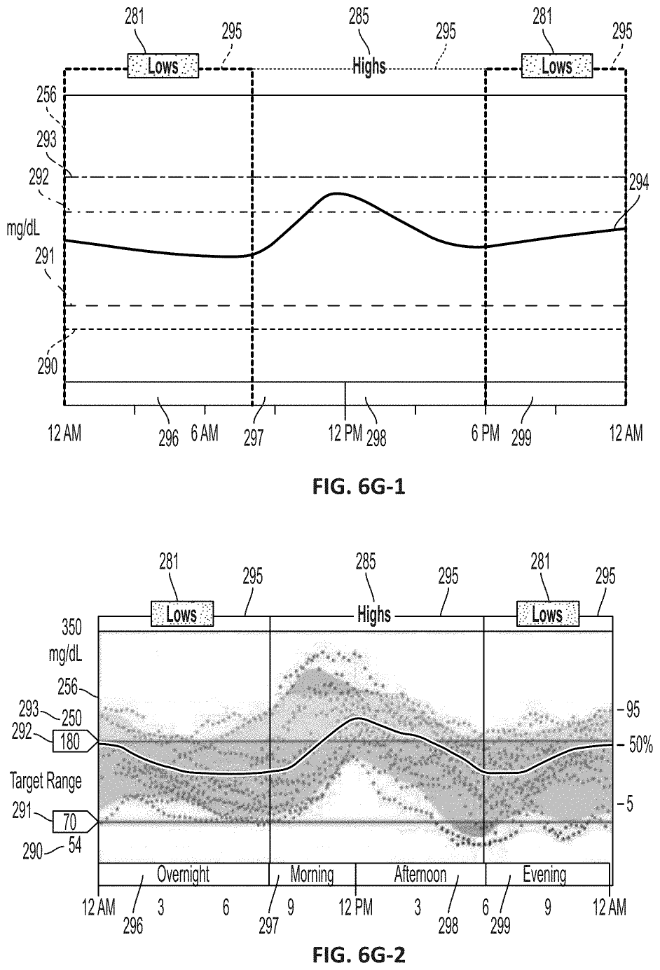

The most important patterns section 278 may identify the most important pattern for the time period 264 determined by an algorithm, including but not limited to, the GPA algorithm described elsewhere in this application. The most important patterns section 278 may identify the pattern(s) as, including but not limited to, “Lows,” “Highs with Some lows,” and “Highs.” The most important patterns section 278 may also identify the periods of the day in which the most important pattern occurred, e.g., overnight, morning, afternoon, and/or evening. Each of the patterns and the periods of the day in which the identified patterns have occurred may be identified in text. e.g., a sentence or phrase, or may each be identified in a tag(s) 280. Where multiple patterns, including a “Lows” pattern, are detected, the glucose patterns report 250 may identify the “Lows” pattern in the most important patterns section 276 so that the clinician may first address these “Lows” patterns before addressing any “Highs with Some Lows” or “Highs,” Where multiple patterns are identified, the patterns identified in the most important patterns section 278 may be prioritized as identifying the “Lows” patterns first, then the “Highs with Some Lows” patterns, and then the “Highs” pattern. The additional patterns, however, may be identified in outlines or boxes in the glucose patterns profile 256 even if they are not identified in the most important patterns statement 278. The pattern(s) tag 280 in the most important patterns section 278 may be color coded to match the color of the outlines or boxes or partial boxes or brackets highlighting these sections in a glucose concentration profile 256 in a glucose patterns section 282. For instance, a tag 280 identifying “Lows” in the evening may be colored red (e.g., red background with white lettering) and a box or partial box surrounding the evening period of the glucose concentration profile 256 may have a red line color and the color-coded red tag identifying lows may be located at the top of the box. The time period(s) that the most important pattern occurs in may be listed next to the tag 280. The time period(s) may be displayed as text according to the order on the glucose concentration profile 256, e.g., from left to right “Overnight,” “Morning,” “Afternoon,” and “Evening.” Alternatively, the time period(s) may be identified in tags (not shown), but in a different color than the pattern tag. For instance, the time period tag may be gray. If the pattern occurs in all four time periods of the day, the most important patterns section 278 may include two tags “All Day” and “Overnight.” If the pattern occurs in all of the time periods of the day except for “Overnight,” the most important patterns section 278 may include a single tag that says “All Day” or it may include three tags for “Morning,” “Afternoon,” and “Evening.” If a pattern occurs in multiple time periods, a single box or partial box may outline adjacent time periods with a single label (see, e.g., FIGS. 6C-6E).

Medication considerations 260 can also be provided in the glucose patterns report 250 if the patient's current therapy (e.g., basal plus RA insulin, basal only, basal plus SU, etc.) is known. Medication guidance can be provided in the form of text recommendations. General advice regarding titrating an insulin dose can be provided based on the identified high and low glucose patterns, which are highlighted with boxes 258 in the glucose concentration profile 256. This general advice, however, may have been determined without access to data as to the actual insulin doses administered. The recommendations can generally follow the rule of mitigating any low patterns first before mitigating high patterns. If the glucose patterns report contains suggestions for insulin dose titration(s), the glucose patterns report 250 could also include a suggestion that the patient is a good candidate for the DGS 100, facilitating a conversation between the HCP and patient before transitioning to the learning period.

The medications consideration section 260 may include different statements and/or observations relating to medications administered during the time period 264. The medications consideration section 260 may include a question asking whether the medications are contributing to lows. Alternatively, the medications consideration section 260 may include a statement that medication added to address highs may worsen lows. Alternatively, the medications consideration section 260 may include a statement that if the patient is starting or adjusting medication to address highs, consider how the medication could induce lows. The medications consideration section 260 may also include a statement advising that the clinician and/or patient should consider different therapy to address glucose variability. The medications consideration section 260 may also state that for T1 patients, consider adjusting insulin. For 12 patients, the medications consideration section may include statements advising that for T2 patients currently taking insulin or sulfonylurea, consider adjusting medication; or for other T2 patients, consider adjusting medication or starting medication other than insulin or sulfonylurea. The medications consideration section 260 may also state that for other T2 patients, consider starting insulin.

The medications consideration section 260 may include, but are not limited to, one or more statements relating to the following topics:
Medications contributing to lows?
Medication added to address highs may worsen lows.
If starting or adjusting medication to address highs, consider how the medication could induce lows.
Consider different therapy to address glucose variability.
For T1 patients, consider adjusting insulin.
For T2 patients currently taking insulin or sulfonylurea, consider adjusting medication.
For other T2 patients, consider starting insulin.
For other T2 patients, consider adjusting medication or starting medication other than insulin or sulfonylurea.

The lifestyle considerations section 284 may include a variability statement 286, an excursion statement 288, and self-care considerations 262, If it is determined that the variability for a time period(s) is low, a statement regarding variability may not be included. In some embodiments, the variability statement 286 may not be displayed if no patterns were detected in any of the time periods. In some embodiments, the variability statement 286 may be displayed when it is determined that there is high variability and it is also determined that a pattern(s) exists. If it is determined that the variability for a time period(s) is high, the glucose patterns report 250 may include a variability statement 286. The variability statement 286 may state that lows are often associated with high glucose variability. The variability statement 286 may alternatively or in addition to state that highs are often associated with high glucose variability. The variability statement 286 may also state that certain behaviors may contribute to high glucose variability, and may then include a list of the certain behaviors. The variability statement 286 may also state that certain behaviors may contribute to glucose variability, and may then include a list of the certain behaviors that contribute to glucose variability.

The variability statement 286 may include, but are not limited to, one or more statements relating to the following topics:
Lows are often associated with high glucose variability.
The following behaviors may contribute to glucose variability.
The following behaviors may contribute to high glucose variability.
Highs are often associated with high glucose variability.

The determination of variability is described elsewhere in this application. Alternative determinations for determining variability are described in WO 2014/145335 and WO 2014/106263, both of which are hereby expressly incorporated by reference in their entirety for all purposes.

As seen in FIG. 6A-3, the excursion statement 288 may be included in the glucose patterns report 250 when an excursion is detected. An excursion may be an instance of a glucose level below a very low threshold is detected. The very low threshold may be between about 50 mg/dL, and about 65 mg/dL, alternatively between about 50 and about 60 mg/dL, alternatively about 53 mg/dL, alternatively about 54 mg/dL, alternatively about 55 mg/dL, or as described in other parts of this application. If an excursion is detected, the excursion statement 288 may suggest that the clinician discuss with your patient occasional hypoglycemia occurrences below the very low threshold, and may refer to a Weekly Summary Report or a Daily View Report, which may list the excursions below the very low threshold (e.g., below about 54 mg/dL). Alternatively, the excursion statement 288 may state that occasional hypoglycemia occurrences below the very low threshold 290, and may refer to a Weekly Summary Report or a Daily View Report (e.g., “Occasional hypoglycemia occurrences below 54 mg/dL. See Daily View report.”). The excursion statement 288 may be included in the glucose patterns report 250 when one or more excursions below the very low threshold was detected. In some embodiments, the excursion statement may not be included in the glucose patterns report 250 if a low pattern was detected even if one or more excursions below the very low threshold was detected. In some embodiments, the excursion statement 288 may be displayed when a “Highs” pattern, a “Highs with Some Lows” pattern, or no pattern was detected.

Self-care considerations 262 may also be included in the glucose patterns report 250. Self-care considerations 262 can be displayed in the glucose patterns report 250 when the GPA algorithm has identified patterns with high variability. Alternatively, the glucose concentration profile 256 might have such high variability that the logic behind the report cannot make specific suggestions, instead defaulting that the user consult with their HCP on lifestyle or therapy changes.

The self-care considerations 262 displayed in the glucose patterns report 250 may depend on the type of patterns detected, the amount of variability, the median glucose value, the presence or absence of hypoglycemia risks, and the presence or absence of excursions. The self-care considerations 262 may include one or more statements relating to the following topics:
Meals sometimes missed or vary in carbohydrates?
Activity level varies daily?
Alcohol consumption varies daily?
Medication sometimes missed?
Meals or snacks often high in carbohydrates?
Meals or snacks sometimes high in carbohydrates?

The glucose patterns section 282 may include a glucose concentration profile 256, which displays the glucose data across a 24-hour period. The glucose concentration profile 256 may be an ambulatory glucose profile (AGP). Alternatively, the glucose patterns profile may display various data points as points or dots on the graph. The points or dots may or may not be connected with a line(s) to show an analyte curve for each day. Exemplary glucose concentration profiles 256 are depicted in FIGS. 6B-1-6G-2. The glucose concentration profile 256 may be a graph of the glucose data for the time period 264 of the report 250, where the various data points of the graph can be color-coded to correspond to which concentration range the glucose analyte level falls within (not shown), The color-coding may correspond to the color-coding the of TIR display 252. The boxes or partial boxes 295 surrounding different portions of the glucose concentration profile 256 highlight patterns (e.g., “Highs,” “Lows,” and “Highs with Some Lows”) that were detected. The various thresholds and boundaries for different concentration ranges may be highlighted in the glucose concentration profile 256, including labeling or otherwise highlighting (e.g., text label and/or horizontal lines) the very low threshold 290, the low threshold 291, the high threshold 292, and the very high threshold 293. The target or goal range between the low threshold 291 and the high threshold 292 may also be labeled on the glucose concentration profile 256, e.g., shaded or highlighted in a different color (e.g., green). The median glucose levels for each time may be highlighted with a solid line 294 and labeled as the median or 50%. The median glucose line 294 may change colors depending on the median value in that portion of the line. For example, the median glucose line 294 may be colored dark maroon or dark red for those median values that are below the very low threshold, may be colored red for those median values that fall between the very low threshold and the low threshold, may be colored green for those median values that fall between the low threshold and the high threshold, and may be colored yellow for those median values that fall between the high threshold and the very high threshold, and may be colored orange for those median values that are above the very high threshold. The four time periods may be Overnight 296 (e.g., about 12 am to about 8 am), Morning 297 (e.g., about 8 am to about 12 pm), Afternoon 298 (e.g., about 12 pm to about 6 pm), and Evening 299 (e.g., about 6 pm to about 12 am).

For each of the time periods, the algorithm may determine that that one of three possible patterns exist or that no pattern exists in the time period. Thus, each of the four time periods may be determined to have the following patterns assigned: (1) Lows 281, (2) Highs with some Lows 283, (3) Highs 285, or (4) no adverse patterns detected. Each pattern identified may be labeled above the appropriate section in the graph with a colored tag and the time period may be outlined with a box or partial box, which may also be color-coded. “Low” 281 patterns may appear as red (see, e.g., FIGS. 6B-1-6B-2). “Highs with some Lows” 283 may appear as an amber or maroon color (see, e.g., FIGS. 6E-1-6E-2). “Highs” 285 may appear in an orange color (see, e.g., FIGS. 6C-1-6C-2 and FIGS. 6D-1-6D-2). As seen in FIGS. 6B-1-6B-2, where multiple patterns are detected, multiple boxes or multiple partial boxes 295 may display, all of the patterns in relevant sections on the glucose concentration profile 256. In some embodiments, the “Lows” pattern 281 may be prioritized and highlighted in a different color, e.g., red, as compared to the boxes and labels for the other patterns. As seen in FIGS. 6C-1-6C-2, if the same pattern occurs in two or more adjacent time periods, a single box or partial box 295 may outline all of the relevant time periods, with a single label on top of the box. As seen in FIGS. 6D-1-6D-2 and FIGS. 6E-1-6E-2, if the same pattern occurs in all of the time periods of the day (Overnight, Morning, Afternoon, and Evening), a single box or partial box 295 may, outline all of the time periods, with a single label for the box 295.

In some embodiments, the patterns may be determined according to the GPA algorithm, discussed elsewhere in the specification. A plurality of variables may be used to determine the output of the GPA algorithm and the content of the glucose patterns report 250. The variables may include, but are not limited to, the priority pattern, additional patterns in combination with the priority pattern, variability (high or low), median glucose (e.g., above or below 180 mg/dL), moderate hypoglycemia risk (yes or no), and excursions below 54 mg/dL (yes or no). The layout and text associated with each element may vary depending on the output of the GPA algorithm.

The particular guidance text displayed on any given report may be based on a combination of the three patterns described herein, a “hidden” pattern defined as a “moderate hypo risk” pattern determined according to the GPA algorithm discussed elsewhere in the specification, glucose variability, and hyperglycemia as defined by the overall glucose median compared to a 180 mg/dL threshold. In some embodiments, the patterns and metrics described here may be replaced by similar patterns and metrics. In some embodiments, a low pattern may be replaced by a calculation where the number of low events (e.g., glucose less than 70 mg/dL) exceeds a threshold. For example, the threshold may require that the number of low events may be at least 4 low events in 14 days. In some embodiments, the variability metric may be replaced by any other common glucose variability metric. In some embodiments, the overall hyperglycemia metric may be defined as a mean glucose instead of median. In some embodiments, the “Highs with some Lows” pattern may be replaced with a calculation of about 2-3 low events and about 4 or greater high events, where a high event may be defined as glucose >180 mg/dL, that occur in the time-of-day period for 14 days. Thus, the guidance may be driven by comparable patterns and metrics.

TABLE 1 below outlines an exemplary mapping of guidance or look-up table that may be provided in the glucose patterns report. The inputs include: (1) whether or not there is high variability, (2) the determination that there is a Lows, Highs with Some Lows, Highs, or no pattern, (3) whether or not there is a risk of hypoglycemia, and (4) whether or not the glucose median (G_{med}) is greater or less than 180 mg/dL, TABLE 1 may define the guidance text outputs based on the inputs. The outputs may include (1) medication considerations, (2) Lifestyle statements and considerations, (3) low excursions, and (4) most important pattern identification. In other embodiments, the look up table may have fewer inputs. In some embodiments, the table may exclude, e.g., overall hyperglycemia and the corresponding guidance, or additional inputs. Various scenarios outlined in TABLE 1 are described in more detail below.

TABLE 1
Exemplary Glucose Mapping Lookup Table.

| | | | | | | Outputs | | | | | | |
|---|---|---|---|---|---|---|---|---|---|---|---|---|
| | | | | | | | Lifestyle | | | | | |
| | | | | | | | Statement | Lifestyle | | | | |
| | | | | | | | (display | Consider. | | | | |
| | | | | | | | if | (display | | | Allow | |
| Inputs | | | | | | Medication. | any | if any | Display | Display | display | Most |
| High | Low | H/L | High | Hypo | G_{med} | Consider- | High | High | Med. | Lifestyle | <54 mg/dL | Impt |
| Var | Pattern | Pattern | Pattern | Risk | >180 | ations | Var) | Var) | Consid.? | Consid.? | Excursion? | Pattern |
| None | None | None | None | — | — | <none> | <none> | <none> | No | No | Yes | No |
| | | | | | | | | | | | | Pattern |
| None | None | None | Any | None | No | A1 | <none> | B5 | Yes | Yes | Yes | Highs |
| | | | | | | A2 | | | | | | |
| | | | | | | A3 | | | | | | |
| None | None | None | Any | None | Yes | A1 | <none> | B5 | Yes | Yes | Yes | Highs |
| | | | | | | A2 | | | | | | |
| | | | | | | A4 | | | | | | |
| None | None | None | Any | Any | — | A5 | <none> | B5 | Yes | Yes | Yes | Highs |
| None | None | Any | — | — | — | A5 | B1 | B7 | Yes | Yes | No | Highs |
| | | | | | | A6 | | B8 | | | | with |
| | | | | | | | | B9 | | | | some |
| | | | | | | | | B10 | | | | Lows |
| None | Any | None | None | — | — | A7 | <none> | N/A | Yes | Yes | No | Lows |
| None | Any | None | Any | — | — | A7 | <none> | N/A | Yes | Yes | No | Lows |
| | | | | | | A8 | | | | | | |
| None | Any | Any | — | — | — | A7 | B3 | B8 | Yes | Yes | No | Lows |
| | | | | | | A8 | B2 | B9 | | | | |
| | | | | | | B10 | | | | | | |
| Any | None | None | None | — | — | <none> | <none> | <none> | No | No | Yes | No |
| | | | | | | | | | | | | Pattern |
| Any | None | None | Any | None | No | A1 | B4 | B7 | Yes | Yes | Yes | Highs |
| | | | | | | A2 | B2 | B6 | | | | |
| | | | | | | A3 | | | | | | |
| Any | None | None | Any | None | Yes | A1 | B4 | B7 | Yes | Yes | Yes | Highs |
| | | | | | | A2 | B2 | B6 | | | | |
| | | | | | | A4 | | | | | | |
| Any | None | None | Any | Any | — | A5 | B4 | B7 | Yes | Yes | Yes | Highs |
| | | | | | | | B2 | B6 | | | | |
| Any | None | Any | — | — | — | A5 | B1 | B7 | Yes | Yes | No | Highs |
| | | | | | | A6 | | B8 | | | | with |
| | | | | | | | | B9 | | | | some |
| | | | | | | | | B10 | | | | Lows |
| Any | Any | — | — | — | — | A7 | B3 | B8 | Yes | Yes | No | Lows |
| | | | | | | A8 | B2 | B9 | | | | |
| | | | | | | | | B10 | | | | |

“Any” = at least one of the 4 TOD periods
“None” = 0 of the 4 TOD periods
G_{med} = median glucose concentration

### Medication Considerations:

Statement A1: For T1 patients, consider adjusting insulin.
Statement A2: For T2 patients currently taking insulin or sulfonylurea, consider adjusting medication.
Statement A3: For other T2 patients, consider adjusting medication or starting medication other than insulin or sulfonylurea.
Statement A4: For other T2 patients, consider starting a new medication such as insulin.
Statement A5: If starting or adjusting medication to address highs, consider how the medication could induce lows.
Statement A6: Consider different therapy to address glucose variability.
Statement A7: Medications contributing to lows?
Statement A8: Medication added to address highs may worsen lows.

### Lifestyle Statements and Considerations

Statement B1: The following behaviors may contribute to high glucose variability.
Statement B2: The following behaviors may contribute to glucose variability.
Statement B3: Lows are often associated with high glucose variability.
Statement B4: Highs are often associated with high glucose variability.
Statement B5: Meals or snacks often high in carbohydrates?
Statement B6: Meals or snacks sometimes high in carbohydrates?
Statement B7: Medication sometimes missed?
Statement B8: Meals sometimes missed or vary in carbohydrates?
Statement B9: Activity level varies daily?
Statement B10: Alcohol consumption varies daily?

As seen in FIGS. 6A-1-6A-4, the layout of the glucose patterns report 250 may have the time period 264 and time that the CGM is active 266, for example, may appear at a top of the report, e.g., under the title of the report. In some embodiments, the average scans/views per day may also appear at the top of the report The time period 264, time that the CGM is active 266, and the average scans/views per day 268 may be listed next to each other, and may be separated by lines or outlined by boxes. The glucose metrics section 270 and TIR section 272 may appear side-by-side underneath the listing of the period 264, time that the CGM is active 266, and the average scans/views per day 268. The glucose metrics section 270 may display the average glucose value 271a with the goal value 271b and a display of the GMI 273a with the goal value 273b. In some embodiments, the average glucose value with the goal value may be displayed above the display of the GMI with the goal value. The TIR display section 272 may be displayed on the left and the glucose metrics section 270 may be displayed on the right. Alternatively, the TIR display section 272 may be displayed on the right and the glucose metrics section 270 may be displayed on the left. Beneath the glucose metrics section 270 and the TIR display section 272, the glucose patterns report 250 may include the considerations for the clinician section 276. The consideration for the clinicians section 276 may include an identification of the most important pattern 278 at the top of the section 276, and the medications considerations 260 and lifestyle considerations 284 may be listed side-by-side underneath the most important pattern 278. Within the lifestyle considerations section 284, when all three statements are reports, the variability statement 286 may be listed first, the self-care considerations 262 may be listed second, and the excursion statement 288 may be listed last. Alternatively, each of the three statements 286, 262, and 288, may be listed in different orders, e.g., with the self-care considerations 262 listed first, middle, or last, with the variability, statement 286 listed first, middle, or last, and with the excursion statement 288 listed first, middle, or last. The glucose patterns section 282, which may include the glucose concentration profile 256, may appear underneath the considerations for the clinician 276. Thus, in one embodiment, the glucose metrics section 270 and the TIR display section 272 may appear in a top third of the report 250, the considerations for clinician 276 may appear in a middle third of the report 250, and the glucose patterns section 282 may appear in a bottom third of the report. Alternatively, in other embodiments, the glucose metrics section 270 and the TIR display section 272 may appear in any of the top, middle; or bottom third of the report 250; the considerations for clinician 276 may appear in the top, middle, or bottom third of the report 250; and the glucose patterns section 282 may appear in the top, middle, or bottom third of the report.

In some embodiments, where no patterns were found and no excursions were detected during the time period 264, the considerations for the clinician 276 may contain a statement that says no adverse glucose patterns were detected. Moreover, the glucose concentration profile may not contain any boxes highlighting any of the time periods of the day.

In some embodiments, where no patterns were found but at least one excursion was detected during the time period 264, the considerations for the clinician 276 may include a most important patterns statement 278 that states that no adverse glucose patterns were detected. The considerations for the clinician 276 may also include an excursion statement 288 that may suggest to the clinician to discuss with the patient occasional hypoglycemia occurrences below the very low threshold, and refer the clinician to see an additional report, e.g., a weekly summary report. The glucose concentration profile may not contain any boxes highlighting any of the time periods of the day but may contain some data points that are highlighted as dark red or maroon, which are below the very low threshold 290, which correspond to the one or more excursions detected.

In some embodiments, where a “Lows” pattern 281 in a time period and low variability was detected, the considerations for the clinician 276 may include a most important patterns statement 278 that may highlight the “Lows” pattern 281 in a tag colored red, and may also include tags for the time periods of day that the “Lows” pattern occurred. If the “Lows” pattern 281 occurred in every time period of the day, the most important patterns statement 278 may include two tags “All Day” and “Overnight.” As seen in FIGS. 6F-1-6F-2, if the “Lows” pattern 281 occurred for every time period of the day, the glucose concentration profile may contain a single box 295, e.g., a red box, highlighting the whole graph with a “Lows” heading at the top. Alternatively, as seen in FIGS. 6G-1-6G-2, if the “Lows” pattern 281 occurred in at least two time periods that are not adjacent, then those time periods would be highlighted by separate boxes or partial boxes 295.

In some embodiments, where a “Lows” pattern 281 in at least one time period and high variability was detected, or where a “Lows” pattern 281 and any other patterns were detected, the considerations for the clinician 276 may include a most important patterns statement 278 that may highlight the “Lows” pattern 281 in a tag 280 colored red, and may also an identification for the times of day that the “Lows” pattern occurred, which may be colored a different color such as gray. The medication considerations 260 may include statements for consideration by the clinician in determining treatment for the “Lows” pattern. The statements may include, but are not limited to: “Medications contributing to lows?” and “Medication added to address highs may worsen lows.” The considerations for the clinician 276 may also include a variability statement 286 regarding the high variability detected. The statements may include, but are not limited to “Lows are often associated with high glucose variability” and “The following behaviors may contribute to glucose variability,” which may be followed by a list of behaviors.” The considerations for the clinician 276 may also include self care considerations 262. The statements regarding self-care 262 may include, but are not limited to: “Meals sometimes missed or vary in carbohydrates?”; “Activity level varies daily?”; and “Alcohol consumption varies daily?” In the glucose concentration profile, as seen for example in FIGS. 6G-1-6G-2, the time periods with the “Lows” patterns may be highlighted with red boxes, with a “Lows” heading at the top. In some embodiments in which adjacent time periods have the same pattern, a single box may outline the adjacent time periods with the same patterns (see, FIGS. 6F-1-6F-2). As seen in FIGS. 6G-1-6G-2, if different types of patterns are detected in the time period, all of the patterns may be identified with boxes outlining the relevant time periods of the day and appropriate heading labels identifying the types of patterns. In some embodiments, where a “Lows” pattern 281 is detected along with a “Highs” 285 and/or “Highs with Some Lows” 283 patterns, then the “Lows” pattern 281 may be highlighted in a different color, e.g., red, compared to the other patterns, e.g., gray (see, e.g., FIGS. 6B-1-6B-2 and FIGS. 6G-1-6G-2).

In some embodiments, where a “High with some Lows” pattern 283 and high variability is detected, or where a “High with some Lows” pattern 283 and a “Highs” pattern 285 is detected, the considerations for the clinician 276 may include a most important patterns statement 278 that may highlight the “High with some Lows” pattern 283 in a tag 280 colored red, and may also include an identification for the times of day that the “High with some Lows” pattern 283 occurred. In some embodiments, the “High with some Lows” pattern may be prioritized over the “Highs” pattern as more important for the clinician to address first. The medication considerations 260 may include statements for consideration by the clinician in determining treatment for the lows pattern. The statements may include, but are not limited to: “If starting or adjusting medication to address highs, consider how the medication could induce lows” and “Consider different therapy to address glucose variability.” The considerations for the clinician 276 may also include a variability statement 286 regarding high variability detected. The statements may include, but are not limited to: “The following behaviors may contribute to high glucose variability,” which may be followed by a list of behaviors. The considerations for the clinician 276 may also include self-care considerations 262. The statements regarding self-care may include, but are not limited to: “Medication sometimes missed?”; “Meals sometimes missed or vary in carbohydrates?”; “Activity level varies daily?”; and “Alcohol consumption varies daily?” In the glucose concentration profile 256, the time periods with the “High with some Lows” patterns 283 may be highlighted with dark red boxes or partial boxes, with a “High with some Lows” heading at the top (see, e.g., FIGS. 6E-1-6E-2), and the time periods with “Highs” patterns 285 are highlighted with boxes 295, e.g., gray boxes, with a “Highs” heading at the top of the box.

In some embodiments, where only a. “Highs” pattern 285 is detected, with low variability, a median glucose <180 mg/dL, no hypo risks, and no excursions were detected, the considerations for the clinician 276 may include a most important patterns statement 278 that identifies the “Highs” pattern 285 and the time periods in which the pattern is detected. To differentiate a “Highs” pattern 285 from a “Lows” 281 and “High with some Lows” 283 patterns, each of the “Highs” pattern 285 may be highlighted in orange, while the “Lows” pattern 281 may be highlighted in red and the “High with some Lows” patterns 283 may be highlighted in dark red/maroon when that particular pattern is detected and reported and identified as the most important pattern. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider adjusting medication or starting medication other than insulin or sulfonylurea.” Because low variability was detected, the glucose patterns report 250 may not contain a variability statement 286. The considerations for the clinician 276 may also include self-care considerations 262. The statements regarding self-care may include, but are not limited to: “Meals or snacks often high in carbohydrates?” In the glucose concentration profile, the time periods with the “Highs” patterns 285 may be highlighted with boxes, e.g., orange boxes. As seen in FIGS. 6D-1-6D-2, where adjacent time periods are all determined to have a “Highs” pattern 285, then a single box 295 may outline the adjacent time periods.

In some embodiments, where only a “Highs” pattern 285 is detected, with low variability, a median glucose >180 mg/dL, no hypo risks, and no excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern. The statements may include, but are not limited to: “For T1 patients, consider adjusting insulin.”; “For T2 patients currently taking insulin or sulfonylurea, consider adjusting medication.”; and “For other T2 patients, consider starting insulin.” The considerations for the clinician 276 may also include self-care considerations 262. The statements regarding self-care may include, but are not limited to: “Meals or snacks often high in carbohydrates?” In the glucose concentration profile, the time periods with the “Highs” patterns 285 may be highlighted with boxes, e.g., orange boxes (see, e.g., FIGS. 6C-1-6C-2 and FIGS. 6D-1-6D-2). Where adjacent time periods are all determined to have a “Highs” pattern 285, then a single box or partial box 295 may outline the adjacent time periods (see, e.g., FIGS. 6C-1-6C-2 and FIGS. 6D-1-6D-2).

In some embodiments, where only a “Highs” pattern is detected, with low variability, any median glucose value, moderate hypo risk, and no excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “If starting or adjusting medication to address highs, consider how the medication could induce lows.” The considerations for the clinician 276 may also include self-care considerations 262. The statements regarding self-care may include, but are not limited to: “Meals or snacks often high in carbohydrates?” In the glucose concentration profile, the time periods with the “Highs” patterns may be highlighted with orange boxes (see, e.g., FIGS. 6C-1-6C-2 and FIGS. 6D-1-6D-2). Where adjacent time periods are all determined to have a “Highs” pattern, then a single box may outline the adjacent time periods (see, e.g., FIGS. 6C-1-6C-2 and FIGS. 6D-1-6D-2).

In some embodiments, where only a “Highs” pattern 285 is detected, with high variability, a median glucose <180 mg/dL, no hypo risks, and no excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern. The statements may include, but are not limited to: “For T1 patients, consider adjusting insulin.”; “For T2 patients currently taking insulin or sulfonylurea, consider adjusting medication.”; and “For other T2 patients, consider adjusting medication or starting medication other than insulin or sulfonylurea.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self-care considerations 262. The statements regarding self-care may include: “Medication sometimes missed?” and “Meals sometimes missed or vary in carbohydrates?” In the glucose concentration profile, the time periods with the “Highs” patterns 285 may be highlighted with boxes 295, e.g., orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 is detected, with high variability, a median glucose >180 mg/dL, no hypo risks, and no excursions were detected, the considerations for the clinician 276 may include a most important patterns statement 278 that identifies the “Highs” pattern and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider starting insulin.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self care considerations 262. The statements regarding self-care may include, but are not limited to: “Medication sometimes missed?” and “Meals or snacks sometimes high in carbohydrates?” In the glucose concentration profile 256, the time periods with the “Highs” patterns may be highlighted with orange boxes 295 (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a. “Highs” pattern 285 is detected, with high variability, any median glucose value, no hypo risks, and no excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern. The statements may include, but are not limited to: “If starting or adjusting medication to address highs, consider how the medication could induce lows.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self-care considerations 262. The statements regarding self-care may include: “Medication sometimes missed?” and “Meals or snacks sometimes high in carbohydrates?” In the glucose concentration profile 256, the time periods with the “Highs” patterns 285 may be highlighted with boxes, e.g., orange boxes (see, e.g., FIGS. 6C-1-6C-2 and FIGS. 6D-1-6D-2), Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a. “Highs” pattern 285 is detected, with low variability, median glucose <180, no hypo risk, and low excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider adjusting medication or starting medication other than insulin or sulfonylurea.” Because low variability was detected, the glucose patterns report 250 may not contain a variability statement 286. The considerations for the clinician 276 may also include self-care considerations 262. The statements regarding self-care may include, but are not limited to: “Meals or snacks sometimes high in carbohydrates?” The considerations for the clinician 276 may also include an excursion statement 288, The statement may include, but is not limited to: “Occasional hypoglycemia occurrences below 54 mg/dL. See Weekly Summary report.” In the glucose concentration profile 256, the time periods with the “Highs” patterns may be highlighted with orange boxes, e.g., orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 is detected, with low variability, median glucose >180, no hypo risk, and low excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider starting insulin.” Because low variability was detected, the glucose patterns report 250 may not contain a variability statement 286. The considerations for the clinician 276 may also include self-care considerations 262. The statements regarding self-care may include: “Meals or snacks often high in carbohydrates?” The considerations for the clinician 276 may also include an excursion statement 288. The statement may include, but is not limited to: “Occasional hypoglycemia occurrences below 54 mg/dL. See Weekly Summary report.” In the glucose concentration profile 256, the time periods with the “Highs” patterns 285 may be highlighted with boxes, e.g., orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 285 is detected, with low variability, any median glucose value, moderate hypo risk, and low excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “If starting or adjusting medication to address highs, consider how the medication could induce lows.” Because low variability was detected, the glucose patterns report 250 may not contain a variability statement 286. The considerations for the clinician 276 may also include self care considerations 262. The statements regarding self-care may include, but are not limited to: “Meals or snacks often high in carbohydrates?” The considerations for the clinician 276 may also include an excursion statement 288. The statement may include, but is not limited to: “Occasional hypoglycemia occurrences below 54 mg/dL. See Weekly Summary report.” In the glucose concentration profile 256, the time periods with the “Highs” patterns 285 may be highlighted with boxes, e.g., orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 is detected, with high variability, median glucose <180, no hypo risk, and low excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider adjusting medication or starting medication other than insulin or sulfonylurea.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self-care considerations 262. The statements regarding self-care may include, but are not limited to: “Medication sometimes missed?” and “Meals or snacks sometimes high in carbohydrates?” The considerations for the clinician 276 may also include an excursion statement 288. The statement may include, but is not limited to: “Occasional hypoglycemia occurrences below 54 mg/dL. See Weekly Summary report.” In the glucose concentration profile 256, the time periods with the “Highs” patterns 285 may be highlighted with boxes, e.g., orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 is detected, with high variability, median glucose >180, no hypo risk, and low excursions were detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “For T1 patients, consider adjusting insulin.”; “For T2 patients currently taking insulin or sulfonylurea, consider adjusting medication.”; and “For other T2 patients, consider starting insulin.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self-care considerations 262. The statements regarding self-care may include, but are not limited to: “Medication sometimes missed?” and “Meals or snacks sometimes high in carbohydrates?” The considerations for the clinician 276 may also include an excursion statement 288. The statement may include, but is not limited to: “Occasional hypoglycemia occurrences below 54 mg/dL. See Weekly Summary report.” In the glucose concentration profile 256, the time periods with the “Highs” patterns 285 may be highlighted with orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

In some embodiments, where only a “Highs” pattern 285 is detected, with high variability, any median glucose value, moderate hypo risk, and low excursions detected, the considerations for the clinician 276 may include a most important patterns statement 278 that identifies the “Highs” pattern 285 and the time periods in which the pattern is detected. The “Highs” pattern tag 280 may be highlighted in orange, while the time periods may be identified in text form. The medication considerations 260 may include statements for consideration by the clinician in determining treatment for the “Highs” pattern 285. The statements may include, but are not limited to: “If starting or adjusting medication to address highs, consider how the medication could induce lows.” The considerations for the clinician 276 may also include a variability statement 286 regarding high variability detected. The statements may include, but are not limited to: “Highs are often associated with high glucose variability” and “The following behaviors may contribute to high glucose variability,” which may be followed by a list of behaviors. The considerations for the clinician 276 may also include self-care considerations 262. The statements regarding self-care may include, but are not limited to: “Medication sometimes missed?” and “Meals or snacks sometimes high in carbohydrates?” The considerations for the clinician 276 may also include an excursion statement 288. The statement may include, but is not limited to: “Occasional hypoglycemia occurrences below 54 mg/dL. See Weekly Summary report.” In the glucose concentration profile 256, the time periods with the “Highs” patterns 285 may be highlighted with orange boxes (see, e.g., FIGS. 6C-1-6C-2 and FIGS. 6D-1-6D-2). Where adjacent time periods are all determined to have a “Highs” pattern 285, then a single box 295 may outline the adjacent time periods (see, e.g., FIGS. 6C-1-6C-2 and FIGS. 6D-1-6D-2).

As seen in FIGS. 6B-1-6B-2, where multiple types of patterns are detected, all of the patterns may be identified in the glucose concentration profile 256. In some embodiments, the glucose concentration profile 256 may include up to 3 patterns per patient. The boxes or partial boxes or brackets 295 outlining the various patterns may be prioritized. The order of priority from first to last may be “Lows,” “Highs with Some Lows,” and “Highs.” The various patterns may be color-coded based on priority. In some embodiments, the highest priority pattern of the patterns that are detected may be the only pattern highlighted with a color. The remaining lower priority pattern(s) may be colored a different color, e.g., gray or black. For example, where at least one of each of a “Lows” 281, “Highs with Some Lows” 283, and “Highs” 285 pattern is detected, the “Lows” pattern 281 may be displayed in red (in a partial box 295 and label), while the “Highs with Some Lows” 283 and “Highs” 285 patterns may be displayed on the glucose concentration profile 256 in gray. In other embodiments, where at least one of each of a “Highs with Some Lows” 283 and “Highs” 285 pattern is detected, the “Highs with Some Lows” 283 may be displayed in dark red or maroon (in a partial box 295 and label), while the “Highs” patterns 285 may be displayed on the glucose concentration profile 256 in gray.

If a patient is found to have met all of the TIR target goals for a time period, but a “Lows” pattern is detected for at least one time period, then the report may still identify the “Lows” pattern in all of the appropriate sections, including the boxes or partial boxes or brackets 295 outlining the “Lows” pattern on the glucose concentration profile 256 and the most important patterns statement 278, In an alternate embodiment, if a patient is found to have met all of the TIR target goals for a time period, no pattern may be identified or highlighted in the report.

In embodiments where only a single pattern is detected, the single pattern may be displayed on the glucose concentration profile 256 in color. For example, the “Lows” pattern 281 may be displayed in red (in a partial box 295 and label), the “Highs with Some Lows” pattern 283 may be displayed in dark red or maroon (in a partial box 285 and label), and the “Highs” pattern 285 may be displayed in orange (in a partial box 295 and label).

Learning Method

Manual configuration of the DGS 100 can require time by an HCP, who may not have sufficient time available. In addition, even if HCP time is available, configuration may be complex and is potentially error prone. To mitigate these issues, a patient parameter initialization (PI) module where setup is not required, or only minimal setup is required, can be included in the DGA. The PI module learns a patient's dosing strategy, which can comprise, for example, basal only, basal plus one, basal plus two, etc., and parameterizes the patient's medication dosing practice for configuring dose guidance settings by the DGA.

According to an aspect of the embodiments, the PI module's learning process can include automatically configuring patient dose guidance settings from observed data. Once the settings are successfully learned, the DGS 100 can enter a guidance mode, wherein the patient can ask for dose guidance and receive notifications about dosing. During the learning process that precedes the guidance mode, the DGA can process glucose and insulin data collected by the patient's SCD 102, UID 202, and/or other devices, and determine dosing information based on the processed data.

Dosing information can include, for example, dose regimen, meal-dose type, dose parameters, and dose range. Dose regimens may include, for example, basal dose plus BF, basal plus LU, basal plus DI, basal plus BF/LU, basal plus BF/DI, basal plus LU/DI, and basal plus 3, wherein BF indicates “breakfast,” LU indicates “lunch,” and DI indicates “dinner.” Additional regimens can also be included, e.g., afternoon snack doses. Meal-dose type can include, for example, fixed meal dose or variable meal dose. Dose parameters can include, for example, a nominal fixed dose or carbohydrate ratio for each meal, a premeal correction factor (CF) and a post-meal CF. Dose range may include an estimate of the lowest meal dose. CF is also known as insulin sensitivity factor. It is a ratio that reports how much 1 U of insulin will lower blood glucose in either fasting or premeal state. The DGA may have two CF values to account for insulin sensitivity differences between fasting and premeal physiology: premeal and postmeal. CF has units of (mg/dL)/Unit insulin.

For each of the above dosing information types, the DGA can determine whether the cumulative data are sufficient or insufficient for determining the dosing information. In some embodiments, the patient's SCD 102 can be configured to operate for a predefined time period, for example, 14 days. In these embodiments, the DGA can determine after the predetermined time period (or earlier if the sensor stops working prior to end of the period), whether the available analyte and dosing data are sufficient to determine each of the above dosing information. If so, the DGA can perform the parameterization method 300 and allow the start of the dose guidance mode. In alternative embodiments, periodically during the learning period (such as once per day), the DGA can determine if data are sufficient to determine each of the above dosing information. In either case, when the collected data are sufficient, the DGA can end the learning period, perform the parametrization, and begin the guidance period. If not, then the DGA may continue with the learning process.

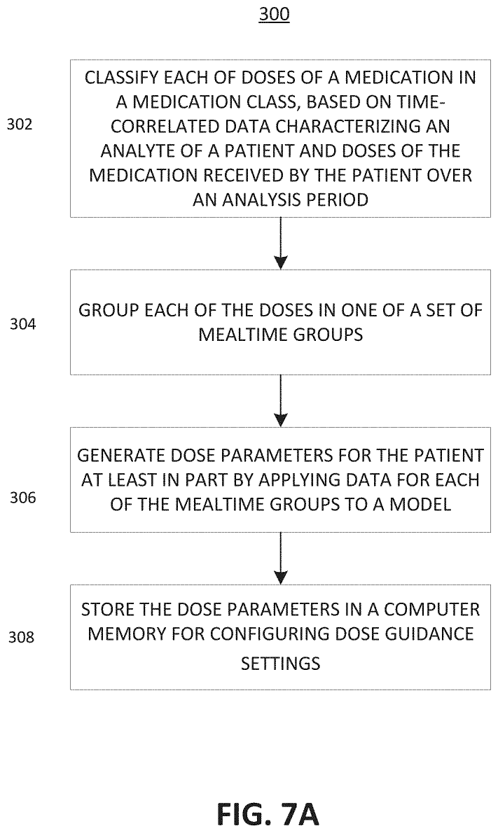

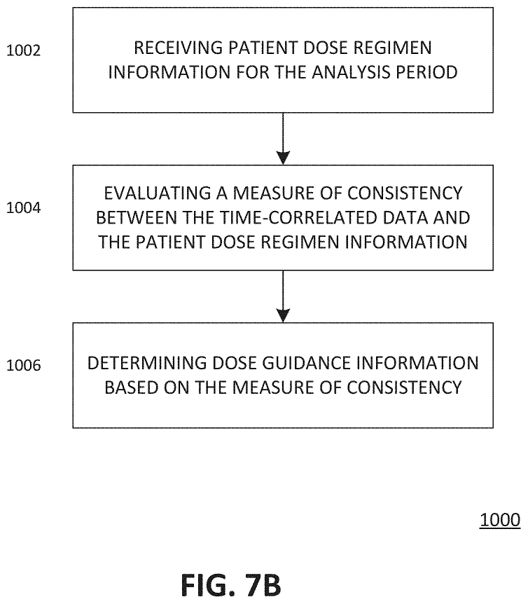

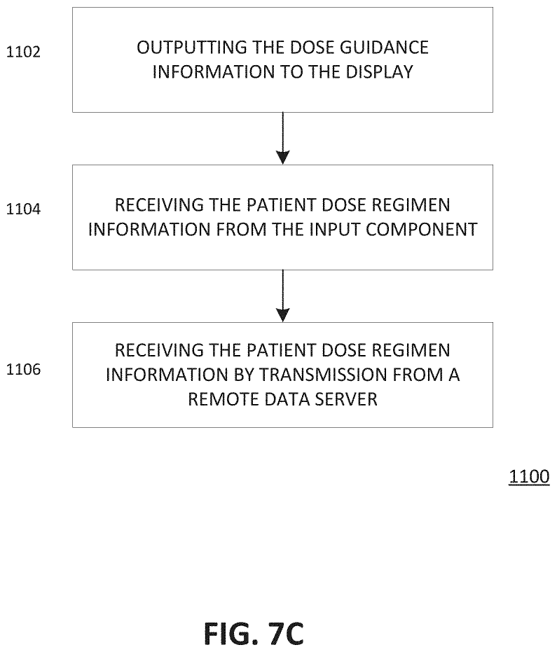

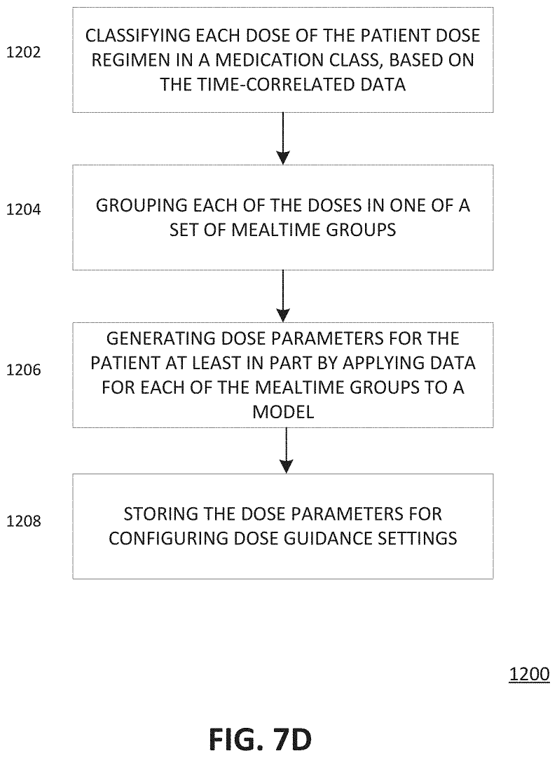

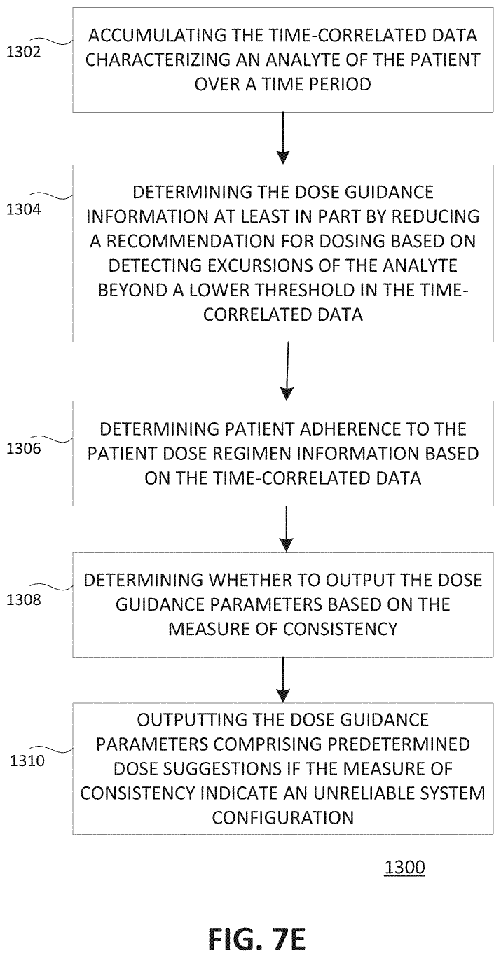

Referring to FIG. 7A, a DGA can be configured to perform the method 300 on a suitable computing device, for example the UID 200, SCD 102, MDD 152, either alone or in any combination. Program instructions for performing the method 300 can be grouped in a PI module or any other suitable code configuration. In overview, the method 300 can include, by the DGA at step 302, classifying each of doses of medication received by a patient over an analysis period, based on data characterizing an analyte of the patient and the doses of a medication received by the patient over the analysis period. The method 300 can further include, at step 304, grouping each of the doses in one of a set of mealtime groups. The method can further include, at step 306, generating dose parameters for the patient at least in part by applying data for each of the mealtime groups to a model. The method can include, at step 308, storing the dose parameters in a computer memory for configuring dose guidance settings. In embodiments described herein, the analyte can be glucose, or can include an indicator of the patient's glucose level, and the medication can be, or can include, insulin. The dose guidance setting can be used by the DGA for developing dose guidance, or provided to an interface device, for example the UID 200 or health care practitioner's terminal, for output. More detailed aspects of each of the operations in the method 300 are described below. As used herein, “PI module” refers to a portion or portions of the DGA that perform the operations of the method 300 and any ancillary operations. A PI module is not limited to a particular configuration, and can encompass various arrangements of computer code.

In an aspect, the classifying operation 302 can include classifying each dose of medication (e.g., insulin) as one of a meal dose, a correction dose, and/or an ambiguous dose. If the DGA cannot classify a medication dose as a meal dose or correction dose to a defined degree of confidence, the DGA can classify the dose as ambiguous, and can omit said dose from use in generating dose parameters for dose guidance.

The DGA can perform medication dose classification by a sequence of two operations, referred to herein as feature extraction and classification. Relating this to FIG. 7A, the classifying operation 302 can include generating a feature matrix correlating a set of classification features to each of the doses. In some embodiments, the DGA can configure a vector of insulin injection timestamps, a data file that includes analyte measurements from the patient's SCD 102, and the results from a meal detection algorithm module discussed elsewhere herein, as input to a function that outputs a feature matrix for insulin dose classification. The number of rows of the feature matrix can indicate a quantity of injections, or equivalent medication dosing events, during the relevant analysis period. Each row in the feature matrix can be, or can include, a feature vector for a single dosing event. In embodiments for classifying insulin injections, each vector can include elements as described below, referred to herein as classification features. The DGA can determine each of the elements of the feature vector based on a corresponding segment of glucose monitoring data in a time range, for example, between −2.5 and 1.5 hours, relative to the insulin injection time.

In embodiments, the classification features can include a medication time for each dose, e.g., a time of day that an insulin injection is recorded by the MDD 152 or recorded by the patient using the UID 200.

The classification features can further include a time-filtered analyte value, for example, a glucose value filtered using a Savitsky-Golay filter, a low-pass filter, a band-pass filter, a nonparametric smoothing filter such as locally estimated scatterplot smoothing, or other filters. In an aspect, the Savitsky-Golay filter can be of order 2, frame length 7 on a 15 minute sampling interval.

The classification features can further include a rate of change of the analyte value closest to the time of medication, for example, a rate of change of the analyte (e.g., glucose) value computed by linear regression of five analyte data points (e.g., using a 15 minute sampling interval) centered at the data point closest to the medication (e.g., injection) time.

The classification features can further include a left Area-Under-Curve (AUC) metric indicating an integrated difference between analyte values and the analyte value closest to the time of medication over an interval prior to the medication time. For example, to obtain the left AUC metric, the DGA can compute the left AUC metric by collecting all data points from the filtered analyte data within a time window (e.g., 2.5 hours), counting back from the injection time, then computing a difference between the mean analyte value of the collected data points and the data point closest to the injection time (i.e., the reference data point), and computing the incremental AUC on the left by multiplying the difference by the duration of the time window.

The classification features can further include a right AUC metric, indicating an integrated difference between analyte values the analyte value closest to the time of medication over an interval after the medication time. For example, the DGA can compute the right AUC metric by collecting all data points from the filtered analyte data within a time window (e.g., 1.5 hours), counting back from the injection time, then computing a difference between the mean analyte value of the collected data points and the data point closest to the injection time (the reference data point), and computing the incremental AUC on the right by multiplying the difference by the duration of the time window.

The classification features can further include time elapsed between medication times. For example, the DGA can, for each injection time, compute an elapsed time between the previous and the current injection time by subtracting the previous injection time from the current injection time. For the first injection time in the insulin log, the DGA can compute the elapsed time from the first SCG time data point to the current injection time because there is no previous injection time available. In addition, and for further example, the DGA can compute the elapsed time between the current and the next injection time by subtracting the current injection time from the next injection time. For the last injection time in the insulin log, the DGA can compute the elapsed time from the current injection time to the last SCG time data point because there is no next injection time available. In both the backward and forward computations, if the elapsed time is larger than a predetermined maximum value, e.g., 12 hours, the DGA can set the elapsed time value equal to the maximum time.

The classification features can further include probability of a meal starting within a defined interval prior to the medication time, for example, the maximum of the probability of meal start within a time window (e.g., 1.5 hours) prior to the injection. This probability can be computed by a meal detection module, described elsewhere herein.

The classification features can further include a most probable interval of time elapsed since the most recent meal, for example, an elapsed time from the maximum meal start probability point (e.g., determined by a meal detection module) relative to the injection time.

The classification features can further include probability of a meal starting within a defined interval after the medication time, for example, a maximum of the probability of meal start (determined by a meal detection module) within 2 hours after the injection.

The classification features can further include a most probable interval of time until the next meal, for example, a predicted elapsed time from the injection time to the maximum meal start probability point (e.g., determined by a meal detection module) after the meal injection.

As noted, computing some of the classification features includes estimating a time for each meal eaten by the patient during the analysis period, and methods for estimating mealtimes are described in more detail below. In brief, estimating the time for each meal can further include, by the DGA, generating a feature matrix based on the time-correlated analyte data, wherein the feature matrix correlates a set of analyte (e.g., glucose) data features to each of distinct regions classed as rising, fall-preceding, and falling. The set of analyte data features can be, or can include a maximal analyte rate of change, a maximal analyte acceleration, an analyte value at the maximal analyte acceleration point, a duration of the region, a height of the region, a maximal deceleration, an average rate of the change in the region, and a time of the maximal analyte acceleration. The estimating can further include generating estimated mealtimes based on the feature matrix, using an algorithm as described below.

More detailed aspects of a retrospective mealtime detection algorithm for use in the method 300, or for other uses, are described in the following paragraphs. Afterwards, description of other aspects of the method 300 is continued. A DGA can perform retrospective mealtime detection based on time-correlated analyte data by executing one or more code modules, for example, a feature extraction module and a meal detection module. A feature extraction module, when executed by the DGA, can cause the DGA to receive a glucose time series as input and output a feature matrix to be passed to the retrospective meal detection module to detect glucose excursions in response to meal events.

A DGA can perform feature extraction using the following operations as described below, which may be divided into a sequence of three sub-operations: smoothing, segmenting, and extracting.

In a smoothing sub-operation, the DGA can smooth an analyte (e.g., glucose) time series using a Savitzky-Golay filter (order 2) and compute a rate of change and acceleration at each analyte data point. The frame length parameter for the filter may be the number of the data points collected in a first time interval, e.g., 60 minutes; therefore, the sampling is interval dependent. The DGA can compute the rate of change by taking the average of the backward and forward difference in the smoothed analyte values between the point of interest and the points that are a second interval (e.g., 15 minutes) before and after the point of interest, wherein the second interval is less than the first interval, for example, equal to one-fourth of the first interval. Similarly, the DGA can compute the acceleration by taking an average of the backward and forward difference in the analyte rate of change between the point of interest and the points that are the second interval (e.g., 15 minutes) before and after it.

In a segmenting sub-operation, the DGA can segment the smoothed analyte trace into monotonically increasing (i.e., rising) and decreasing (i.e., falling) regions. Each rising region is considered as a candidate of a glucose excursion in response to a meal event.

In an extracting sub-operation, the DGA can extract the features from the data, for example, sixteen (16) features that may be or may include features from each rising region (e.g., eight (8) features), the preceding falling region (e.g., four (4) features), and the following falling region (e.g., four (4) features). Features that the DGA can extract from the rising features can include, for example: 1) the maximal analyte rate of change, 2) the maximal analyte acceleration, 3) the analyte value at the maximal analyte acceleration point (the reference point), 4) the duration of the rising region (the elapsed time from the reference point the to the last point of the region, 5) the height of the region (the difference in the smoothed analyte value between the last point and the reference point), 6) the maximal deceleration (the negative acceleration with maximal absolute value), 7) the average rate of the change in the region (height/duration), and 8) the time of the data of the reference point. For further example, four (4) features extracted from the preceding and the following falling regions may include: 1) the height of the falling region, 2) the duration of the falling region, 3) the average rate of the region (height/duration), and 4) the maximal absolute value of the glucose rate of change. The number of rows in the feature matrix output by the feature extraction module may be the same as the number of rising regions in smoothed glucose time series.

According to another aspect of the embodiments, the retrospective meal detection module can take the feature matrix, as input, and output binary detection results for each rising region. Such output can include: a binary classification result, and a probability value of each rising region being an analyte (e.g., glucose) excursion in response to a meal event. The DGA can assign a probability value for each rising region to its reference point. In some embodiments, for example, a pre-trained machine learning model for meal detection can be implemented using RandomForestClassifier by scikit learn (https://scikit-learn.org/stable/modules/generated/sklearn.ensemble.RandomForestClassifier.html). The meal detection module may detect meal-induced postprandial glucose excursions based on a multitude of decision trees constructed and optimized during the training process. In alternative embodiments, the DGA can build a pre-trained model based on an alternative classification algorithm, for example, gradient boosting, ada boost, artificial neural network, linear discriminant analysis, and extra tree.

Referring again to the method 300 of FIG. 7A, the classification operation 302 can take the feature matrix of a patient as input and output a binary classification result for each relevant medication event (e.g., for each insulin injection). For example, the DGA can output binary data ‘1’ signifying a meal dose and ‘0’ signifying non-meal dose. According to some embodiments, the classification operation 302 can use meal detection results, in which case a meal detection can be performed prior to an insulin dose classification. As noted for retrospective mealtime detection, the classification operation 302 can include a pre-trained machine learning model, for example a model implemented using RandomForestClassifier by scikit learn (referenced above). The machine learning model implemented by the DGA can perform classification based on tree building rules and thresholds for various features in each decision tree which were optimized during the training process. Alternatively, the model can also be trained by other machine learning algorithms including gradient boosting, ada boost, artificial neural network, linear discriminant analysis, and extra tree. After the DGA successfully classifies each dose, it can proceed to determining the dose regimen and dose parameters.

At step 304, the method 300 can include the DGA grouping each of the doses in one of a set of mealtime groups or clusters. For example, the DGA can determine a dosing strategy by clustering analysis of the medication (e.g., injection) times of meal doses. The DGA can execute a clustering module, implemented using a K-mean algorithm together with the elbow method, that takes the injection times, as input, and outputs the optimal number of clusters K (maximum 3) and the cluster index for each injection time. The optimal number of clusters K can be the number of meal doses taken by the patient per day. Using the cluster index of each injection, the DGA can group the meal dose into K groups according to the cluster indexes.

The DGA can identify these groups as breakfast, lunch or dinner (B, L, D) as follows: for each group, the DGA can determine a typical time-of-day (TOD) by calculating the median TOD for the group. Alternatively, the DGA can use some other centroid metric. If K=3, then the DGA can associate breakfast with the group after the longest period between typical group TODs. Then, the next group is lunch, and the last group is dinner. If K=2, the DGA can estimate which group is associated with breakfast, lunch or dinner using assumed rules about the time between each meal. For instance, if the two groups are more than six (6) hours apart from each other, then the DGA can identify the groups as breakfast and dinner. Otherwise, if a first group occurs before 10 am, then the DGA can identify the groups as breakfast and lunch; otherwise, as lunch and dinner. In an alternative embodiment, the DGA can prompt the user to identify the meal associated with each typical time, after the DGA identifies the typical times for meal events. For further example, in alternative embodiments, the DGA can combine the two methods described here by estimating the meal associations and then prompting the user to confirm. Further alternative methods can include analyses of glucose data to identify meals and cluster mealtimes to detect typical mealtimes. This can be useful for distinguishing meals in the case of K=2; that is, identifying the meal where a dose is not taken.

Once the doses are grouped in mealtime clusters, at step 306, the DGA can perform generating dose parameters for the patient at least in part by applying data for each of the mealtime groups to a model. For example, for each meal group (B, L, D), the DGA can pair each corresponding set of pre-meal glucose levels with the corresponding meal dose amount. The DGA can fit each group with a suitable model, for example, a linear function with zero slope, a linear function with non-zero slope, a piece-wise linear function joined at a single point, or a nonlinear function that approximates a joined piecewise model but with smooth curvature around the joint point. Other models are also suitable.

The DGA can perform model fitting and parameter estimation by minimizing the sum of square residual (SSR) in terms of model parameters. Then, the DGA can use a search algorithm to find the optimal parameters that results in the smallest value of the SSR. For linear models, the DGA can use a Nelder-Mead simplex method for fitting. For a non-linear model, the DGA can use a Levenberg-Marquardt algorithm. That is, the DGA can use the Nelder-Mead simplex numerical optimization method for linear models and the Levenberg-Marquardt optimization method for non-linear models. Alternative methods of fitting the data to these models are also possible.

When the number of iterations during optimization exceeds the convergence criteria, the model fails to fit, and the DGA can exclude the model that failed to fit as a candidate model. Additionally, the DGA can apply certain rules to minimize uncertainty in parameter estimation, for example, by validating an estimated correction factor by requiring at least three pre-meal glucose datapoints greater than the estimated threshold glucose; validating an estimated fixed dose by requiring at least three pre-meal glucose datapoints less than the estimated threshold glucose; requiring a 95% confidence interval of the parameter intercept to exclude zero; or requiring a 95% confidence interval of the model slope to exclude zero.

Insufficient data can lead to a failure of fitting a model and, consequently, the exclusion of a particular model as a candidate model. The DGA can evaluate each model with Akaike Information Criterion (AIC), and choose the model with the lowest AIC value as the preferred model for each meal group.

Once it has selected the models for each mealtime cluster, the DGA can then determine dose parameters including, for example, fixed dose insulin amount, target glucose level, and a correction factor based on a selected model for each mealtime cluster. The DGA can determine the target glucose level and the correction factor as a single value each for all groups, as described in more detail in the following paragraph. In alternative embodiments, the DGA can determine the target glucose level and the correction factor separately for each group and use the separately determined parameters for downstream dose guidance operations.

According to another aspect of the embodiments, the DGA can form a combined data group for obtaining a more accurate correction factor for the patient. For example, after fitting the dose data to various models for each meal group to select the best model and estimate a fixed inulin dose amount, the DGA can deduct the fixed dose insulin amount from an associated meal dose of each meal group. Remaining non-zero values correspond to doses that have correction amounts. Those non-zero values can then be combined from all three meal groups (B, L, D) to form a combined group. If the fixed dose insulin amount cannot be determined for a group, the DGA can exclude data of the group from the combined group. The system can then repeat the operations for finding a best-fitting model for the combined group, or can use the same model identified when the groups were analyzed separately. The use of this combined group approach assumes the patient has the same (or constant) correction factor and target glucose across all meals, and the combined group provides a larger sample size for potentially more accurate fitting. The DGA has completed the estimation of dose parameters after it determines the target glucose level and correction factor based on the best-fitting model. Then, at step 308, the DGA can store the dose parameters in a computer memory for configuring dose guidance settings.

In an additional aspect, the DGA can determine whether the patient is potentially carbohydrate-counting (e.g., varying their meal dose to account for carbohydrate consumption) by comparing the AIC value of the preferred model to a threshold, such as 50, or 75, or 100. If the AIC value is larger than the threshold, the DGA can determine that the patient is carbohydrate counting and ask the patient to confirm via the UID 200.

In alternative embodiments, one or more of the operations described above may be omitted and replaced by requesting that the patient or HCP provide information manually, or by extracting information from another source such as an EMR or another software program. Nonetheless, the method 300 should be useful for various applications without information beyond what an SCD and MDD can provide.

### Alternative Learning Method

In an alternative embodiment, DGS 100 (e.g., SCD 102, display device 120, or MDD 152) can be configured using an automatic or semiautomatic learning method that classifies and characterizes medication doses based on patient input and the patient's GPA analyzed over a learning period. An alternative learning embodiment encompasses regimen inputs from the user, processing of recorded glucose measurements (glucose reading and associated date/time stamp) and insulin dosing information (dose amount and associated date/time stamp). The inputs and algorithms, however, may be optimized to reduce burdens on patients and HCPs. For example, in the presently described embodiment, HCP involvement may not be required to initiate dose guidance. This is advantageous because HCPs often do not have time to configure prior systems for the patient. In addition, the present embodiment provides an additional safety mechanism compared to the common practice of having a patient or HCP directly configure the dose guidance system (or, more commonly termed, the bolus calculator). Specifically, because connected insulin pens are becoming more common and have been used recently in studies, it is apparent that many MDI patients are non-adherent to their dose regimen, such as missing meal doses, dosing late, or taking less insulin than required. The first two are likely due to inconvenience or forgetfulness, whereas the third may arise from fear of the hypoglycemia, coupled with lack of self-confidence competency to manage hypoglycemia.

A fundamental problem with common practice is that the dose guidance system may be configured based on what the patient or HCP thinks is necessary based on their glycemic profile. That profile, however, may be based on a prior regimen that had poor adherence. For example, an HCP may recommend that a patient increase their fixed insulin dose for breakfast from 10 to 12 to mitigate high post-breakfast glucose, whereas this high glucose was actually caused by the patient missing 50% of their breakfast doses or underdosing by often only taking 8 units. If the patient suddenly decides to increase their adherence, this could cause a problem with hypoglycemia. Therefore, it is important that the dose guidance setup process include analysis of prior adherence before dose guidance is presented to the patient.

As described previously, the patient or HCP may enter all key dose guidance parameters or just a portion of them. Here, as an example, it is assumed that the patient enters the following portion of the dose guidance parameters—other examples may be contemplated. Specifically, the system provides a means for entering, prior to providing dose guidance, the following parameters: typical fixed doses for breakfast, lunch and dinner; and typical time of day when breakfast (and/or each meal) takes place.

The foregoing exemplifies a compact set of parameters that should be easy enough for most patients to understand and be able to enter correctly. It avoids complex parameters, like correction factor, that many patients do not understand. Alternatively, the system may allow patients to optionally enter more parameters, for patients who have more sophisticated dose regimens and/or understand these regimen details enough to enter the parameters correctly. In another embodiment, the patient may enter information about their level of adherence, such as how often they miss doses at certain times of day or how often they skip meals and therefore don't need to dose insulin. Another embodiment is a system where the patient is prompted to manually classify doses that are recorded. This type of system, however, will likely require a high degree of interaction with the patient for no immediately received value, so may be less preferred for most patients.

During a learning mode for analyzing patient's adherence to a user-entered regimen, the system will acquire glucose data and insulin dose information over a period, for example, two weeks. If the system does not initiate dose guidance after this period, then it may allow the patient to initiate a subsequent period for observation.

At the end of the learning period, the system processes the entered regimen parameters and the observed glucose and insulin dose information, using an analytical algorithm executed by a processor. The algorithm may include classifying doses as previously described. In an alternative, or in addition, the classifying may be based on dosing information entered by the user, and may take into account absolute time of day and relative time between measured analyte (e.g., glucose) data and user-supplied dose data. The classification may, in an alternative or in addition, be based on dynamic relationships between the different data inputs, for example, rate of change of glucose. The system may perform the classifying using a classification model, which may be developed and trained using common machine learning techniques applied to clinical data and simulated data.

Once the system processor classifies the doses, it associates each dose with one of several meal events during the day. Clustering analysis previously described, is one method for performing the association. For example, the system may associate with breakfast one of the clusters whose time-of-day central measure (e.g., median, mean), is closest in time to the user entered value for breakfast; the next cluster in time with lunch and the next with dinner.

An alternative clustering method may include glucose data as well as dose information in algorithmic cluster determination for identifying the clusters and which doses are associated with which cluster. In addition, the processor may further include data indicating absolute time of day and relative time between glucose and dose data in its cluster analysis. For instance, a dose of 10 units in the morning may be associated with the lunch cluster if 10 units is a common dose for this cluster and not for the breakfast cluster, and if the dose was taken late enough in the morning. The association may also be based on dynamic relationships between the different data inputs, such as rate of change of glucose. The determination is made by a classification model, where this classification model may be developed and trained using common machine learning techniques applied to clinical data and simulated data.

Once the clustering process is completed, then the system estimates the regimen parameters, as described previously. The parameter estimation process may include a determination of a degree of confidence in the parameter estimate, using standard numerical analysis technique. For the subsequent description, for simplicity the degree of confidence may be described as the confidence interval; however, there are other common measures of confidence that may be used. These parameters are referred to herein as the “learned” regimen parameters, which includes the parameter value and associated confidence interval.

Once the regimen is learned, the system may perform the following checks between the entered parameters and the learned parameters. The system processor may check the confidence interval (CI) for the learned meal dose parameters and typical dose times by comparing to a maximum threshold value appropriate for each parameter. If the CI exceeds that threshold, then the processor may flag the system configuration as suspect. For the meal dose amounts, an appropriate maximum CI is +/−30%, or 20%, or 50%; for the typical meal dose times, an appropriate maximum CI is +/−½ hour, 1 hour, 2 hours.

In another aspect, the system processor may compare the learned parameters to the user entered parameters; specifically, the dose amounts for breakfast, lunch and dinner. If any of the parameters differ between entered and learned by more than the confidence interval (or some multiple of the confidence interval), then the processor may flag the system configuration as suspect.

The system processor may further compare the entered typical breakfast dose time with the learned meal dose time for the dose cluster closest in time. If these parameters differ by more than the processor may flag the confidence interval (or some multiple of the confidence interval) as suspect.

In addition, the system may check the additional learned parameters for reasonableness. For instance, the largest gap between the learned typical meal dose times should occur prior the learned typical meal dose time, to ensure that the overnight period is properly accounted for. If any reasonableness check fails, then the system configuration may be flagged as suspect. Some parameters do not have to be estimated with a high degree of confidence for the system configuration to not be flagged as suspect. For the target glucose (GT), pre-meal CF and post-meal CF, if the CI is within a maximum threshold appropriate for that parameter, then the learned value is used in the dose guidance regimen; otherwise, a conservative default value is used. An appropriate maximum CI threshold for GT and CF is +/−2, 5, or 10 mg/dL or +/−5, 10 or 20 mg/dL/unit, respectively. Appropriate default values for GT, CF premeal or postmeal CF are 120, 125, or 130 mg/dL, or 40, or 50 mg/dL/unit, or 60, or 70 mg/dL per unit.

In addition, the system may assess the acquired data for patient compliance with their entered regimen. For example, a processor of system may estimate the frequency of missed meals (or meals that have a small impact on post-prandial glucose levels). This is useful for calculating subsequent metrics. This estimate may be calculated using a model developed using machine learning or other techniques from glucose data and insulin dose data and meal records—this model may be trained with clinical data, real-world data, or simulated data. Generally, during a time-of-day period when a meal dose is expected, if an insulin dose does not occur and the glycemic pattern during this time did not show a rise in glucose, this is indicative of a missed meal.

The system processor may estimate the frequency of missed meal doses each for breakfast, lunch and dinner. For example, the frequency may be calculated as (number of periods missing a dose—number of periods tagged as a missed meal), divided by (number of periods total—number of tagged as a missed meal).

In another aspect, the system may estimate for each meal (breakfast, lunch and dinner) the difference between the median dose and the entered dose; that is, how much the patient is under or over dosing compared to what they entered. Prior to initiating dose guidance, the system processor may output to a display device or equivalent device the dose adherence findings. Similarly, the system may report to the patient the estimated impact on their glycemic management and A1 c due to the lack of adherence; or conversely, indicate to the patient potential improvements in their glucose metrics, like average glucose or time-in-range, or in lab measurements like A1 c, if they were to correct their adherence issues. This information can be generated by the processor from a model developed by correlating each diabetes control measure with adherence measures for specific regimens. A simple model would be based on real or simulated population data, and correlation parameters could be found to relate adherence measures to glycemic measures which have been in turn correlated to A1 c measurements. More advanced models could be developed and implemented by a system processor for relating specific patient characteristics like regimen followed and glycemic profile to adherence measures.

In summary of the foregoing, and by way of additional example, a DGS 100 or component thereof (e.g., one or more of an SCD 102, display device 120, or MDD 152) for parameterizing a patient's medication dosing practice for configuring dose guidance settings may include an input component configured to receive measured analyte data, meal data, and medication dosing data, a display component configured to visually present information, and one or more processors coupled with the input, the display, and a memory. The memory may hold instructions and time-correlated data characterizing an analyte of the patient over an analysis period, wherein the instructions, when executed by the one or more processors, cause the apparatus to perform a method 1000, as shown in FIG. 7B. In an aspect, the medication may be, or may include, insulin.

The method 1000 may include, at 1002, receiving, by the one or more processors, the patient dose regimen information for the analysis period. The one or more processors may hold the patient dose regimen information in a memory for processing. The method 1000 may further include, at 1004, evaluating a measure of consistency between the time-correlated data and the patient dose regimen information. In an aspect, the patient dose regimen information may be, or may include, typical fixed medication doses taken at mealtimes and a typical time of day when breakfast is eaten. For further example, the patient dose regimen information may be, or may include, information defining a frequency of patient compliance with scheduled doses or meals. In some embodiments, a patient dose regimen may include information relating to the type, amount and/or timing of medication. In particular, information relating to a schedule for dosing one or more medications.

A measure of consistency may include any numerical measure for comparing consistency between data sets, for example, a mean and standard deviation, or an inter-quartile range (IQR). Evaluating a measure of consistency may include comparing to a fixed or variable threshold. A measure of consistency may also be, e.g., a measure of the variation between the time-correlated data and expected data calculated based on the patient dose regimen. A variable threshold may be determined using a machine learning or other algorithm. More specific examples are provided herein above and below.

The method 1000 may further include, at 1006, determining dose guidance information based on the measure of consistency. Once determined, the processor may output the dose guidance information to a display or other output device for use by the patient or HCP, or store in a memory for later use. In an aspect, the dose guidance information may be, or may include, dose guidance information as exemplified herein above. Dose guidance may also be information that varies the type, quantity or timing of a dosage of a particular medication.

The method 1000 may include additional operations 1100, 1200, and/or 1300 diagrammed in FIGS. 7C-7E. The additional operations may be performed by the one or more processors of the DGS 100 in any operable order, and the presence of absence of any one or more of the operations shown in any figure does not necessarily imply a corresponding presence or absence of other operations shown in the figure. Instructions for performing the method 1000 and/or any one or more of the additional operations 1100, 1200 and 1300 may be held in a memory for execution by the one or more processors of a DGS.

Operations 1100 of the method 1000, as shown in FIG. 7C, may include at 1102, outputting the dose guidance information to the display or other output device. The method may further include, at 1104, receiving the patient dose regimen information from the input component, for example, via an input from a touchscreen of a display device. In an alternative, or in addition, the method may include at 1106 receiving the patient dose regimen information by transmission from a remote data server.

The method 1000 may further include the operations 1200 for evaluating a measure of consistency, as shown in FIG. 7D. The method 1000 may include, at 1202, classifying each dose of the patient dose regimen in a medication class, based on the time-correlated data. The method may include, at 1204, grouping each of the doses in one of a set of mealtime groups, for example, breakfast, lunch, and dinner, based on a time of day and/or other factors. Alternatively, the method may include grouping each of the doses by time periods, e.g., an hourly analysis. The method may include, at 1206, generating dose parameters for the patient at least in part by applying data for each of the mealtime groups to a model. In some embodiments, the model may be based on historic data from the user or from a population study. The method may further include, at 1208, storing the dose parameters for configuring the dose guidance settings.

The method 1000 may include further operations 1300, as shown in FIG. 7E. The method 1000 may include, at 1302, accumulating the time-correlated data characterizing an analyte of the patient over a time period, for example, 10 days, 14 days, or 21 days, prior to the evaluating the measure of consistency. The method may include, at 1304, determining the dose guidance information at least in part by reducing a recommendation for dosing based on detecting excursions of the analyte beyond a lower threshold in the time-correlated data. The recommendation should correlate to a meal dose which with the excursions are associated.

The method may include, at 1306, determining patient adherence to the patient dose regimen information based on the time-correlated data. The method may include, at 1308, determining whether to output the dose guidance parameters based on the measure of consistency. For example, if the measure of consistency indicates sufficient patient adherence, the dose guidance information is provided as determined by the DGS. If adherence is marginally consistent, then the DGS provides dose guidance but with warnings about adherence. If adherence is not consistent, then the DGS provides information to the patient or HCP about what needs to be corrected before guidance can be provided.

In an aspect, at 1310, the method may include outputting the dose guidance parameters comprising predetermined dose suggestions if the measure of consistency indicates an unreliable system configuration, such as unreliable data. The predetermined dose suggestions may be retrieved from a memory as a fallback when dose guidance is not available by the method in use.

### HCP Engagement During the Learning Period

If the system configuration is tagged as suspect, dose guidance may not be automatically initiated. The DGS 100 may provide two options for the patient: (a) address deficiencies described in the learning/adherence report and repeat the learning period, or (b) review the results/outputs of the learning period on their next visit to their HCP. For the second option, the system will allow the HCP to configure the system and initiate dose guidance or, counsel the patient on how to address the deficiencies outlined in the report and repeat the learning period.

Many HCPs lack the time to acquire a web portal account and configure patients' dose guidance systems. Therefore, the DGS 100 may provide a highly efficient means to facilitate HCP assistance with this configuration. When the learning period has completed and the system configuration is tagged as suspect, the DGA may display a control feature that will allow the patient to initiate the following process that facilitates HCP configuration of the system.

When the patient has their next contact (e.g., in person or via telehealth) with their HCP, the patient may press a button or select an option that will start this process. The DGA may then display a URL and/or a code for the HCP to enter into their web browser. The code may be randomly generated by the DGA and associated with that patient. When the HCP enters the URL into their internet browser, the DGA may open a webpage that provides the UI control for entering these codes. The code, for example, may be 4 alpha-numeric characters or any other acceptable form. The DGA may only treat this code as valid for a limited amount of time, e.g., 5 minutes or 15 minutes. When the HCP enters the code, the DGA may check if the code is valid, and if it is valid, provide the HCP with access to the patient's dose guidance learning and configuration information. In an alternative embodiment, the HCP may be additionally required to enter their medical license, and the system may only give them access if the entered license matches the formatting requirements for a medical license. The HCP's browser may store the medical license number for subsequent interactions with this web page.

When the HCP has access, the DGA may display a GUI report 1330, an example of which is presented in FIGS. 9A-1 and 9A-2, which provides information regarding what the patient entered 1332 into the DGS 100 and the DGS 100 observed and learned values 1334 determined during the learning period. The parameters listed in the report 1330 may include dose amounts 1342, dose times 1341 (times at which various doses were or are typically taken), dosing time ranges 1344 (start and end dosing times for each meal dose), and correction parameters 1345. The dose amounts 1342 may include amounts for a basal dose and each meal (breakfast, lunch, and dinner) dose. The correction parameters 1345 may include target glucose (mg/dL), pre-meal correction factor (mg/dL/U), and post-meal correction factor (mg/dL/U). The patient-entered data 1332 may include dose amounts 1342, dosing times 1341 (e.g., times at which a basal dose and each meal (breakfast, lunch, and dinner) dose are typically taken), and the dosing time ranges 1344 (e.g., start and end times defining a time range during which meal doses are typically taken for each meal). The observed or learned values 1334 may include the dose amounts 1342 for the basal dose and each meal dose, dosing times 1341 for the basal dose and each meal dose, dosing time ranges 1344 (the observed start and end time of each meal dosing time range), and correction parameters 1345 (a target glucose, a pre-meal correction factor, and a post-meal correction factor). The report 1330 may also provide information and/or warnings about adherence issues, e.g., in the observation notes 1336 for any or all of the parameters listed. The report may also provide conservative values 1340 for the correction parameters 1345 or any other parameter listed in the report 1330. For example, if the adherence observations indicate underdosing, then a warning message may be provided indicating to the HCP that it may be safer to set the initial dose regimen to the lower learned dose amounts (e.g., the conservative values) 1340. The report 1330 may also provide a means for the HCP to enter the dose regimen parameters manually, or to copy the values from other appropriate parts to the report, e.g., by entering values in the fields under initial therapy for dose guidance 1338. The report may also include a means for the HCP to approve this initial regimen; when the HCP selects the approval UI feature 1337, the system may download this initial dose regimen to the patient's device and initiate dose guidance. The HCP may also reject the proposed therapy in report 1330 by selecting a reject therapy option 1339. If the dose regimen parameters are not completely filled out or are out of range, an error message may be provided to the HCP. In another alternative embodiment, a control feature in the DGA may initiate the same process on the patient's phone (i.e., a user interface that enables entry of dose guidance parameters), except for the display of the URL and the code for the HCP to enter. In yet another embodiment, rather than (or in addition to) displaying a URL and code, the system may provide a UI means to enter an email address, where the patient may enter, for instance, the email address of the HCP, and the system may then send an email to this address with a hyperlink embedded, that when selected by the user receiving the email, may open on the users web browser the reports webpage with the patient's data and information.

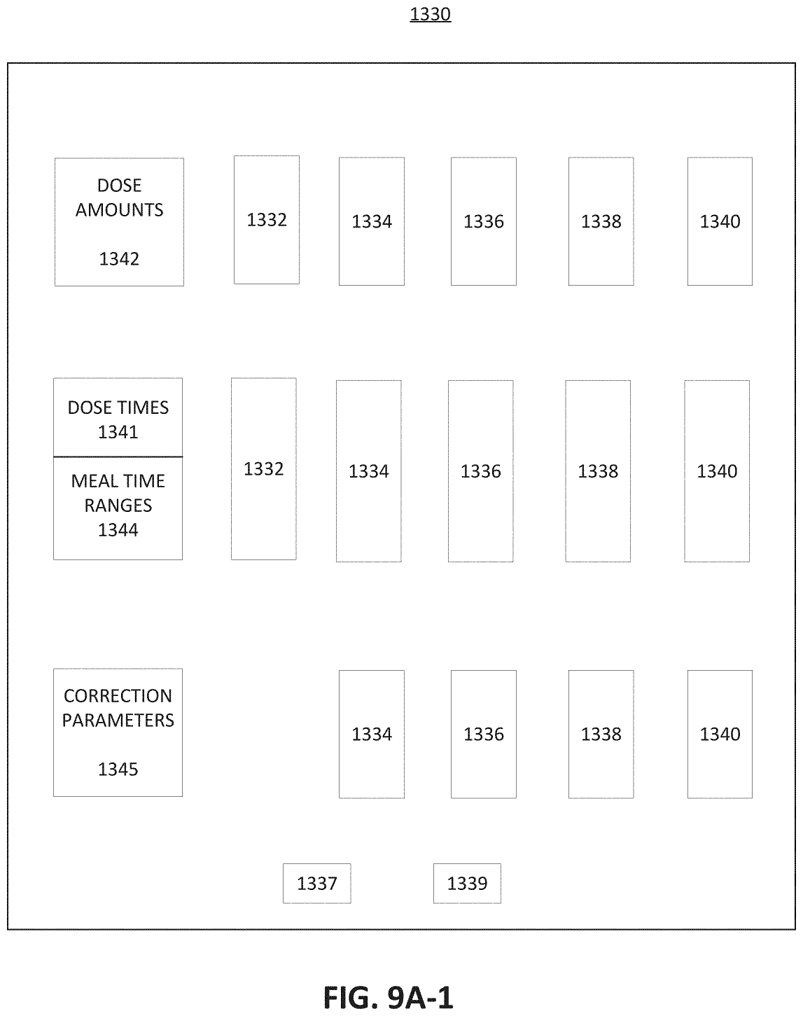

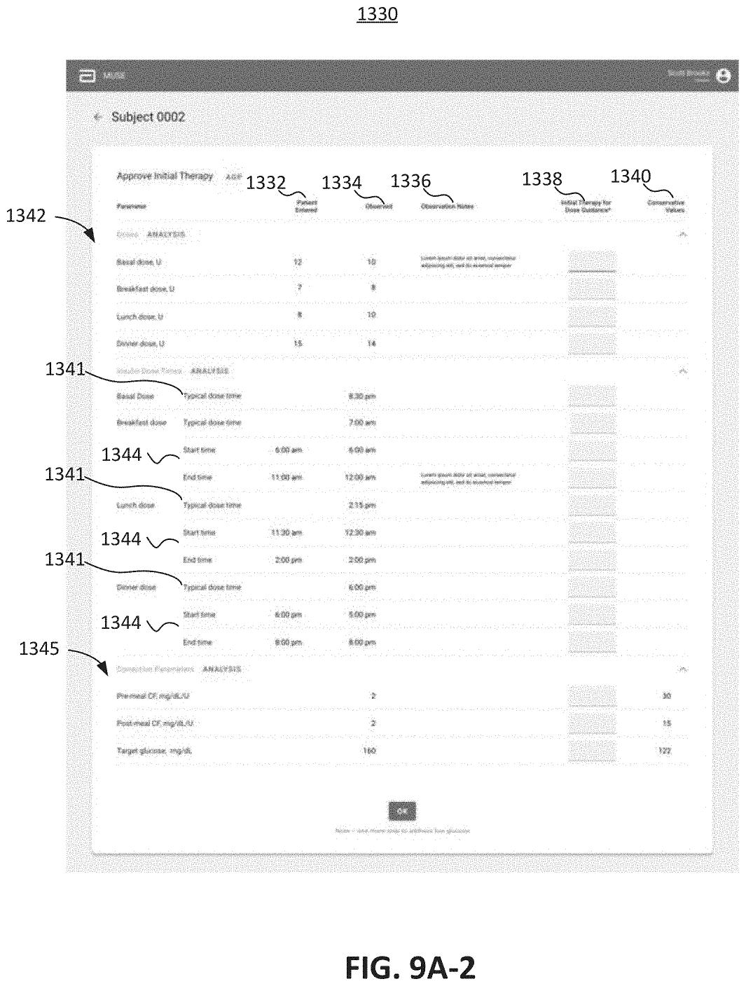

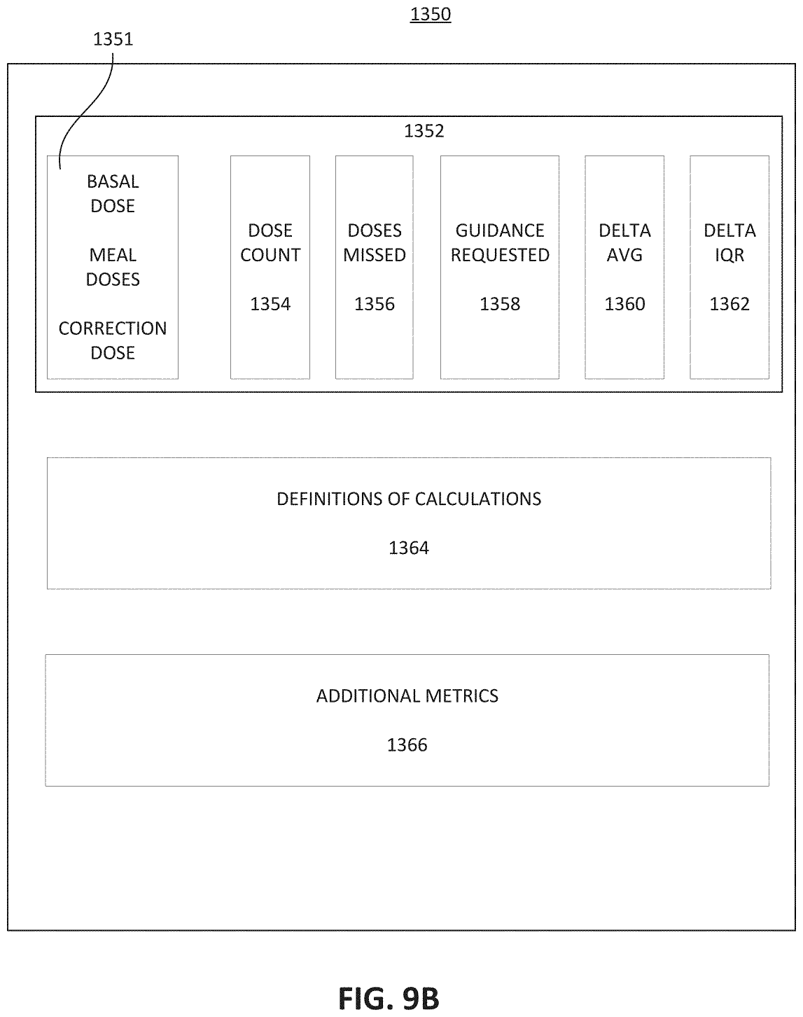

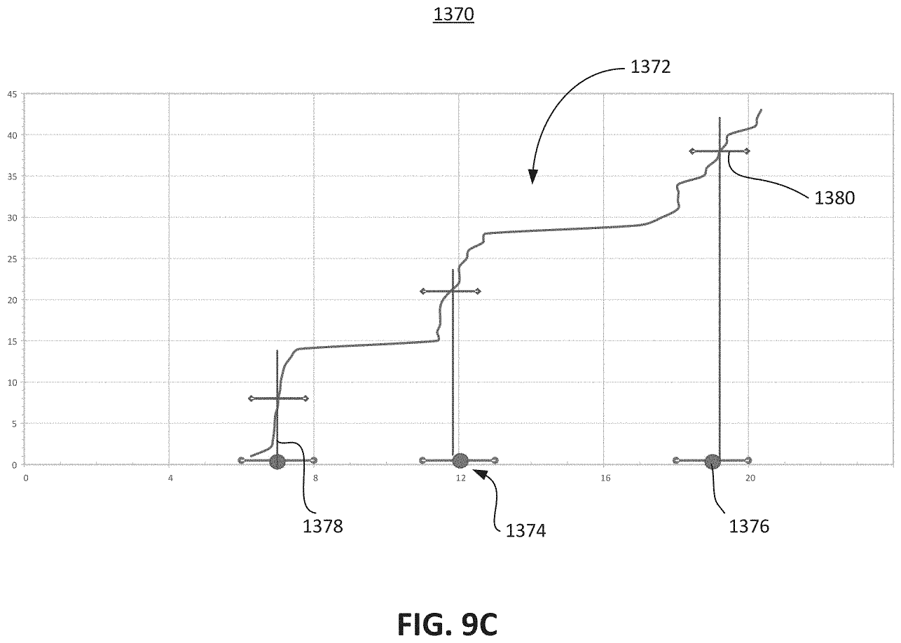

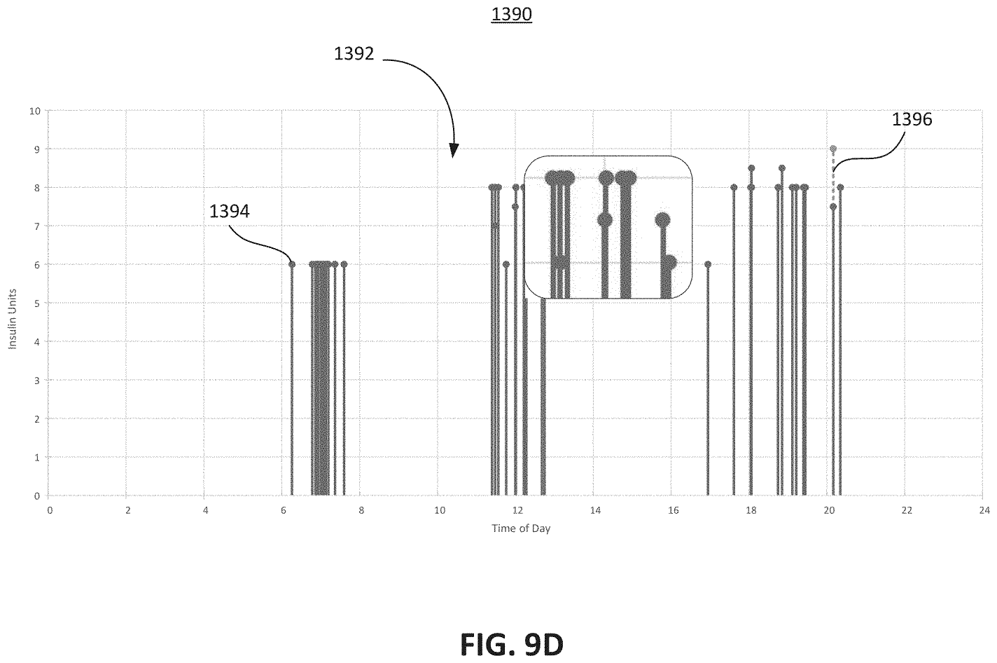

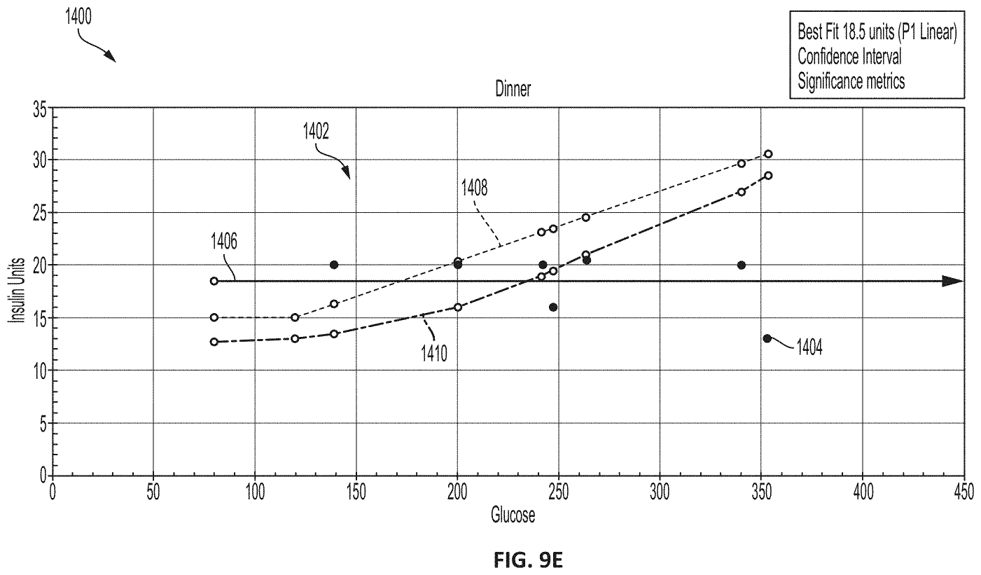

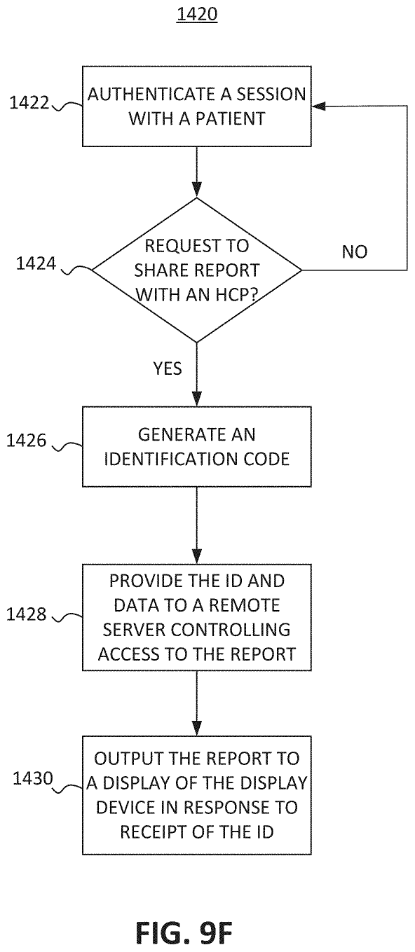

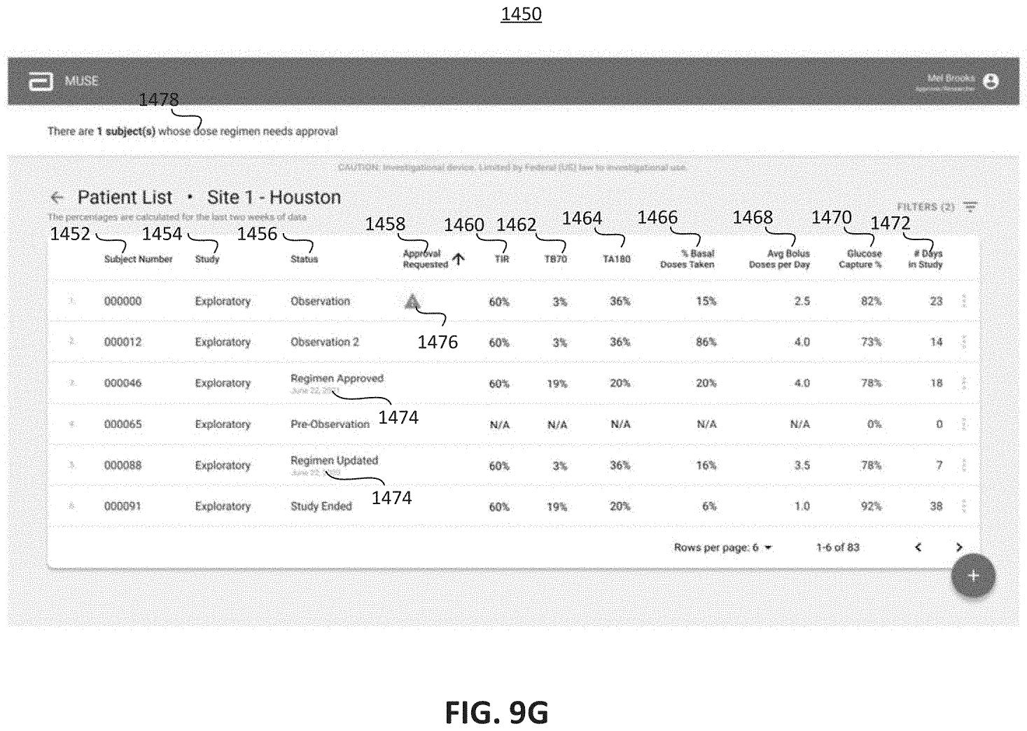

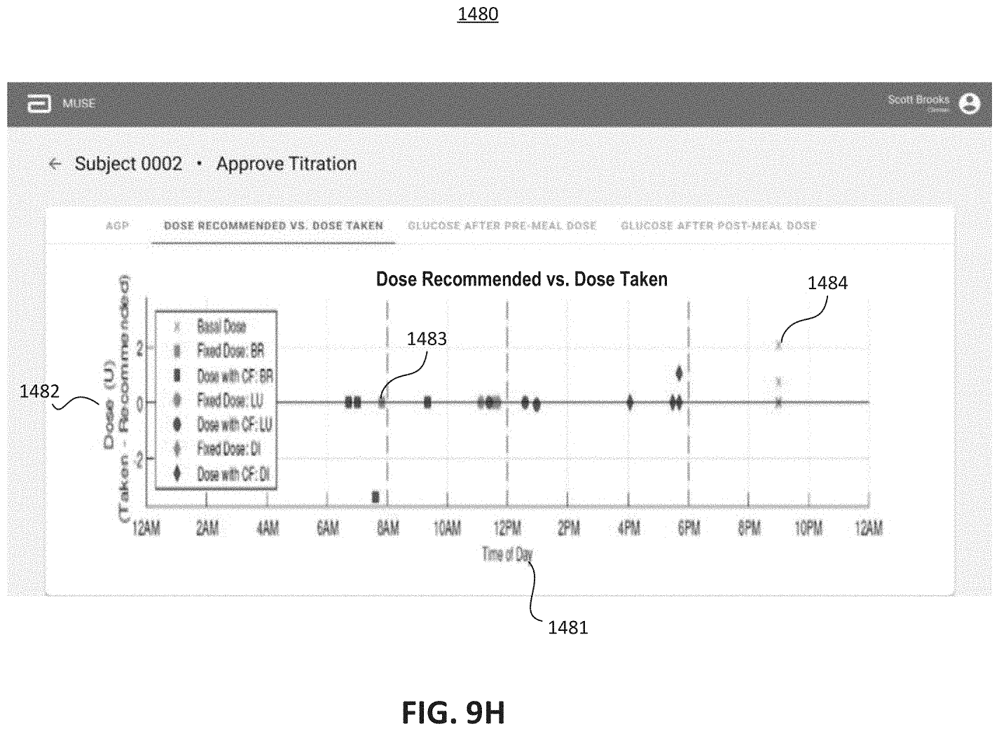

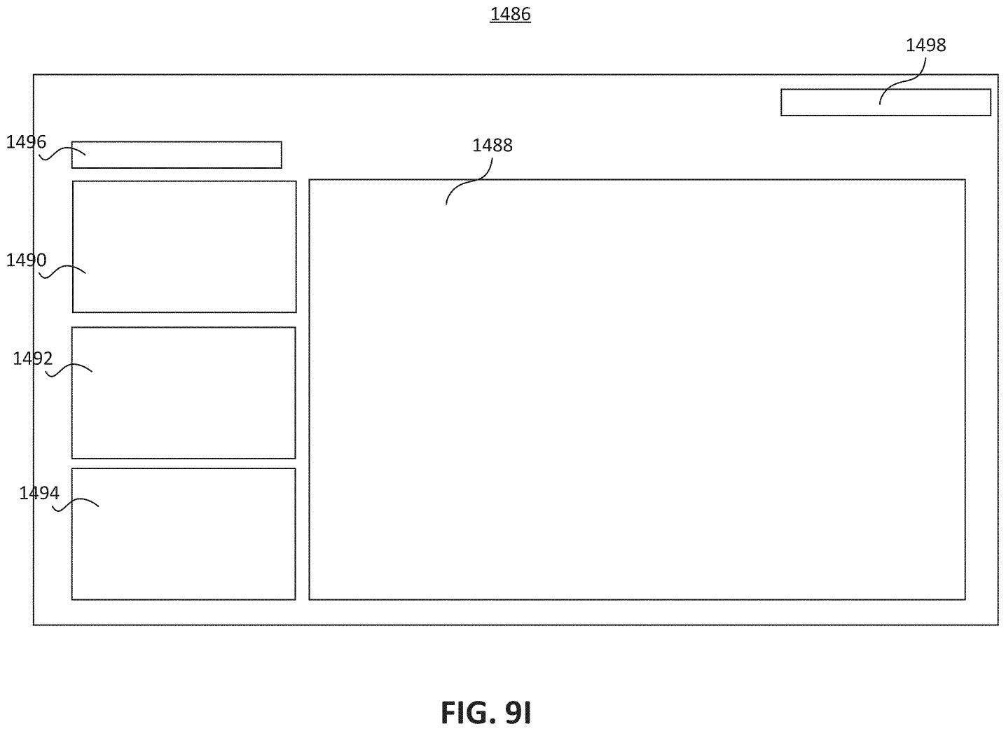

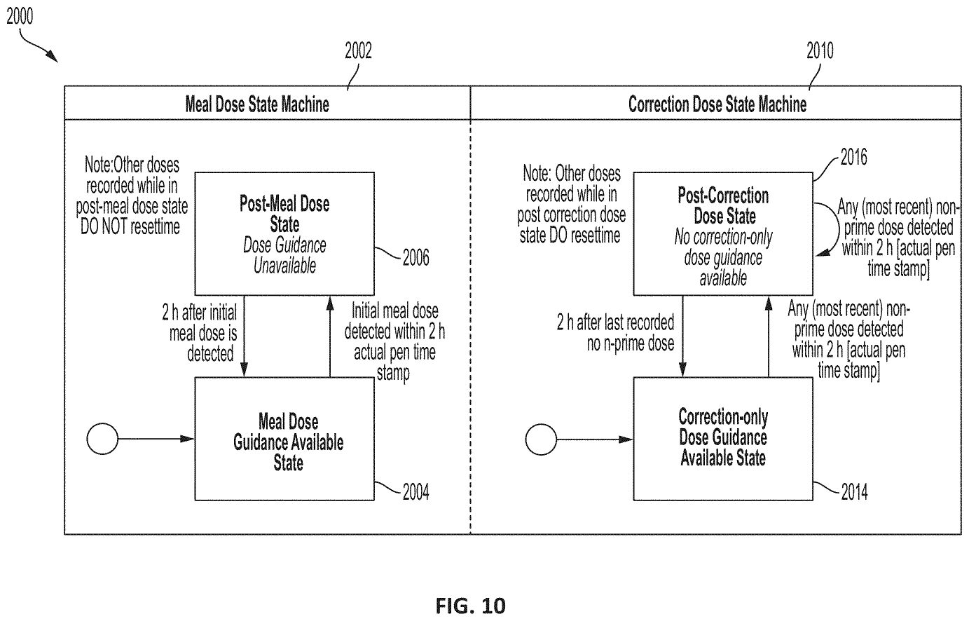

In some embodiments, the DGA may provide a status summary of patients under the care of an HCP or a treatment site. The DGA may display a GUI report 1450, an example of which is presented in FIG. 9G, which provides information regarding all of the patients under the care of a HCP or a treatment site, along with a summary of various statistics related to the patients' diabetes management. The report 1450 may include records for a plurality of subjects, including columns or fields for subject identifiers 1452, type of study 1454, status of the subject 1456, approval request pending 1458, time in range (TIR) 1460, time below a low threshold 1462 (e.g., 70 mg/dL (TB70)), time above a high threshold 1464 (e.g., 180 mg/dL (TA180)), % of basal doses taken 1466, an average number of bolus doses taken per day 1468, an amount of glucose capture (%) 1470, and a number of days of the subject in the study 1472. Any of the fields may include a filter option, which allows the HCP to sort the order of records as needed. The report 1450 may display at least one of, alternatively at least two of, alternatively at least three of, alternatively at least four of, alternatively at least five of, alternatively at least six of the time in range, time below a low threshold, time above a high threshold, percentage of basal doses taken, and average bolus doses taken per day.

The report 1450 may display a column containing a subject identifier 1452, such as subject numbers or names of the subjects.

The report 1450 may display a column containing a type of study 1454 in which the subject is enrolled. The study may be an exploratory study or other type of study.

The report 1450 may display a column containing a status 1456 of the subject. The status may be one of “pre-observation,” “observation 1,” “observation 2,” “regimen approved,” “regimen updated,” or “study ended”. If the status is “regimen approved” or “regimen updated,” a date 1474 that the regimen was approved or updated may be listed below the status. In some embodiments, the date 1474 may be grayed out or a lighter font than the status.

The report 1450 may display a column regarding a status of an approval request 1458. The approval request column 1458 may include an icon 1476 (e.g., an orange triangle with an “!” in the center) indicating that action needs to be taken by the HCP if there is an outstanding request awaiting review and approval by the HCP. The report may also contain a statement 1478 indicating how many subjects have dose regimens that need approval by the HCP.

The report 1450 may display a column reporting the amount of time that the subject's glucose levels were within a target range (“time in range” or “TIR”) 1460, as described elsewhere in this application. The TIR may be calculated for the days that the subject was participating in the study and/or the days that the subject was on the current dosing regimen.

The report 1450 may display a column reporting the amount of time that the subject's glucose levels were below a low threshold, e.g., 70 mg/dL (“time below 70 mg/dL” or “TB70”) 1462. The time below the low threshold may be calculated for the days that the subject was participating in the study and/or the days that the subject was on the current dosing regimen. In some embodiments, the low threshold may be about 60 mg/dL, alternatively about 65 mg/dL, alternatively about 70 mg/dL, alternatively about 75 mg/dL, alternatively about 80 mg/dL, alternatively about 85 mg/dL, alternatively about 90 mg/dL.

The report 1450 may display a column reporting the amount of time that the subject's glucose levels were above a high threshold, e.g., 180 mg/dL (“time above 180 mg/dL” or “TA180”) 1464. The time above the high threshold may be calculated for the days that the subject was participating in the study and/or the days that the subject was on the current dosing regimen. In some embodiments, the high threshold may be about 170 mg/dL, alternatively about 175 mg/dL, alternatively about 180 mg/dL, alternatively about 185 mg/dL, alternatively about 190 mg/dL, alternatively about 195 mg/dL, alternatively about 200 mg/dL.

The report 1450 may display a column reporting an amount of basal doses taken 1466, e.g., as a percentage. The % of basal doses taken 1466 may be calculated for the days that the subject was participating in the study and/or the days that the subject was on the current dosing regimen.

The report 1450 may display a column reporting an average number of bolus doses taken per day 1468. The average number of bolus doses taken per day 1468 may be calculated for the days that the subject was participating in the study and/or the days that the subject was on the current dosing regimen.

The report 1450 may display a column reporting an amount of glucose capture 1470, e.g., as a percentage. The glucose capture percentage 1470 may be calculated as the percentage of glucose readings received by the system from a sensor during a set time period, e.g., the days that the subject was participating in the study and/or the days that the subject was on the current dosing regimen (e.g., before a titration).

The report 1450 may display a column reporting the amount of days that that subject has been enrolled in the study 1472.

In some embodiments, the DGA may provide a report summarizing the approved therapies along with statistical analysis of the glucose level and doses differing from the approved therapies. The DGA may display a GUI report 1486, an example of which is presented in FIG. 9I, which provides information regarding the approved therapies 1488 for a particular patient 1496, along with an AGP plot 1490, a graph of missed doses 1492, and a graph of user overrides 1494. The clinician 1498 may toggle back to a complete list of their patients to view another patient's GUI report 1486.

The approved therapies 1488 may be presented in a table that includes columns for dates that the therapy was approved, the type of dosing strategy (e.g., initial, titration, manual override), amount of various insulin doses, the length of the therapy period (e.g., in days), the average number of bolus doses per day, the number of low glucose events, TIR (%), time below a low threshold (e.g., TB70(%)), time above a high threshold (e.g., TA180(%)), and an amount of glucose capture (%). The type of dosing strategy may include, but is not limited to, initial, titration, and manual override. The types of various insulin doses for which amounts are reported include, but are not limited to, basal doses; fixed doses for breakfast, lunch, and dinner; and doses with a correction factor for breakfast, lunch, and dinner. The percentages in the table may be calculated for an indicated time period of data, e.g., the last two weeks of data, for the period in which the subject was enrolled in the study, or for a period that the subject was on a particular dosing strategy.

The AGP graph 1490 depicts an ambulatory glucose profile (AGP) graph display the hourly 5^{th}, 25^{th}, 50^{th} (median), 75^{th}, and 95^{th} percentiles of glucose readings, presented over the “typical” 24-hour day based on all days within the selected timeframe. Alternatively, the AGP may display other percentiles, such as the hourly 10^{th}, 25^{th}, 50^{th} (median), 75^{th}, and 90^{th} percentiles of glucose readings, presented over the “typical” 24-hour day based on all days within the selected timeframe. The AGP graph may also include two horizontal lines, which indicate the boundaries of the target range. For example, a first line may correspond to the lower boundary of the target range (e.g., 70 mg/dL) and a second line may correspond to an upper boundary of the target range (e.g., 180 mg/dL). The first and second lines may also be color-coded. Data points falling below the lower boundary may be colored a different color than the other data points, e.g., red, to highlight these data points. Thus, the AGP graph easily illustrates the amount of time spent in (or amount of readings falling within) the target range and outside of the target range. Other exemplary AGP graphs can be found in, e.g., US 2018/0235524, US 2014/0188400, US 2014/0350369, US 2018/0226150 all of which are expressly incorporated by reference it their entirety for all purposes.

The graph of missed doses 1492 graphs the percentage of doses missed of the total number of doses received or administered over a time period for each of basal, breakfast, lunch, and dinner doses. This graph can easily show the HCP if the subject is missing a large number of a certain type of dose and recommend corrective measure. The percentages in the graph may be calculated for an indicated time period of data, e.g., the last two weeks of data, for the period in which the subject was enrolled in the study, or for a period that the subject was on a particular dosing strategy. The graph may be configurable such that the HCP may select a desired time window from a drop down menu. The graph of missed doses 1492 may be a bar graph wherein one axis is the type of dose (basal, breakfast, lunch, and dinner doses) and the other axis is percentage of missed doses from total doses.

The graph of user overrides 1494 graphs the percentage of doses in which the subject did not take the recommended dose for each of basal, breakfast, lunch, and dinner doses of the total number of doses received or administered over. This graph can easily show the HCP if the subject is disregarding the recommended dose for a certain type of dose or meal and recommend corrective measure. The percentages in the graph may be calculated for an indicated time period of data, e.g., the last two weeks of data, for the period in which the subject was enrolled in the study, or for a period that the subject was on a particular dosing strategy. The graph may be configurable such that the HCP may select a desired time window from a drop-down menu. The graph of user overrides 1494 may be a bar graph wherein one axis is the type of dose (basal, breakfast, lunch, and dinner doses) and the other axis is percentage of user override doses of total doses.

In some embodiments, the DGA may provide a graph that illustrates a correlation between the recommended dose amounts and the actual amount of the dose taken by the subject. The DGA may display a dose recommended vs. dose taken GUI 1480, an example of which is presented in FIG. 9H, that graphs the dose taken at various times during the day vs. the dose taken relative to the dose recommended. As seen in GUI 1480, the x-axis 1481 may be time in minutes, hours, days, or weeks. In some embodiments, the graph my span from 12 AM to 12 PM. The graph may display data accumulated over a time period of several days, weeks, or months, and may graph the doses on the same graph. For example, the graph may include all of the doses administered during the time period that the subject is in a particular study period or subject to a particular dosing regimen.

The y-axis 1482 may be the difference between the dose taken and the recommended dose. If the dose taken is the same as the dose recommended, then the y-coordinate for that dose would be zero and the x-coordinate would be the time that the dose was administered or taken by the user. If the dose taken was X1 units greater than the dose recommended, then the y-coordinate for that dose would be X1. (For example, if the dose taken was 2 units greater than the dose recommended, then the y-coordinate for that dose would be 2.) If the dose taken was X2 unit less than the dose recommended, then the y-coordinate for that dose would be −X2. (For example, if the dose taken was 1 unit less than the dose recommended, then the y-coordinate for that dose would be −1.) For example, if the dose taken was the same as the recommended dose for a fixed breakfast dose at 7:50 AM, then the corresponding point 1483 on the graph would be (7:50 AM, 0). If a basal dose taken at 9 PM was 2 units greater than the recommended basal dose, then the corresponding point 1484 on the graph would be (9 PM, 2).

The doses that may be presented in the graph include, but are not limited to, basal doses; fixed doses for breakfast, lunch, and dinner; and doses with a correction factor for breakfast, lunch, and dinner.

In some embodiments, as seen in FIG. 9B, a report 1350 detailing regimen adherence may also be displayed. The report 1350 may be accessible by the patient, HCP, or other interested party. The adherence report 1350 may cover a time period (e.g., a week, alternatively 2 weeks, alternatively for the learning period duration) and include a table 1352, which lists different analytics for each of the different types of doses 1351. The different types of doses 1351 may include basal, breakfast, lunch, dinner, and correction (e.g., post-meal correction) doses. The table 1352 may list the doses count 1354 of the doses taken of the different dose types, doses missed 1356, guidance requested 1358, delta average of the of the taken dose and guidance dose 1360, and delta IQR of the taken and guidance doses 1362, for each of basal, breakfast, lunch, dinner, and/or postmeal correction.

Definitions and/or explanations 1364 of each of these categories may be listed below the table 1352. The dose count 1354 may be a simple count of the dose types over the relevant time period (e.g., the most recent week). The doses missed 1356 may be reported as a percentage and, for the basal dose for the most recent week, may be calculated by (7—basal count dose)/7. The doses missed 1356 for the meal doses may be calculated as (missed dose detections without associated dose)/(all missed dose detections). The doses missed 1356 for the postmeal corrections may be calculated as (postmeal correction alerts without associated dose)/(all postmeal correction alerts). The guidance requested 1358 may be reported in units of insulin and may be calculated as (doses associated with guidance display)/(all doses). The delta average (Taken-Guidance) 1360 may be reported in units of insulin and calculated by, for doses associated with guidance, calculate the average difference. The delta IQR (Taken-Guidance) 1362 may be calculated by, for doses associated with guidance, calculate the IQR. The delta IQR has the standard meaning to those in the art and refers to the difference between the third and first quartiles.

Additional metrics 1366, and statistics relating to these metrics, may also be listed in the report 1350, including late dose frequency, postmeal frequency, auto vs. manual dose classification, non-meal and non-postmeal correction frequency, snack dose frequency, percent doses taken per guidance (which may be dose linked with guidance in time, not just aggregated), and percent doses taken per alert.

FIG. 9C illustrates an exemplary plot 1370 associated with the clustering analysis to determine meal periods, which can optionally be displayed to the HCP. The graph 1372 plots the cumulative count of all of the doses in the learning period, versus time-of-day (e.g. by ascending time of day) when the dose occurred. The user-entered meal time range values 1374, along with the user-entered meal time halfway point 1376, may be indicated in the plot 1370, e.g., near the x-axis, if available. The rapidly increasing portions of the curve indicate each meal-time cluster. In an alternative embodiment, the graph may be plotted as a histogram or pie chart of the count. The learned typical meal time 1378 may be indicated by a vertical line in the plot 1370. The line segment 1378 has a length equal to the amount of insulin injected. If there are more than one injections within a 15 minute period, the length is equal to the sum of all of the injected amounts—this is necessary because sometime patient will inject multiple times for a single meal, for instance if the insulin pen they are using contains less than the full amount needed to cover the required dose and the patient refills the pen (or gets a new full pen) and completes the require dose amount; or some patients require an insulin dose amount more than the injection dose capacity for the pen so multiple injections are needed; or some patients experience pain with injecting large amounts of insulin in one injection so they dose with multiple injections. The learned meal time range 1380 may be indicated by a horizontal line located at an intersection of the typical time and dose lines.

FIG. 9D illustrates an exemplary plot 1390 associated with meal dose clustering and dose amounts. The graph 1392 plots each dose amount of all of the doses in the learning period, versus the TOD when the dose occurred. Each dose amount is indicated by a dot 1394, to indicate different dose amounts at the same time of day. The thickness of the bar associated with a time of day may be changed to correspond to the number of doses, in order to illustrate when identical dose amounts occur at the same time of day. As seen in the in-laid window, the dot 1394 and thickness of the bars allows a viewer to distinguish varying doses taken at the same time of the day. Doses taken within a short period of time, e.g., within about 15 minutes, on the same day may be added together (e.g., 7.5+1.5=9 units). The additional dose may be indicated with a different color or type of line 1396. Alternative means may be employed to illustrate this.

A user interface control may be provided to toggle between showing the results for all data vs. only showing the results for doses associated with premeal glucose below some threshold, e.g., 150 mg/dL. Alternatively, a user interface control may also be provided to allow the user to set the threshold.

FIG. 9E illustrates an exemplary plot 1400 associated with a premeal correction factor determination. The graph 1402 plots a dot 1404 for each insulin dose amount for all of the initial meal doses in the learning period vs. the pre-meal glucose associated with each dose for a particular meal. The curve for each model fit performed in the learning analyses is overlayed, along with the associated learned parameters. The models may include P1 zero-slope model (no corrections) 1406, P2 piecewise linear function 1408, and P3 non-linear function 1410. The best fit curve may be highlighted in some way, e.g., with a thicker curve (compare 1406 to 108 and 1410). The DGA may provide four plots, one for each meal and one overall, as described previously.

If the DGS fails to adequately learn the patient's dosing regimen, instead of enabling the dose guidance more, the DGA may provide a means to share a report with their HCP regarding the learning status. Note that this method may also be used to share any type of report. A method 1420 for facilitating efficient access by an HCP to a report generated by the DGS 100 while protecting a patient's health data privacy is described in FIG. 9F.

At step 1422, a session with the patient is authenticated by at least one processor of a portable display device. The authentication may be accomplished when the patient logs into the DGA or other known methods of authentication. Most commonly, this function is served by the authentication function provided by the patient's smartphone.

At step 1424, the at least one processor may determine if an input has been received from the patient during the session that indicates a request to share the EMR(s) with an HCP. The DGA may provide a user interface selectable feature to allow the user to indicate the desire to share the report. If it is determined that a request to share the EMR was made, at step 1426, the at least one processor may generate an report-sharing identification code (ID). The ID may be an alphanumeric code, a numeric code, or any other appropriate format. This code may be displayed by the DGA, along with the URL of a remote report-access server.

At step 1428, the at least one processor may provide the ID, along with data needed to generate the report, to the remote report-access server controlling access to the report. At step 1430, the HCP may then engage a standard web browser on their PC, and enter the URL displayed by the DGA, as appropriate. The report-access server may then cause the browser to display a screen to accept the ID. The HCP may enter the ID, the browser may send this ID to the server and the server may send the report to the browser if the ID matches that the ID sent from the DGA and the ID has been received before some period of time has elapsed since it was sent from the DGA, e.g., 20 minutes.

In another aspect, the method 1420 may also include the step of determining whether the DGS fails a condition of consistency between the patient entered regimen and the learned regimen. If it determined that the EMR fails a condition for consistency, the method may include the step of providing the patient with an option to provide the learning results report to the HCP. In one embodiment, the step of generating the report-access ID may be conditioned on a determination that the EMR fails the condition for consistency.

In another aspect, the method may include the remote server creating a user interface, e.g., a web page, addressed at least in part by the ID for displaying the report. The report may include determinations of dosing parameters for a medication administered to the patient at times during a defined period and a measure of consistency of the determinations with patient-supplied dosing information for the medication, as seen in, e.g., FIGS. 9A-1-9A-2.

An alternative embodiment includes the DGA providing a user interface means for the patient to generate a report directly for display on the DGA—this report may be shown to the HCP. It is to be understood that each of these steps is optional and are not necessarily required in the method.

### Titration Opportunity Following Learning

During the Learning Period, insulin doses may not be fully characterized like they may be during the Guidance Period. Therefore, the initial titration using the data from the Learning Period may be different than the titration during the Guidance Period. Specifically, the DGS 100 may only titrate fixed doses during the Learning Period and CF may not be titrated. Additionally, titrations may only be performed if low patterns are detected by the fixed dose titration algorithm. Any low pattern detected may trigger a dose reduction in the insulin dose preceding that time period as described in the fixed dose titration section above. The GPA algorithm maybe used to evaluate the fixed meal time periods. Bedtime may be defined as 6 hours after the fixed dinner dose time or about 6 hours prior to the fixed breakfast time, whichever happens earlier.

### User Feedback During Learning Period

Example embodiments of methods for acquiring user feedback during or after a learning period of the DGA will now be described. The user can be prompted for feedback during the initial learning phase with the DGA. User feedback can provide an indication to the user that the system is making progress. The DGA can prompt the user for feedback (e.g., input or confirmation) as to any aspect of dose guidance, including a lack of information about an aspect of administered doses, analyte history, patient behavior or activities, dosing strategy generally, the type of a particular dose, confirmation that a DGA determined (e.g., learned by the system) dose type or strategy is correct, and others.

During (or after) the learning period, the DGA can output a prompt or other indication on UID 200 that requests user feedback. This feedback can concern dosing strategies, for example, strategies pertaining to insulin action type (e.g., long-acting and/or short or rapid-acting). If the feedback (or other determination) indicates a long-acting strategy is being used, then, for a first time period, e.g., the first three (3) days, the DGA can monitor the basal dosing pattern of the patient to categorize each dose or dosing pattern as a single or split dose type, and/or to characterize it the dose by time period (e.g., morning single dose, evening single dose, or split dose (e.g., both morning and evening). The DGA can also determine a tendency about the dose amount (e.g., median, mean) and the associated dose variability. From this information, the DGA can develop an expected basal dose. After the first time period, the user can be prompted for feedback if the actual dose administered (e.g., automatically registered by MDD 152 or input by the user) is different than expected.

According to one aspect of the embodiments, the user can be prompted in many different circumstances. For example, the DGA can be configured to detect missed doses, such as where a user did not administer a basal or bolus dose during a time period in which a prior basal or bolus dose was administered. If a missed dose is detected, the DGA can be configured to request input from the user regarding whether a basal dose was administered in the time period. According to some embodiments, the DGA can also be configured to detect differences in dose timing. For example, the DGA can be configured to detect when the user administers a basal dose in a different time of day period than the time of day period that a prior basal dose was administered (e.g., a basal dose that is usually administered in the morning was administered in the evening). When such a difference in timing of administration is detected, the DGA can be configured to request input from the user regarding whether a basal dose was administered in a different time period. In another aspect of the embodiments, the DGA can also be configured to detect when extra doses have been administered. For example, the DGA can be configured to detect a change in the number of basal doses administered in a day. In yet another aspect of the embodiments, the DGA can be configured to detect if a dosing strategy on a first day (e.g., one basal dose was administered) is different from a dosing strategy on a second day (e.g., two basal doses were administered). When a different dosing strategy is detected, the DGA can be configured to request input from the user regarding whether the user has adopted the dosing strategy used on the second day as a new dosing strategy. In yet another aspect of the embodiments, the DGA can also be configured to detect if a different dose amount was administered. For example, the DGA can be configured to detect whether a first amount of a dose administered in a time of day period is different (smaller or larger) than a prior dose administered in the time of day period on a previous day. When a different dosage amount is detected, the DGA can be configured to request input from the user regarding whether the user has changed the dosage amount.

The user response to these prompts can allow the DGA to either confirm that it has identified a correct pattern (e.g., the user confirms that they missed taking their morning basal dose, but they normally would take it) or offers the opportunity for the user to correct the pattern (e.g., the user informs the DGA that they adjust the basal dose based on their glucose before taking).

For rapid acting insulin dosing strategies, in addition to the above prompts, the DGA can also include a prompt regarding dose classification. The dose classifications can include, but are not limited to, bolus, correction, split-dose, bolus+correction, and bolus+carbohydrate counting+correction classifications.

The user can be prompted in many different circumstances concerning the dosing of rapid-acting insulin. The DGA can be configured to detect if a dose was administered that is not associated with a meal. For example, the DGA can be configured to determine if a dose was taken in a time period in which a meal was not identified or detected. If the DGA detects a dose was taken and a meal was not detected within a time period of the administration (e.g., within about 1 hour of administration), the DGA can request input from the user regarding a reason why the dose was administered (e.g., because they ate a meal, because they were lowering their glucose, or because they were finishing up an earlier meal dose). The DGA can also be configured to detect if a meal dose does not match a prior meal dose associated with the same meal type. For example, the DGA can be configured to determine if a bolus dose associated with a first meal type and administered in a time of day period is not the same as a prior bolus dose associated with the first meal type and administered in the time of day period on a previous day. Where such a difference in bolus doses is detected, the DGA can be configured to determine a reason for the different dose. For example, the DGA can be configured to determine a difference in a pre-meal glucose values associated with the bolus dose and the prior bolus dose to determine whether the difference detected is a correction. The DGA can also request input from the user regarding the reason for the difference in bolus doses (e.g., because they ate less/more food and/or they were correcting for hyperglycemia, and/or they were correcting for other factors).

In addition to enabling the DGA to determine what type of rapid-acting doses are being taken throughout the day, this enables the DGA to facilitate when to expect a dose. After a period of learning where no prompts are provided, the DGA can provide these prompts to the user in the case where the dose differs from the expected dose, in order to refine the DGA's model of the user's dosing strategy.

For both long-acting and rapid-acting doses, the DGA can aim to minimize the number of prompts as time goes on, and as the user responds. Emphasis can be placed on prompting frequently in the beginning stages, and tapering off as repeat patterns are observed.

Glucose Pattern Anal sis acid Meal Bolus Tina/ion far MD1 Insulin Dosing Therapies

Example embodiments of methods for determining meal bolus titrations will now be described. The system can provide titration guidance for multiple daily injection (MDI) dosing therapies once it learns (or is configured with) the patient's current dosing strategies. For patients using fixed meal dosing, the fixed dose amounts (e.g., for breakfast, lunch, dinner, snack, etc.) can be titrated. For patients who are carbohydrate counting, the carbohydrate ratio can be titrated, for these same meals or for different times of the day. Patients who use experiential dosing can titrate their doses on a per meal basis. Titration guidance by the DGA can provide a recommendation to change the dose or carbohydrate ratio in a particular direction. The amount of the change can be a suitable percentage change, for example, 5%, 10%, 15%, etc. Dose guidance can also include starting a meal dose. For example, if a patient is on a basal plus one (e.g., lunch dose) regimen), and breakfast shows a high pattern, the DGA can provide a recommendation to administer a RA insulin for breakfast.

The DGA can require that a dosing category be defined such as Time-of-Day (TOD) period, meal type (e.g., breakfast), and composition of the meal (e.g., cereal with milk). For example, the dosing category can be time-of-day, defined by time-periods associated with meal insulin doses for time-of-day periods. For further example, a post-breakfast time period can be defined as starting when a meal insulin dose is taken in a defined period of day, for example, between 5 am and 10 am, and ending either after a defined post-meal period (e.g., six (6) hours later), or when the next meal insulin dose is taken, whichever is earlier. One or more metrics can be required to define whether a post-meal glycemic response is nominal or requires correction, or to rank a post-meal glycemic pattern as more or less favorable than another. Likelihood of low glucose (LLG) metric and the median glucose can be used to quantify the degree of hypoglycemia risk and hyperglycemia risk, respectively.

U.S. Patent Publ. No. 2018/0188400 (the '400 publication), which is incorporated by reference herein for all purposes, describes examples of an implementation for deriving and determining risk metrics that can be utilized in glucose pattern analysis (GPA) for the DGA embodiments. This implementation, among other things, utilizes central tendency (e.g., mean, median, etc.) and variability data from the multi-day period to determine a risk metric corresponding to a degree of hypoglycemia risk (“hypo risk”). This implementation is summarized herein, and a more exhaustive description of the implementation and variations therefrom can be obtained by reference to the '400 publication.

Alternatives to the implementation described in the '400 publication are set forth in U.S. Patent Publ. No 2014/0350369, which is also incorporated by reference herein for all purposes. For example, instead of using median and variability, the method could employ any two statistical measures that define a distribution of data. As described in the '369 publication, the statistical measures could be based on a glucose target range (e.g., G_{LOW}=70 mg/dL and G_{HIGH}=140 mg/dL). Common measures related to the target range are time in the target range (TIR), time above target (t_{AT}), and time below target (t_{BT}). If the glucose data is modeled as a distribution (e.g., a gamma distribution), for predefined thresholds G_{LOW} and G_{HIGH}, then t_{AT} and t_{BT} can be calculated. For the thresholds, an algorithm can also define t_{BT_HYPO} in which if exceeded by t_{BT}, then the patient may be determined to be a high hypoglycemia risk. For example, high hypoglycemia risk can be defined as whenever t_{BT} is greater than 5% for G_{LOW}=70 mg/dL. Similarly, a metric t_{AT_HYPER} can be defined in which if exceed by t_{AT}, then the patient can be determined to be at high hyperglycemia risk. The degree of hypoglycemia and hyperglycemia risk can be adjusted by adjusting either G_{LOW} or t_{BT_HYPO}, or G_{HIGH} or t_{AT_HYPER}, respectively. Any two of the three measures, TIR, t_{BT}, and t_{AT} can be used to define a control grid. These alternatives (and others) can be used to determine risk metrics for the DGA embodiments described herein.

The DGA embodiments described herein can operate based on a quantitative assessment of the user's analyte data during a TOD period. This quantitative assessment can be performed in various ways. For example, the embodiments described herein can assess the analyte data over a multi-day period to determine one or more metrics that are descriptive of relevant risks exhibited by that analyte data for a corresponding TOD. These metrics can then be used to classify the analyte data from the TOD period as one of multiple patterns. For example, these patterns can be indicative of a common or prevalent glucose behavior or trend for that TOD. Any number of two or more patterns can be utilized by the DGA embodiments. For ease of reference herein, these patterns are referred to as glucose pattern types and the embodiments described herein will make reference to an implementation utilizing three glucose pattern types (e.g., a low pattern, a high/low pattern, and a high pattern), although other implementations may utilize only two types or more than three types, and those types may differ from those described herein.

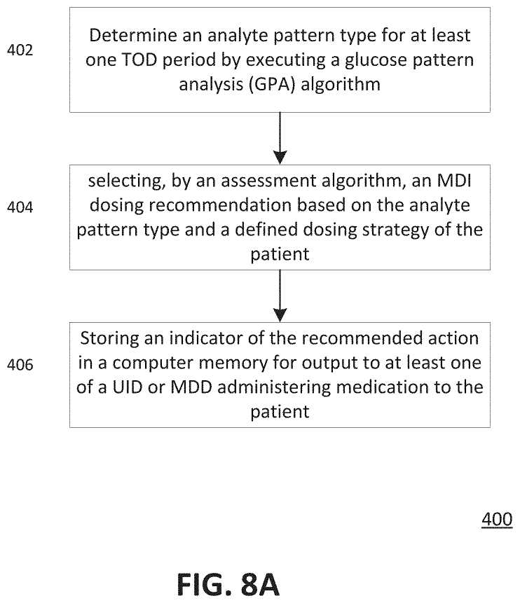

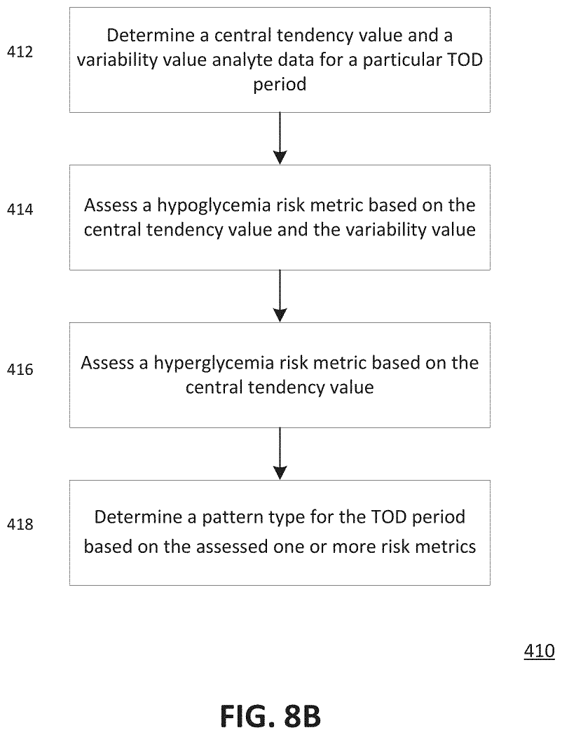

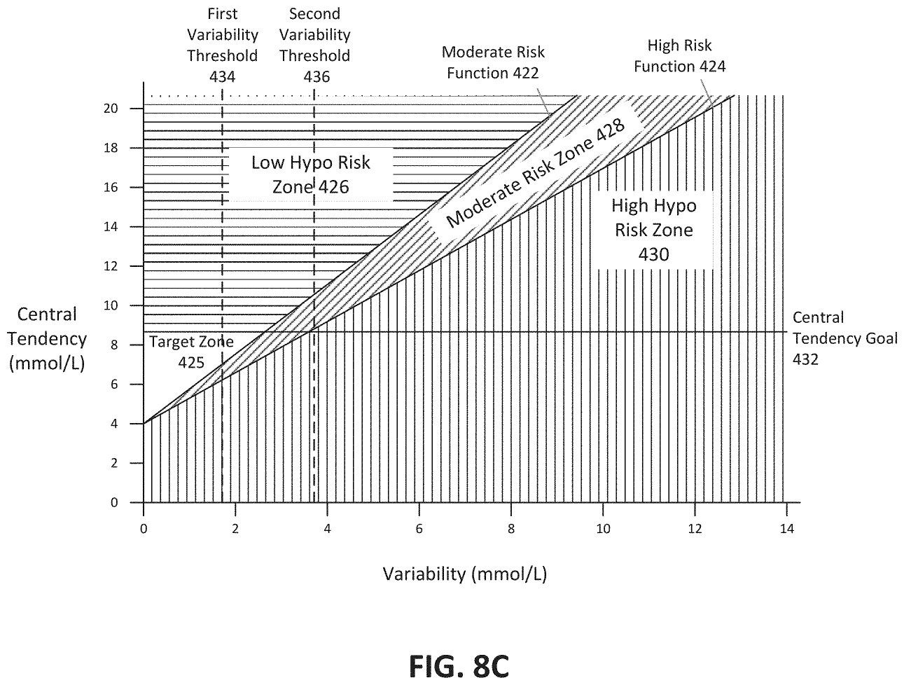

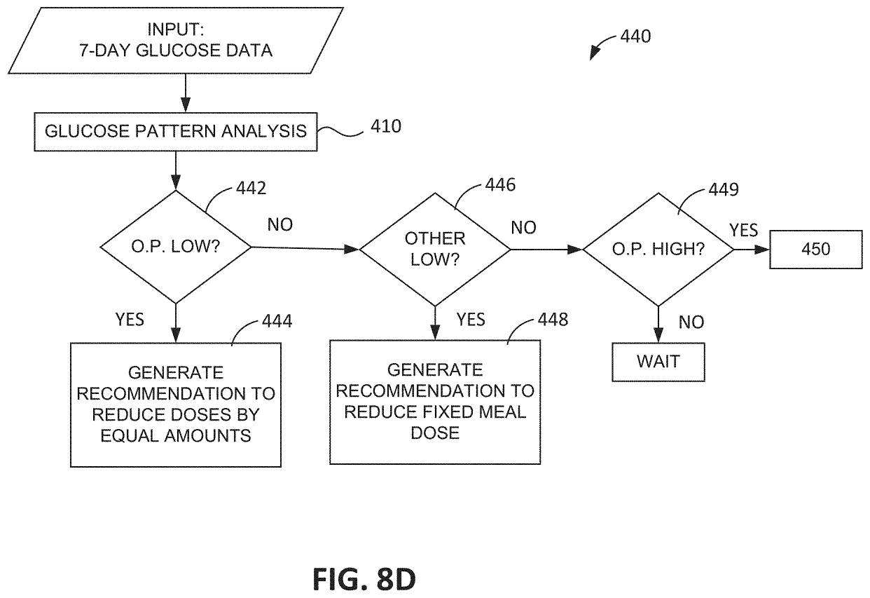

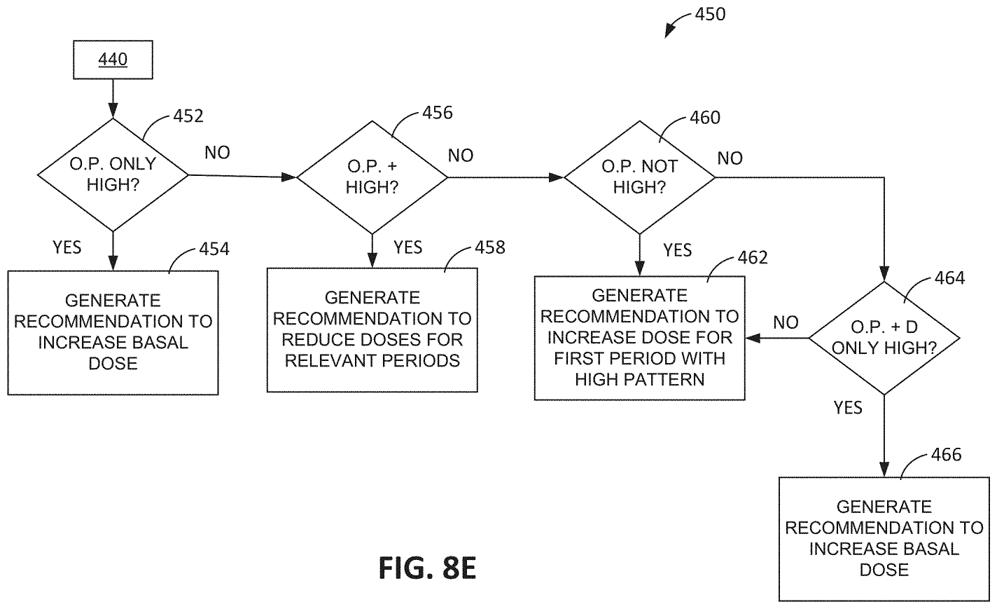

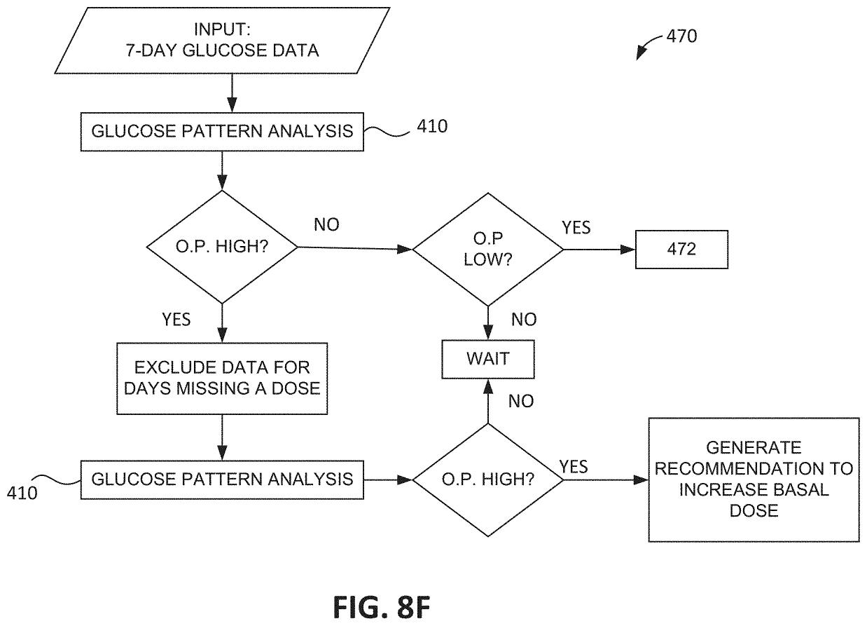

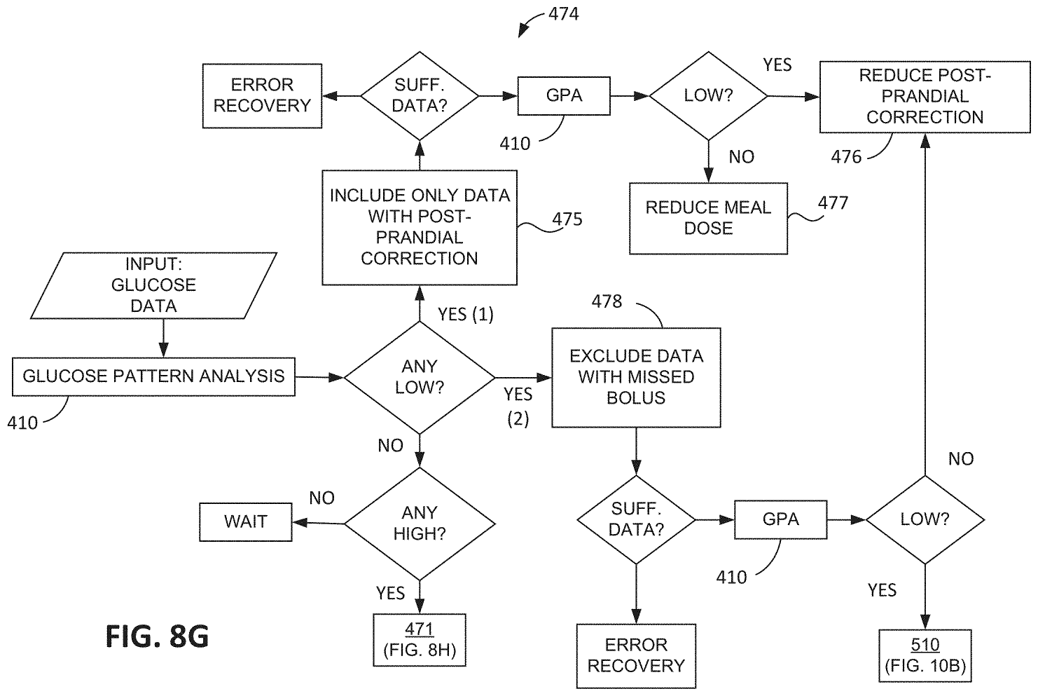

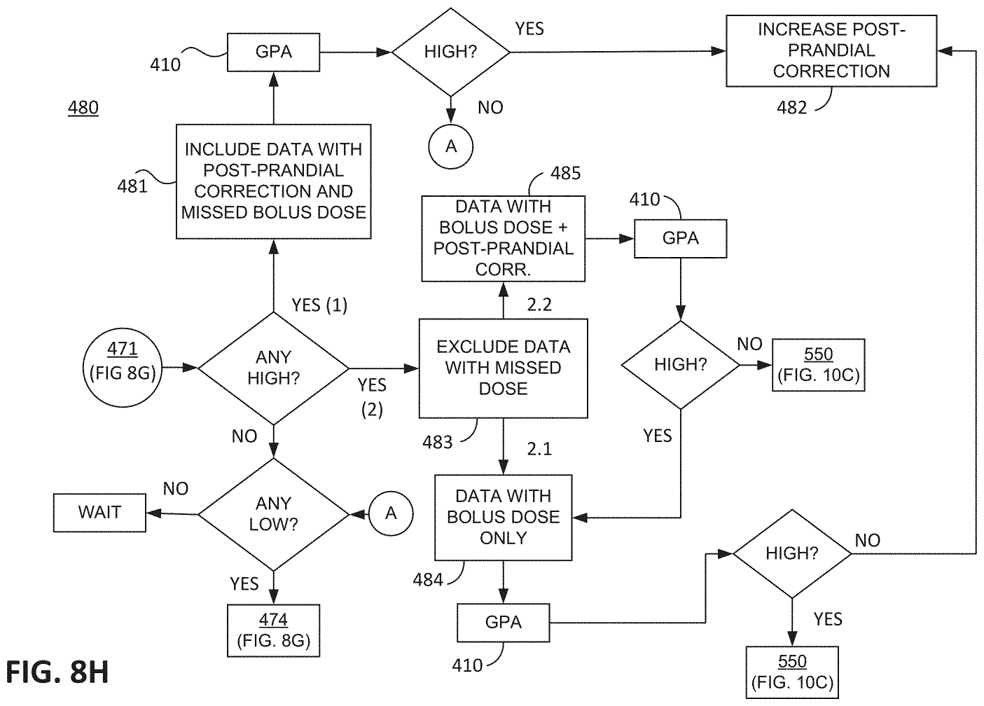

Using fixed meal doses for example, once the DGA has learned the dosing strategy and the dose or carbohydrate ratio amounts, then titration assessment can begin, which can be categorized into four titration categories: overnight, post-breakfast, post-lunch and post-dinner. For each of these categories, the DGA can map the two metrics described above (LLG and median glucose) to the four logic “pattern” variables per the GPA method described below. FIG. 8A shows operations of an example method 400 by a DGA for assessing a meal bolus titration for multiple daily injection (MDI) dosing therapy. The method 400 can include, at 402, determining, by a DGA, an analyte pattern type for the at least one TOD by executing a glucose pattern analysis (GPA) algorithm that receives, as input, time-correlated analyte data originating from a sensor control device worn by a patient over an analysis period. The method 400 can further include, at 404, selecting by the DGA executing a recommendation algorithm, an MDI dosing recommendation based on the analyte pattern type and a defined dosing strategy of the patient for the analysis period. The method 400 can further include, at 406, storing, by the DGA an indicator of the recommended action in a computer memory for output to at least one of a UID 200 or an MDD 152 administering medication to the patient. A UID 200 can use the indicator of the recommended action to control a user interface, for example, by causing a human-readable expression of the indicator to appear on a display, or by generating an audio output expressing the indicator in a human language. An MDD 152 can use the indicator to adjust or maintain a next relevant dose administration. Further details of the method 400 are described below.

FIG. 8B is a flowchart depicting an example embodiment of a GPA method 410 that can be implemented as the GPA algorithm referenced in 402. Method 410 can be performed for a particular TOD period that can be an entire day (e.g., a 24 hour period), or a portion of a day that is delineated by time blocks (e.g., three 8 hour periods) or the user's activities (e.g., meals, exercise, sleep, etc.). In many embodiments, multiple TOD periods can correspond to meals (e.g., post-breakfast, post-lunch, post-dinner) and sleep (e.g., overnight). These TOD periods can correspond to fixed times of the day where the activity would normally occur (e.g., post-breakfast from 5 am to 10 am), where such time blocks can be set by the user, or can be contingent on the meal or activity actually having been performed as determined by the automated detection of the meal or activity, or by a user indication of such (e.g., with UID 200).

A DGA can perform the method 410 independently for each TOD period to arrive at a separate pattern assessment for that period. At 412, the DGA can determine a central tendency value and a variability value from the user's analyte data for the particular TOD period. The user's analyte data may be available from the user's own records or those of the user's healthcare professional, or the user's analyte data may have been collected by DGS 100, for example. The analyte data preferably spans a multi-day period (e.g., two days, two weeks, one month, etc.) such that sufficient data exists within the TOD period to make a reliable determination. In other embodiments, the method can be performed in real-time on limited data. The DGA can use any type of central tendency metric that correlates to a central tendency of the data including, but not limited to, a median or mean value. Any desired variability metric can also be used including, but not limited to, variability ranges that span the entire data set (e.g., from the minimal value to the maximum value), variability ranges that span a majority of the data but less than the entire data set so as to lessen the significance of outliers (e.g., from the 90^{th} percentile to the 10^{th} percentile, from the 75^{th} percentile to the 25^{th} percentile), or variability ranges that target a specific asymmetrical range (e.g., low range variability, which can span a range, e.g., from or in proximity with the central tendency value to a lower value of data, e.g., the 25^{th} percentile, the 10^{th} percentile, or the minimal value). The selection of the metrics to represent the central tendency and variability can vary based on the implementation.

At 414, the DGA can assess a risk of hypoglycemia (“hypo risk”) metric based on the central tendency value and the variability value. One such methodology for determining hypo risk is described with respect to FIG. 8C, showing an example embodiment of a framework for determining hypo risk and other metrics. While FIG. 8C is intended to convey the framework to the reader, however, this framework can be implemented electronically in numerous different ways, such as with a software algorithm (e.g., a mathematical formula, a set of if-else statements, etc.), a lookup table, firmware, a combination thereof, or otherwise.

FIG. 8C is a graph of central tendency versus variability (e.g., low range variability) that can be used to evaluate or identify a region or zone that holds or corresponds to a determined central tendency and variability data pair for a particular TOD. Any number of two or more zones can be used. In this embodiment the data pair can correspond to a target zone 425 or one of three hypo risk zones: a low zone 426, a moderate zone 428, or a high zone 430. A first hypo risk function (e.g., a curved or linear boundary), referred to as moderate risk function 422, differentiates between low zone 426 and moderate zone 428. A second hypo risk function, referred to as high risk function 424, differentiates between moderate zone 428 and high zone 430. The central tendency and variability data pair can be evaluated against or compared to the zones to determine a hypo risk metric for the corresponding TOD period.

The hypo risk functions 422 and 424 can be implemented in the DGA explicitly as a mathematical function (e.g., a polynomial) or can be implemented implicitly, such as by defining each zone by the pairs it contains, use of a lookup table, set of if-else statements, threshold comparisons, or otherwise. The hypo risk functions 422 and 424 can be preloaded into the DGA, or can be downloaded from trusted computer system 480, or can be set by another party such as the HCP. Once implemented in the DGA, the hypo risk functions 422 and 424 can be treated as fixed or can be adjusted by the user or HCP. Example methodologies for determining the hypo risk function are described in the '400 publication.

At 416, the DGA can assess a hyperglycemia risk metric (“hyper risk”) based on the central tendency value. In this embodiment, the hyper risk can be evaluated by comparison of the central tendency value for the particular TOD period to a central tendency goal or threshold 432. The magnitude and/or sign of the difference of the central tendency value from the goal 432 can identify the amount of hyper risk. For example, a low hyper risk can be present if the central tendency value is less than the goal 432 (e.g., a negative value). A moderate hyper risk can be present if the central tendency value exceeds the goal 432 (e.g., a positive value) by less than a threshold amount (e.g., 5 percent, 10 percent, etc.). A high hyper risk can be present if the central tendency value exceeds the goal 432 by a value greater than the threshold amount. The use of three discrete groupings for hyper risk (e.g., low, moderate, high) is an example and any number of two or more groupings can be used.

In other embodiments, the DGA can assess a hyperglycemia risk metric, at 416, before assessing a hypoglycemia risk, at 414. Alternatively, in another embodiment, the assessments of hypoglycemia risk, at 414, and hyperglycemia risk, at 416, can be done in parallel at the same time.

Other metrics such as variability risk can also be assessed. For example, a variability value less than a first variability threshold 434 can be indicative of a low variability risk, a variability value greater than the first variability threshold 434 and less than a second variability threshold 436 can be indicative of a moderate variability risk, and a variability value greater than the second variability threshold 436 can be indicative of a high variability risk. Again, the use of three discrete groupings for variability risk is an example. The DGA can use any number of two or more groupings.

At step 418, the DGA can determine a pattern type for the TOD period based on the assessed one or more risk metrics. In one example embodiment, pattern determination can be assessed with the hypo risk metrics and the hyper risk metric. If the hypo risk metric is high, then the pattern can be set as a low pattern (or “Lows” pattern). Otherwise, if the hypo risk is moderate and the hyper risk is either high or moderate then the pattern can be set as a high/low (or moderate) pattern (or “Lows with Some Highs” or “Highs with Some Lows”). Otherwise, if the hyper risk is high or moderate and the hypo-risk is low, then the pattern can be set as a high pattern (or “Highs” pattern). If both the hyper risk and hypo risk are low, then the pattern identified can be No Problem (e.g., an “OK” message is displayed our outputted) (or “no pattern”).

Thus, method 410 is one example of how the DGA can output one of multiple pattern types for each TOD period. The number of pattern types in the pattern types themselves can vary from those described in this embodiment (e.g., low, high/low, high). Once pattern type for the TOD period has been determined, the DGA can store an indicator of the pattern type in a memory location for use in determining a titration recommendation. Referring again to FIG. 8A, the DGA can proceed, at 404, to determine a titration recommendation once completing the GPA for each relevant TOD period.

The recommendation method can branch depending on the pattern type (e.g., low, high/low, high) and other factors including the TOD period, dosing strategy, compliance with the strategy (e.g., whether a dose is missing), and whether sufficient data is available for making an assessment. The DGA does not make titration recommendations until enough data are available for the corresponding TOD period, for example, the DGA can omit assessment and generate an error message if less than a threshold amount of data is available, e.g., less than a threshold number (e.g., five) of separate days with more than a minimum portion (e.g., 90%) of the data available.

FIGS. 8D-8H show example branches of a recommendation algorithm or method for determining a dose titration recommendation based on the input information described above. Other branches can also be useful. FIG. 8D shows a recommendation method branch 440 for a TOD with sufficient data available and possible causes for a low pattern type including one or more of a higher than optimal basal dose, meal dose, premeal correction dose, or post-prandial dose. At 442, the DGA evaluates whether the pattern type for an overnight TOD period is low. If the pattern is low, at 444, the DGA generates a recommendation to reduce all relevant doses, including at least a basal dose and optionally, one or more of a meal dose, premeal correction dose, or post-prandial dose, by an equal amount, for example, 10%. Titration recommendation rules for the low patterns can include, for overnight TOD periods, generating a recommendation to reduce the long acting insulin dose(s) or basal rate at 444. At 446, if any other TOD period has a low pattern, the DGA can generate a recommendation, at 448, to reduce the fixed meal dose for the relevant TOD period only.

In this embodiment 440, if there is at least one low pattern, then no titration guidance for any high pattern TOD period is provided. The idea here is to emphasize prevention of hypoglycemia and to only increase doses when the risk of hypoglycemia is low in all TOD periods. Also, it is possible that in some situations, when a TOD period has a high pattern, this could be caused by a prior TOD period having a low pattern, and the patient is overeating to compensate—so addressing the low pattern can in itself help address a subsequent high pattern. At 449, if the pattern is not high, the process 440 waits or terminates without generating a recommendation or passes to a high pattern evaluation 450.

Accordingly, for high/low patterns, the DGA generates no titration guidance. If there are no TOD periods where titration guidance can be given and data is sufficient for all time periods, then the DGA can provide a message to the patient indicating that they need to address glucose variability before further titration guidance can be given. Also, the DGA can provide a report to the patient's HCP to consider alternative medications or therapies that can address glucose variability.

FIG. 8E shows operations by a DGA for generating titration recommendations for high patterns when there are no low pattern TOD periods. At 452, if the overnight period has a high pattern and there is no other period with moderate risk of hypoglycemia, the DGA can increase the long acting insulin dose(s) or basal rate recommendation at 454. At 456, if the overnight period has a high pattern, and there is at least one other non-dinner period with moderate risk of hypoglycemia, then at 458, the DGA can decrease the meal insulin dose associated with any period with moderate risk of hypoglycemia. At 460, if the overnight TOD period has no moderate risk of hypoglycemia and no high pattern, then at 462, the DGA can generate a recommendation to increase the meal insulin dose associated with the first TOD period with a high pattern. At 464, if the overnight period has a moderate risk of hypoglycemia, and the only post-meal period with a high pattern is dinner, at 466 the DGA can generate a recommendation to increase the long acting insulin dose(s) or basal rate. If the overnight period has a moderate risk of hypoglycemia, but not the post-dinner period, at 462 the DGA can generate a recommendation to increase the meal insulin dose associated with the first TOD period with a high pattern.

In alternative embodiments, the pre-meal glucose can be higher or lower than a target glucose (for example, 120 mg/dL). The glucose data for each meal that contributes to the calculation of the hypo and hyper risk metrics can be modified to compensate for the effects from a prior meal or condition that affects glucose that is not due to the current meal. The DGA can modify these data by subtracting the offset so the resulting starting glucose is the target level. Alternatively, the DGA can modify these data by a “triangle” function where, for the meal start time, the difference between the meal start glucose and the target glucose is subtracted, but this modification is reduced over time; either linearly for a defined period (e.g., three (3) hours), or another decay function.

Alternatively, this function can itself be a function of meal start glucose level or glucose trend, and/or when the previous meal dose was taken.

According to another aspect of the embodiments, algorithms for generating meal bolus titration recommendations can become more complex when additional aspects are factored in, such as, for example, missed meal doses, missed basal doses, post-prandial corrections, and pre-meal corrections. Algorithms for providing appropriate recommendations if these factors are present can require excluding some data, while still meeting data sufficiency thresholds after excluding data to provide guidance.

For example, referring to FIG. 8F, if a high pattern is detected at 461 with some days missing the meal dose, then the days with the missing meal dose are excluded 463 and the GPA analysis 410 is repeated. If a high pattern is subsequently detected 465, then the dose can be increased 467, based on the patterns identified in the other TODs, or wait for further input or return at 469. Alternatively, the system could only evaluate for high patterns using data where days with the missing meal dose are excluded. An algorithm 470 with this branching pattern is diagrammed in FIG. 8F. If the system detects a low pattern at 473, it may perform a low pattern algorithm 472 described in the following paragraph. If the system does not detect a high pattern or a low pattern, it may revert to block 469 for further input or return.

At 472, for missed meal doses, if the DGA detects a low pattern in a TOD period, and if the meal dose was missed for some of the days during this TOD, then the DGA can generate a recommendation to reduce the dose. The recommendation can include, for example, reducing the fixed portion or the correction-dose portion.

Regarding missed basal doses, if the DGA detects a low pattern 473 in the overnight TOD, missing basal doses should not impact the dose titration logic. Likewise, if a low pattern is detected in a TOD other than the overnight period, the missing basal doses should not impact the dose titration logic.

If the DGA detects a high pattern 461 in a TOD using data that includes at least one day (or TOD) with a missed basal dose, then data for any day or days (or TOD(s)) with the missing basal dose can be excluded 463 and the pattern analysis 410 is repeated. Subsequent actions can depend on the particular TOD in which the high pattern was detected. For example, if the DGA detects a high pattern in the overnight TOD in data including at least one day with a missed basal dose, then data for any day or days with the missing basal dose can be excluded and the pattern analysis is repeated. If a high pattern in the overnight TOD is detected when the day(s) with the missed basal dose(s) are excluded, then the basal dose can be increased, as the overnight TOD results can be used as a guide for adjusting basal dose. If a high pattern is detected in a TOD other than the overnight period, then the days with the missing basal dose can be excluded and the pattern analysis repeated. If a high pattern is detected when the day(s) with the missed dose(s) are excluded, then the meal dose associated with the TOD with the high pattern can be analyzed for titration as described herein. In either case, the logic flow 470 is as diagrammed in FIG. 8F.

FIG. 8G shows an example for a logic flow 474 for developing recommendations with post-prandial corrections. If after GPA 410 the DGA detects a low pattern 479 for a TOD with some days including a post-meal correction, then the following analyses can be used to titrate the correction or meal dose. At 475, if the DGA first detects a low pattern 479 it can exclude data for the days without a post-meal correction, first testing that sufficient data is available at 487. If sufficient data is not available, the DGA may perform an error recovery routine 489, for example, displaying an error message. If sufficient data is available, the DGA may repeat the pattern analysis 410. If, subsequently, the DGA detects a low pattern, it can reduce the post-prandial correction dose (that is, increase the correction factor) at 476, subject to pattern analysis findings in the other TODs. If, subsequently, the DGA does not detect a low pattern, it can reduce the meal dose at 477.

For all embodiments described herein, modification (e.g., titration) of a correction dose in one direction can be achieved by modification of the correction factor in the opposite direction. The two parameters are inversely related, such that an increase in correction dose can be achieved by a decrease in correction factor, and a decrease in correction dose can be achieved by an increase in correction factor. Thus, in all of the embodiments described herein, the DGA can recommend or implement a correction either by modification of the correction factor or by modification of the correction dose. Thus, to the extent modification or titration of a correction factor is described herein, the embodiments can be configured to achieve the same effect by an inverse modification of the correction dose and, conversely, to the extent modification or titration of a correction dose is described herein, the embodiments can be configured to achieve the same effect by an inverse modification of the correction factor. Given this interchangeability, both options are available for every embodiment described herein although both options will not be described for every embodiment solely for ease of description.

In addition, or in an alternative, starting with an original data set 491, at 478 the DGA can exclude days with missed meal doses. After finding sufficient data at 487, if the pattern analysis 410 of these data with days with post-meal corrections excluded does not indicate a low pattern at 490, then the DGA can recommend reducing the post-prandial correction dose 476. Otherwise, the DGA can implement the logic 510 of FIG. 10B of U.S. application Ser. No. 16/944,736, published as US 2021/0050085, which is hereby expressly incorporated by reference in its entirety for all purposes, which can result in a recommendation to reduce either the meal time insulin or pre-meal correction portions of the dose guidance. If the DGA does not detect lows at 479 and does not detect any high glucose pattern at 492, it may wait for further input or return at 469. If the DGA does detect a high pattern at 492, then it may implement the process 480 at block 471 (FIG. 8H).

Referring to FIG. 8H, if the DGA detects a high pattern 493 for a TOD with some days including a post-meal correction, then it can implement the following procedure 480 to develop a recommendation for titrating the correction and meal doses. At 481, the DGA can include data for days with a missed dose and with a post-meal correction, and repeat the pattern analysis 410. If subsequently the DGA detects a high pattern at 494, it can increase the post-prandial correction dose (that is, decrease the correction factor) at 482, subject to pattern analysis findings in the other TODs. If it does not detect a high pattern at 494, it may check for a low pattern at 495 and revert to 474 of FIG. 8G if detecting a low pattern, or else wait for further input or return at 469. Although not shown in FIG. 8H, after excluding any data for GPA 410 and before performing the GPA, the DGA may test for sufficiency of data and perform an error recovery routine if available data is insufficient.

In an alternative, or in addition, starting with the original data set at 493, if the pattern analysis of the data excluding days with missed meal doses at 483 indicates a high pattern along either one of branches 2.1 or 2.2, the DGA can continue the procedure 480 as follows. On branch 2.1, if pattern analysis 410 of data for days excluding post-meal corrections at 484 (i.e., data with bolus dose only) does not indicate a high pattern at 497, then the DGA can generate a recommendation to increase the post-prandial correction dose at 482, subject to pattern analysis of the other TODs. Otherwise the DGA can generate a recommendation to increase either the meal time insulin or pre-meal correction portions, following the procedure 550 of FIG. 10C of U.S. application Ser. No. 16/944,736, published as US 2021/0050085, which was previously incorporated by reference in its entirety.

On branch 2.2, if the pattern analysis on the data with only days with post-meal corrections included at 485 does not indicate a high pattern at 496, then the DGA can increase either the meal time insulin or pre-meal correction portions, following the procedure 550 of FIG. 10C of U.S. application Ser. No. 16/944,736, published as US 2021/0050085, which was previously incorporated by reference in its entirety. If a high pattern is not detected at 496, the DGA may revert to block 484.

If correction factor titration recommendations from the different TODs are conflicting, and if the patient is currently using the same correction factor for all TODs, then the DGA can increase the correction factor. The procedure 480 can increase the meal dose first, if all three components, meal dose, pre-meal correction, and post-prandial correction are less than optimal. Pre-meal correction can be up-titrated after the meal dose has been titrated. Post-prandial correction can be up-titrated after meal dose and premeal correction have been corrected.

If during subsequent analysis, the TODs for which the DGA generated an ‘increase correction factor’ recommendation by the foregoing method, now will result in a recommendation of ‘no change in correction factor.’ Conversely, if TODs with a ‘decrease correction factor’ recommendation by the foregoing method still receive a recommendation to ‘decrease correction factor’, then the different TODs will likely be optimized by use of different correction factors.

While FIGS. 8D-8H show aspects of various recommendation algorithms 404 for use in the method 400, it should be appreciated that these are examples. Various other algorithms may also be suitable.

### Guidance Period

### Algorithm Description

During the Guidance Period, the DGS 100 can (1) provide the user with insulin dose recommendations for meals as well as post-meal corrections, and (2) titrate originally learned dose settings from the Learning Period to improve glycemic control.

Before the Guidance Period can begin, insulin dose settings may need to be initialized. Initialization may be achieved either through successful learning or through manual entry of initialized values if learning is not successful. Once the insulin dose settings are initialized, the user may receive insulin dose advice in multiple ways. The user may request a specific meal dose through the DGA. Alternatively, the DGS 100 may detect and notify the user of a missed meal dose event. If the user confirms the missed meal dose event, the DGS 100 may supply a “late meal dose” recommendation to account for the fact that insulin dosing is occurring after meal start instead of the prescribed administration at or before meal start. Alternatively, the DGS 100 may detect and notify the user if glucose between meal doses is too high. If the user confirms that the glucose between meal doses is too high, the DGS 100 may suggest a “post-meal correction dose” to bring the user's glucose into a target or goal range before the next meal dose.

The frequency with which subroutines for dose suggestions are processed may vary. Those subroutines associated with detecting missed meal doses and high post-meal glucose may continuously run while those subroutines associated with dose calculations may only run when requested by the user. Implementation of when mealtime or post-meal dose suggestions may be invoked may be governed by a state transition diagram depicted in FIG. 10. Periodically, the DGS 100 may titrate fixed dose and correction factor values to improve a user's glycemia. These titration algorithms may be processed daily and may operate independently of the dosing algorithm discussed above.

### Customized Settings

In some embodiments, the user may customize certain settings. For example, in some embodiments, the user may indicate that certain data should be disregarded by the algorithm. Alternatively, the user may be sick or taking a new medication, and the user may want to disregard a period of time. The DGS may include a “vacation mode” that allows the user to inform the DGS that the DGA should ignore certain past or future days or time periods where their behavior may be or was atypical. For example, if the user is going on a cruise or would like to exclude Labor Day Weekend, the user may select a period of time for the DGS to disregard in determining titrations.

The user in some embodiments, the also adjust previously selected dose amounts and time periods if there has been a change in the user's routine. In some embodiments, if the time that the user normally eats a meal has changed, the user may enter a new time period for that meal so that the DGS may correctly log the meal dose.

In some embodiments, the use may also enter a dose adjustment as a result of a new medication. In some embodiments, this change may only be changed if the user's HCP has advised them to enter the change. For instance, the HCP may have prescribed a new oral diabetes medication that the DGS needs to consider. For example, a 10% increase for glucose-raising agents (e.g., steroids) or a 10% decrease in glucose-lowering agents (i.e., metformin) may need to be entered. In the event of such a change, the DGS may adjust the indicated dose immediately and the DFA may continue to learn and adjust based on the user's glucose results after the dosing change.

### State Transition Diagrams for Dosing Algorithms

The DGS 100 may recommend two types of rapid acting insulin doses to a user—meal doses and correction doses. Meal doses are doses that requested by the user to address glucose increases in response to a meal. Correction doses are doses that are recommended by the DGS 100 to correct for high glucose between meal doses.

When the functions that calculate these meal and correction doses may be called is determined by state transition diagrams. The state transition diagrams that govern insulin dosing, known together as the Dose Guidance State Machine (DGSM) 2000, is presented in FIG. 10.

The current state of dose guidance is determined by two factors: time since the dose was delivered and the classification of recent past doses. Insulin dose time is defined by the timestamp that the system receives from the connected insulin pen. Classification is dependent upon the rules for defining insulin doses in other portions of the application discussing real time dose classification.

Meal Dose State Machine 2002—A waiting state denoted as “Meal Dose Guidance Available State” 2004 may be defined as being greater than 2 h since the initial meal insulin dose. In the Meal Dose Guidance Available State 2004, a user can receive meal dose guidance by querying the DGS 100 for a dose recommendation. Once an insulin dose (including timestamp and amount) is received by the DGS 100 and is correctly classified as an initial meal dose, a transition occurs to the “Post-Meal Dose State” 2006, where the DGS 100 will remain for a period of time, e.g., 2 hours, following the timestamp reported by the connected pen. In this Post-Meal Dose State 2006, meal doses may not be recommended to the user. Any extra doses received by the DGS 100 while in the Post-Meal Dose State 2006 may also be considered meal doses and may not affect the time spent in this state.

Correction Dose State Machine 2010—Criteria for triggering a correction dose notification are defined in other sections of this application. Once any non-prime dose is received and classified as a correction dose, a transition occurs from the waiting state (correction-only dose guidance available) state 2014 to the post-correction dose state 2016, where correction-only dose guidance may not be available. The DGS 100 may remain in this state for a period of time, e.g., 2 hours, from the timestamp reported by the connected pen. Correction doses may not be recommended while in the post-correction dose state 2016. In the event that any non-prime insulin dose is recorded in the post-correction dose state 2016, a timer for the period of time, e.g., 2 hours, will restart at the timestamp of the new dose. Priming before administering a dose of insulin removes air from the needle and cartridge, ensuring that the correct amount of insulin is administered with the full dose. A prime dose is part of a safety test in which a small dose of insulin (e.g., 2 units) is shot upwards into the air, and the user can watch to ensure that insulin comes out of the tip of the needle.

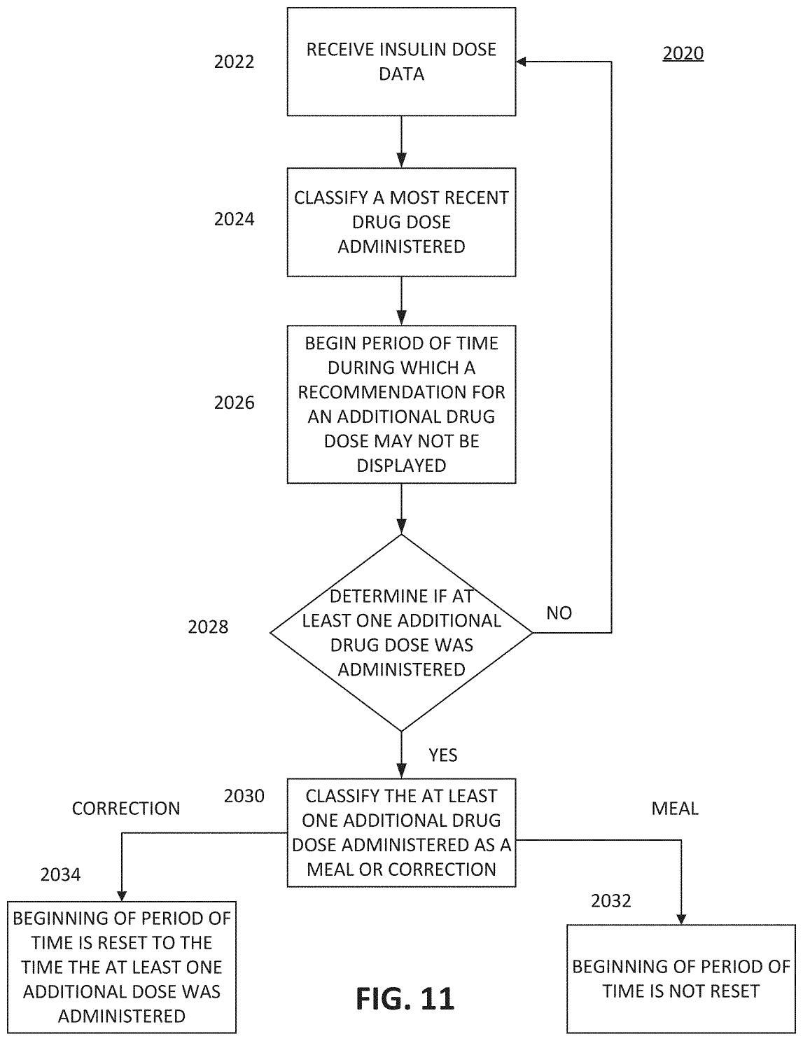

As seen in FIG. 11, in exemplary method 2020, beginning with step 2022, the DGS 100 can receive or otherwise access insulin data of the subject (e.g., from MDD 152). For example, the DGA can check for the latest insulin delivery information by requesting delivery information from different sources, including, but not limited to, the MDD 152, the MDD-associated application, or the interface that stores the latest insulin delivery information (such as the MDD application web server), or by checking the memory of the various applications for the latest insulin delivery information.

At step 2024, the DGS 100 may classify the most recent drug dose administered as one of a certain category of doses. For example, the most recent dose administered may be a meal dose, a correction dose, or a prime dose. The most recent dose administered may be automatically classified by the DGS 100 or manually classified by the user as explained elsewhere in this application. If the most recent dose is a prime dose, then no further action may be taken. If the most recent dose administered is a meal dose or a correction dose, then at step 2026, the DGS 100 enters a period of time during which a recommendation for an additional drug dose may not be displayed. The period of time may be between about 1 to about 3 hours, alternatively between about 1.5 to about 2.5 hours, alternatively about 1.5 hours, alternatively about 2 hours, alternatively about 2.5 hours, alternatively about 3 hours.

At step 2028, the DGS 100 may determine if at least one additional drug dose was administered, i.e., after the time that the earlier “most recent drug dose” was administered. If at least one additional drug dose was administered, at step 2030, the DGS 100 may classify the at least one additional drug dose as either a meal dose or a correction dose. If the at least one additional drug dose was a meal dose, at step 2032, no change is made to the period of time during which a recommendation for an additional drug dose may not be displayed, i.e., the start of the period of time is still the time at which the most recent drug dose was administered. If, however, the at least one additional drug dose was a correction dose, at step 2034, the beginning of the period of time during which a recommendation for an additional drug dose may not be displayed may be restarted and set to start at the time that the at least one additional drug dose was administered.

### Meal Dose Calculation Algorithm

Interaction with the User Interface

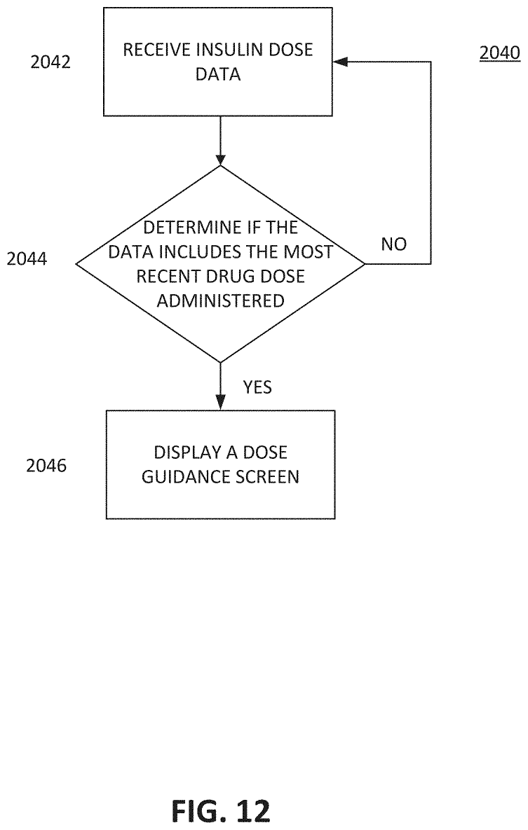

The DGS 100 may only display the dose guidance screen when the dose guidance state machine (DGSM) and the dose classification state machine DCSM are up-to-date. As seen in FIG. 12, in exemplary method 2040, beginning with step 2042, the DGS 100 can receive or otherwise access insulin data of the subject (e.g., from MDD 152). For example, the DGA can check for the latest insulin delivery information by requesting delivery information from different sources, including, but not limited to, the MDD 152, the MDD-associated application, or the interface that stores the latest insulin delivery information (such as the MDD application web server), or by checking the memory of the various applications for the latest insulin delivery information. The DGA may record a dose when the DGA receives the insulin dose information (e.g., dose amount and time administered) electronically.

In step 2044, the DGS 100 may determine if the data that was received includes the most recent drug dose data administered, i.e., the data is “up to date.” To be up to date, the DGSM may need (a) the most recent recorded insulin dose to be confirmed by the user, and/or (b) all recorded doses since a reset time may need to be classified, either automatically by (treses) the system or manually by the user. The confirmation may be performed manually, e.g., the DGS 100 may prompt the user to confirm that the last dose recorded was truly the last dose. Alternatively, the confirmation may be performed by the DGA interrogating the connected pen to ensure that it has the most recent dose information. In some embodiments, the display device 120 can be configured to transmit a request for data (e.g., interrogation) to the MDD 152 via a wired or wireless communication link. In response to the received request, the MDD 152 can transmit data to the display device 120. In some embodiments, for example, the display device 120 can be configured to communicate with the insulin pen according to a Near Filed Communication (NFC) protocol. In other embodiments, the MDD 152 can autonomously send data to the reader device over a wired or wireless communication link. The MDD 152 can be configured to transmit according to a schedule, based on a triggering event or condition, and/or when it comes into a wireless communication range of the reader device. In some embodiments, the MDD 152 can be configured to communicate with the display device 120 according to a Bluetooth or Bluetooth Low Energy networking protocol. Those of skill in the art, however, will recognize that other wireless communication protocols may be implemented (e.g., infrared, UHF, 802.11x, etc.). The DCSM may be reset at tᵣₑₛₑₜ. The reset time (tᵣₑₛₑₜ) may be set to midnight, but alternatively, may be a different TOD depending on the dose regimen timing parameters. It is to be understood that each of these conditions is optional and are not necessarily required.

In step 2046, a screen may be displayed if it is determined that the DGS 100 has data relating to the most recent drug dose administered. The screen may be a home screen of the DGA that may include a dose guidance recommendation. The screen may only be displayed for a predetermined time period after user confirmation of the most recent dose. The predetermined time period may be at least about 10 minutes, alternatively at least about 15 minutes, alternatively at least about 20 minutes, alternatively about 15 minutes. If a bolus record is recorded while the home screen, or any of its dependent flow screens, are active, then the DGA may exit these screens and request a new user confirmation (if the dose is not automatically classified) and subsequent flows and logic so the state machines may be updated before the home screen may be displayed again.

Dose Classification State Machine

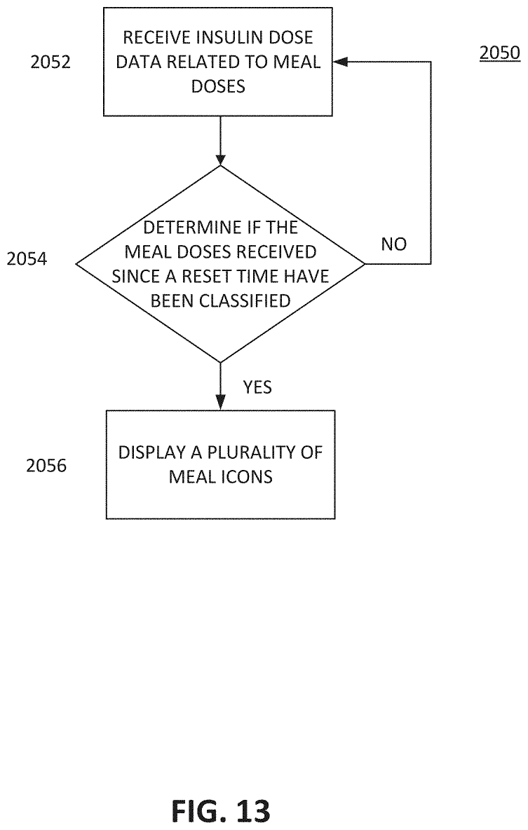

The DCSM 2000 has information needed to determine how to display meal carousel icons within the DGA home screen, and how icons function when selected. As seen in FIG. 13, in exemplary method 2050, beginning with step 2052, the DGS 100 may receive or otherwise access insulin data of the subject (e.g., from MDD 152). For example, the DGA can check for the latest insulin dose data by requesting delivery information from different sources, including, but not limited to, the MDD 152, the MDD-associated application, or the interface that stores the latest insulin delivery information (such as the MDD application web server), or by checking the memory of the various applications for the latest insulin delivery information. The insulin dose data may be related to meal doses administered since a reset time.

In step 2054, the DGS 100 may determine if the meal doses that were received since the reset time have been classified. The meal doses may be classified as associated with a particular meal, i.e., breakfast, lunch, or dinner. As discussed with respect to other methods and embodiments described herein, the meal doses may be either classified automatically by the DGS 100 or manually by the user.

In step 2056, if it is determined that all of the meal doses received since the reset time have been classified, then the DGS 100 may display a screen that includes a plurality of meal icons. The plurality of meal icons may include an icon each for breakfast, lunch, and dinner. Each of the meal icons may have a first appearance or presentation associated with a first state and a second appearance or presentation associated with a second state. The first appearance or presentation may be associated with a first state in which a meal dose administered since the reset time has been classified as a meal type corresponding to the breakfast, lunch, or dinner icon. The second appearance or presentation may be associated with a second state in which a meal dose administered since the reset time has not been classified as a meal type corresponding to the breakfast, lunch, or dinner icon. For example, a meal icon (one each for breakfast, lunch, or dinner) may be “shaded” (first state) if an associated meal record exists since tᵣₑₛₑₜ for that meal. If selected, the shaded meal icon may display the recorded dose information and provide an option for displaying the meal dose guidance calculation, if available. If no associated meal record exists within this time period (since tᵣₑₛₑₜ) (second state), then the icon may not be shaded and when selected, a meal dose calculation may be displayed if available. When the time has crossed treses, then all icons may be reset to unshaded (second state) and may be available for displaying a meal dose calculation.

The variable treses may be determined by the regimen parameters, dinner dose time range and breakfast dose time range. If the dinner dose time range is the last meal dose time range before midnight, then treses will equal midnight (this is the most likely case). Otherwise, tᵣₑₛₑₜ should be set to the halfway point between the end of the dinner dose time range and the start of the breakfast dose time range.

Meal Dose Calculation

When a meal icon has been selected and the meal dose calculation is to be displayed, the DGA may determine if a normal meal dose calculation should be used or if a late dose calculation should be used to determine the appropriate dose recommendation. In order to determine which meal dose calculation is appropriate and to retrieve the associated calculation result, the DGA may determine if a missed dose alert, which is described elsewhere in this application, is active.

The dose recommendation displayed may be based on the late meal dose calculation if a missed meal dose alert is asserted. Additionally, no insulin doses may have been recorded in the past two hours. It is to be understood that each of these conditions is optional and are not necessarily required. Note that the DGA may check to ensure that all of the alerts are rescinded before dose guidance is provided, in order to avoid race conditions; for example, where the dose has not been received until the user is prompted to connect or scan the MDD 152, and an alert may have not yet been rescinded If the above conditions are not met, the dose displayed by the DGA may be based on the normal meal dose calculation.

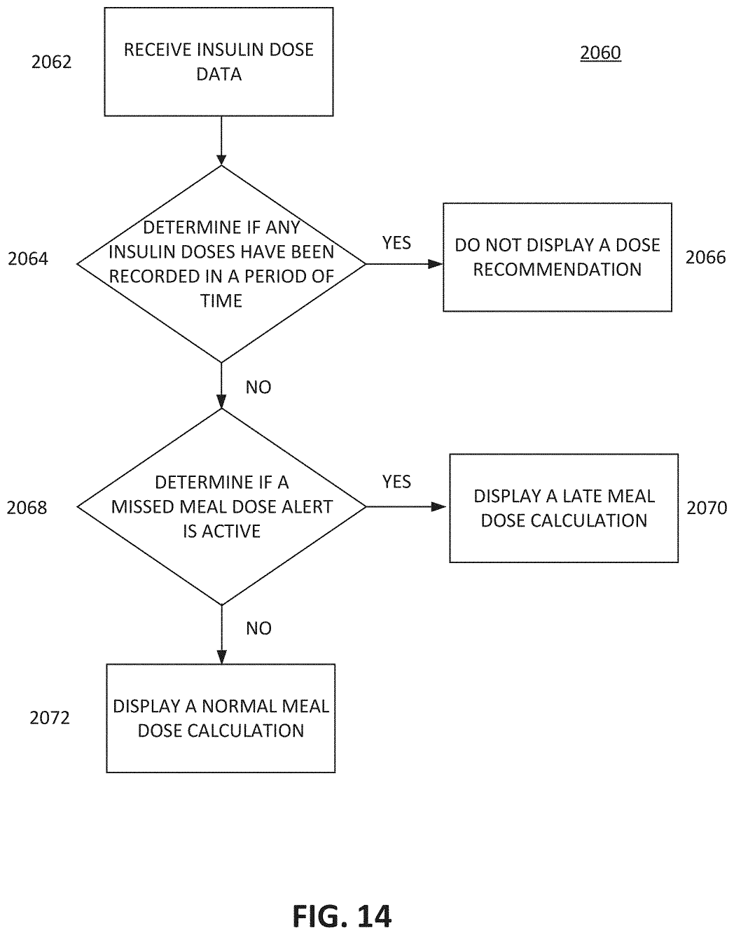

As seen in FIG. 14, in exemplary method 2060, beginning with step 2062, the DGS 100 can receive or otherwise access insulin data of the subject (e.g., from MDD 152). For example, the DGA can check for the latest insulin dose data by requesting delivery information from different sources, including, but not limited to, the MDD 152, the MDD-associated application, or the interface that stores the latest insulin delivery information (such as the MDD application web server), or by checking the memory of the various applications for the latest insulin delivery information.

In step 2064, the DGA can determine if any insulin doses have been recorded for a period of time, e.g., 2 hours. As seen in step 2066, if any insulin doses have been recorded in the last 2 hours, then the DGA may not display a dose recommendation.

If an insulin dose has not been recorded in the last 2 hours, then in step 2068, the DGA may determine if a missed dose alert is active. If a missed dose alert is active or has been asserted, then in step 2070, the DGA may display a dose recommendation based on a late meal dose calculation. If a missed dose alert is not active or has not been asserted, then in step 2072, the DGA may display a dose recommendation based on a normal meal dose calculation.

A normal meal dose calculation (i.e., one that is not a late meal dose calculation) may be based on a recent glucose level (e.g., scanned or streaming glucose) and may be represented by the logic and equations below:

If Gₚᵣₘ≤G_{T}

I_{guide}=I_{fixed}−IOB (Eq. 1)

Else

I_{guide}=I_{fixed}−IOB+CorrectionAdj+TrendAdj (Eq. 2)

Where
Gₚᵣₘ=current scanned glucose value
G_{T}=target glucose (regimen parameter lookup)
I_{guide}=insulin dose calculation result
I_{fixed}=fixed insulin dose associated with breakfast, lunch or dinner as applicable (regimen parameter lookup)
IOB=insulin on board (calculated in insulin on board module)
CFₚᵣₘ=correction factor pre-meal (regimen parameter lookup)
CorrectionAdj=(Gₚᵣₘ−G_{T})/CFₚᵣₘ
TrendAdj=trend adjustment (based on Kudva, et al. table lookup), based on CF=CFₚᵣₘ

The definition of glucose trend as well as TrendAdj by CFₚᵣₘ are explained in the below tables from Kudva, et al. “Approach to Using Trend Arrows in the FreeStyle Libre Flash Glucose Monitoring Systems in Adults.” J Endocr Soc. 2018; 2(12):1320-1337, which is hereby expressly incorporated by reference in its entirety for all purposes.

The calculation of I_{guide} may be rounded to the nearest unit of insulin according to standard rounding rules. If the calculated dose is negative, then the displayed dose may be to be set to zero. In some embodiments, CorrectionAdj may be negative if premeal glucose is less than the target glucose. Note that in an alternative embodiment, the IOB value is only subtracted from the correction and trend adjustment. For instance, if there is no correction and trend adjustment, then Iguide=Ifixed. If there is an adjustment, then the IOB is subtracted from that value before adding to Ifixed. If the adjustment minus the IOB is less than zero, then Iguide=I_{fixed}.

As an additional safety measure to the timeout described above (see, e.g., step 2046 of FIG. 12), the glucose data received by the DGS according to a schedule or based upon a triggering event or condition and/or when it comes into wireless communication with a reader device may be used to determine if a meal dose recommendation is to be calculated and presented to the user. In some embodiments, if a user's current glucose at the time of a meal dose calculation request is below a threshold value, then the DGS may not present a calculated dose suggestion to the user. Instead, the DGS may present a warning to the user about their current glucose levels, suggesting that they raise their current glucose values before administering an insulin dose. In some embodiments, a dose guidance is not displayed if the current glucose value is determined to be below the threshold value. This additional safety measure is to avoid hypoglycemia resulting from dosing insulin at too low of a glucose value. This threshold value may be configurable based upon a user's tolerance of hypoglycemia.

Insulin on board (JOB) is a measure of residual active insulin remaining in a user's bloodstream following an injection. IOB is subtracted from a current calculated dose to account for active insulin from previous injections to avoid insulin-induced hypoglycemia. Calculated by multiplying a previous dose amount by a fraction that denotes the percent of insulin remaining at that point in time post-injection. IOB has units of insulin (U).

TABLE 2
Definition of glucose rate of change
trend arrow bins from Kudva, et al.

| Trend | | |
|---|---|---|
| Arrow | Glucose Direction | Change in Glucose |
| [symbol] | Rising quickly | Glucose is rising quickly |
| | | Increasing >2 mg/dL/min or |
| | | >60 mg/dL in 30 minutes |
| [symbol] | Rising | Glucose is rising |
| | | Increasing 1-2 mg/dL/min or |
| | | 30-60 mg/dL in 30 minutes |
| [symbol] | Changing slowly | Glucose is changing slowly |
| | | Not increasing/decreasing |
| | | >1 mg/dL/min |
| [symbol] | Falling | Glucose is falling |
| | | Decreasing 1-2 mg/dL/min or |
| | | 30-60 mg/dL in 30 minutes |
| [symbol] | Falling quickly | Glucose is falling quickly |
| | | Decreasing >2 mg/dL/min or |
| | | >60 mg/dL in 30 minutes |

TABLE 3A
Table of TrendAdj, trend-based post-meal mealtime insulin
dose adjustments defined in Kudva, et al.
Insulin dose adjustments using trend arrows in adults
(post-meal corrections ≥3 hours post-meal)
Insulin Dose Adjustments

| Trend | Correction Factor (CF) | | | |
|---|---|---|---|---|
| Arrows | <25 | 25 ≤ 50 | 50 ≤ 75 | ≥75 |
| [symbol] | +3.5 units | +2.5 units | +1.5 units | +1.0 units |
| [symbol] | +2.5 units | +1.5 units | +1.0 units | +0.5 units |
| [symbol] | No adjustment | No adjustment | No adjustment | No adjustment |
| [symbol] | −2.5 units | −1.5 units | −1.0 units | −0.5 units |
| [symbol] | −3.5 units | −2.5 units | −1.5 units | −1.0 units |

TABLE 3B
Table of TrendAdj, trend-based pre-meal mealtime insulin
dose adjustments defined in Kudva, et al.
Insulin dose adjustments using trend arrows in adults
(pre-meal corrections
Insulin Dose Adjustments

| Trend | Correction Factor (CF) | | | |
|---|---|---|---|---|
| Arrows | <25 | 25 ≤ 50 | 50 ≤ 75 | ≥75 |
| [symbol] | No adjustment | No adjustment | No adjustment | No adjustment |
| [symbol] | No adjustment | No adjustment | No adjustment | No adjustment |
| [symbol] | No adjustment | No adjustment | No adjustment | No adjustment |
| [symbol] | −2.5 units | −1.5 units | −1.0 units | −0.5 units |
| [symbol] | −3.5 units | −2.5 units | −1.5 units | −1.0 units |

The meal dose calculation may be triggered by a user request for a meal dose in the DGA. The input data streams may include scan glucose at the time of the request, scan glucose trend data at the time of the request, IOB at the time of the request, and a check for a late dose. The input parameters may be a fixed dose for a requested meal, mealtime CF, and target glucose. The outputs may be a suggested mealtime insulin dose.

Late Meal Dose Calculation

Both normal and late meal dose amounts may be calculated when a person selects a meal dose within the DGA. Presentation of the late meal dose value may be dependent on the DGS 100 detecting a missed meal dose event, as described elsewhere in this application.

A late meal dose calculation may be the same as for the normal meal dose calculation explained above, except that Gₚᵣₘ and TrendAdj may be calculated using continuous streaming glucose data, e.g., once-per-minute streaming glucose, rather than scanned glucose. For a late meal dose calculation, Gₚᵣₘ may be the glucose value at the estimated meal start time and TrendAdj may be determined using the glucose trend at the estimated meal start time. The estimated meal start time may be calculated based upon once-per-minute streaming data and may be an output from the missed meal dose detection algorithm, which is described elsewhere in this application. Moreover, for a late meal dose calculation, IOB may be calculated according to the time that a late meal dose is requested by the user, not the estimated meal start time. If the late meal dose calculation result is less than the normal meal dose calculation result, then the DGS 100 may display the lower value.

As an additional safety measure to the timeout described above (see, e.g., step 2046 of FIG. 12), the glucose data received by the DGS according to a schedule or based upon a triggering event or condition and/or when it comes into wireless communication with a reader device may be used to determine if a late meal dose recommendation is to be calculated and presented to the user. In some embodiments, if a user's glucose at the estimated meal time was below a threshold value, then the DGS may not present a calculated dose suggestion to the user. Instead, the DGS may present a warning to the user to correct low glucose before dosing insulin in place of a dose recommendation. This additional safety measure is to avoid hypoglycemia resulting from dosing insulin at too low of a glucose value. This threshold value may be configurable based upon a user's tolerance of hypoglycemia.

The late meal dose calculation may be triggered by a user responding to a missed meal dose notification, where the user selects a meal and scans glucose. The input data streams may include an estimated meal start time, a historic glucose at the estimated meal start time, and the IOB at the time of the requested late dose. The input parameters may be a fixed dose for a requested missed meal, mealtime CF, and target glucose. The outputs may be a suggested mealtime insulin dose at the estimated meal start.

Dose Calculation Explanation Display

When the user selects the dose amount displayed, a popup screen may show an explanation of how the dose amount was calculated. The dose amount may be displayed for two components: I_{fixed} and I_{guide}-I_{fixed}. Explanatory text may also be displayed according to TABLE 4.

TABLE 4
Explanatory text to be displayed with dose calculations.

| | | Breakfast | | |
|---|---|---|---|---|
| | | Suggested Dose | Lunch | Dinner |
| | | Breakdown | Suggested Dose | Suggested Dose |
| | | To cover food | Breakdown | Breakdown |
| | | Amount of insulin | To cover food | To cover food |
| | | to cover good for | Amount of insulin | Amount of insulin |
| | Logic | your typical | to cover good for | to cover good for |
| | if logic for | breakfast | your typical lunch | your typical dinner |
| | any inputs | and | and | and |
| | below are not | adjustment(s), | adjustment(s), | adjustment(s), |
| | “none” or | which | which | which |
| | “else” | include(s) . . . | include(s) . . . | include(s) . . . |
| Inputs | Else | {skip} | {skip} | {skip} |
| Correction | | | | |
| Adj | | | | |
| | Positive | additional insulin | additional insulin | additional insulin |
| | | to address high | to address high | to address high |
| | | mealtime glucose | mealtime glucose | mealtime glucose |
| | Else | {skip} | {skip} | {skip} |
| Trend Adj | | | | |
| | Positive | more insulin to | more insulin to | more insulin to |
| | | address upward | address upward | address upward |
| | | glucose trend | glucose trend | glucose trend |
| | Negative | less insulin to | less insulin to | less insulin to |
| | | address downward | address | address downward |
| | | glucose trend | downward | glucose trend |
| | | | glucose trend | |
| | None | {skip} | {skip} | {skip} |
| Late Dose | | | | |
| Adjustment | | | | |
| | Positive | less insulin | less insulin | less insulin |
| | | because the dose is | because the dose | because the dose is |
| | | after meal start | is after meal start | after meal start |
| | None | {skip} | {skip} | {skip} |
| IOB | | | | |
| Adjustment | | | | |
| | Positive | less insulin to | less insulin to | less insulin to |
| | | account for insulin | account for | account for insulin |
| | | still in your body | insulin still in | still in your body |
| | | from earlier doses | your body from | from earlier doses |
| | | | earlier doses | |
| | Else | {skip} | {skip} | {skip} |

Tracking and Tagging Meals

In some embodiments, users may be able to tag meals that have resulted in variable glucose levels, e.g., glucose levels above or below the target range. In some embodiments, based on the tagged meal and the associated post-prandial glucose data from a plurality of the tagged meals, the DGS may provide a new suggested dose that is specific for the tagged meal.

In some embodiments, a user may eat the same pancakes and eggs breakfast at a local restaurant once a week. Every time they eat the pancakes and eggs breakfast, however, their post-prandial glucose levels go out of their target range. In some embodiments, the DGS may notify the user that their meal dose did not get the user back to their target after the meal and may prompt the user to tag or track this meal. The user may enter a description tag for this meal as a favorite.

Thereafter, after the user eats or consumes the same meal, the user can tag the meal and the DGS may associate the meal dose administered in association with the tagged meal and the post-prandial glucose data set associated with the meal with this tag. Once the DGS has acquired a large enough data set, the DGS may determine a specific dose recommendation for this tagged meal based at least on the previous doses administered and the post-prandial glucose data set associated with the tagged meal. The DGS may also consider the user's current glucose level at the time that the request for a dose recommendation was made in the determination of the dose recommendation for the tagged meal. The DGS may require data from at least three meals, alternatively at least four meals, alternatively at least 5 meals, before the DGS may calculate a recommended dose for the tagged meal of pancake and eggs. The tags may be for any food and/or drink that is consumed at breakfast, lunch, dinner, or a snack. The dose recommendation may include multiple components, such as a base dose and a correction dose. The base dose may be the user's standard or typical meal dose and the correction dose may account for the user's previous responses to the same tagged meal. In some embodiments, the correction dose may account for the user's current glucose level at the time that the request for the dose recommendation was made.

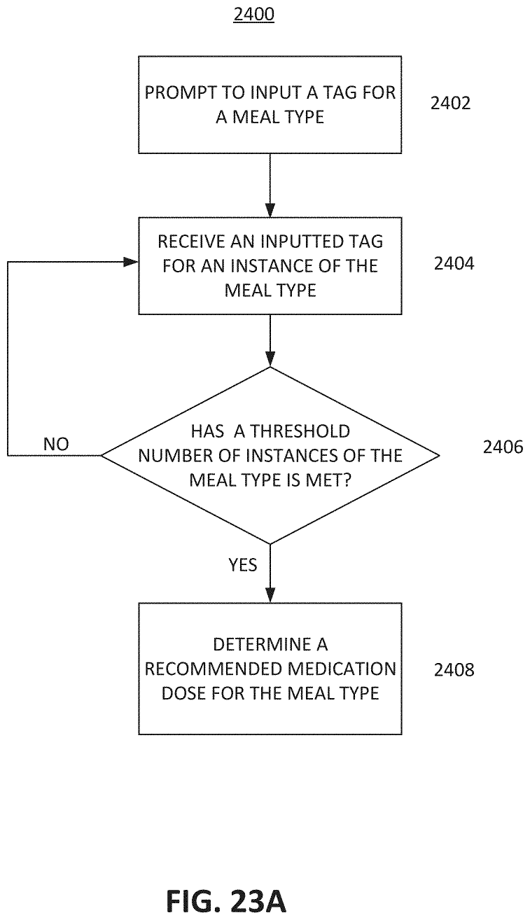

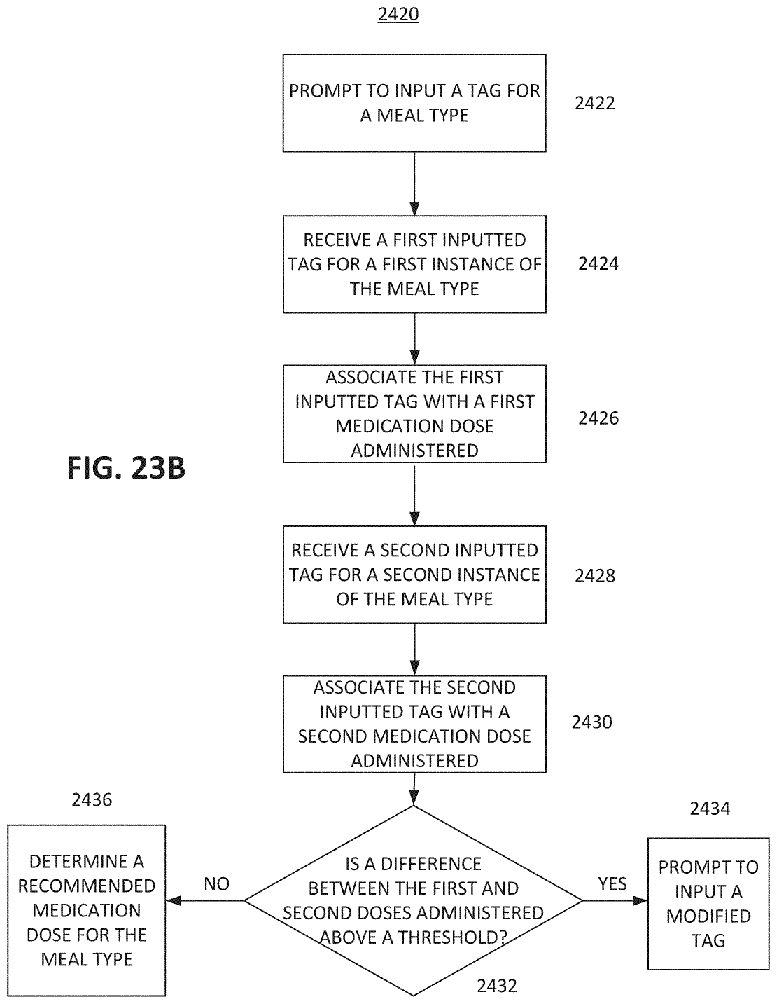

In one exemplary embodiment, in method 2400 as seen in FIG. 23A, in a first step 2402, the DGS may prompt the user to input a tag associated with a meal type. For example, the meal type may be a description of specific food and/or drinks consumed by the user during a meal or snack such as “a cheeseburger and fries,” “two slices of cheese pizza,” “eggs and pancakes,” or “an apple and a diet soda.”

In step 2404, the DGS may receive an inputted tag for an instance of the meal type. The user may enter a description tag for this meal as a favorite. Alternatively, if the meal type has already been entered in the DGS, then the user may select the tag from, e.g., a list of favorite meal types.

In step 2406, the DGS may determine if a threshold number of instances of tags of the meal type have been received by the DGS. For example, the DGS may have a requirement of a minimum number of three tags of a particular meal type. If the DGS determines that only two instances of a tag for the meal type has been received, then the DGS may not calculate a recommended medication dose for the meal type. If, however, the DGS determines that three or more instances of a tag for the meal type has been received, then the DGS may calculate a recommended medication dose of the meal type, as indicated in step 2408.

In some embodiments, after the user tags the meal, the DGS may detect that the medication dose administered is different than the medication doses administered during the previous consumption of the same meal. If the DGS detects a significant difference in medication dose amounts, such as a 1 unit difference, alternatively a 2 unit difference, alternatively a 3 unit difference, alternatively a 4 unit difference, alternatively a 5 unit difference, alternatively a difference between about 1 and 5 units, alternatively a difference between about 2 and 5 units, alternatively a difference between about 3 and 5 units, the DGS may prompt the user. In some embodiments, the DGS may prompt the user to create a new tag. The new tag may include a description in the tag to account for the different medication dosage. For instance, the new tag may include a size of the meal or another reason why the medication dosage was different, e.g., exercising before the meal. In some embodiments, the DFS may prompt the user in real time, i.e., within 5 minutes, alternatively within 4 minutes, alternatively within 3 minutes, alternatively within 2 minutes, alternatively within 1 minute of the user tagging the meal type.

In one exemplary embodiment, in method 2420 as seen in FIG. 23B, in a first step 2422, the DGS may prompt the user to input a tag associated with a meal type. For example, the meal type may be a description of specific food and/or drinks consumed by the user during a meal or snack such as “a cheeseburger and fries,” “two slices of cheese pizza,” “eggs and pancakes,” or “an apple and a diet soda.”

In step 2424, the DGS may receive a first inputted tag for a first instance of the meal type. The user may enter a description tag for this meal as a favorite. Alternatively, if the meal type has already been entered in the DGS, then the user may select the tag from, e.g., a list of favorite meal types.

In step 2426, the DGS may associate the first inputted tag with a first amount of medication administered for the first instance of the meal type. In some embodiments, the first inputted tag may also be associated with a first post-prandial analyte data set for the first instance of the meal type.

In step 2428, the DGS may receive a second inputted tag for a second instance of the meal type. The user may select the tag from, e.g., a list of favorite meal types.

In step 2430, the DGS may associate the second inputted tag with a second amount of medication administered for the second instance of the meal type. In some embodiments, the second inputted tag may also be associated with a second post-prandial analyte data set for the second instance of the meal type.

In step 2432, the DGS may determine if a difference between the first and second doses administered is above a threshold. If the difference is above a threshold, in step 2434, the DGS may prompt the user to input a modified tag that may account for the different dose.

In some embodiments, if the DGS has enough data to determine a recommendation, if the difference is below the threshold, in step 2436, the DGS may output a recommended dose for the meal type.

Missed Meal Dose Alerts

Both the missed meal dose calculation function and the missed dose alert function use the missed meal dose detection algorithm. The missed dose alert function calls the missed dose detector every minute. When an alert is asserted, which may also be referred to as an active alert or an activated alert, it may be presented in a lock screen notification or an in-app modal, and may also be presented in the notification center or notification banner of the display device 120. The alert may be rescinded under certain conditions, i.e., the alert may become inactive or inactivated or may be removed from the notification center or notification banner of the display device 120 if it is determined that the alert should no longer be active.

A missed meal dose alert may be asserted if the following conditions are met:
A missed meal dose condition may have been detected for consecutive minutes (e.g., 5 minutes or 5 consecutive positive missed meal dose events) OR a missed meal dose alert is currently asserted.
An insulin dose has not been recorded within the past 2 hours.
An insulin dose has not been recorded within 45 minutes prior to an estimated meal start time.
The estimated meal start time is within the past 2 hours.
A correction dose alert is NOT asserted.

In some embodiments, the missed meal dose alert may not be asserted unless all of these conditions are met. In other embodiments, only one or more of these conditions may be met before the missed meal alert is asserted. It is to be understood that each of these conditions is optional and are not necessarily required.

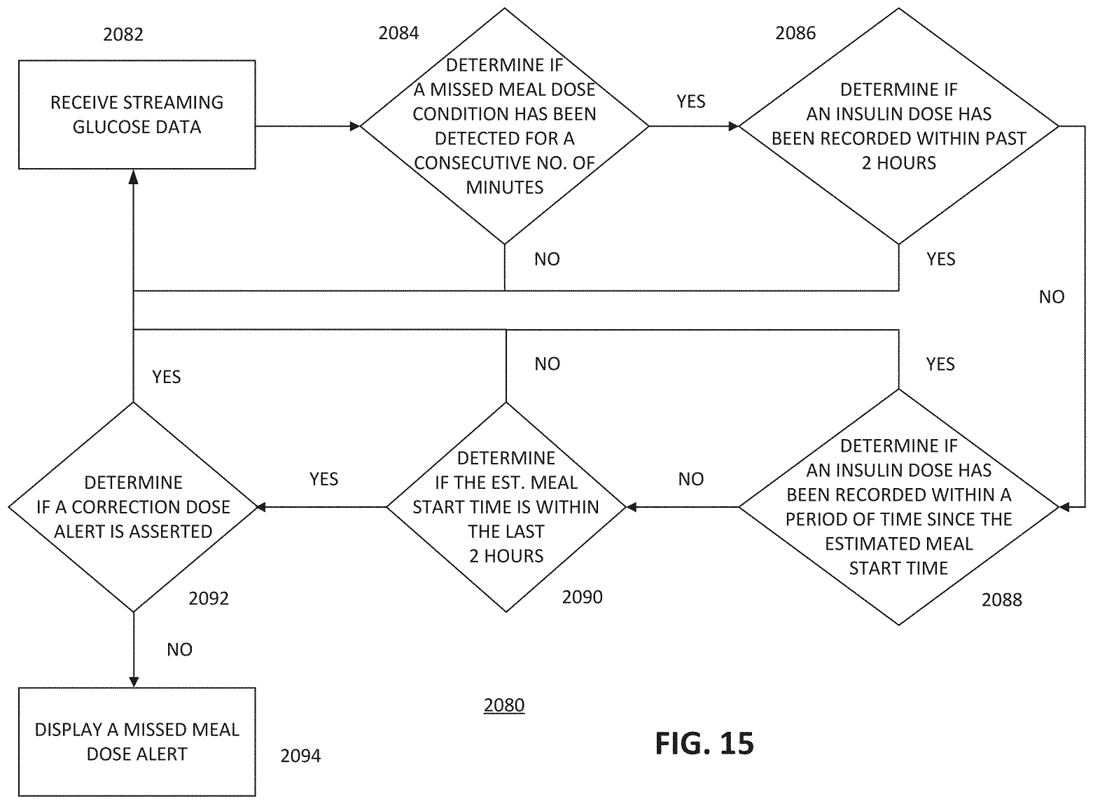

As seen in FIG. 15, in exemplary method 2080, beginning with step 2082, the DGA may receive streaming glucose data from a sensor control device 102.

In step 2084, the DGA may determine, at a current time, if a meal dose has been missed in association with a meal having an estimated meal start time by detecting a missed meal dose condition for a consecutive number of minutes (e.g., 5 minutes or 5 consecutive positive missed meal dose events). Alternatively, the DGA may determine if a missed meal dose alert is currently asserted (not shown).

If step 2084 is answered in the affirmative, in step 2086, the DGA may determine if an insulin dose has been recorded within a previous period of time, e.g., within the past two hours.

If step 2086 is answered in the negative, in step 2088, the DGA may determine if an insulin dose has been recorded within a period of time (e.g., 30 minutes, alternatively 45 minutes, alternatively 60 minutes) prior to the estimated start time of the meal.

If step 2088 is answered in the negative, in step 2090, the DGA may determine if the estimated meal start time is within the past two hours, i.e., two hours since the current time.

If step 2090 is answered in the affirmative, in step 2092, the DGA may determine if a correction dose alert is currently asserted. The DGA may prevent the assertion of a missed meal dose alert at the same time as the assertion of a correction dose alert.

If step 2092 is answered in the negative, in step 2094, the DGA may display or assert a missed meal dose alert.

It is to be understood that each of the conditions mentioned in the above exemplary method is optional and are not necessarily required for the missed meal dose alert to be asserted.

If the missed meal dose alert condition is asserted, then the DGS 100 may present a lock screen notification to notify the user of a missed meal dose alert. If the missed meal dose alert condition is rescinded, the DGS 100 may then remove the missed meal dose alert notification.

The missed meal dose alert may be rescinded if it is currently asserted and the following conditions are met:
A missed meal dose condition has not been detected for the past 15 minutes
OR one or more of the following conditions are met:
   An insulin dose has been recorded in the last 2 hours
   OR an insulin dose has been recorded within 45 minutes prior to the estimated meal start time
   OR the estimated meal start time is more than 2 hours old

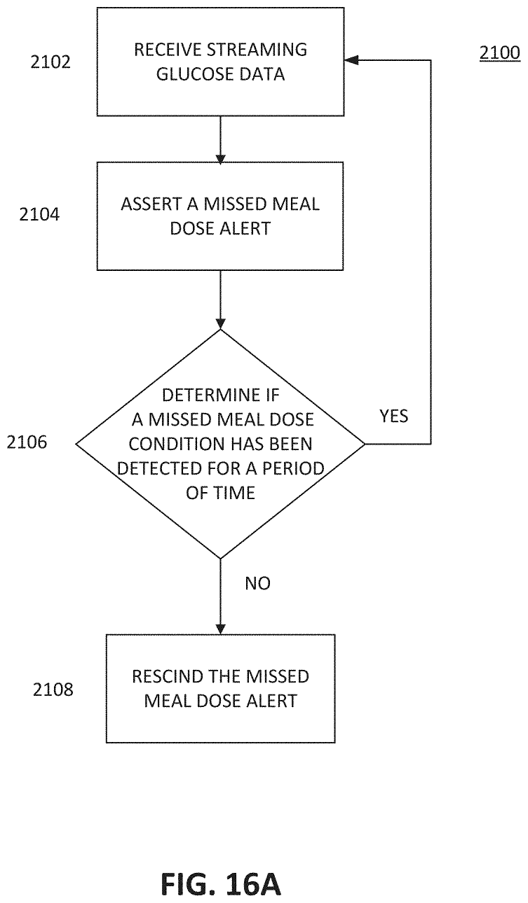

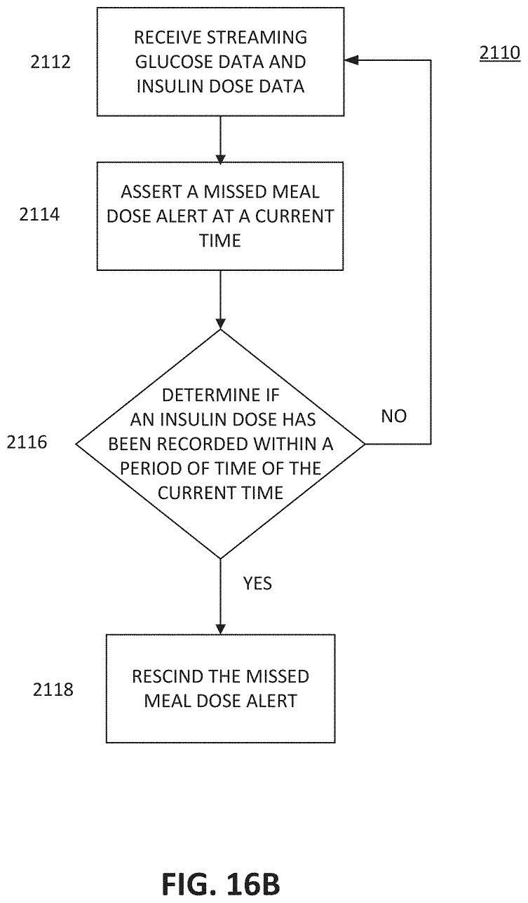

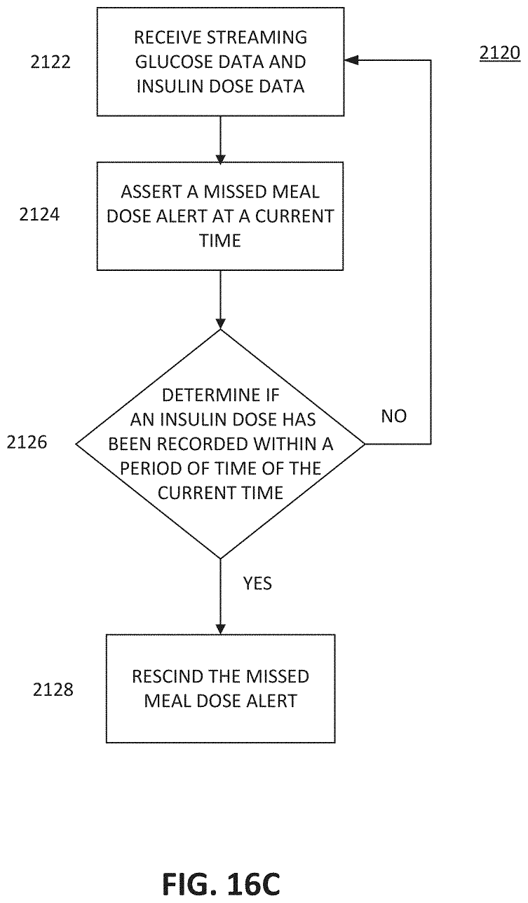

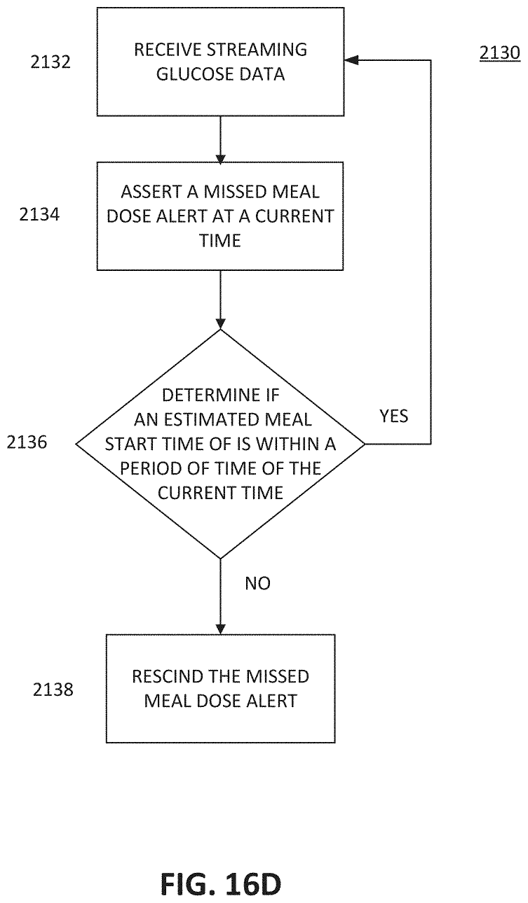

As seen in FIG. 16A, in exemplary method 2100, beginning with step 2102, the DGA may receive streaming glucose data from a sensor control device 102.

In step 2104, the DGS 100 may assert a missed meal dose alert. The conditions under which missed meal dose alerts may be asserted are described elsewhere in this application.

In step 2106, the DGS 100 may determine if a missed meal dose condition has been detected for a consecutive number of minutes after the missed meal dose has been asserted. For example, the DGS 100 may determine if a missed meal dose condition has not been detected for the past 10 minutes, alternatively the past 15 minutes, alternatively the past 20 minutes.

If step 2106 is answered in the negative, then in step 2108, the DGS 100 may rescind the missed meal dose alert.

Where streaming glucose data is unavailable such that the missed alert condition could not be calculated for, e.g., the past 14 minutes and then at minute 15, the missed alert condition is calculated and not asserted, then the DGS 100 may rescind the missed meal dose alert.

As seen in FIG. 16B, in exemplary method 2110, beginning with step 2112, the DGA may receive streaming glucose data from a sensor control device 102 and insulin dose data from an MDD 152 or other ways previously described in other embodiments.

In step 2114, the DGS 100 may assert a missed meal dose alert. The conditions under which missed meal dose alerts may be asserted are described elsewhere in this application.

In step 2116, the DGS 100 may determine if an insulin dose has been recorded within a period of time of the current time. The period of time may be about 1.5 hours, alternatively about 2 hours, alternatively about 2.5 hours.

If step 2116 is answered in the positive, then in step 2108, the DGS 100 may rescind the missed meal dose alert.

As seen in FIG. 16C, in exemplary method 2120, beginning with step 2122, the DGA may receive streaming glucose data from a sensor control device 102 and insulin dose data from an MDD 152 or other ways previously described in other embodiments.

In step 2124, the DGS 100 may assert a missed meal dose alert. The conditions under which missed meal dose alerts are asserted are described elsewhere in this application.

In step 2126, the DGS 100 may determine if an insulin dose has been recorded within a period of time of the estimated meal start time. The period of time may be about 30 minutes, alternatively about 45 minutes, alternatively about 60 minutes.

If step 2126 is answered in the positive, then in step 2128, the DGS 100 may rescind the missed meal dose alert.

As seen in FIG. 16D, in exemplary method 2130, beginning with step 2132, the DGA may receive streaming glucose data from a sensor control device 102.

In step 2134, the DGS 100 may assert a missed meal dose alert, wherein the missed meal dose alert relates to a missed meal having an estimated start time. The conditions under which missed meal dose alerts are asserted are described elsewhere in this application.

In step 2136, the DGS 100 may determine if the estimated meal start time is within a period of time of the current time. The period of time may be about 1.5 hours, alternatively about 2 hour, alternatively about 2.5 hours.

If step 2126 is answered in the negative, then in step 2128, the DGS 100 may rescind the missed meal dose alert.

Missed Meal Dose Condition Detection

A missed meal dose condition detection module of the DGS 100 received streaming glucose data as input. The missed meal dose condition detection module may be called once per minute (e.g., after each minute of streaming glucose is received) to estimate if a meal dose has been missed and, if so, estimate the meal start time. The missed meal dose condition detection module may also be called on demand to estimate a meal start time of the missed meal.

The missed dose detector uses streaming glucose since the last recorded insulin meal dose, up to about 4 hours total, alternatively about 4.5 hours total, alternatively about 5 hours total (the insulin action time used by the IOB calculator).

The algorithms describing real time meal detection and missed meal dose detection are described elsewhere in this application.

The missed meal dose detection may be triggered by the DGA detecting a meal event. The input data streams may include a detected meal event, an estimated meal start time, and a most recent insulin dose amount and timestamp. The outputs may be a tagged meal event without a dose and notification to the user.

Correction-Only Dose Calculation

Within the DGA, post-meal correction-only insulin dose guidance may be available if (1) the DGSM is in the correction-only guidance available state, and (2) a correction dose alert is asserted.

The display state of the DGA notification (which may be graphically represented as a lightbulb) may only be determined when the dose guidance display is initiated (or if another glucose scan is performed). For instance, the display state may not be updated whenever a new streaming glucose value is received. Moreover, in one embodiment, it may be required for all alerts to be resolved before dose guidance is provided, in order to avoid race conditions.

The correction dose calculation may be triggered by a user responding to a correction dose notification from the correction dose detector and scans. The input data streams may include the scan glucose, the scan trend arrow, and the IOB at the time of the most recent scan. The input parameters may be the target glucose and post-meal CF. The outputs may be a suggested calculation of a correction dose.

Correction—Dose Alert

When a correction dose alert is asserted, which may also be referred to as an active alert or an activated alert, it may be presented in a lock screen notification or an in-app modal, and may also be presented in the notification center or notification banner of the display device 120. The alert may be rescinded under certain conditions, i.e., the alert may become inactive or inactivated or may be removed from the notification center or notification banner of the display device 120 if it is determined that the alert should no longer be active.

A correction dose alert may be asserted if the following conditions are met:
A correction dose condition has been ASSERTED for all of the past 5 minutes OR the correction dose alert is currently asserted
An insulin dose has not been recorded within the past 2 hours
A missed meal dose alert is NOT asserted. (prevent correction and missed meal dose alerts from coinciding)

In some embodiments, the correction dose alert may not be asserted unless all of these conditions are met. In other embodiments, only one or more of these conditions may be met before the correction dose alert is asserted. It is to be understood that each of these conditions is optional and are not necessarily required.

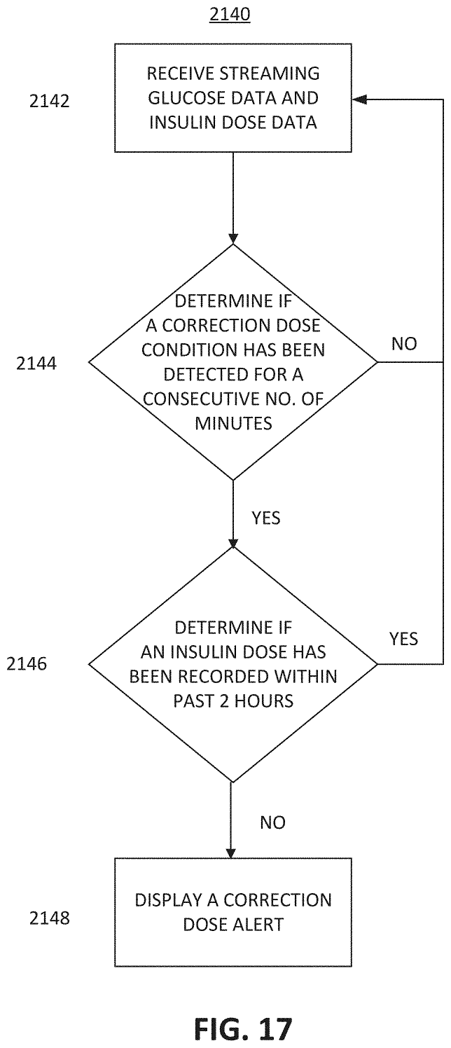

As seen in FIG. 17, in exemplary method 2140, beginning with step 2142, the DGA may receive streaming glucose data from a sensor control device 102 and insulin dose data from an MDD 152 or other ways previously described in other embodiments.

In step 2144, the DGA may determine, at a current time, if a correction dose condition has been asserted for a consecutive number of minutes (e.g., 5 minutes or 5 consecutive positive missed meal dose events). In some embodiments, a correction dose condition may be a condition that indicates a user requires a correction dose. Alternatively, the DGA may determine if a correction dose alert is currently asserted (not shown).

If step 2144 is answered in the affirmative, in step 2146, the DGA may determine if an insulin dose has been recorded within a previous period of time, e.g., within the past two hours.

If step 2146 is answered in the negative, in step 2148, the DGA may display or assert a correction dose alert.

If the missed correction dose alert condition is asserted, then the DGS 100 may present a lock screen notification to notify the user of a correction dose alert. If the correction dose alert condition is rescinded, the DGS 100 may then remove the correction dose alert notification.

The DGS 100 may rescind the correction dose alert if it is currently asserted and either of the following conditions are met:
The correction dose condition has not been detected for any of the past 15 minutes
OR the IOB calculation has changed since the last time the correction dose alert condition was calculated)
OR an insulin dose has been recorded in the past 2 hours

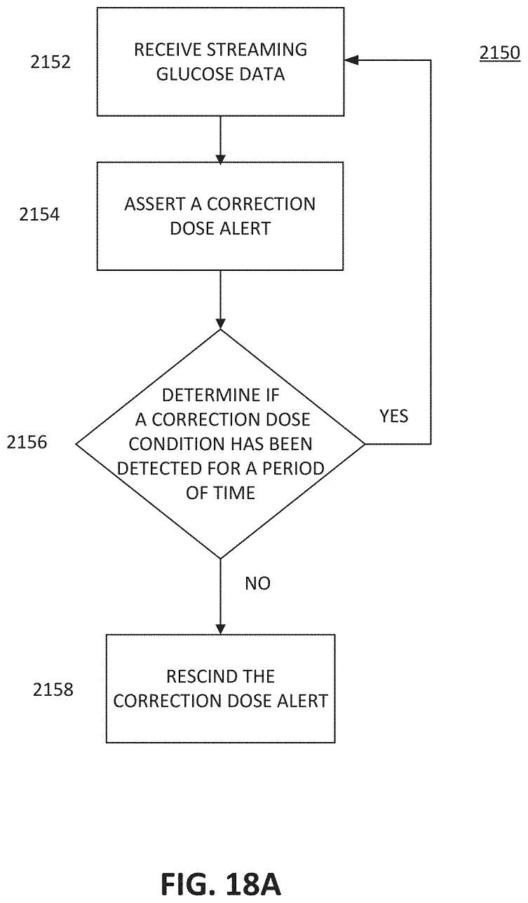

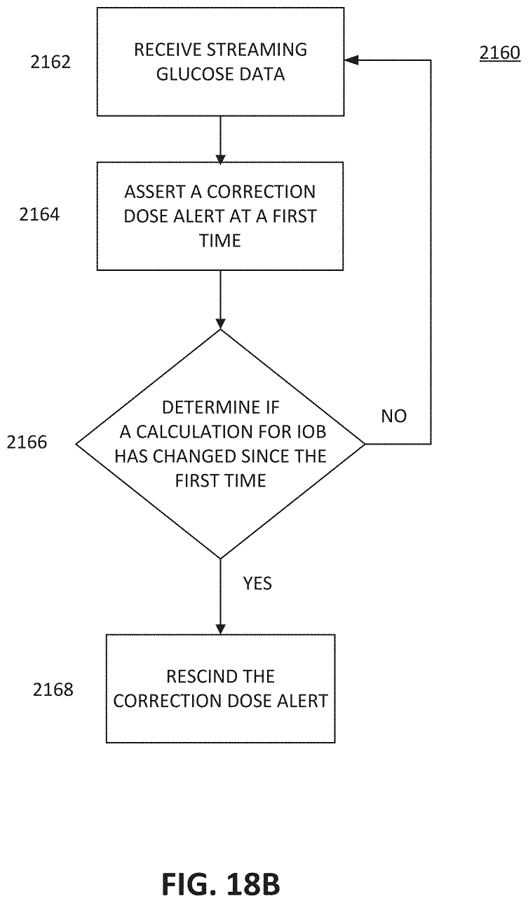

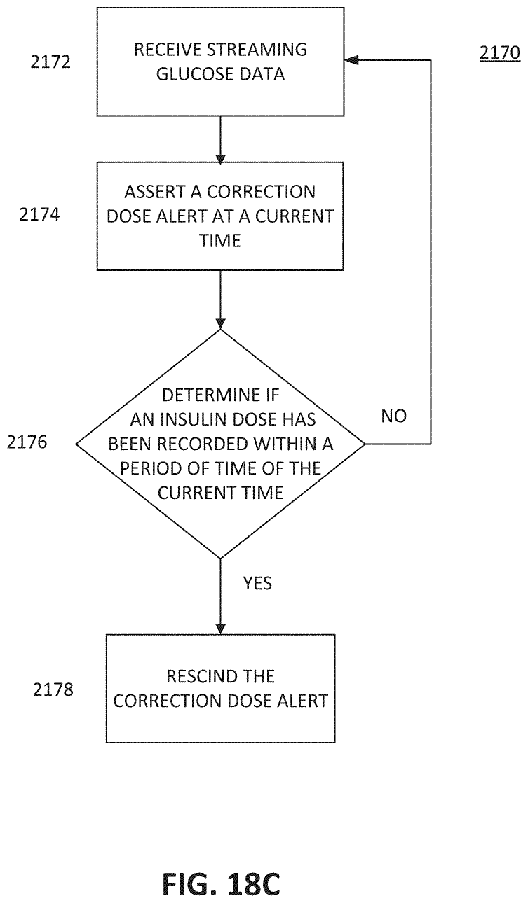

As seen in FIG. 18A, in exemplary method 2150, beginning with step 2152, the DGA may receive streaming glucose data from a sensor control device 102.

In step 2154, the DGS 100 may assert a correction dose alert. The conditions under which correction alerts are asserted are described elsewhere in this application.

In step 2156, the DGS 100 may determine if a correction dose condition has been detected for a consecutive number of minutes after the correction dose has been asserted. For example, the DGS 100 may determine if a correction dose condition has not been detected for the past 10 minutes, alternatively the past 15 minutes, alternatively the past 20 minutes.

If step 2156 is answered in the negative, then in step 2158, the DGS 100 may rescind the correction dose alert.

Where streaming glucose data is unavailable such that the correction dose condition could not be calculated for, e.g., the past 14 minutes and then at minute 15, the correction dose condition is calculated and not asserted, then the DGS 100 may rescind the correction dose alert.

As seen in FIG. 18B, in exemplary method 2160, beginning with step 2162, the DGA may receive streaming glucose data from a sensor control device 102.

In step 2164, the DGS 100 may assert a correction dose alert at a first time. The conditions under which correction alerts are asserted are described elsewhere in this application.

In step 2166, the DGS 100 may determine if a calculation for insulin on board (JOB) has changed since the first time.

If step 2166 is answered in the affirmative, then in step 2168, the DGS 100 may rescind the correction dose alert.

As seen in FIG. 18C, in exemplary method 2170, beginning with step 2172, the DGA may receive streaming glucose data from a sensor control device 102.

In step 2174, the DGS 100 may assert a correction dose alert. The conditions under which correction alerts are asserted are described elsewhere in this application.

In step 2176, the DGS 100 may determine if an insulin dose has been recorded within a period of time of the current time. The period of time may be about 1.5 hours, alternatively about 2 hour, alternatively about 2.5 hours.

If step 2176 is answered in the affirmative, then in step 2178, the DGS 100 may rescind the correction dose alert.

In some embodiments, the user may customize the conditions under which a correction dose alert is presented to the user. In the settings of the DGS, the user may set a minimum threshold for a correction dose alert such that they are only alerted if the correction dose amount is above a minimum dose amount. For example, the user may indicate that they only want to receive a correction alert if the suggested corrected dose is at least about 2 units. The minimum dose amount may be about 1 unit or more, alternatively about 2 units or more, alternatively about 3 units or more, alternatively about 4 units or more, alternatively about 5 units or more, alternatively about 6 units or more. Allowing the user to set a minimum correction dose amount may prevent the user from being overloaded with alerts (alert fatigue), such that the user may focus only on correcting conditions that are more serious and require a bigger correction dose. This allows the user to only receive alerts if they need a minimum amount of units.

Correction Dose Detector

The correction dose condition detector may indicate if the condition is right for a correction dose. The correction dose condition detector may also provide the result of the calculation for the correction dose. The correction dose calculation may also use streaming glucose data, e.g., one-minute streaming glucose. Thus, the correction dose calculation may match or coincide with the correction dose alert condition, which also uses streaming glucose.

An exemplary method for determining a correction dose presented below in pseudo-code.

```
Correction Dose == FALSE, unless
    if t_{dose} > 3 hr AND ≤ 4 hr
      if G_{corr} > 180 mg/dL for the last 60 consecutive minutes
        if G_{trend} > 0 mg/dL/min for the last 5 consecutive
  minutes
          if Iₘᵢₙ − IOB ≥ 0.5
            then Correction Dose Needed ==
  TRUE
          end
        end
      end
    end
    OR
    if t_{dose} > 4 hr
      if G_{corr} > 180 mg/dL for the last 60 consecutive minutes
      if G_{trend} > 0 mg/dL/min for the last 5 consecutive minutes
        if Iₘᵢₙ +TrendAdjPost − IOB ≥ 0.5
            then Correction Dose Needed ==
  TRUE
        end
      end
      end
    end
```

where
T_{dose}=time since last recorded insulin dose (non prime, if this attribute is available)
G_{corr}=current streaming glucose value (current meaning at the time of initial display)
G_{T}=target glucose (regimen parameter lookup)
G_{trend}=current streaming glucose trend value (current meaning at the time of initial display)
I_{guide}=insulin dose calculation result
Iₘᵢₙ=minimum correction dose that a user wants to be notified of. This value will be configured by the user upon onboarding of the mobile app.

Once Correction Dose Needed==TRUE in pseudo-code above, the correction dose calculation may then proceed as follows:

```
if t_{dose} > 2 hr AND < 4 hr
  I_{guide} = CorrectionAdjPost − IOB
elseif t_{dose} > 4 hr
  I_{guide} = CorrectionAdjPost + TrendAdjPost − IOB
else
  I_{guide} = 0
end
```

where
IOB=insulin on board at time of dose calculation
CFₚₒₛₜ=correction factor post-meal (regimen parameter lookup)
CorrectionAdjPost=(G_{corr}-G_{T})/CFₚₒₛₜ
TrendAdjPost=trend adjustment (Kudva table lookup), based on CF=CFₚₒₛₜ

The definition of glucose trend as well as TrendAdj by CFₚₒₛₜ are explained in TABLES 1 and 2, as adapted from Kudva, et al., which was previously incorporated by reference in its entirety.

The calculation of I_{guide} may be rounded to the nearest integer according to standard rounding rules.

If I_{guide}≤Iₘᵢₙ, then I_{guide}=0.

The correction dose determination may be continuously running. The input data streams may include streaming glucose, streaming trend arrow, time since last classified meal dose, and time since last classified correction dose. The input parameters may include a minimum correction dose amount, which may be configured by the user. The outputs may include a notification to the user.

Correction Alert and Missed Meal Dose Alert Interaction

The alert processing for both correction and missed meal dose alerts may be performed every minute using the streaming glucose data. The missed meal dose alert processing may be performed before the correction alert processing in order to ensure that if conditions are such that both alerts would initiate assertion, then the missed meal dose alert would prevail (thus preventing the correction dose alert from being asserted).

Insulin on Board (JOB) Management and Calculation

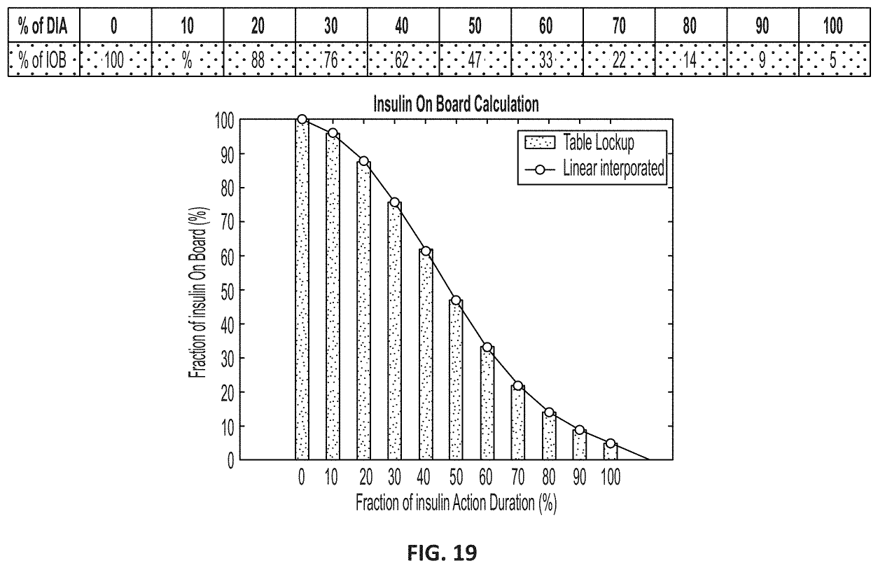

To prevent dose recommendations based upon stale glucose data, the elapsed time between the most recent scan and the dose request may be no longer than five minutes. IOB may be calculated according to a duration of insulin action of 4.5 h for rapid acting insulin. The IOB value to be subtracted in Eq. 1 and 2 is the % IOB at the time of the meal request multiplied by the previous insulin dose amounts. For example, according to information in FIG. 19, a dose of 10 U administered at 12:00 will have a remaining IOB of 4.7 U at 14:15. Further details are supplied in FIG. 19. The current IOB is the sum of each of these IOB portion calculations for each insulin dose less than 4.5 h old.

The duration of insulin action (DIA) is the estimated time that a given insulin injection will confer its glucose-lowering effect. The DGS 100 assumes a DIA of 4.5 h for rapid acting prandial insulin and 24 h for long acting basal insulin. If the DIA of an insulin dose is 4.5 h, then the IOB is zero at 4.5 h following the injection. The units of DIA is hours.

The IOB calculation may be triggered whenever a dose (mealtime or correction) is calculated. The input data streams may include insulin dose amounts and timestamps for doses less than or equal to 4.5 hours and an IOB lookup table. The outputs may be remaining insulin units in circulation from previous injections.

### Real Time Meal Detection

In many embodiments, the DGA can be configured to detect missed meal doses using a real-time meal detection algorithm. Systems and processes for real-time detection of missed meal doses and subsequent alerting of the patient are described herein. The process for detecting missed meal doses can be executed periodically (e.g., whenever new glucose data are available to the system). Alternatively, the process can be executed whenever it is appropriate to provide “missed dose” alerts to the patient, or whenever the alert has been enabled. The use may enable or disable the alerts if they become too cumbersome.

In one example embodiment, real-time meal detection can be performed by a feature extraction module and a meal detection module. The feature extraction module can receive CGM datapoints one at a time as the datapoints become available. When the feature extraction module detects that a glucose value is increasing, the feature extraction module can extract a plurality of features and can pass the plurality of features to the meal detection module for meal detection.

In one embodiment, the feature extraction module is configured to perform data smoothing each time a new glucose data point is received by fitting the data within a time window and counting backwards from the current data point using a quadratic function. The time window can be about 60 minutes. The feature extraction module can be configured to store a fitted value at a center of the time window as a current smoothed data. The feature extraction module can also be configured to store coefficients of the linear and quadratic terms of the fitted value at the center of the time window as the most recent glucose rate of change and acceleration values, respectively. In addition to being configured to store the fitted values at the center point, the feature extraction module can also be configured to store a fitted value at the most recent point for the feature extraction. The feature extraction module can be configured to compare the current smoothed glucose value with a previous smoothed glucose value (e.g., the smoothed glucose value immediately preceding the current smoothed glucose value) to determine whether the smoothed glucose data is rising or falling. The feature extraction module can be configured to extract a plurality of features and thereafter pass the plurality of features to the meal detection module after the feature extraction module determines a rise in a comparison of the current and previous smoothed glucose values.

The feature extraction module can be configured to extract a plurality of features from two segments in the smoothed data. The two segments can be a current rising segment and a previous falling segment. The plurality of features extracted from the current rising segment can include, but are not limited to, 1) the maximal acceleration, 2) the time of the maximal acceleration point, 3) the glucose value at the maximal acceleration point, 4) the height computed by the difference in the glucose values between current time point (the fitted value) and the maximal acceleration point (the reference point), 5) the duration of the current segment computed by the elapsed time from the reference point to the current point, 6) the average rising rate of the current segment computed by dividing the height by the duration, 7) the maximum increase of acceleration (the increase in the acceleration at a given time point is obtained by subtracting the acceleration of the point by that of the previous point), and 8) the incremental area under the curve (subtracting the glucose value of the reference point from the mean glucose value, and subsequently multiplying the difference by the duration of the segment). The plurality of features extracted from the previous falling segment can include, but are not limited to: 1) duration, 2) height, 3) the average falling rate (height/duration), 4) the maximum falling rate (the maximum of the absolute value of the rate of change), and 5) the maximum deceleration (the maximum of the absolute value of the acceleration). The feature extraction module can be configured to pass the plurality of extracted features to the meal dose module.

The meal detection module can be configured to receive a feature vector as input and can be configured to output a binary detection result indicating whether the current rising segment is a meal response glucose excursion or not. The meal detection module can also be configured to output a probability value with the binary detection result. In one embodiment, a pretrained machine learning model in the meal detection module can be implemented using RandomForestClassifier by scikit learn (https://scikit-learn.org/stable/modules/generated/sklearn.ensemble.RandomForestClassifier.html). The meal detection module can be configured to detect a meal start based on tree building rules and a feature threshold for each feature in each tree, which can be optimized during a training process. In one embodiment, pretrained models can also be built based on alternative classification algorithms including gradient boosting, ada boost, artificial neural network, linear discriminant analysis, and extra tree.

The meal detection model can also be configured to estimate a start time of a meal if a meal is detected. In one embodiment, the start time of the meal can be estimated as the time point at which there is a maximum increase of glucose value acceleration, tracing back from the detection point within a time window size of about 1.25 hours. For example, if a missed meal was detected by the algorithm at 1:15 pm, the model can track back to about 12:00 pm to determine a meal start. Glucose value acceleration at each point can be computed by fitting five data points centered at the data point of interest using a quadratic function, i.e. y=ax²+bx+c. The fitted parameter “a” is the acceleration at the point of interest. An increase of glucose value acceleration at a time point k can be defined by a(k+1)−a(k).

The meal detection model can also be configured to output a notification on UID 200 regarding a missed dose to a user if a mealtime insulin dose is not detected to have occurred within a period of time around the estimated start of the meal. In one embodiment, if the estimated meal start is less than two hours ago, the notification can also indicate that the patient can still get dose guidance for the meal and dose late.

Details of other types of meal detection methods and algorithms are described in U.S. Patent Publ. No. 2017/0185748 and PCT Application Serial No. PCT/US2020/12134, which are hereby expressly incorporated by reference in their entirety.

The real time meal detection may be continuously running. The input data streams may include streaming glucose. The outputs may be a meal event and an estimated meal start.

### Real Time Dose Classification

To drive the dose guidance state transition diagrams, which are described elsewhere in this application, as well as track dose concordance for dosing reports, the algorithm may classify incoming doses from the connected pen in real time.

Doses may be automatically classified by the algorithm if certain criteria are met. Dose may only be automatically classified as a specific meal dose (breakfast, lunch, or dinner) or a correction dose. Automated meal dose and correction dose classification have their own logic for classification.

The automatic real time dose classification may be continuously running. The input data streams may include an insulin dose timestamp, insulin dose amount, and previous dose recommendation (meal or correction). The input parameters may include insulin dose time windows. The outputs may be doses classified as meal or correction.

Meal Doses

For automated meal dose classification, the following criteria may be used to determine if a given dose may be automatically associated with a specific meal (breakfast, lunch or dinner). The logic is presented below in pseudocode:
If the insulin dose timestamp from the connected pen is within ≤20 minutes of a time when the user requested a meal dose in the mobile app &&
   the dose amount is exactly the same as the recommended dose amount &&
   the timestamp of the meal dose request falls within the approved meal dose time range &&
   the dose was not taken in the post-meal state
      Then the dose is classified as the dose associated with the meal request
Else, the dose is classified as ambiguous.

In one embodiment, all the above conditions must be true for the dose to be automatically classified to a given meal. If any of the above are false, the dose may be marked as ambiguous, requiring the user to manually classify the dose before using the app for subsequent dose guidance. This manual classification may be performed via an entry in the DGA. In other embodiments, only one or more of these conditions may be met in order for the dose to be automatically classified. It is to be understood that each of these conditions is optional and are not necessarily required.

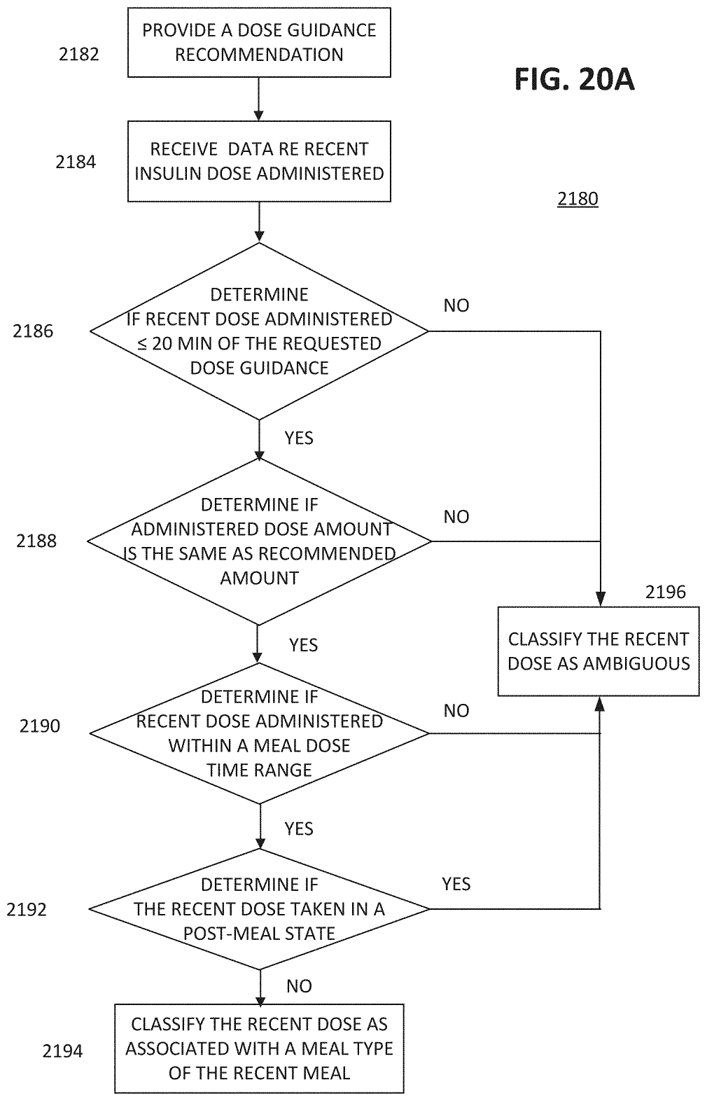

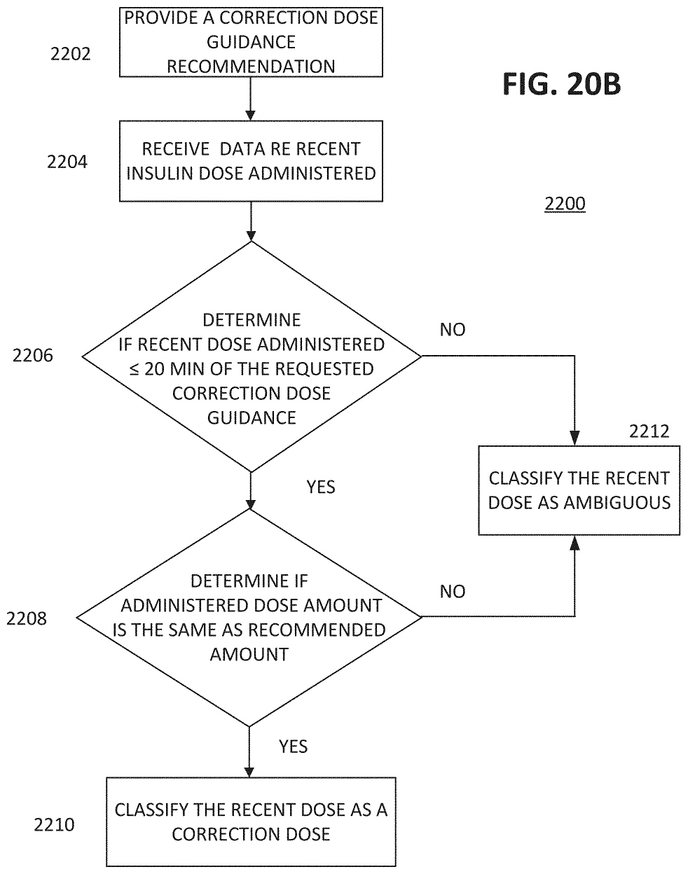

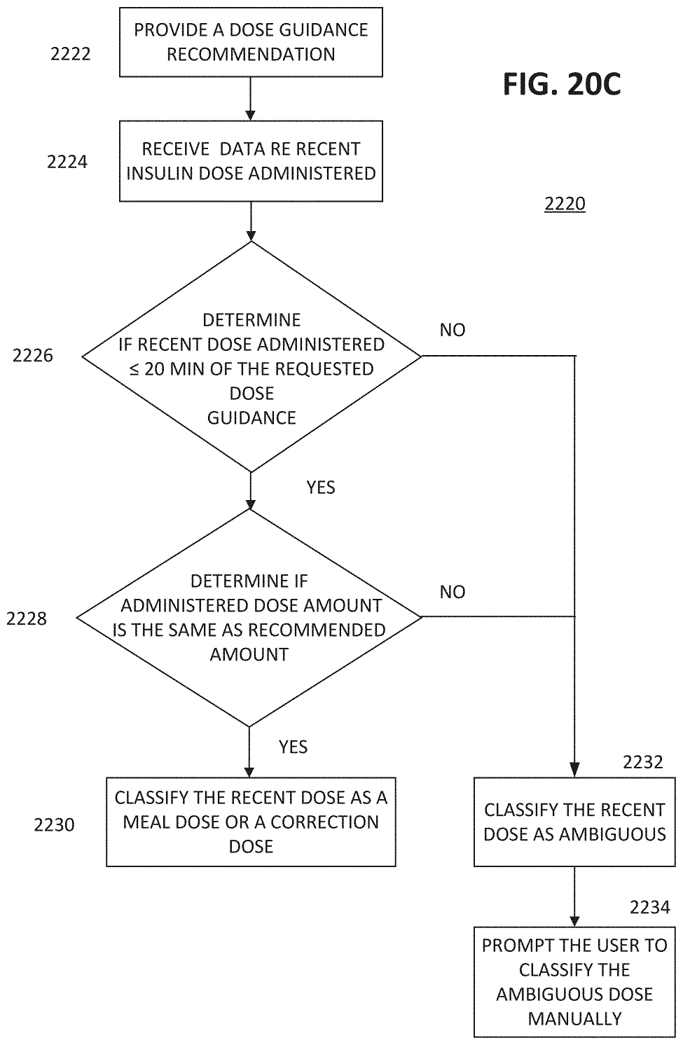

As seen in FIG. 20A, in exemplary method 2180, beginning with step 2182, the DGA may provide a dose recommendation guidance for a meal requested by a user at a request time, wherein the meal has a meal type and the dose recommendation guidance comprises a recommended dose amount.

In step 2184, the DGA may receive insulin dose data of a user from a connected insulin delivery device. The insulin dose data may include a recent dose comprising an insulin amount and a timestamp.

In step 2186, the DGA may determine if the timestamp of the recent dose is within a period of time of the request time. The period of time may be about 15 minutes, alternatively about 20 minutes, alternatively about 25 minutes, alternatively about 30 minutes.

In step 2188, the DGA may determine if the insulin amount of the recent dose is the same as the recommended dose amount of the meal dose guidance recommendation.

In step 2190, the DGA may determine if the timestamp of the recent dose is within a determined meal dose time range for the meal type of the recent meal.

In step 2192, the DGA may determine if the recent dose was taken while the user was in a post-meal state. If the most recent dose is taken while the Dose Guidance State Machine is in the post-meal dose state (i.e., the previous dose has been associated with a meal, triggering a transition to the post-meal dose state), the recent dose may be associated with the same meal as the previous dose.

In step 2194, the DGA may classify the recent dose as associated with the meal type of the recent meal if the above steps 2186-2190 are answered in the affirmative and step 2192 is answered in the negative. If any of the above conditions are not met, then the DGA may classify the recent dose as ambiguous (see step 2196) and prompt the user to manually classify the recent dose.

As an example, if a user requests a lunch dose at 12:00 PM and takes a dose that agrees with the automatic classification logic above, it may be classified as a lunch dose. This classification may trigger a transition from the waiting state to the post-meal dose state at a time starting from the injection time. The algorithm may remain in the post-meal dose state for two hours. If another dose is received at 1:00 PM (while the user is in the post-meal dose state), it may be automatically labeled as a lunch dose. Notably, when in the post-meal dose state, the algorithm may not supply dose guidance for the user. Any dose received in this period may be based solely on user discretion.

In other embodiments, only one or more of these conditions may be met in order for the dose to be automatically classified. It is to be understood that each of these conditions is optional and are not necessarily required.

Correction Doses

For automated correction dose classification, the following criteria must all be true for a given dose to be automatically classified as a correction dose. The logic is presented below in pseudocode.
If the dose was delivered within ≤20 minutes of a correction dose amount recommendation in the mobile app &&
   the dose amount is exactly the same as the recommended dose amount
Else, the dose is classified as ambiguous.

In one embodiment, if both of the above conditions are true, the dose may be classified as a correction dose. If any of the above are false, the dose may be marked as ambiguous, requiring manual classification. This manual classification may be performed via an entry in the DGA. In other embodiments, only one or more of these conditions may be met in order for the dose to be automatically classified. It is to be understood that each of these conditions is optional and are not necessarily required.

As seen in FIG. 20B, in exemplary method 2200, beginning with step 2202, the DGA may provide a dose recommendation guidance for a correction at a first time.

In step 2204, the DGA may receive insulin dose data of a user from a connected insulin delivery device. The insulin dose data may include a recent dose comprising an insulin amount and a timestamp.

In step 2206, the DGA may determine if the timestamp of the recent dose is within a period of time of the first time. The period of time may be about 15 minutes, alternatively about 20 minutes, alternatively about 25 minutes, alternatively about 30 minutes.

In step 2208, the DGA may determine if the insulin amount of the recent dose is the same as the recommended dose amount of the correction dose guidance recommendation.

In step 2210, the DGA may classify the recent dose as a correction dose if the above steps 2206-2208 are answered in the affirmative. If any of the above conditions are not met, then the DGA may classify the recent dose as ambiguous (see step 2212) and prompt the user to manually classify the recent dose.

Because correction doses may only be recommended while the system is in the waiting state, this classification may trigger a transition from the waiting state to the post-correction dose state at a time starting from the injection time. The classification may rescind any outstanding missed meal dose or correction notifications that have been presented to the user.

As seen in FIG. 20C, in exemplary method 2220, beginning with step 2222, the DGA may provide a dose recommendation guidance at a first time.

In step 2224, the DGA may receive insulin dose data of a user from a connected insulin delivery device. The insulin dose data may include a recent dose comprising an insulin amount and a timestamp.

In step 2226, the DGA may determine if the timestamp of the recent dose is within a period of time of the first time. The period of time may be about 15 minutes, alternatively about 20 minutes, alternatively about 25 minutes, alternatively about 30 minutes.

In step 2228, the DGA may determine if the insulin amount of the recent dose is the same as the recommended dose amount of the dose guidance recommendation.

In step 2230, the DGA may classify the recent dose as a meal dose or a correction dose. If any of the above conditions are not met, then the DGA may classify the recent dose as ambiguous (see step 2232) and prompt the user to manually classify the recent dose in step 2234.

When a dose is marked as ambiguous, the user may be required to manually classify the dose before using the DGA for subsequent dose guidance. The user may be provided with the following classification options and may be asked to choose one:
Breakfast Dose
Lunch Dose
Dinner Dose
Correction Dose
Ate more than expected for previous meal
Snack Dose
Priming Dose
Dose not taken

In some embodiments, all ambiguous doses within a period of time, e.g., the last about 4 hours, alternatively the last about 4.5 hours alternatively the last about 5 hours, from the time that the user opens the DGA may need to be classified manually before the DGA may provide dose guidance. Unclassified ambiguous doses greater than the period of time may remain as ambiguous. If the period of time, e.g., 4.5 hour window, spans into the previous day, the period of time may be shortened to span from only the time to user opens the DGA to 00:00:00 AM of the current day.

In some embodiment, if the user manually classifies an ambiguous dose as one of “ate more than expected for previous meal”, “snack dose”, “prime dose” or “dose not taken”, there may be no change to the Dose Guidance State Machine.

### Titration Methods

As disclosed in U.S. application Ser. No. 16/944,736, which was previously incorporated by reference in its entirety, a dose guidance system (DGS) 100 (e.g., SCD 102, display device 120, or MDD 152) can be configured using an automatic or semiautomatic learning method that classifies and characterizes medication doses based on patient input and the patient's glucose pattern analysis (GPA) analyzed over a learning period.

A processor of a DGS 100 may use methods as described herein to provide titration guidance for multiple daily injection (MDI) dosing therapies once it learns (or is configured with) the patient's current dosing strategies. For patients using fixed meal dosing, the system may determine guidance information for titrating the fixed dose amounts (e.g., for breakfast, lunch, dinner, snack, etc.). For patients who are carbohydrate counting, the carbohydrate ratio can be titrated, for these same meals or for different times of the day. Patients who use experiential dosing can titrate their doses on a per meal basis. Titration guidance by the DGA can provide a recommendation to change the dose or carbohydrate ratio in a particular direction. The amount of the change can be a suitable percentage change, for example, 5%, 10%, 15%, etc. Dose guidance can also include starting a meal dose. For example, if a patient is on a basal plus one (e.g., lunch dose) regimen), and breakfast shows a high pattern, the DGA can provide a recommendation to administer a RA insulin for breakfast.

As used herein, insulin doses refer to rapid acting insulin doses, unless the insulin doses are explicitly referred to as “long acting” or “basal”.

In an aspect of the methods, several (e.g., six) parameters of the DGS 100 provide information for titrating dose regimen parameters over time, while the DGS 100 is in a dose guidance mode. The several parameters may include, for example: a fixed basal dose, fixed breakfast dose, fixed lunch dose, fixed dinner dose, fixed pre-meal correction factor and fixed post-meal correction factor.

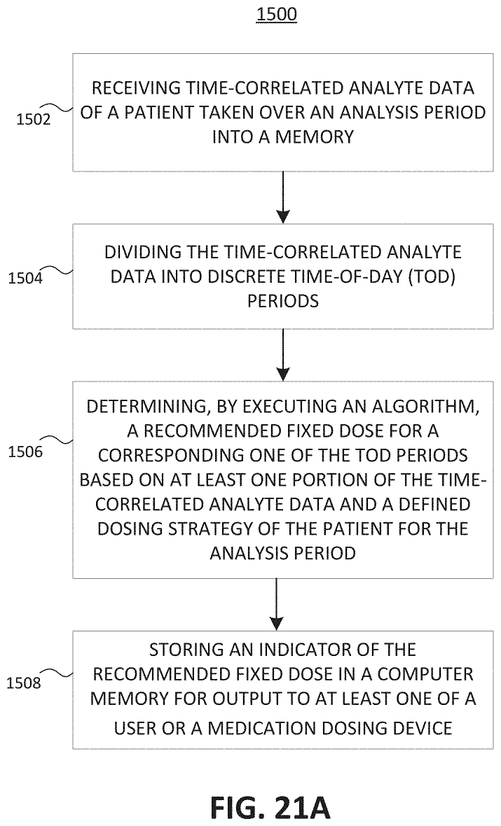

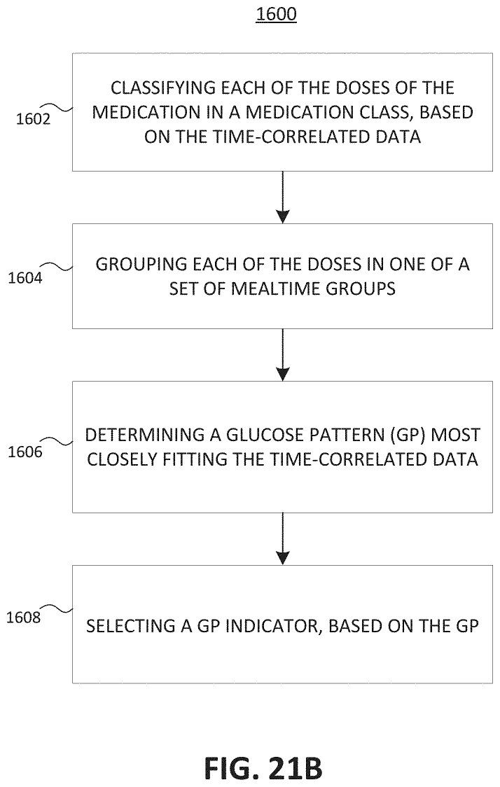

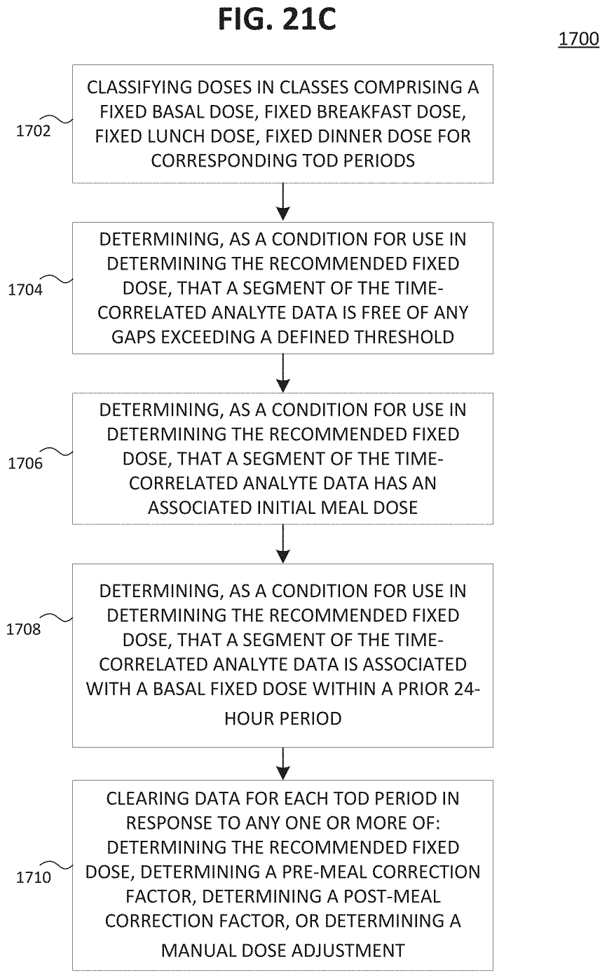

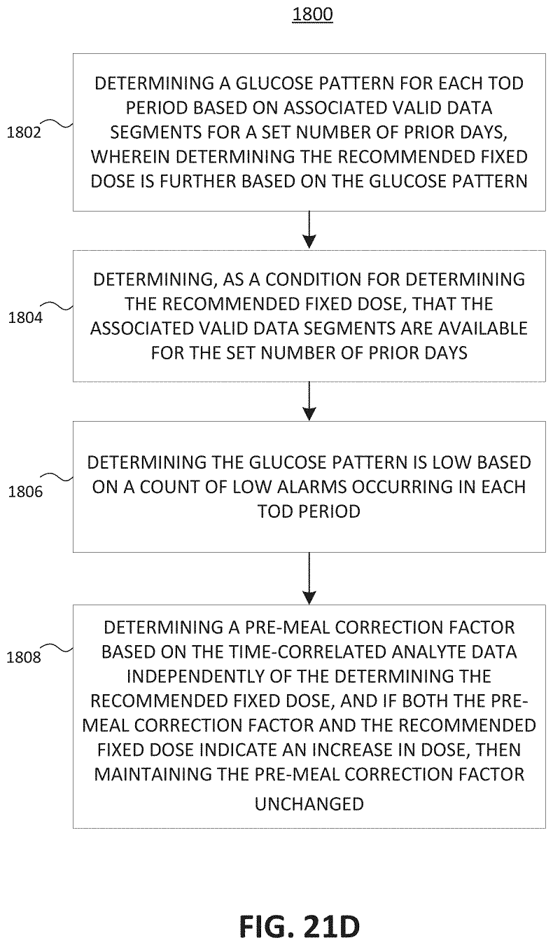

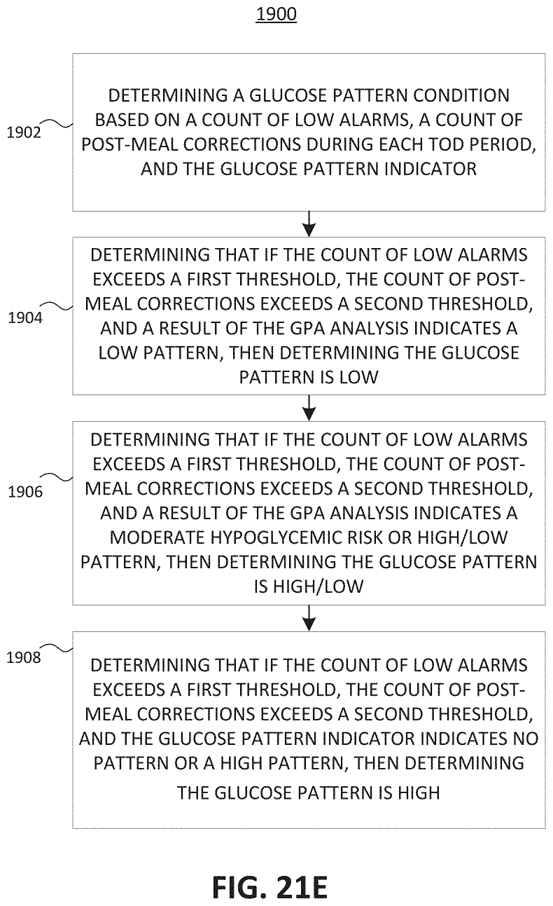

At least one processor of the DGS 100, e.g., a processor of a display device coupled to a memory, a wireless interface to a sensor control device, and a display screen, may include instructions that when executed by the processor cause the DGS 100 to perform a method 1500 for providing dose guidance in response to analyte data, and diagrammed in FIG. 21A. The method 1500 may include, at 1502, receiving time-correlated analyte data of a patient taken over an analysis period into a buffer. The method 1500 may further include, at 1504, dividing the time-correlated analyte data into discrete time-of-day (TOD) periods. The method may further include, at 1506, determining, by executing an algorithm, a recommended fixed dose of the medication for a corresponding one of the TOD periods based on at least one portion of the time-correlated analyte data and a defined dosing strategy of the patient for the analysis period. The method may further include, at 1508, storing an indicator of the recommended fixed dose in a computer memory for output to at least one of a user or a medication dosing device.

The DGS 100 processor may initiate a titration method for adjusting fixed doses based on parameters configured by an authorized user (e.g. an HCP) prior to the system entering the dose guidance mode. The DGS may remain in a dose guidance mode for a definite period, e.g., 7 days, 14 days, or 21 days.

The DGS 100 may titrate the first four parameters (fixed dose titration) using the glucose pattern analysis (GPA) titration methodology as disclosed in U.S. application Ser. No. 16/944,736 and elsewhere in this application, wherein time-of-day periods are demarcated by fixed meal dose times defined by the regimen that is initially set by the authorized user. In an aspect, the fixed pre-meal correction factor (CFₚᵣₑ) parameter and fixed post-meal correction factor (CFₚₒₛₜ) may be calculated by the DGS 100 using independent titration methods. The DGS 100 may execute titration routines periodically, e.g., once per day. For example, the DGS 100 may execute the three different titration routines separately, every day; the fixed dose routine first, then the CFₚᵣₑ routine, then the CFₚₒₛₜ routine. Results from each routine may impact each other as described below for each routine. FIG. 21B illustrates general operations of a method 1600 that uses a glucose pattern indicator for titration of correction factors or fixed doses. Further details of the method 1600 may be as described in U.S. application Ser. No. 16/944,736.

The method 1600 may include, at 1602, classifying each of the medication doses in a medication class, based on the time-correlated data. The method 1600 may further include, at 1604, grouping each of the doses in one of a set of mealtime groups. The method 1600 may further include, at 1606, determining a glucose pattern most closely fitting the time-correlated data, and at 1608, selecting a glucose pattern indicator, based on the glucose pattern. A glucose pattern indicator may be selected from a group consisting, for example, of “HIGH,” “LOW”, “HIGH/LOW” or “NO PATTERN.”

If the authorized HCP approves the titration, then the DGS 100 may update the dose regimen to include the titration and use the resulting updated regimen in subsequent dose guidance. If the authorized HCP fails to approve the titration within the next 24 hours, the DGS 100 may rescind the titration approval request and call the titration routines at the next scheduled titration time. The DGS may issue new titration approval requests thereafter. If the authorized HCP expressly rejects the titration, the DGS may rescind the titration approval request and refrain from calling the titration routines until at least a definite period of several days (e.g., 7 days) has elapsed, and issue new titration approval requests thereafter.

Fixed Dose Titration: The DGS may use a GPA titration method to analyze analyte measurements made at definite intervals over the analysis period, e.g., measurements at 15-minute intervals making up time-correlated analyte (e.g., glucose) data over the learning period. The GPS may segment the data into discrete time-of-day (TOD) periods. The TOD periods may be defined according to the typical meal dose time parameters defined by the authorized HCP as part of the approved dose regimen.

Fixed Dose Titration Inputs: TOD periods, for example, may be defined as follows:

Post-Breakfast TOD Period (TODBF): The starting record is the first glucose record following the fixed breakfast dose time. The ending record is the last glucose record prior to the fixed lunch dose time.

Post-Lunch TOD Period (TODLU): The starting record is the first glucose record following the fixed lunch dose time. The ending record is the last glucose record prior to the fixed dinner dose time.

Post-Dinner TOD Period (TODDI): The starting record is the first glucose record following the fixed dinner dose time. The ending record is the last glucose record prior to bedtime, which the DGS may define, for example, as 6 hours after the fixed dinner dose time or 6 hours prior to the fixed breakfast time, whichever happens earlier.

Overnight TOD Period—first half (TODON1): The overall overnight period may be defined as the time between bedtime and the fixed breakfast dose time. The starting record is the first glucose record following bedtime. The ending record is the last glucose record prior to the midpoint time of the overall overnight period.

Overnight TOD period—second half (TODON2): The starting record is the first glucose record following the midpoint time of the overall overnight period. The ending record is the last glucose record prior to the typical breakfast dose period.

The DGS 100 may impose the following restrictions on user (patient) and authorized HCP entry of fixed meal dose times, for example: the time between the fixed breakfast dose time and the fixed lunch dose time must be no less than 3 hours; the time between the fixed lunch dose time and the fixed dinner dose time must be no less than 3 hours, and the time between the fixed dinner dose time and the fixed breakfast dose time must be no less than 9 hours.

Input Validity

Referring to FIG. 21C, a method 1500 for providing dose guidance in response to analyte data may include additional operations 1700 in any operable order or combination, any one of which may be omitted if not necessary or desired. The operations 1700 may include, at 1702, classifying doses in classes comprising a fixed basal dose, fixed breakfast dose, fixed lunch dose, fixed dinner dose for corresponding ones of the TOD periods. Dose classification (e.g., identifying initial meal doses) is necessary for fixed dose titration to determine the necessity of excluding certain TOD periods. Also, the identification of post-meal correction doses is required for the fixed dose titration algorithm.

A glucose data segment (one of many that would contribute to data associated with a TOD period) may be deemed valid by the processor of the DGS 100 subject to certain conditions, for example, only if all the following conditions are met:

Condition 1: The data segment meets the data sufficiency requirement that there be no gaps in glucose data larger than a threshold value, for example, two consecutive historic glucose values. In a related aspect, the operations 1700 may include, at 1704, determining, as a condition for use in determining the recommended fixed dose, that a segment of the time-correlated analyte data is free of any gaps exceeding a defined threshold.

Condition 2: The data segment has an associated initial meal dose. This condition is TRUE if the initial meal dose falls within the fixed meal dose range defined by the authorized HCP when confirming the initial dose regimen settings. For example, if an initial meal dose falls within the fixed breakfast dose range, that dose will be associated with a post-breakfast TOD for analysis. In a related aspect, the operations 1700 may include, at 1706, the DGS processor determining, as a condition for use in determining the recommended fixed dose, that a segment of the time-correlated analyte data has an associated initial meal dose.

Condition 3: The data segment has a basal dose within the prior day. For example, the operations 1700 may include, at 1708, determining, as a condition for use in determining the recommended fixed dose, that a segment of the time-correlated analyte data is associated with a basal fixed dose within a prior 24-hour period. In an alternative, or in addition, for PM basal regimens, if a basal dose was not recorded then processor may treat the subsequent TOD periods as not valid: overnight, post-breakfast, post-lunch, post-dinner. Similarly, for AM basal regimens, if a basal dose was not recorded, then the subsequent TOD periods may be treated as not valid: post-breakfast, post-lunch, post-dinner, overnight.

The following events should not impact the validity of a data segment for fixed dose titration: a high or low alarm occurring during the segment; a post-meal correction dose occurring during the segment; or other insulin doses occurring during the segment. If a data segment is not valid, then the DGS 100 processor may exclude it from the data set for the TOD period.

In a related aspect, the operations 1700 may include, at 1710, clearing data for each TOD period in response to any one or more of: determining the recommended fixed dose, determining a pre-meal correction factor, determining a post-meal correction factor, or determining a manual dose adjustment. For example, the DGS 100 processor may clear all TOD data buffers, along with low alarm and post-meal correction dose counters, when any of the following occur: when fixed dose titration guidance is issued; when the pre-meal CF titration guidance is issued; when the post-meal CF titration guidance is issued; or when a manual dose adjustment is issued.

Data Processing: The DGS 100 processor may call the fixed dose titration analysis once per day. Every day, the TOD data buffers, low alarm counters, and post-meal correction counters may be updated to include no more than the most recent 14 valid data segments for each TOD period. Of note, a low alarm counter and a high alarm counter may be implemented for every TOD. Referring to FIG. 21D, the method 1500 for providing dose guidance in response to analyte data may include additional operations 1800 in any operable order or combination, any one of which may be omitted if not necessary or desired. The operations 1800 may include, at 1802, determining a glucose pattern for each TOD period based on associated valid data segments for a set number of prior days, wherein determining the recommended fixed dose is further based on the glucose pattern. The operations 1800 may further include at 1804 determining, as a condition for determining the recommended fixed dose, that the associated valid data segments are available for the set number of prior days. The operations 1800 may further include, at 1806, determining the glucose pattern is low based on a count of low alarms occurring in each TOD period.

In a related aspect, fixed doses may be titrated by the number of instances of hyperglycemia or hypoglycemia that a user exhibits within a specific TOD period during given period of time. For example, if the DGS registers a number of hypoglycemic instances within a TOD period over a week that surpasses a predefined threshold, then the insulin dose associated with that TOD may be decreased as a potential remediation. A similar situation may occur to increase an insulin dose if the DGS registers a number of hyperglycemia instances within a TOD period over a week that surpasses a predefined threshold. Both the threshold number and time period may be configurable to make the DGS more or less reactive to instances of glucose dysregulation. The definition and calculation of either a hypo or hyperglycemic instance may be based upon a number of different factors, including but not limited to, crossing a specific threshold as well as a duration of time spent below the threshold.

In a related aspect, the DGS 100 processor may integrate fixed dose and correction factor titration. The operations 1800 may further include, at 1808, determining a pre-meal correction factor based on the time-correlated analyte data independently of the determining the recommended fixed dose, and if both the pre-meal correction factor and the recommended fixed dose indicate an increase in dose, then maintaining the pre-meal correction factor. Methods for fixed dose titration and premeal correction factor titration are described above and below. Both titrations may be performed by the DGS processor(s) independently in parallel. Each module may check for data sufficiency, and if appropriate, titrate daily at the same time. There may be conditions where the fixed dose and premeal CF are titrated on the same day. Some rules may be applied for the situation where both the fixed dose and premeal correction factor titration recommend dose increases. These rules may include, for example: do not decrease premeal CF if fixed dose titration outputs a low pattern during any time of the day; and do not decrease premeal CF if fixed dose titration suggests any fixed dose increase.

Post-Meal Correction Factor Titration: A DGS 100 processor may recalculate the post-meal correction factor using a predetermined scale factor whenever the pre-meal correction factor has changed.

For further examples of how GPA may be used in data processing, the TOD data buffer contents may impact the results under certain conditions, for example when glucose pattern analysis for each TOD requires the associated valid data segments to represent at least 7 different days, and pp-titration fixed dose titration guidance can only be provided if at least 7 different days are represented in ALL TODs. Other, independent data sufficiency requirements may be imposed within the GPA method/module 1600.

For every post-meal TOD, the DGS may maintain a counter that counts the number of low alarms that occur during that TOD. As part of the fixed-dose titration processing, this counter may be checked against a threshold of 4 occurrences—if this threshold is reached or exceeded, then a low pattern is input for the associated TOD to the titration mapping module regardless of the result from the GPA module. As noted above, the operations 1800 may further include, at 1806, determining the glucose pattern is low based on a count of low alarms occurring in each TOD period.

The method 1500 for providing dose guidance in response to analyte data may include additional operations 1900 as shown in FIG. 21E in any operable order or combination, any one of which may be omitted if not necessary or desired. For every post-meal TOD, the DGS 100 may maintain another counter that counts the number of post-meal correction doses that occur during that TOD. The operations 1900 may include, at, 1902, determining a glucose pattern condition based on a count of low alarms, a count of post-meal corrections during each TOD period, and the glucose pattern indicator. As part of the fixed-dose titration processing, the DGS processor may check this counter against a threshold of 4 occurrences—if this threshold is reached or exceeded, the following pattern is input to the titration mapping module, depending on the result from the GPA module: if GPA indicates a low pattern, then a low pattern is input; if GPA indicates a moderate hypo risk or a high/low pattern, then a high/low pattern is input; if GPA indicates no pattern or a high pattern, then a high pattern is input.

Consistent with the foregoing, and by way of addition example, the operations 1900 may further include, at 1904, determining that if the count of low alarms exceeds a first threshold, the count of post-meal corrections exceeds a second threshold, and a result of the GPA analysis indicates a low pattern, then determining the glucose pattern is low. At 1906, the operations may further include determining that if the count of low alarms exceeds a first threshold, the count of post-meal corrections exceeds a second threshold, and a result of the GPA analysis indicates a moderate hypoglycemic risk or high/low pattern, then determining the glucose pattern is high/low. At 1908, the operations may further include determining that if the count of low alarms exceeds a first threshold, the count of post-meal corrections exceeds a second threshold, and the glucose pattern indicator indicates no pattern or a high pattern, then determining the glucose pattern is high.

The DGS processor may adjust the fixed dose regimen as follows for basal, breakfast, lunch and/or dinner when the titration module output indicates a dose increase or decrease for any corresponding TOD. The amounts are described below:

Increase: Titrated fixed dose amount=Current fixed dose amount+Idelta where Idelta varies according to Grisk: 2 U if Grisk ≤15 mg/dL; 4 U if Grisk >15 mg/dL AND Grisk ≤30 mg/dL; 6 U if Grisk >30 mg/dL AND Grisk ≤60 mg/dL; 8 U if Grisk >60 mg/dL Decrease: Titrated fixed dose amount=Current fixed dose amount−10% of current fixed dose amount.

In a related aspect, the operations 1800 may further include, at 1808, determining a pre-meal correction factor based on the time-correlated analyte data independently of the determining the recommended fixed dose, and if both the pre-meal correction factor and the recommended fixed dose indicate an increase in dose, then maintaining the pre-meal correction factor. The DGS processor may calculate a pre-meal correction factor as described, for example in U.S. application Ser. No. 16/944,736.

Processing: A DGS processor may perform CFpre titration analysis once daily. Every day, it may update the CFpre titration glucose data buffer to include the most recent day of valid glucose pairs from data segments. Two aspects of processing have different requirements related to the data segment buffer: proceeding with analysis and performing analysis.

To proceed with CFpre titration analysis for the day, the data buffer should include at least a definite threshold number (e.g., fourteen valid pairs) of pre-dose glucose and post-dose glucose measurements that meet the following conditions: the pairs are associated with new initial meal doses, that is, these pairs have not been used in a prior CFpre titration that has been approved; and the glucose pairs are associated with initial meal doses that have a correction portion, that is, the dose has an additional amount, beyond the fixed dose and the pre-dose glucose is greater than the target glucose defined by the approved regimen. The foregoing conditions only need to be satisfied to initiate an analysis. The analysis itself may use all valid data pairs whether or not these pairs meet the foregoing conditions.

For glucose pairs where the pre-dose glucose value is less than the target glucose defined by the regimen, the pre-dose glucose should be set to the target glucose value prior to processing. This change does not need to occur persistently, only prior to processing. In some embodiments, where the pre-dose glucose value is less than the target glucose defined by the regimen, the pre-dose glucose will not be set to the target glucose value prior to processing. This situation indicates a scenario where the user has a premeal glucose value less than the pre-specified target glucose. As such, the premeal correction to a meal dose is negative to decrease the overall meal dose and avoid hypoglycemia.

The glucose pairs may be clustered according to the meal class that each glucose pair is associated with (breakfast, lunch, or dinner). A mean post-dose glucose value may be calculated for each meal class. The meal class mean may be subtracted from each post-dose glucose value in the respective cluster. Regression may then be performed on the data from all of the corrections.

CFpre titration may be performed in two alternative modes, rapid mode and stable mode. The transition between these two modes may be governed by the mode transition logic. Both may require a linear regression to be performed on the pre-dose glucose v. post-dose glucose pairs retrieved from the CFpre titration buffer. The linear regression may produce two results: a slope estimate and the corresponding p-value to determine if the estimate is statistically significantly different from zero.

Rapid Mode: This mode may be used when the system first starts in the guidance learning period and is executed every day until the mode transition logic, described below, is satisfied. In this mode, CFpre titration output may be determined only by the slope estimate. For example: if slope >0, then CFpre is decreased; else, if slope <=0, then CFpre is increased

Mode Transition Logic: While in Rapid Mode, each approved CFpre titration may be stored in a buffer. Whenever a Rapid Mode CFpre titration is issued, this buffer may be examined. If the five most recent titration changes oscillate consecutively between two CFpre values, the titration scheme may transition to Stable Mode.

Stable Mode: This second mode may be performed every day after the mode transition logic is satisfied. In this mode, the same logic as the Rapid Mode may be followed, except with the additional criteria that the CFpre value will only be changed if p-value <0.05.

CFpre Titration Calculation: When the CFpre titration analysis indicates a change that is then approved by an authorized HCP, the resulting CFpre value may be calculated as follows: if CFpre value is to be increased, then CFpre=CFpre,old*1.33; or if CFpre value is to be decreased, then CFpre=CFpre,old/1.33. CFpre values should only be decreased if the fixed dose titration analysis performed for the same day results in valid pattern findings for all TOD periods, and none of the patterns are low patterns.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

Systems, devices and methods are provided for determining a medication dose for a patient or user. The dose determination can account for recent and/or historical analyte levels of the patient or user. The dose determination can also take into account other information about the patient or user, such as physiological information, dietary information, activity, and/or behavior. Many different dose determination embodiments are set forth, pertaining to a wide array of different aspects of the system or environment in which the embodiments can be implemented. Systems, devices and methods are provided for displaying information related to glucose levels, including a time in range display and a graph of analyte levels containing an identification of a pattern type of a segment of the day.

In many systems, an apparatus for parameterizing a patient's medication dosing practice for configuring dose guidance settings, the apparatus includes: an input component configured to receive measured analyte data, meal data, and medication dosing data; a display component configured to visually present information; and one or more processors coupled with the input, the display, and a memory storing instructions and time-correlated data characterizing an analyte of the patient over an analysis period, wherein the instructions, when executed by the one or more processors, cause the apparatus to perform: receiving patient dose regimen information for the analysis period; evaluating a measure of consistency between the time-correlated data and the patient dose regimen information; and determining dose guidance information based on the measure of consistency.

In some systems, the memory holds further instructions for outputting the dose guidance information to the display.

In some systems, the memory holds further instructions for receiving the patient dose regimen information from the input component.

In some systems, the memory holds further instructions for receiving the patient dose regimen information by transmission from a remote data server.

In some systems, the patient dose regimen information comprises typical fixed medication doses taken at mealtimes and a typical time of day when breakfast is eaten.

In some systems, the patient dose regimen information comprises information defining a frequency of patient compliance with scheduled doses or meals.

In some systems, the medication comprises insulin.

In some systems, the input is wireless communications circuitry.

In some systems, the instructions for evaluating a measure of consistency further includes: classifying each dose of the patient dose regimen in a medication class, based on the time-correlated data; grouping each of the doses in one of a set of mealtime groups; generating dose parameters for the patient at least in part by applying data for each of the mealtime groups to a model; and storing the dose parameters for configuring dose guidance settings.

In some systems, the memory holds further instructions for accumulating the time-correlated data characterizing an analyte of the patient over a time period.

In some systems, the memory holds further instructions for determining the dose guidance information at least in part by reducing a recommendation for dosing based on detecting excursions of the analyte beyond a lower threshold in the time-correlated data. In some systems, the recommendation for dosing is for fixed dosing only.

In some systems, the memory holds further instructions for determining patient adherence to the patient dose regimen information based on the time-correlated data.

In some systems, the memory holds further instructions for determining whether to output the dose guidance parameters based on the measure of consistency.

In some systems, the memory holds further instructions for outputting the dose guidance parameters comprising predetermined dose suggestions if the measure of consistency indicates an unreliable system configuration.

In many methods, a method for facilitating efficient access by a healthcare provider (HCP) to an electronic medical record (EMR) of a patient generated by dose guidance system while protecting patient privacy is described. The method includes the steps of: authenticating, by at least one processor of a portable display device, a session with the patient; generating, by the at least one processor in response to receiving an input during the session from the patient indicating a request to share the EMRs with an HCP, an EMR identification code (ID); providing, by the at least one processor, the EMR ID and data needed to generate the report, to a remote server controlling access to the report; and outputting, by the at least one processor, the report to a display of the portable display device in response to the receipt of the EMR ID. In some embodiments, the portable display device may be a device under the control of the user. In some embodiments, the EMR ID may be shown on a device other than the portable display device. For example, the EMR ID may be sent in a separate message to a separate device for security reasons.

In some methods, the method further includes the step of providing the EMR to the remote server, prior to the authenticating.

In some methods, the method further includes the step of receiving the EMR from the dose guidance system.

In some methods, the method further includes the step of determining whether the EMR fails a condition for consistency with patient input indicating a dose pattern of a tracked medication. In some methods, the method further includes the step of, upon determining that the EMR fails the condition for consistency, providing the patient with an option to provide the EMR to the HCP. In some methods, wherein the generating, the providing, and the outputting are conditioned on the determining that the EMR fails the condition for consistency. In some methods, wherein the generating, the providing, and the outputting are conditioned on the determining that the EMR fails the condition for consistency.

In some methods, the method further includes the step of providing the patient with an option to provide the EMR to the HCP. In some methods, wherein the remote server upon receiving the EMR ID, creates a web page addressed at least in part by the EMR ID for displaying the EMR.

In some methods, wherein the EMR comprises determinations of dosing parameters for a medication administered to the patient at times during a defined period and a measure of consistency of the determinations with patient-supplied dosing information for the medication. In some methods, wherein the medication is insulin.

In many systems, a system for providing dose guidance to a subject is described. The system includes: an input configured to receive dose data from a medication delivery device, wherein the dose data comprises an amount and a time of a most recent drug dose administered; a display configured to visually present a dose guidance; one or more processors coupled with the input, the display, and a memory storing instructions, wherein the instructions, when executed by the one or more processors, cause the one or more processors to: classify the most recent drug dose administered as a meal dose or a correction dose; and in response to a determination that a period of time has elapsed since the time of the most recent drug dose administered, display an additional dose guidance.

In some systems, the period of time is about 2 hours. References herein to a time period of about 2 hours include time periods of greater than 1 hour. Time periods of about 2 hours also include time periods up to about 6 hours, preferably time periods less than 4 hours.

In some systems, the most recent drug dose administered is a meal dose, and wherein the dose data further comprises at least one additional drug dose administered after the most recent drug dose was administered, and wherein the period of time is not reset to a time administered of the at least one additional drug dose.

In some systems, the most recent drug dose administered is not a prime dose.

In some systems, the time of the most recent drug dose administered is a timestamp from a connected drug delivery device.

In some systems, the most recent drug dose administered is a correction dose, and wherein the dose data further comprises at least one additional drug dose administered after the most recent drug dose administered, and wherein a beginning of the period of time is reset to a time administered of the at least one additional drug dose administered.

In some systems, the system further includes a medication delivery device configured to deliver at least one dose of a drug to a subject.

In some systems, the input comprises wireless communications circuitry.

In many methods, a method for providing dose guidance is described. The method includes the steps of: receiving, by an electronic device, drug dose data of a subject from a medication delivery device, wherein the drug dose data comprises an amount and a time of a most recent drug dose administered; classifying the most recent drug dose administered as a meal dose or a correction dose, in response to determining that a period of time has elapsed since the time of the most recent drug dose administered, displaying a dose guidance.

In some methods, the period of time is about 2 hours.

In some methods, the most recent drug dose administered is a meal dose, wherein the drug dose data further comprises at least one additional drug dose administered after the most recent drug dose was administered, and wherein a beginning of the period of time is not reset to a time administered of the at least one additional drug dose.

In some methods, the most recent drug dose administered is not a prime dose.

In some methods, the time of the most recent drug dose administered is a timestamp from a connected drug delivery device.

In some methods, the most recent drug dose administered is a correction dose, wherein the drug dose data further comprises at least one additional drug dose administered after the most recent drug dose administered, and wherein a beginning of the period of time is reset to a time administered of the at least one additional drug dose administered.

In many systems, a system for providing dose guidance to a subject is described. The system includes an input configured to receive dose data from a medication delivery device, wherein the dose data comprises data related to a recent drug dose administered; a display configured to visually present a dose guidance; one or more processors coupled with the input, the display, and a memory storing instructions, wherein the instructions, when executed by the one or more processors, cause the one or more processors to: determine if the recent drug dose administered is a most recent drug dose administered; and in response to a determination that the recent drug dose administered is the most recent drug dose administered, display a screen comprising a dose guidance recommendation.

In some systems, the recent drug dose administered is determined to be the most recent drug dose administered through confirmation from the user.

In some systems, the dose data further comprises data related to at least one drug dose administered since a reset time, and wherein the instructions further cause the one or more processors to, in response to a determination that the at least one drug dose administered since the reset time has been classified, display the screen. In some systems, the dose data related to the at least one drug dose administered since the reset time was classified automatically. In some systems, the system further includes a medication delivery device configured to deliver at least one dose of a drug to a subject, wherein the instructions further cause the one or more processors to transmit one or more wireless interrogation signals to the medication delivery device to determine that a most recent dosage administered has been received. In some systems, the dose data related to the at least one drug dose administered since the reset time has been classified by the user.

In some systems, the instructions further cause the one or more processors to display a prompt for a user to confirm that information related to the most recent drug dose administered was correct. In some systems, the instructions further cause the one or more processors to display the screen comprising the dose guidance recommendation for a period of time that starts after confirmation from the user.

In some systems, the system further includes a medication delivery device configured to deliver at least one dose of a drug to a subject.

In some systems, the input is further configured to receive measured analyte data and a request for dose guidance, and wherein the instructions further cause the one or more processors to: determine if a glucose concentration at a time of receipt of the request for dose guidance is below a low threshold value; and in response to a determination that the glucose concentration at the time of receipt of the request for dose guidance is below the low threshold value, display a screen comprising a message to address a low glucose level before administering medication. In some systems, the instructions further cause the one or more processors to: in response to the determination that the glucose concentration at the time of receipt of the request for dose guidance is below the low threshold value, the dose guidance recommendation is not displayed.

In some systems, the input is further configured to receive measured analyte data and a request for dose guidance after a start time of a meal, and wherein the instructions further cause the one or more processors to: determine if a glucose concentration at an estimated start time of the meal is below a low threshold value; and in response to a determination that the glucose concentration at the estimated start time of the meal is below the low threshold value, display a screen comprising a message to address a low glucose level before administering medication. In some systems, the instructions further cause the one or more processors to: in response to the determination that the glucose concentration at the estimated start time of the meal is below the low threshold value is below the low threshold value, the dose guidance recommendation is not displayed.

In some systems, the input comprises wireless communications circuitry.

In some methods, the input is further configured to receive measured analyte data and a request for dose guidance, and wherein the instructions further cause the one or more processors to: determine if a glucose concentration at a time of receipt of the request for dose guidance is below a low threshold value; and in response to a determination that the glucose concentration at the time of receipt of the request for dose guidance is below the low threshold value, display a screen comprising a message to address a low glucose level before administering medication. In some methods, the instructions further cause the one or more processors to: in response to the determination that the glucose concentration at the time of receipt of the request for dose guidance is below the low threshold value, the dose guidance recommendation is not displayed.

In some methods, the input is further configured to receive measured analyte data and a request for dose guidance after a start time of a meal, and wherein the instructions further cause the one or more processors to: determine if a glucose concentration at an estimated start time of the meal is below a low threshold value; and in response to a determination that the glucose concentration at the estimated start time of the meal is below the low threshold value, display a screen comprising a message to address a low glucose level before administering medication. In some methods, the instructions further cause the one or more processors to: in response to the determination that the glucose concentration at the estimated start time of the meal is below the low threshold value is below the low threshold value, the dose guidance recommendation is not displayed.

In many methods, a method for providing dose guidance is provided. The method includes the steps of: receiving, by an electronic device, dose data of a user from a medication delivery device, wherein the dose data comprises data related to a recent drug dose administered; determining if the recent drug dose administered is a most recent drug dose administered; and in response to a determination that the recent drug dose administered is the most recent drug dose administered, displaying a screen comprising a dose guidance recommendation.

In some methods, the recent drug dose administered is determined to be the most recent drug dose administered through confirmation from the user.

In some methods, the recent drug dose administered was administered after a rest time, and the method further includes the step of: determining if the recent dose administered after the reset time was classified. In some methods, the recent dose administered after the reset time was classified automatically.

In some methods, the method further includes the step of: transmitting one or more wireless interrogation signals to a medication delivery device to determine that a most recent dosage administered has been received.

In some methods, the recent dose administered after the reset time was classified by the user.

In some methods, the method further includes the step of: displaying a prompt for the user to confirm that information related to the most recent drug dose administered is correct.

In some methods, the screen comprising the dose guidance recommendation is only displayed for a period of time that starts after a confirmation from the user that information related to the most recent drug dose administered is correct.

In many systems, a system for providing dose guidance to a subject. The system includes: an input configured to receive dose data from a medication delivery device, wherein the dose data comprises data related to at least one meal dose administered since a reset time; a display configured to visually present a plurality of meal icons; one or more processors coupled with the input, the display, and a memory storing instructions, wherein the instructions, when executed by the one or more processors, cause the one or more processors to: determine if the at least one meal dose administered since the reset time has been classified; and in response to a determination that the at least one meal dose administered since the reset time has been classified, display a screen comprising the plurality of meal icons.

In some systems, the instructions cause the one or more processors to determine if the at least one meal dose administered since the reset time has been classified as one of breakfast, lunch, or dinner.

In some systems, the plurality of meal icons includes a breakfast icon, a lunch icon, and a dinner icon, and wherein each of the breakfast, lunch, and dinner icons comprise a first appearance and a second appearance. In some systems, the first appearance is associated with a first state in which one of the at least one meal dose administered since the reset time has been classified as a meal type corresponding to the breakfast, lunch, or dinner icon, and the second appearance is associated with a second state in which one of the at least one meal dose administered since the reset time has not been classified as a meal type corresponding to the breakfast, lunch, or dinner icon. In some systems, the first appearance comprises a shaded appearance. In some systems, the second appearance comprises an unshaded appearance. In some systems, the second appearance is brighter than the first presentation. The meal icons may be text or alphanumeric characters or may be images. The icon may also change in form between the first and second appearance.

In some systems, the instructions further cause the one or more processors to: classify the at least one meal dose administered since the reset time as a breakfast dose, a lunch dose, or a dinner dose.

In some systems, the instructions further cause the one or more processors to: receive input from a user, wherein the input comprises a classification of the at least one meal dose administered since the reset time as a breakfast dose, a lunch dose, or a dinner dose.

In some systems, the reset time is determined based on at least one time range associated with at least one meal.

In some systems, the reset time is determined based on a time range associated with a dinner dose and a time range associated with a breakfast dose.

In some systems, the reset time is midnight.

In some systems, the reset time is a time that is about halfway between an end of a dinner dose time range and a start of a breakfast dose time range.

In some systems, the system further includes a medication delivery device configured to deliver at least one dose of a drug to a subject.

In some systems, the input comprises wireless communications circuitry.

In many methods, a method for providing dose guidance is described. The method includes the steps of: receiving, by an electronic device, drug dose data of a user from a medication delivery device, wherein the drug dose data comprises data related to at least one meal dose administered since a reset time; determining if the at least one meal dose administered since the reset time has been classified; in response to a determination that the at least one meal dose administered since the reset time has been classified, displaying a screen comprising a plurality of meal icons.

In some methods, the determining step comprises determining if the at least one meal dose administered since the reset time has been classified as one of breakfast, lunch, or dinner.

In some methods, the plurality of meal icons includes a breakfast icon, a lunch icon, and a dinner icon, and wherein each of the breakfast, lunch, and dinner icons comprise a first appearance and a second appearance, wherein the first appearance is associated with a first state in which one of the at least one meal dose administered since the reset time has been classified as a meal type corresponding to the breakfast, lunch, or dinner icon, and the second appearance is associated with a second state in which one of the at least one meal dose administered since the reset time has not been classified as a meal type corresponding to the breakfast, lunch, or dinner icon. In some methods, the first appearance comprises a shaded appearance. In some methods, the second appearance comprises an unshaded appearance. In some methods, the second appearance is brighter than the first presentation.

In some methods, the at least one meal dose administered since the reset time has been classified automatically by one or more processors of a reader device.

In some methods, the at least one meal dose administered since the reset time has been classified by the user.

In some methods, the reset time is determined based on at least one time range associated with at least one meal.

In some methods, the reset time is determined based on a time range associated with dinner dose and a time range associated with breakfast dose.

In some methods, the reset time is midnight.

In some methods, the reset time is a time that is about halfway between an end of a dinner dose time range and a start of a breakfast dose time range.

In many systems, a system for providing dose guidance to a subject is described. The system includes: an input configured to receive dose data from a medication delivery device; a display configured to visually present a dose guidance; one or more processors coupled with the input, the display, and a memory storing instructions, wherein the instructions, when executed by the one or more processors, cause the one or more processors to: determine if a missed dose alert is active; in response to a determination that a missed dose alert is not active, display a dose guidance recommendation based on a normal meal dose calculation; and in response to a determination that a missed dose alert is active, display a dose guidance recommendation based on a late meal dose calculation.

In some systems, the input comprises wireless communications circuitry.

In some systems, the instructions further cause the one or more processors to: determine if the dose data received from the medication delivery device comprises a dose administered within a period of time of the current time; and in response to a determination that the dose data received from the medication delivery device comprises a dose administered within a period of time of the current time, display the dose guidance recommendation based on the late meal dose calculation. In some systems, the instructions cause the one or more processors to: display the dose guidance recommendation based on the late meal dose calculation in response to the determination that the missed dose alert is active and the determination that the dose data received from the medication delivery device comprises a dose administered within a period of time of the current time. In some systems, the period of time is about two hours.

In some systems, the normal meal dose calculation is based on a fixed insulin dose associated with a meal and an amount of insulin on board if a current glucose level of the user is below a target glucose value.

In some systems, the normal meal dose calculation is based on a fixed insulin dose associated with a meal and an amount of insulin on board if a current glucose level of the user is below a target glucose value.

In some systems, each of the normal meal dose calculation and the late meal dose calculation are based on a fixed insulin dose associated with a meal, an amount of insulin on board, a correction adjustment, and a trend adjustment, if a current glucose level of the user is above a target glucose value. In some systems, the normal meal dose calculation is based on a current glucose level determined from scanned glucose data. In some systems, the late meal dose calculation is based on a current glucose level determined from streaming glucose data. In some systems, the late meal dose calculation is further based on a trend adjustment determined from streaming glucose data. In some systems, the late meal dose calculation is based on the amount of insulin on board calculated according to a time of a request for the dose guidance recommendation by the user.

In some systems, the system further includes a medication delivery device configured to deliver at least one dose of a drug to a subject.

In many methods, a method for providing dose guidance is described. The method includes the steps of: receiving, by an electronic device, drug dose data of a user from a medication delivery device; determining if a missed dose alert is active; and in response to a determination that a missed dose alert is not active, display a dose guidance recommendation based on a normal meal dose calculation; and in response to a determination that a missed dose alert is active, display a dose guidance recommendation based on a late meal dose calculation.

In some methods, the method further includes the steps of: determining if the drug dose data comprises data for doses administered within a period of time; displaying the dose guidance recommendation based on the late meal dose calculation in response to a determination that the drug dose data does not include any data for doses administered within a period of time. In some methods, the period of time is about two hours.

In some methods, the normal meal dose calculation is based on a fixed insulin dose associated with a meal and an amount of insulin on board if a current glucose level of the user is below a target glucose value.

In some methods, each of the normal meal dose calculation and the late meal dose calculation are based on a fixed insulin dose associated with a meal, an amount of insulin on board, a correction adjustment, and a trend adjustment, if a current glucose level of the user is above a target glucose value. In some methods, the normal meal dose calculation is based on a current glucose level determined from scanned glucose data. In some methods, the late meal dose calculation is based on a current glucose level determined from streaming glucose data. In some methods, the late meal dose calculation is further based on a trend adjustment determined from streaming glucose data. In some methods, the late meal dose calculation is based on the amount of insulin on board calculated according to a time of a request for the dose guidance recommendation by the user.

In many systems, a system for providing alerts to a subject is described. The system includes: an input configured to receive streaming glucose data from a sensor control device; a display configured to present an alert; one or more processors coupled with the input, the display, and a memory storing instructions, wherein the instructions, when executed by the one or more processors, cause the one or more processors to: determine at a current time if a meal dose has been missed in association with a meal having an estimated meal start time by detecting a missed meal dose condition for a consecutive number of minutes; determine if an insulin dose has not been recorded within about 45 minutes prior to the estimated meal start time; and in response to a detection of a missed meal dose condition for the consecutive number of minutes and a determination that the insulin dose has not been recorded within about 45 minutes prior to the estimated meal start time, display an alert interface relating to the missed meal dose.

In some systems, the consecutive number of minutes is about 5 minutes.

In some systems, the instructions further cause the one or more processors to: determine if a meal dose has been recorded within about 2 hours of the current time; and in response to a determination that the meal dose has been recorded within about 2 hours of the current time, display the alert interface relating to the missed meal dose.

In some systems, the instructions further cause the one or more processors to: determine if a correction dose alert is asserted; and in response to a determination that the correction dose alert is not asserted, display the alert interface relating to the missed meal dose.

In some systems, the system further includes a sensor control device configured to collect data indicative of an analyte level in a subject, the sensor control device comprising an analyte sensor, wherein at least a portion of the analyte sensor is configured to be in fluid contact with a bodily fluid of the subject.

In some systems, the input comprises wireless communications circuitry.

In many methods, a method for alerting a user to a missed meal dose is described. The method includes the steps of: receiving, by an electronic device, streaming glucose data from a sensor control device; determining at a current time if a meal dose has been missed in association with a meal having an estimated meal start time by detecting a missed meal dose condition for a consecutive number of minutes; determining if an insulin dose has not been recorded within about 45 minutes prior to the estimated meal start time; and in response to a detection of a missed meal dose condition for the consecutive number of minutes and a determination that the insulin dose has not been recorded within about 45 minutes prior to the estimated meal start time, displaying an alert interface relating to the missed meal dose.

In some methods, the consecutive number of minutes is about 5 minutes.

In some methods, the method further includes the steps of: if a meal dose has been recorded within about 2 hours of the current time; and in response to a determination that the meal dose has been recorded within about 2 hours of the current time, displaying the alert interface relating to the missed meal dose.

In some methods, the method further includes the steps of: if a meal dose has been recorded within about 2 hours of the current time; and in response to a determination that the meal dose has been recorded within about 2 hours of the current time, displaying the alert interface relating to the missed meal dose.

In some methods, determining if a correction dose alert is asserted; in response to a determination that the correction dose alert is not asserted, displaying the alert interface relating to the missed meal dose.

In many systems, a system for managing alerts is described. The system includes: an input configured to receive streaming glucose data from a sensor control device; a display configured to present an alert; one or more processors coupled with the input, the display, and a memory storing instructions, wherein the instructions, when executed by the one or more processors, cause the one or more processors to: assert a missed meal dose alert; determine if a missed meal dose condition has been detected for a consecutive number of minutes after the missed meal dose alert has been asserted; in response to a determination that the missed meal dose condition has not been detected for the consecutive number of minutes after the missed meal dose alert has been asserted, rescind the missed meal dose alert.

In some systems, the consecutive number of minutes is 15 consecutive minutes.

In some systems, the missed meal dose condition comprises a determination that an insulin dose was not administered within a period of time of an estimated meal start time.

In some systems, the input comprises wireless communications circuitry.

In many methods, a method for rescinding an alert to a missed meal dose is described. The method includes the steps of: receiving, by an electronic device, streaming glucose data from a sensor control device; asserting a missed meal dose alert; determining if a missed meal dose condition has been detected for a consecutive number of minutes after the missed meal dose alert has been asserted; in response to a determination that the missed meal dose condition has not been detected for the consecutive number of minutes after the missed meal dose alert has been asserted, rescinding the missed meal dose alert.

In some methods, the consecutive number of minutes is 15 consecutive minutes.

In some methods, the missed meal dose condition comprises determining that an insulin dose was not administered within a period of time of an estimated meal start time.

In many systems, a system for managing alerts is described. The system includes: an input configured to receive streaming glucose data from a sensor control device and dose data from a medication delivery device; a display configured to present an alert; one or more processors coupled with the input, the display, and a memory storing instructions, wherein the instructions, when executed by the one or more processors, cause the one or more processors to: assert a missed meal dose alert at a current time; determine if an insulin dose has been recorded within about 2 hours of the current time; in response to a determination that the insulin dose has been recorded within about 2 hours of the current time, rescind the missed meal dose alert.

In many methods, a method for rescinding an alert to a missed meal dose is described. The method includes the steps of: receiving, by an electronic device, streaming glucose data from a sensor control device; asserting a missed meal dose alert at a current time; determining if an insulin dose has been recorded within about 2 hours of the current time; and in response to a determination that the insulin dose has been recorded within about 2 hours of the current time, rescinding the missed meal dose alert.

In many systems, a system for managing alerts is described. The system includes: an input configured to receive streaming glucose data from a sensor control device and dose data from a medication delivery device; a display configured to present an alert; one or more processors coupled with the input, the display, and a memory storing instructions, wherein the instructions, when executed by the one or more processors, cause the one or more processors to: assert a missed meal dose alert, wherein the missed meal dose alert relates to a missed meal having an estimated start time; determine if an insulin dose has been recorded within about 45 minutes of the estimated meal start time; and in response to a determination that the insulin dose has been recorded within about 45 minutes of the estimated meal start time, rescind the missed meal dose alert.

In many methods, a method for rescinding an alert to a missed meal dose is described. The method includes the steps of: receiving, by an electronic device, streaming glucose data from a sensor control device; asserting a missed meal dose alert, wherein the missed meal dose alert relates to a missed meal having an estimated start time; determining if an insulin dose has been recorded within about 45 minutes of the estimated meal start time; and in response to a determination that the insulin dose has been recorded within about 45 minutes of the estimated meal start time, rescinding the missed meal dose alert.

In many systems, a system for managing alerts is described. The system includes: an input configured to receive streaming glucose data from a sensor control device; a display configured to present an alert; one or more processors coupled with the input, the display, and a memory storing instructions, wherein the instructions, when executed by the one or more processors, cause the one or more processors to: assert a missed meal dose alert at a current time, wherein the missed meal dose alert relates to a missed meal having an estimated start time; determine if the estimated meal start time is within about 2 hours of the current time; in response to a determination that the estimated meal start time did not occur within about 2 hours of the current time, rescind the missed meal dose alert.

In some systems, the input comprises wireless communications circuitry.

In many methods, a method for rescinding an alert to a missed meal dose is described. The method includes the steps of: receiving, by an electronic device, streaming glucose data from a sensor control device; asserting a missed meal dose alert at a current time, wherein the missed meal dose alert relates to a missed meal having an estimated start time; determining if the estimated meal start time is within about 2 hours of the current time; and in response to a determination that the estimated meal start time did not occur within about 2 hours of the current time, rescinding the missed meal dose alert.

In many systems, a system for providing dose guidance to a subject is described. The system includes: an input configured to receive dose data from an insulin delivery device; a display configured to visually present a dose guidance; one or more processors coupled with the input, the display, and a memory storing instructions, wherein the instructions, when executed by the one or more processors, cause the one or more processors to: determine if a correction dose alert has been asserted at a current time; determine if an insulin dose has not been administered to the subject within about 2 hours of the current time; and in response to a determination that the correction dose has been asserted and a determination that the insulin dose has not been administered to the subject within about 2 hours of the current time, display a correction dose guidance.

In some systems, the instructions further cause the one or more processors to: determine if the correction dose alert has been resolved; and in response to a determination that the correction dose alert has been resolved, display the correction dose guidance.

In some systems, the input comprises wireless communications circuitry.

In many methods, a method for recommending correction doses is described. The method includes the steps of: receiving, by an electronic device, insulin dose data of a subject from an insulin delivery device; determining if a correction dose alert has been asserted at a current time; determining if an insulin dose has not been administered to the subject within about 2 hours of the current time; and displaying a correction dose guidance, wherein the correction dose guidance is displayed in response to a determination that the correction dose has been asserted and a determination that the insulin dose has not been administered to the subject within about 2 hours of the current time.

In some methods, the method further includes the step of determining if the correction dose alert has been resolved, wherein the correction dose guidance is only displayed if the correction dose alert has been resolved.

In many systems, a system for providing alerts to a subject is described. The system includes: an input configured to receive streaming glucose data from a sensor control device and dose data from an insulin delivery device; a display configured to visually present an alert; one or more processors coupled with the input, the display, and a memory storing instructions, wherein the instructions, when executed by the one or more processors, cause the one or more processors to: determine at a current time if a correction dose condition has been asserted for a consecutive number of minutes; determine if an insulin dose has not been recorded within about 2 hours of the current time; and in response to a determination that the correction dose condition has been asserted for a consecutive number of minutes and a determination that the insulin dose has not been recorded, or alternatively received, within about 2 hours of the current time, display an alert interface relating to the correction dose alert.

In some systems, the consecutive number of minutes is about 5 minutes.

In some systems, the instructions further cause the one or more processors to: determine if a missed meal dose alert is asserted; in response to a determination that the missed dose alert is not asserted, display the alert interface relating to the correction dose alert; and in response to a determination that the missed dose alert is asserted, not display the alert interface relating to the correction dose alert.

In some systems, the input comprises wireless communications circuitry.

In many methods, a method for alerting a user regarding a correction dose is described. The method includes the steps of: receiving, by an electronic device, streaming glucose data from a sensor control device and insulin dose data of a subject from an insulin delivery device; determining at a current time if a correction dose condition has been asserted for a consecutive number of minutes; determining if an insulin dose has not been recorded within about 2 hours of the current time; and in response to a determination that the correction dose condition has been asserted for a consecutive number of minutes and a determination that the insulin dose has not been recorded, alternatively received, within about 2 hours of the current time, displaying an alert interface relating to the correction dose alert.

In some methods, the consecutive number of minutes is about 5 minutes.

In some methods, the method further includes the step of determining if a missed meal dose alert is not asserted before displaying the alert interface relating to the correction dose alert. In some methods, the alert interface relating to the correction dose alert is only displayed in response to a determination that the missed meal dose alert is not asserted.

In many systems, a system for managing alerts is described. The system includes: an input configured to receive streaming glucose data from a sensor control device; a display configured to present an alert; one or more processors coupled with the input, the display, and a memory storing instructions, wherein the instructions, when executed by the one or more processors, cause the one or more processors to: assert a correction dose alert; determine if a correction dose condition has been detected for a consecutive number of minutes after the correction dose has been asserted; in response to a determination that the correction dose condition has not been detected for the consecutive number of minutes after the correction dose has been asserted, rescind the correction dose alert.

In some systems, the consecutive number of minutes is 15 consecutive minutes.

In some systems, the input comprises wireless communications circuitry.

In many methods, a method for rescinding an alert to a correction dose is described. The method includes the steps of: receiving, by an electronic device, streaming glucose data from a sensor control device; asserting a correction dose alert; determining if a correction dose condition has been detected for a consecutive number of minutes after the correction dose has been asserted; in response to a determination that the correction dose condition has not been detected for the consecutive number of minutes after the correction dose has been asserted, rescinding the correction dose alert.

In some methods, the consecutive number of minutes is 15 consecutive minutes.

In many systems, a system for managing alerts is described. The system includes: an input configured to receive streaming glucose data from a sensor control device; a display configured to present an alert; one or more processors coupled with the input, the display, and a memory storing instructions, wherein the instructions, when executed by the one or more processors, cause the one or more processors to: assert a correction dose alert, wherein the correction dose alert was first asserted at a first time; determine if a calculation for insulin on board (JOB) has changed since the first time; and in response to a determination that the calculation for insulin on board (JOB) has changed since the first time, rescind the correction dose alert.

In some systems, the input comprises wireless communications circuitry.

In many methods, a method for rescinding an alert to a correction dose is described. The method includes the steps of: receiving, by an electronic device, streaming glucose data from a sensor control device; asserting a correction dose alert, wherein the correction dose alert was first asserted at a first time; determining if a calculation for insulin on board (JOB) has changed since the first time; in response to a determination that the calculation for insulin on board (JOB) has changed since the first time, rescind the correction dose alert.

In many systems, a system for managing alerts is described. The system includes: an input configured to receive streaming glucose data from a sensor control device and dose data from an insulin delivery device; a display configured to visually present a dose guidance; one or more processors coupled with the input, the display, and a memory storing instructions, wherein the instructions, when executed by the one or more processors, cause the one or more processors to: assert a correction dose alert at a current time; determine if an insulin dose has been recorded within a period of time of the current time; and in response to a determination that the insulin dose has been recorded within the period of time of the current time, rescind the correction dose alert.

In some systems, the input comprises wireless communications circuitry.

In many methods, a method for rescinding an alert to a correction dose is described. The method includes the seps of: receiving, by an electronic device, streaming glucose data from a sensor control device and insulin dose data of a subject from an insulin delivery device; asserting a correction dose alert at a current time; determining if an insulin dose has been recorded within a period of time of the current time; and rescinding the correction dose alert if it is determined that the insulin dose has been recorded within the period of time of the current time.

In some methods, the period of time is about 2 hours.

In many systems, a system for classifying doses is described. The system includes an input configured to receive insulin dose data of a user from a connected insulin delivery device, wherein the insulin dose data comprises a recent dose comprising an insulin amount and a timestamp; a display configured to visually present a dose guidance; one or more processors coupled with the input, the display, and a memory storing instructions, wherein the instructions, when executed by the one or more processors, cause the one or more processors to: provide a dose recommendation guidance for a meal requested by a user at a request time, wherein the meal has a meal type and the dose recommendation guidance comprises a recommended dose amount; determine if the timestamp of the recent dose is within a period of time of the request time; determine if the insulin amount of the recent dose is the same as the recommended dose amount of the meal dose guidance recommendation; and in response to a determination that the timestamp of the recent dose is within a period of time of the request time and a determination that the insulin amount of the recent dose is the same as the recommended dose amount of the meal dose guidance recommendation, classify the recent dose as associated with the meal type of the recent meal.

In some systems, the period of time is less than or equal to about 20 minutes.

In some systems, the meal type is selected from the group consisting of breakfast, lunch, and dinner.

In some systems, the instructions further cause the one or more processors to: determine if the timestamp of the recent dose is within a determined meal dose time range for the meal type of the recent meal; and in response to a determination that the timestamp of the recent dose is within the determined meal dose time range for the meal type of the recent meal, classify the recent dose as associated with the meal type of the recent meal.

In some systems, the instructions further cause the one or more processors to: determine if the recent dose was taken while the user was in a post-meal state; and in response to a determination that the recent dose was taken while the user was not in the post-meal state, classify the recent dose as associated with the meal type of the recent meal. In some systems, in the post-meal state, a previous dose administered within about 2 hours of the request time has been associated with the meal type of the recent meal.

In some systems, the input comprises wireless communications circuitry.

In many methods, a method for classifying doses from a connected insulin delivery device is described. The method includes the steps of: providing a dose recommendation guidance for a meal requested by a user at a request time, wherein the meal has a meal type and the dose recommendation guidance comprises a recommended dose amount; receiving, by an electronic device, insulin dose data of a user from the connected insulin delivery device, wherein the insulin dose data comprises a recent dose comprising an insulin amount and a timestamp; determining if the timestamp of the recent dose is within a period of time of the request time; determining if the insulin amount of the recent dose is the same as the recommended dose amount of the meal dose guidance recommendation; and in response to a determination that the timestamp of the recent dose is within a period of time of the request time and a determination that the insulin amount of the recent dose is the same as the recommended dose amount of the meal dose guidance recommendation, classifying the recent dose as associated with the meal type of the recent meal.

In some methods, the period of time is less than or equal to about 20 minutes.

In some methods, the meal type is selected from the group consisting of breakfast, lunch, and dinner.

In some methods, the method further includes the step of determining if the timestamp of the recent dose is within a determined meal dose time range for the meal type of the recent meal.

In some methods, the method further includes the step of determining if the recent dose was taken while the user was in a post-meal state. In some methods, in a post-meal state, a previous dose administered within about 2 hours of the request time has been associated with the meal type of the recent meal.

In many systems, a system for classifying doses is described. The system includes: an input configured to receive insulin dose data of a user from a connected insulin delivery device, wherein the insulin dose data comprises a recent dose comprising an insulin amount and a timestamp; a display configured to visually present a dose guidance; one or more processors coupled with the input, the display, and a memory storing instructions, wherein the instructions, when executed by the one or more processors, cause the one or more processors to: provide a dose recommendation guidance at a first time; determine if the timestamp of the recent dose is within a period of time of the first time; determine if the insulin amount of the recent dose is the same as the recommended dose amount of the dose recommendation guidance; and classify the recent dose as a meal dose, a correction dose, or as ambiguous.

In some systems, the period of time is less than or equal to about 20 minutes.

In some systems, the dose recommendation guidance was a correction dose recommendation guidance, and wherein the recent dose is classified as a correction dose.

In some systems, the dose recommendation guidance was a dose recommendation guidance for a meal, wherein the meal has a meal type, and wherein the recent dose is classified as a dose for the meal type. In some systems, the instructions further cause the one or more processors to: determine if the timestamp of the recent dose is within a determined meal dose time range for the meal type of the recent meal. In some systems, the instructions further cause the one or more processors to: determine if the recent dose was taken while the user was in a post-meal state.

In some systems, the recent dose is classified as ambiguous.

In some systems, the instructions further cause the one or more processors to prompt the user to classify the recent dose manually. In some systems, the instructions further cause the one or more processors to: prompt the user to classify the recent dose manually by prompting the user to select a classification from the group consisting of a breakfast dose, a lunch dose, a dinner dose, and a correction dose. In some methods, the instructions further cause the one or more processors to: prompt the user to classify the recent dose manually by prompting the user to select a classification from the group consisting of a snack dose, a priming dose, and a dose not taken. In some methods, the instructions further cause the one or more processors to: prompt the user to classify the recent dose manually by prompting the user to select a classification relating to eating more than expected from a previous meal.

In some systems, the recent dose that was classified as ambiguous must be classified as a classification other than ambiguous before providing an additional dose guidance recommendation.

In some systems, the input comprises wireless communications circuitry.

In many methods, a method for classifying doses from a connected insulin delivery device is described. The method includes the steps of: providing a dose recommendation guidance at a first time; receiving, by an electronic device, insulin dose data of a user from the connected insulin delivery device, wherein the insulin dose data comprises a recent dose comprising an insulin amount and a timestamp; determining if the timestamp of the recent dose is within a period of time of the first time; determining if the insulin amount of the recent dose is the same as the recommended dose amount of the dose recommendation guidance; and classifying the recent dose as a meal dose, a correction dose, or as ambiguous.

In some methods, the period of time is less than or equal to about 20 minutes.

In some methods, the dose recommendation guidance was a correction dose recommendation guidance, and wherein the recent dose is classified as a correction dose.

In some methods, the dose recommendation guidance was a dose recommendation guidance for a meal, wherein the meal has a meal type, and wherein the recent dose is classified as a dose for the meal type. In some methods, the method further includes the step of determining if the timestamp of the recent dose is within a determined meal dose time range for the meal type of the recent meal. In some methods, the method further includes the step of determining if the recent dose was taken while the user was in a post-meal state.

In some methods, the recent dose is classified as ambiguous. In some methods, the method further includes the step prompting the user to classify the recent dose manually. In some methods, the step of prompting the user to classify the recent dose manually comprises prompting the use to select a classification from the group consisting of a breakfast dose, a lunch dose, a dinner dose, and a correction dose. In some methods, the step of prompting the user to classify the recent dose manually comprises prompting the use to select a classification from the group consisting of a snack dose, a priming dose, and a dose not taken. In some methods, the step of prompting the user to classify the recent dose manually comprises prompting the use to select a classification relating to eating more than expected from a previous meal. In some methods, the recent dose that was classified as ambiguous must be classified as a classification other than ambiguous before providing an additional dose guidance recommendation.

In many systems, an apparatus for providing dose guidance in response to analyte data is described. The apparatus includes: an input configured to receive measured analyte data, meal data, and medication dosing data; a display configured to visually present information; and one or more processors coupled with the input, the display, and a memory storing instructions, wherein the instructions, when executed by the one or more processors, cause the apparatus to perform: receiving time-correlated analyte data of a patient taken over an analysis period into a buffer; dividing the time-correlated analyte data into discrete time-of-day (TOD) periods; determining, by executing an algorithm, a recommended fixed dose of the medication for a corresponding one of the TOD periods based on at least one portion of the time-correlated analyte data and a defined dosing strategy of the patient for the analysis period; and storing an indicator of the recommended fixed dose in a computer memory for output to at least one of a user or a medication dosing device.

In some systems, the memory holds further instructions for determining the recommended fixed dose at least in part by: classifying each of the medication doses in a medication class, based on the time-correlated data; grouping each of the doses in one of a set of mealtime groups; determining a glucose pattern most closely fitting the time-correlated data; and selecting a glucose pattern indicator, based on the glucose pattern.

In some systems, the memory holds further instructions for classifying doses in classes comprising a fixed basal dose, fixed breakfast dose, fixed lunch dose, fixed dinner dose for corresponding TOD periods.

In some systems, the memory holds further instructions for determining, as a condition for use in determining the recommended fixed dose, that a segment of the time-correlated analyte data is free of any gaps exceeding a defined threshold.

In some systems, the memory holds further instructions for determining, as a condition for use in determining the recommended fixed dose, that a segment of the time-correlated analyte data has an associated initial meal dose.

In some systems, the memory holds further instructions for determining, as a condition for use in determining the recommended fixed dose, that a segment of the time-correlated analyte data is associated with a basal fixed dose within a prior 24-hour period.

In some systems, the memory holds further instructions for clearing data for each TOD period in response to any one or more of: determining the recommended fixed dose, determining a pre-meal correction factor, determining a post-meal correction factor, or determining a manual dose adjustment.

In some systems, the memory holds further instructions for determining a glucose pattern for each TOD period based on associated valid data segments for a set number of prior days, wherein determining the recommended fixed dose is further based on the glucose pattern. In some systems, the memory holds further instructions for determining, as a condition for determining the recommended fixed dose, that the associated valid data segments are available for the set number of prior days. In some systems, the memory holds further instructions for determining the glucose pattern is low based on a count of low alarms occurring in each TOD period. In some systems, the memory holds further instructions for determining the glucose pattern is low based on a count of hypoglycemic instances in each TOD period. In some systems, the memory holds further instructions for determining the glucose pattern is high based on a count of hyperglycemic instances in each TOD period. In some systems, the memory holds further instructions for determining a pre-meal correction factor based on the time-correlated analyte data independently of the determining the recommended fixed dose, and if both the pre-meal correction factor and the recommended fixed dose indicate an increase in dose, then maintaining the pre-meal correction factor. In some embodiments, the memory holds further instructions for determining the glucose pattern is low based on a count of hypoglycemic instances in each TOD period. In some embodiments, the memory holds further instructions for determining the glucose pattern is high based on a count of hyperglycemic instances in each TOD period.

In some systems, the memory holds further instructions for determining a glucose pattern condition based on a count of low alarms, a count of post-meal corrections during each TOD period, and the glucose pattern indicator. In some systems, the memory holds further instructions for determining that if the count of low alarms exceeds a first threshold, the count of post-meal corrections exceeds a second threshold, and a result of the GPA method indicates a low pattern, then determining the glucose pattern is low. In some systems, the memory holds further instructions for determining that if the count of low alarms exceeds a first threshold, the count of post-meal corrections exceeds a second threshold, and a result of the GPS method indicates a moderate hypoglycemic risk or high/low pattern, then determining the glucose pattern is high/low. In some systems, the memory holds further instructions for determining that if the count of low alarms exceeds a first threshold, the count of post-meal corrections exceeds a second threshold, and the glucose pattern indicator indicates no pattern or a high pattern, then determining the glucose pattern is high.

In some systems, the input is wireless communications circuitry.

In some embodiments, the at least on portion of the time-correlated analyte data does not include a portion of time-correlated analyte data selected to be excluded by a user.

In many systems, a medication delivery device is described. The device includes: an input configured to receive a query for dose data for a period of time, wherein the dose data comprises an amount and a time of all doses delivered during the period of time; one or more processors coupled with the input and a memory storing instructions, wherein the instructions, when executed by the one or more processors, cause the one or more processors to: store data for doses administered during the period of time to create stored data; determine if the stored data includes all doses delivered during the period of time; and in response to a determination that the stored data does not include all doses delivered during the period of time, create an indication of incomplete dose data.

In some systems, the instructions further cause the one or more processors to transmit the indication of incomplete dose data in response to the query for dose data.

In some systems, the indication of incomplete dose data is a counter value.

In some systems, the indication of incomplete dose data is based on a counter value.

In some systems, the indication of incomplete dose data is based on a comparison of a counter value and an estimated counter value. In some systems, the estimated counter value is calculated based on a prior counter value and an elapsed time since the prior counter value was received.

In some systems, the medication delivery device is a connected insulin pen, and wherein the connected insulin pen is configured to transmit dose data wirelessly.

In some systems, the medication delivery device is an insulin pen and a connected pen cap, wherein the connected pen cap is configured to transmit dose data wirelessly.

In some systems, the indication of incomplete dose data is based on a detection that a pen cap was not attached to an insulin pen for a different period of time. In some systems, the detection that the pen cap was not attached to the insulin pen for the different period of time comprises a determination that the insulin pen contained a first amount of insulin before the pen cap was not attached and a second amount of insulin after the pen cap was reattached to the insulin pen, wherein the first amount is different than the second amount.

In some systems, the input is wireless communications circuitry.

In many embodiments, a method of transferring data includes the steps of: receiving a query for dose data for a period of time, wherein the dose data comprises an amount and a time of all doses delivered during the period of time; storing data for doses administered during the period of time to create stored data; determining if the stored data includes all doses delivered during the period of time; and in response to a determination that the stored data does not include all doses delivered during the period of time, creating an indication of incomplete dose data.

In some embodiments, the method further includes the step of transmitting the indication of incomplete dose data in response to the query for dose data.

In some embodiments, the indication of incomplete dose data is a counter value.

In some embodiments, the indication of incomplete dose data is based on a counter value.

In some embodiments, the indication of incomplete dose data is based on a comparison of a counter value and an estimated counter value. In some embodiments, the estimated counter value is calculated based on a prior counter value and an elapsed time since the prior counter value was received.

In some embodiments, the indication of incomplete dose data is based on a detection that a pen cap was not attached to an insulin pen for a different period of time. In some embodiments, the detection that the pen cap was not attached to the insulin pen for the different period of time comprises a determination that the insulin pen contained a first amount of insulin before the pen cap was not attached and a second amount of insulin after the pen cap was reattached to the insulin pen, wherein the first amount is different than the second amount.

In some embodiments, the query for dose data from the period of time was sent from an application that provides dose guidance.

In many embodiments, a system for providing dose guidance to a subject includes: an input configured to receive dose data and an indication of incomplete dose data from a medication delivery device, wherein the dose data comprises data related to at least one dose administered during a period of time; a display configured to visually present a dose guidance; one or more processors coupled with the input, the display, and a memory storing instructions, wherein the instructions, when executed by the one or more processors, cause the one or more processors to: query the medication delivery device for the dose data comprising data related to at least one dose administered during the period of time; determine if the indication of incomplete dose data is received from the medication delivery device; in response to a determination that the indication of incomplete dose data is received, output a prompt seeking confirmation that the dose data received for the period of time includes dose data for all doses administered during the period of time; and in response to a determination that the indication of incomplete dose data is not received, calculate a dose guidance.

In some embodiments, the instructions further cause the one or more processors to output the dose guidance on the display.

In some embodiments, the indication of incomplete dose data is based on a counter value.

In some embodiments, the indication of incomplete dose data is based on a comparison of a counter value and an estimated counter value. In some embodiments, the estimated counter value is calculated based on a prior counter value and an elapsed time since the prior counter value was received.

In some embodiments, the medication delivery device is a connected insulin pen, wherein the connected insulin pen is configured to transmit dose data wirelessly.

In some embodiments, the medication delivery device is an insulin pen and a connected pen cap, wherein the connected pen cap is configured to transmit dose data wirelessly.

In some embodiments, the system further comprises a medication delivery device, and wherein the medication delivery device further comprises: an input configured to receive the query for the dose data comprising data related to at least one dose administered during the period of time; one or more processors coupled with the input and a memory storing instructions, wherein the instructions, when executed by the one or more processors, cause the one or more processors to: store data for doses administered during the period of time to create stored data; determine if the stored data includes all doses delivered during the period of time; in response to a determination that the stored data does not include all doses delivered during the period of time, create the indication of incomplete dose data; and in response to the query for the dose data and to the determination that the stored data does not include all doses delivered during the period of time, transmit the indication of incomplete dose data.

In some embodiments, the input is wireless communications circuitry.

In many embodiments, a method for providing dose guidance to a subject includes the steps of: receiving dose data and an indication of incomplete dose data from a medication delivery device, wherein the dose data comprises data related to at least one dose administered during a period of time; querying the medication delivery device for the dose data comprising data related to at least one dose administered during the period of time; determining if the indication of incomplete dose data is received from the medication delivery device; in response to a determination that the indication of incomplete dose data is received, outputting a prompt seeking confirmation that the dose data received for the period of time includes dose data for all doses administered during the period of time; and in response to a determination that the indication of incomplete dose data is not received, calculating a dose guidance.

In some embodiments, the method further includes the step of outputting the dose guidance on the display.

In some embodiments, the indication of incomplete dose data is based on a counter value.

In some embodiments, the indication of incomplete dose data is based on a comparison of a counter value and an estimated counter value. In some embodiments, the estimated counter value is calculated based on a prior counter value and an elapsed time since the prior counter value was received.

In some embodiments, the medication delivery device is a connected insulin pen, wherein the connected insulin pen is configured to transmit dose data wirelessly.

In some embodiments, the medication delivery device is an insulin pen and a connected pen cap, wherein the connected pen cap is configured to transmit dose data wirelessly.

In many embodiments, a method for recommending a dose for a meal includes the steps of: prompting a user to input a tag associated with a meal type; receiving an inputted tag for an instance of the meal type; associating the inputted tag with an amount of medication administered for the instance of the meal type and a post-prandial analyte data set for the instance of the meal type; determining whether a threshold number of instances associated with the meal type is met; and determining a recommended medication dose for the meal type if the threshold number of instances is met.

In some embodiments, the recommended medication dose for the meal type is based at least in part on the amount of medication administered for the instance of the meal type and the post-prandial analyte data set for the instance of the meal type.

In some embodiments, the recommended medication dose for the meal type is based at least in part on a plurality of amounts of medication administered for a plurality of instances of the meal type and a plurality of post-prandial analyte data sets for the plurality of instances of the meal type. In some embodiments, the instance of the meal type is a first instance of the meal type, and wherein the plurality of instances of the meal type includes the first instance of the meal type.

In some embodiments, the method further includes the step of receiving analyte data from a sensor control device. In some embodiments, the recommended medication dose for the meal type is based at least in part on the analyte data received from the sensor control device.

In some embodiments, the method further includes the step of visually outputting to a display the recommended medication dose for the meal type.

In some embodiments, the method further includes the step of prompting the user with an option to track the meal type. In some embodiments, wherein the prompting of the user to input the tag associated with the meal type occurs in response to the user selecting the option to track the meal type.

In many embodiments, a system for determining a recommended medication dose includes: one or more processors coupled with a memory for storing instructions, wherein the instructions, when executed by the one or more processors, cause the one or more processors to: prompt a user to input a tag associated with a meal type, receive an inputted tag for an instance of the meal type, associate the inputted tag with an amount of medication administered for the instance of the meal type and a post-prandial analyte data set for the instance of the meal type, determine whether a threshold number of instances associated with the meal type is met, and determine the recommended medication dose for the meal type if the threshold number of instances is met.

In some embodiments, the recommended medication dose for the meal type is based at least in part on the amount of medication administered for the instance of the meal type and the post-prandial analyte data set for the instance of the meal type.

In some embodiments, the recommended medication dose for the meal type is based at least in part on a plurality of amounts of medication administered for a plurality of instances of the meal type and a plurality of post-prandial analyte data sets for the plurality of instances of the meal type. In some embodiments, the instance of the meal type is a first instance of the meal type, and wherein the plurality of instances of the meal type includes the first instance of the meal type.

In some embodiments, the system further includes wireless communication circuitry configured to receive data indicative of an analyte level from a sensor control device. In some embodiments, the recommended medication dose for the meal type is based at least in part on the data indicative of the analyte level received from the sensor control device.

In some embodiments, the instructions, when executed by the one or more processors, further cause the one or more processors to visually output to a display the recommended medication dose for the meal type.

In some embodiments, the instructions, when executed by the one or more processors, further cause the one or more processors to prompt the user with an option to track the meal type. In some embodiments, the instructions, when executed by the one or more processors, cause the one or more processors to prompt the user to input the tag associated with the meal type only if the user has selected the option to track the meal type.

In many embodiments, a system for smart meal tagging includes: one or more processors coupled with a memory for storing instructions, wherein the instructions, when executed by the one or more processors, cause the one or more processors to: prompt a user to input a tag associated with a meal type, receive an inputted tag for an instance of a first meal type, wherein the first meal type is associated with one or more previously inputted tags, determine whether a meal type characteristic of the instance of the first meal type exceeds a meal type characteristic threshold, and associate the inputted tag with a second meal type, wherein the second meal type is different from the first meal type.

In some embodiments, the meal type characteristic of the instance is based at least in part on a difference between a meal size associated with the one or more previously inputted tags and a meal size of the inputted tag for the instance.

In some embodiments, the meal type characteristic of the instance is based at least in part on a difference between an amount of medication associated with the one or more previously inputted tags and an amount of medication associated with the inputted tag for the instance.

In some embodiments, the instructions, when executed by the one or more processors, further cause the one or more processors to prompt the user with an option to create the second tag if the meal type characteristic of the instance of the first meal type exceeds a meal type characteristic threshold.

In some embodiments, the instructions, when executed by the one or more processors, cause the one or more processors to associate the inputted tag with the second meal type only if the user has selected the option to create the second tag.

In some embodiments, the instructions, when executed by the one or more processors, further cause the one or more processors to disassociate the inputted tag for the instance from the first meal type if the meal type characteristic of the instance of the first meal type exceeds a meal type characteristic threshold. In some embodiments, the instructions, when executed by the one or more processors, cause the one or more processors to disassociate the inputted tag for the instance from the first meal type before the inputted tag is associated with the second meal type.

In many embodiments, a method for recommending a dose for a meal includes the steps of: prompting a user to input a tag associated with a meal type; receiving a first inputted tag for a first instance of the meal type; associating the first inputted tag with a first amount of medication administered for the first instance of the meal type; receiving a second inputted tag for a second instance of the meal type; associating the second inputted tag with a second amount of medication administered for the second instance of the meal type and a second post-prandial analyte data set for the instance of the meal type; and in response to a determination that a difference between the second amount of medication and the first amount of medication is greater than a predetermined threshold difference, prompting the user to input a modified tag associated with the meal type.

In some embodiments, the modified tag comprises a different size of the meal.

In some embodiments, the predetermined threshold difference is at least about 2 units.

In some embodiments, the prompting of the user to input the modified tag associated with the meal type occurs in real time.

In some embodiments, the prompting of the user to input the modified tag associated with the meal type occurs within about 5 minutes or less of receiving the second inputted tag.

In some embodiments, the prompting of the user to input the modified tag associated with the meal type occurs within about 2 minutes or less of receiving the second inputted tag.

In some embodiments, the method further includes the steps of: associating the first inputted tag with a first post-prandial analyte data set for the first instance of the meal type; and associating the second inputted tag with a second post-prandial analyte data set for the second instance of the meal type.

In many embodiments, a system for meal tagging includes one or more processors coupled with a memory for storing instructions, wherein the instructions, when executed by the one or more processors, cause the one or more processors to: prompt a user to input a tag associated with a meal type, receive a first inputted tag for a first instance of the meal type, associate the first inputted tag with a first amount of medication administered for the first instance of the meal type, receive a second inputted tag for a second instance of the meal type; associate the second inputted tag with a second amount of medication administered for the second instance of the meal type, and in response to a determination that a difference between the second amount of medication and the first amount of medication is greater than a predetermined threshold difference, prompt the user to input a modified tag associated with the meal type.

In some embodiments, the modified tag comprises a different size of the meal.

In some embodiments, the predetermined threshold difference is at least about 2 units.

In some embodiments, the instructions, when executed by the one or more processors, further cause the one or more processors to prompt the user to input the modified tag associated with the meal type occurs in real time.

In some embodiments, the instructions, when executed by the one or more processors, further cause the one or more processors to prompt the user to input the modified tag associated with the meal type within about 5 minutes or less of receiving the second inputted tag.

In some embodiments, the instructions, when executed by the one or more processors, further cause the one or more processors to prompt the user to input the modified tag associated with the meal type within about 2 minutes or less of receiving the second inputted tag.

In some embodiments, the instructions, when executed by the one or more processors, further cause the one or more processors to associate the first inputted tag with a first post-prandial analyte data set for the first instance of the meal type and associate the second inputted tag with a second post-prandial analyte data set for the second instance of the meal type.

In many embodiments, an analyte monitoring system includes: a sensor control device comprising an analyte sensor, wherein at least a portion of the analyte sensor is configured to be in fluid contact with a bodily fluid of a subject; and a reader device. The reader device includes wireless communication circuitry configured to receive analyte levels from the sensor control device; and one or more processors coupled to a memory, the memory storing instructions that, when executed by the one or more processors, cause the one or more processors to: determine a pattern type for at least one time segment of a day based on a hypo risk metric and a hyper risk metric for the at least one time segment of the day; and output to a display to a user interface comprising: at least one glucose metric determined for a time period based on the analyte levels received from the sensor control device; a time in range display comprising a graph of time in ranges comprising a plurality of graph portions, wherein each graph portion of the plurality of graph portions indicates an amount of time that a user's analyte level is within a predefined analyte range associated with each graph portion, wherein the plurality of graph portions comprises at least 4 graph portions; and a graph comprising a plot of analyte levels of the user across a horizontal representation of a plurality of time segments of a day and an identification of the determined pattern type for the at least one time segment.

In some embodiments, the at least one glucose metric comprises a glucose average.

In some embodiments, the at least one glucose metric comprises a glucose management indicator.

In some embodiments, the instructions further cause the one or more processors to output a display to the user interface comprising a goal value corresponding to the at least one glucose metric.

In some embodiments, the plurality of graph portions comprises at least 5 graph portions.

In some embodiments, the plurality of graph portions comprises at least four graph portions selected from a group consisting of: a graph portion below a very low threshold, a graph portion between a very low threshold and a low threshold, a graph portion between a low threshold and a high threshold, a graph portion between a high threshold and a very high threshold, and a graph portion above a very high threshold.

In some embodiments, the time in range display further comprises a description of the predefined analyte range associated with each graph portion.

In some embodiments, the time in range display further comprises a value for each graph portion of the plurality of graph portions that relates to the amount of time that the user's analyte level was within the predefined analyte range associated with the graph portion during the time period. In some embodiments, the value is a percentage value.

In some embodiments, the time in range display further comprises a combined value for at least two graph portions of the plurality of graph portions that relates to a sum of the amount of time that the user's analyte level was within each of the predefined analyte ranges associated with at least two graph portions during the time period.

In some embodiments, the graph of the time in ranges comprises a histogram. In some embodiments, each graph portion of the histogram are arranged in a vertical layout, wherein a graph portion below a very low threshold is located below a graph portion between a very low threshold and a low threshold, which is locate below a graph portion between a low threshold and a high threshold, which is located below a graph portion between a high threshold and a very high threshold, which is located below a graph portion above a very high threshold.

In some embodiments, the identification of the determined pattern type for the at least one time segment comprises at least a partial outline of the time segment on the graph. In some embodiments, the identification of the determined pattern type for the at least one time segment further comprises a label of the determined pattern type.

In some embodiments, the pattern type is at least one of a lows pattern, a highs with some lows pattern, a highs pattern, or no pattern.

In some embodiments, the graph comprises a plurality of determined pattern types, and wherein an identification of a single pattern type is visibly distinct from other identifications of the plurality of determined pattern types.

In some embodiments, the instructions further cause the one or more processors to output a display to the user interface an identification of a most important pattern type, wherein the most important pattern type is one of the pattern type determined for the at least one time segment of the day. In some embodiments, the identification of the most important pattern type is displayed on the graph. In some embodiments, the identification of the determined pattern type for the at least one time segment comprises a plurality of identifications of a determined pattern type for each of the at least one time segment, and wherein the identification of the most important pattern type is visibly distinct from other identifications of the plurality of identifications. In some embodiments, the pattern type determined for the at least one time segment of the day comprises a plurality of pattern types for a plurality of time segments of the day. In some embodiments, the plurality of pattern types comprises at least two of a lows pattern, a highs with some lows pattern, a highs pattern, or no pattern. In some embodiments, if the plurality of pattern types comprises a lows pattern, the identification of the most important pattern type comprises an identification of the lows pattern. In some embodiments, if the plurality of pattern types comprises a highs with some lows pattern and does not comprise a lows pattern, the identification of the most important pattern type comprises an identification of the highs with some lows pattern. In some embodiments, if the plurality of pattern types comprises a highs pattern and does not comprise a highs with some lows pattern or a lows pattern, the identification of the most important pattern type comprises an identification of the highs pattern.

In some embodiments, the instructions further cause the one or more processors to output a display to the user interface comprising an identification of at least one time segment of the day that was determined to have the most important pattern type. In some embodiments, the display of the identification of the most important pattern type and the identification of the at least one time segment of the day that was determined to have the most important pattern type comprises a tag for the identification of the most important pattern type and at least one tag for the identification of the at least one time segment of the day that was determined to have the most important pattern type. In some embodiments, the tag for the identification of the most important pattern type is a different color that the at least one tag for the identification of the at least one time segment of the day that was determined to have the most important pattern type.

In some embodiments, the instructions further cause the one or more processors to: determine a variability of at least one time segment of the day; and output a display to the user interface comprising a statement relating to variability if the determined variability is high. In some embodiments, the statement relating to variability comprises an identification of behaviors that may contribute to glucose variability.

In some embodiments, the instructions further cause the one or more processors to output a display to the user interface comprising a statement relating to an excursion below a very low threshold. In some embodiments, the very low threshold is between about 50 mg/dL and about 58 mg/dL. In some embodiments, the very low threshold is about 54 mg/dL.

In some embodiments, the instructions further cause the one or more processors to output a display to the user interface comprising statements relating to medication considerations. In some embodiments, the statements relating to medication considerations comprise suggestions to adjust a medication. In some embodiments, the statements relating to medication considerations comprise suggestions related to medications contributing to low glucose levels.

In some embodiments, the instructions further cause the one or more processors to output a display to the user interface comprising statements relating to lifestyle considerations. In some embodiments, the statements relating to lifestyle considerations comprise statements relating to at least one of missed meals, carbohydrates, activity level, alcohol, and medication.

In some embodiments, the time period is 14 days.

In many embodiments, a method for displaying information related to glucose levels in a subject includes the steps of: receiving analyte levels from a sensor control device; determining a pattern type for at least one time segment of a day based on a hypo risk metric and a hyper risk metric for the at least one time segment of the day; and displaying a user interface comprising: at least one glucose metric determined for a time period based on the analyte levels received from the sensor control device; a time in range display comprising a graph of time in ranges comprising a plurality of graph portions, wherein each graph portion of the plurality of graph portions indicates an amount of time that a user's analyte level is within a predefined analyte range associated with each graph portion, wherein the plurality of graph portions comprises at least 4 graph portions; and a graph comprising a plot of analyte levels of the user across a horizontal representation of a plurality of time segments of a day and an identification of the determined pattern type for the at least one time segment.

In some embodiments, the at least one glucose metric comprises a glucose average.

In some embodiments, the at least one glucose metric comprises a glucose management indicator.

In some embodiments, the instructions further cause the one or more processors to output a display to the user interface comprising a goal value corresponding to the at least one glucose metric.

In some embodiments, the plurality of graph portions comprises at least 5 graph portions.

In some embodiments, the plurality of graph portions comprises at least four graph portions selected from a group consisting of: a graph portion below a very low threshold, a graph portion between a very low threshold and a low threshold, a graph portion between a low threshold and a high threshold, a graph portion between a high threshold and a very high threshold, and a graph portion above a very high threshold.

In some embodiments, the time in range display further comprises a description of the predefined analyte range associated with each graph portion.

In some embodiments, the time in range display further comprises a value for each graph portion of the plurality of graph portions that relates to the amount of time that the user's analyte level was within the predefined analyte range associated with the graph portion during the time period. In some embodiments, the value is a percentage value.

In some embodiments, the time in range display further comprises a combined value for at least two graph portions of the plurality of graph portions that relates to a sum of the amount of time that the user's analyte level was within each of the predefined analyte ranges associated with the at least two graph portions during the time period.

In some embodiments, the graph of the time in ranges comprises a histogram. In some embodiments, each graph portion of the histogram are arranged in a vertical layout, wherein a graph portion below a very low threshold is located below a graph portion between a very low threshold and a low threshold, which is locate below a graph portion between a low threshold and a high threshold, which is located below a graph portion between a high threshold and a very high threshold, which is located below a graph portion above a very high threshold.

In some embodiments, the identification of the determined pattern type for the at least one time segment comprises at least a partial outline of the time segment on the graph.

In some embodiments, the identification of the determined pattern type for the at least one time segment further comprises a label of the determined pattern type.

In some embodiments, the pattern type is at least one of a lows pattern, a highs with some lows pattern, a highs pattern, or no pattern.

In some embodiments, the graph comprises a plurality of determined pattern types, and wherein an identification of a single pattern type is visibly distinct from other identifications of the plurality of determined pattern types.

In some embodiments, the instructions further cause the one or more processors to output a display to the user interface an identification of a most important pattern type, wherein the most important pattern type is one of the pattern type determined for the at least one time segment of the day. In some embodiments, the identification of the most important pattern type is displayed on the graph. In some embodiments, the identification of the determined pattern type for the at least one time segment comprises a plurality of identifications of a determined pattern type for each of the at least one time segment, and wherein the identification of the most important pattern type is visibly distinct from other identifications of the plurality of identifications. In some embodiments, the pattern type determined for the at least one time segment of the day comprises a plurality of pattern types for a plurality of time segments of the day. In some embodiments, the plurality of pattern types comprises at least two of a lows pattern, a highs with some lows pattern, a highs pattern, or no pattern. In some embodiments, if the plurality of pattern types comprises a lows pattern, the identification of the most important pattern type comprises an identification of the lows pattern. In some embodiments, if the plurality of pattern types comprises a highs with some lows pattern and does not comprise a lows pattern, the identification of the most important pattern type comprises an identification of the highs with some lows pattern. In some embodiments, if the plurality of pattern types comprises a highs pattern and does not comprise a highs with some lows pattern or a lows pattern, the identification of the most important pattern type comprises an identification of the highs pattern.

In some embodiments, the instructions further cause the one or more processors to output a display to the user interface comprising an identification of at least one time segment of the day that was determined to have the most important pattern type.

In some embodiments, the display of the identification of the most important pattern type and the identification of the at least one time segment of the day that was determined to have the most important pattern type comprises a tag for the identification of the most important pattern type and at least one tag for the identification of the at least one time segment of the day that was determined to have the most important pattern type. In some embodiments, the tag for the identification of the most important pattern type is a different color that the at least one tag for the identification of the at least one time segment of the day that was determined to have the most important pattern type.

In some embodiments, the instructions further cause the one or more processors to: determine a variability of at least one time segment of the day; and output a display to the user interface comprising a statement relating to variability if the determined variability is high. In some embodiments, the statement relating to variability comprises an identification of behaviors that may contribute to glucose variability.

In some embodiments, the instructions further cause the one or more processors to output a display to the user interface comprising a statement relating to an excursion below a very low threshold. In some embodiments, the very low threshold is between about 50 mg/dL and about 58 mg/dL. In some embodiments, the very low threshold is about 54 mg/dL.

In some embodiments, the instructions further cause the one or more processors to output a display to the user interface comprising statements relating to medication considerations. In some embodiments, the statements relating to medication considerations comprise suggestions to adjust a medication. In some embodiments, the statements relating to medication considerations comprise suggestions related to medications contributing to low glucose levels.

In some embodiments, the instructions further cause the one or more processors to output a display to the user interface comprising statements relating to lifestyle considerations.

In some embodiments, the statements relating to lifestyle considerations comprise statements relating to at least one of missed meals, carbohydrates, activity level, alcohol, and medication.

In some embodiments, the time period is 14 days.

In many embodiments, an apparatus for displaying metrics relating to a subject includes: an input configured to receive drug dosing data; a display configured to visually present information; and one or more processors coupled with the input, the display, and a memory storing instructions and doses of a medication received by the subject over a period of time and recommended doses of the medication for the subject over the period of time, wherein the instructions, when executed by the one or more processors, cause the apparatus to: display a graph plotting a plurality of medication doses taken by the subject at a plurality of times, wherein the graph comprises an x-axis of time and a y-axis of a difference between a dose taken by the subject and a dose recommended for the subject.

In some embodiments, the plurality of medication doses comprises at least one of basal doses, fixed meal doses, and meal doses with a correction factor. In some embodiments, the fixed meal doses comprise at least one of fixed breakfast doses, fixed lunch doses, and fixed dinner doses. In some embodiments, the meal doses with a correction factor comprise at least one of breakfast doses with a correction factor, fixed lunch doses with a correction factor, and fixed dinner doses with a correction factor.

In some embodiments, the difference between the dose taken by the subject and the dose recommended is in units.

In some embodiments, the input comprises wireless communications circuitry.

In many embodiments, an apparatus for displaying metrics relating to a subject includes: an input configured to receive measured analyte data and drug dosing data; a display configured to visually present information; and one or more processors coupled with the input, the display, and a memory storing instructions and time-correlated data characterizing an analyte of the subject, doses of a medication received by the subject over a period of time, and recommended doses of the medication for the subject over the period of time, wherein the instructions, when executed by the one or more processors, cause the apparatus to: display a summary of therapies for the subject comprising dose amounts administered during a time period and analyte metrics determined from the received measured analyte data; display a graphic summarizing missed doses during the time period; and display a graphic summarizing override doses, wherein override doses comprise doses received by the subject at a time that are a different amount than a recommended dose for the time.

In some embodiments, the graphic summarizing missed doses comprises a graphic representation of percentages of missed doses for a plurality of dose types. In some embodiments, each percentage of the percentages of missed for the plurality of dose types is calculated as a percentage of missed doses of the dose type of a total number of doses of the dose type during a time period. In some embodiments, the plurality of dose types comprises at least one of basal doses, breakfast doses, lunch doses, and dinner doses.

In some embodiments, the graphic summarizing missed doses is a bar graph.

In some embodiments, the graphic summarizing override doses is a bar graph.

In some embodiments, the input comprises wireless communications circuitry.

In many embodiments, an apparatus for displaying metrics relating to a subject includes: an input configured to receive measured analyte data from a plurality of subjects, drug dosing data from a plurality of subjects, and data related to dosing recommendations for the plurality of subjects; a display configured to visually present information; and one or more processors coupled with the input, the display, and a memory storing instructions and time-correlated data characterizing an analyte of each of the plurality of subjects, doses of a medication received by each of the plurality of subjects over a period of time, and the data related to dosing recommendations for the plurality of subjects, when executed by the one or more processors, cause the apparatus to: display a summary of analyte metrics for the each of the plurality of subjects, wherein the analyte metrics comprise at least two of time in range, time below a low threshold, time above a high threshold, percentage of basal doses taken, and average bolus doses taken per day; and display a summary of information related to dosing recommendations, wherein the information related to dosing recommendations comprises an indication of a dosing recommendation for a subject of the plurality of subjects needing approval from a health care provider.

In some embodiments, the summary of analyte metrics comprises at least three of time in range, time below a low threshold, time above a high threshold, percentage of basal doses taken, and average bolus doses taken per day.

In some embodiments, the low threshold is about 70 mg/dL.

In some embodiments, the high threshold is about 180 mg/dL.

In some embodiments, the indication of the dosing recommendation is an icon.

In some embodiments, the indication of the dosing recommendation is a statement indicating a number of dosing recommendations needing approval.

In some embodiments, the input comprises wireless communications circuitry.

In many embodiments, an apparatus for displaying treatment information relating to a subject includes: an input configured to receive measured analyte data and drug dosing data; a display configured to visually present information; and one or more processors coupled with the input, the display, and a memory storing instructions and time-correlated data characterizing an analyte of the subject, doses of a medication received by the subject over a period of time, and meal times of the subject, wherein the instructions, when executed by the one or more processors, cause the apparatus to: receive estimated dose parameters and estimated meal dosing time ranges from the subject; determine a representative amount of each of a plurality of basal doses and a plurality of meal doses taken by the subject during a period of time based on the drug dosing data; determine representative meal dosing time ranges for the subject during the time period based on the drug dosing data; determine recommended dose amounts for at least one of a basal dose, a breakfast dose, a lunch dose, and a dinner dose; display the estimated dose parameters and estimated meal dosing time ranges received from the subject; display the representative amount of each of a plurality of basal doses and a plurality of meal doses and the representative meal dosing time ranges; and display the recommended dose amounts for at least one of the basal dose, the breakfast dose, the lunch dose, and the dinner dose.

In some embodiments, the plurality of meal doses comprises a plurality of breakfast doses, a plurality of lunch doses, and a plurality of dinner doses, and wherein the instructions, when executed by the one or more processors, cause the apparatus to determine an average amount of each of the plurality of basal doses, plurality of breakfast doses, the plurality of lunch doses, and the plurality of dinner doses.

In some embodiments, the estimated dose parameters comprise estimated amounts for a basal dose, a breakfast dose, a lunch dose, and a dinner dose. In some embodiments, the estimated dose parameter further comprises estimated times that the subject takes the basal dose, the breakfast dose, the lunch dose, and the dinner dose.

In some embodiments, the estimated meal dosing time ranges comprise an estimated dosing start time and an estimated dosing end time for each of breakfast, lunch, and dinner.

In some embodiments, the representative amount of each of the plurality of basal doses and the plurality of meal doses comprise an average of each of the plurality of basal doses and the plurality of meal doses taken by the subject during a period of time.

In some embodiments, the representative amount of each of the plurality of basal doses and the plurality of meal doses comprise a mode of each of the plurality of basal doses and the plurality of meal doses taken by the subject during a period of time.

In some embodiments, the instructions, when executed by the one or more processors, further cause the apparatus to: determine a pre-meal correction factor and a post-meal correction factor based on the measured analyte data and the drug dosing data; and display the pre-meal correction factor and the post-meal correction factor.

In some embodiments, the representative meal dosing time ranges are displayed adjacent to the estimated meal dosing time ranges.

In some embodiments, the representative amount of each of the plurality of basal doses and the plurality of meal doses is displayed adjacent to the estimated dose parameters.

In some embodiments, the instructions, when executed by the one or more processors, further cause the apparatus to: determine a conservative value for at least one of a basal dose, a breakfast dose, a lunch dose, and a dinner dose, wherein the conservative value is lower than a corresponding determined representative amount of each of the plurality of basal doses and a plurality of meal doses; and display the determined conservative value.

In many embodiments, the input comprises wireless communication circuitry.

For any of the methods described herein, the methods can be executed on at least one processor of a remote device, e.g., a server, phone/receiver, on the medication delivery device, or on the glucose monitoring device.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

### CLAUSES

Exemplary embodiments are set out in the following numbered clauses.

Clause 1. An apparatus for parameterizing a patient's medication dosing practice for configuring dose guidance settings, the apparatus comprising:

an input component configured to receive measured analyte data, meal data, and medication dosing data;

a display component configured to visually present information; and

one or more processors coupled with the input, the display, and a memory storing instructions and time-correlated data characterizing an analyte of the patient over an analysis period, wherein the instructions, when executed by the one or more processors, cause the apparatus to perform:
receiving patient dose regimen information for the analysis period;
evaluating a measure of consistency between the time-correlated data and the patient dose regimen information; and
determining dose guidance information based on the measure of consistency.

Clause 2. The apparatus of clause 1, wherein the memory holds further instructions for outputting the dose guidance information to the display.

Clause 3. The apparatus of clause 1, wherein the memory holds further instructions for receiving the patient dose regimen information from the input component.

Clause 4. The apparatus of clause 1, wherein the memory holds further instructions for receiving the patient dose regimen information by transmission from a remote data server.

Clause 5. The apparatus of clause 1, wherein the patient dose regimen information comprises typical fixed medication doses taken at mealtimes and a typical time of day when breakfast is eaten.

Clause 6. The apparatus of clause 1, wherein the patient dose regimen information comprises information defining a frequency of patient compliance with scheduled doses or meals.

Clause 7. The apparatus of clause 1, wherein the medication comprises insulin.

Clause 8. The apparatus of clause 1, wherein the instructions for evaluating a measure of consistency further comprise:

classifying each dose of the patient dose regimen in a medication class, based on the time-correlated data;

grouping each of the doses in one of a set of mealtime groups;

generating dose parameters for the patient at least in part by applying data for each of the mealtime groups to a model; and

storing the dose parameters for configuring dose guidance settings.

Clause 9. The apparatus of clause 1, wherein the memory holds further instructions for accumulating the time-correlated data characterizing an analyte of the patient over a time period.

Clause 10. The apparatus of clause 1, wherein the memory holds further instructions for determining the dose guidance information at least in part by reducing a recommendation for dosing based on detecting excursions of the analyte beyond a lower threshold in the time-correlated data.

Clause 11. The apparatus of clause 10, wherein the recommendation for dosing is for fixed dosing only.

Clause 12. The apparatus of clause 1, wherein the memory holds further instructions for determining patient adherence to the patient dose regimen information based on the time-correlated data.

Clause 13. The apparatus of clause 1, wherein the memory holds further instructions for determining whether to output the dose guidance parameters based on the measure of consistency.

Clause 14. The apparatus of clause 1, wherein the memory holds further instructions for outputting the dose guidance parameters comprising predetermined dose suggestions if the measure of consistency indicates an unreliable system configuration.

Clause 15. The apparatus of clause 1, wherein the input comprises wireless communications circuitry.

Clause 16. A method for facilitating efficient access by a healthcare provider (HCP) to an electronic medical record (EMR) of a patient generated by dose guidance system while protecting patient privacy, the method comprising:

authenticating, by at least one processor of a portable display device, a session with the patient;

generating, by the at least one processor in response to receiving an input during the session from the patient indicating a request to share the EMRs with an HCP, an EMR identification code (ID);

providing, by the at least one processor, the EMR ID to a remote server controlling access to the EMR; and

outputting, by the at least one processor, the EMR ID to a display of the portable display device.

Clause 17. The method of clause 16, further comprising providing the EMR to the remote server, prior to the authenticating.

Clause 18. The method of clause 16, further comprising receiving the EMR from the dose guidance system.

Clause 19. The method of clause 16, further comprising determining whether the EMR fails a condition for consistency with patient input indicating a dose pattern of a tracked medication.

Clause 20. The method of clause 19, further comprising, upon determining that the EMR fails the condition for consistency, providing the patient with an option to provide the EMR to the HCP.

Clause 21. The method of clause 19, wherein the generating, the providing, and the outputting are conditioned on the determining that the EMR fails the condition for consistency.

Clause 22. The method of clause 20, wherein the generating, the providing, and the outputting are conditioned on the determining that the EMR fails the condition for consistency.

Clause 23. The method of clause 16, further comprising providing the patient with an option to provide the EMR to the HCP.

Clause 24. The method of clause 17, wherein the remote server upon receiving the EMR ID, creates a web page addressed at least in part by the EMR ID for displaying the EMR.

Clause 25. The method of clause 16, wherein the EMR comprises determinations of dosing parameters for a medication administered to the patient at times during a defined period and a measure of consistency of the determinations with patient-supplied dosing information for the medication.

Clause 26. The method of clause 25, wherein the medication is insulin.

Clause 27. A system for providing dose guidance to a subject, the system comprising:

an input configured to receive dose data from a medication delivery device, wherein the dose data comprises an amount and a time of a most recent drug dose administered;

a display configured to visually present a dose guidance;

one or more processors coupled with the input, the display, and a memory storing instructions, wherein the instructions, when executed by the one or more processors, cause the one or more processors to:
classify the most recent drug dose administered as a meal dose or a correction dose; and
in response to a determination that a period of time has elapsed since the time of the most recent drug dose administered, display an additional dose guidance.

Clause 28. The system of clause 27, wherein the period of time is about 2 hours.

Clause 29. The system of clause 27, wherein the most recent drug dose administered is a meal dose, and wherein the dose data further comprises at least one additional drug dose administered after the most recent drug dose was administered, and wherein the period of time is not reset to a time administered of the at least one additional drug dose.

Clause 30. The system of clause 27, wherein the most recent drug dose administered is not a prime dose.

Clause 31. The system of clause 27, wherein the time of the most recent drug dose administered is a timestamp from a connected drug delivery device.

Clause 32. The system of clause 27, wherein the most recent drug dose administered is a correction dose, and wherein the dose data further comprises at least one additional drug dose administered after the most recent drug dose administered, and wherein a beginning of the period of time is reset to a time administered of the at least one additional drug dose administered.

Clause 33. The system of clause 27, further comprising a medication delivery device configured to deliver at least one dose of a drug to a subject.

Clause 34. The system of clause 27, wherein the input comprises wireless communications circuitry.

Clause 35. A method for providing dose guidance, the method comprising:

receiving, by an electronic device, drug dose data of a subject from a medication delivery device, wherein the drug dose data comprises an amount and a time of a most recent drug dose administered;

classifying the most recent drug dose administered as a meal dose or a correction dose,

in response to determining that a period of time has elapsed since the time of the most recent drug dose administered, displaying a dose guidance.

Clause 36. The method of clause 35, wherein the period of time is about 2 hours.

Clause 37. The method of clause 35, wherein the most recent drug dose administered is a meal dose, wherein the drug dose data further comprises at least one additional drug dose administered after the most recent drug dose was administered, and wherein a beginning of the period of time is not reset to a time administered of the at least one additional drug dose.

Clause 38. The method of clause 35, wherein the most recent drug dose administered is not a prime dose.

Clause 39. The method of clause 35, wherein the time of the most recent drug dose administered is a timestamp from a connected drug delivery device.

Clause 40. The method of clause 35, wherein the most recent drug dose administered is a correction dose, wherein the drug dose data further comprises at least one additional drug dose administered after the most recent drug dose administered, and wherein a beginning of the period of time is reset to a time administered of the at least one additional drug dose administered.

Clause 41. A system for providing dose guidance to a subject, the system comprising:

an input configured to receive dose data from a medication delivery device, wherein the dose data comprises data related to a recent drug dose administered;

a display configured to visually present a dose guidance;

one or more processors coupled with the input, the display, and a memory storing instructions, wherein the instructions, when executed by the one or more processors, cause the one or more processors to:
determine if the recent drug dose administered is a most recent drug dose administered; and
in response to a determination that the recent drug dose administered is the most recent drug dose administered, display a screen comprising a dose guidance recommendation.

Clause 42. The system of clause 41, wherein the recent drug dose administered is determined to be the most recent drug dose administered through confirmation from the user.

Clause 43. The system of clause 41, wherein the dose data further comprises data related to at least one drug dose administered since a reset time, and wherein the instructions further cause the one or more processors to, in response to a determination that the at least one drug dose administered since the reset time has been classified, display the screen.

Clause 44. The system of clause 43, wherein the dose data related to the at least one drug dose administered since the reset time was classified automatically.

Clause 45. The system of clause 44, further comprising a medication delivery device configured to deliver at least one dose of a drug to a subject, wherein the instructions further cause the one or more processors to transmit one or more wireless interrogation signals to the medication delivery device to determine that a most recent dosage administered has been received.

Clause 46. The system of clause 43, wherein the dose data related to the at least one drug dose administered since the reset time has been classified by the user.

Clause 47. The system of clause 41, wherein the instructions further cause the one or more processors to display a prompt for a user to confirm that information related to the most recent drug dose administered was correct.

Clause 48. The system of clause 47, wherein the instructions further cause the one or more processors to display the screen comprising the dose guidance recommendation for a period of time that starts after confirmation from the user.

Clause 49. The system of clause 41, further comprising a medication delivery device configured to deliver at least one dose of a drug to a subject.

Clause 50. The system of clause 41, wherein the input is further configured to receive measured analyte data and a request for dose guidance, and wherein the instructions further cause the one or more processors to:
determine if a glucose concentration at a time of receipt of the request for dose guidance is below a low threshold value; and
in response to a determination that the glucose concentration at the time of receipt of the request for dose guidance is below the low threshold value, display a screen comprising a message to address a low glucose level before administering medication.

Clause 51. The system of clause 50, wherein the instructions further cause the one or more processors to:
in response to the determination that the glucose concentration at the time of receipt of the request for dose guidance is below the low threshold value, the dose guidance recommendation is not displayed.

Clause 52. The system of clause 41, wherein the input is further configured to receive measured analyte data and a request for dose guidance after a start time of a meal, and wherein the instructions further cause the one or more processors to:
determine if a glucose concentration at an estimated start time of the meal is below a low threshold value; and
in response to a determination that the glucose concentration at the estimated start time of the meal is below the low threshold value, display a screen comprising a message to address a low glucose level before administering medication.

Clause 53. The system of clause 52, wherein the instructions further cause the one or more processors to:
in response to the determination that the glucose concentration at the estimated start time of the meal is below the low threshold value is below the low threshold value, the dose guidance recommendation is not displayed.

Clause 54. The system of clause 41, wherein the input comprises wireless communications circuitry.

Clause 55. A method for providing dose guidance, the method comprising:

receiving, by an electronic device, dose data of a user from a medication delivery device, wherein the dose data comprises data related to a recent drug dose administered;

determining if the recent drug dose administered is a most recent drug dose administered;

in response to a determination that the recent drug dose administered is the most recent drug dose administered, displaying a screen comprising a dose guidance recommendation.

Clause 56. The method of clause 55, wherein the recent drug dose administered is determined to be the most recent drug dose administered through confirmation from the user.

Clause 57. The method of clause 55, wherein the recent drug dose administered was administered after a rest time, wherein the method further comprises the step of:

determining if the recent dose administered after the reset time was classified.

Clause 58. The method of clause 57, wherein the recent dose administered after the reset time was classified automatically.

Clause 59. The method of clause 55, wherein the method further comprises the step of:

transmitting one or more wireless interrogation signals to a medication delivery device to determine that a most recent dosage administered has been received.

Clause 60. The method of clause 57, wherein the recent dose administered after the reset time was classified by the user.

Clause 61. The method of clause 60, further comprising the step of displaying a prompt for the user to confirm that information related to the most recent drug dose administered is correct.

Clause 62. The method of clause 55, wherein the screen comprising the dose guidance recommendation is only displayed for a period of time that starts after a confirmation from the user that information related to the most recent drug dose administered is correct.

Clause 63. A system for providing dose guidance to a subject, the system comprising:

an input configured to receive dose data from a medication delivery device, wherein the dose data comprises data related to at least one meal dose administered since a reset time;

a display configured to visually present a plurality of meal icons;

one or more processors coupled with the input, the display, and a memory storing instructions, wherein the instructions, when executed by the one or more processors, cause the one or more processors to:
determine if the at least one meal dose administered since the reset time has been classified; and
in response to a determination that the at least one meal dose administered since the reset time has been classified, display a screen comprising the plurality of meal icons.

Clause 64. The system of clause 63, wherein the instructions cause the one or more processors to determine if the at least one meal dose administered since the reset time has been classified as one of breakfast, lunch, or dinner.

Clause 65. The system of clause 63, wherein the plurality of meal icons includes a breakfast icon, a lunch icon, and a dinner icon, and wherein each of the breakfast, lunch, and dinner icons comprise a first appearance and a second appearance, wherein the first appearance is associated with a first state in which one of the at least one meal dose administered since the reset time has been classified as a meal type corresponding to the breakfast, lunch, or dinner icon, and the second appearance is associated with a second state in which one of the at least one meal dose administered since the reset time has not been classified as a meal type corresponding to the breakfast, lunch, or dinner icon.

Clause 66. The system of clause 65, wherein the first appearance comprises a shaded appearance.

Clause 67. The system of clause 65, wherein the second appearance comprises an unshaded appearance.

Clause 68. The system of clause 65, wherein the second appearance is brighter than the first presentation.

Clause 69. The system of clause 63, wherein the instructions further cause the one or more processors to:

classify the at least one meal dose administered since the reset time as a breakfast dose, a lunch dose, or a dinner dose.

Clause 70. The system of clause 63, wherein the instructions further cause the one or more processors to:

receive input from a user, wherein the input comprises a classification of the at least one meal dose administered since the reset time as a breakfast dose, a lunch dose, or a dinner dose.

Clause 71. The system of clause 63, wherein the reset time is determined based on at least one time range associated with at least one meal.

Clause 72. The system of clause 63, wherein the reset time is determined based on a time range associated with a dinner dose and a time range associated with a breakfast dose.

Clause 73. The system of clause 63, wherein the reset time is midnight.

Clause 74. The system of clause 63, wherein the reset time is a time that is about halfway between an end of a dinner dose time range and a start of a breakfast dose time range.

Clause 75. The system of clause 63, further comprising a medication delivery device configured to deliver at least one dose of a drug to a subject.

Clause 76. The system of clause 63, wherein the input comprises wireless communications circuitry.

Clause 77. A method for providing dose guidance, the method comprising:

receiving, by an electronic device, drug dose data of a user from a medication delivery device, wherein the drug dose data comprises data related to at least one meal dose administered since a reset time;

determining if the at least one meal dose administered since the reset time has been classified;

in response to a determination that the at least one meal dose administered since the reset time has been classified, displaying a screen comprising a plurality of meal icons.

Clause 78. The method of clause 77, wherein the determining step comprises determining if the at least one meal dose administered since the reset time has been classified as one of breakfast, lunch, or dinner.

Clause 79. The method of clause 77, wherein the plurality of meal icons includes a breakfast icon, a lunch icon, and a dinner icon, and wherein each of the breakfast, lunch, and dinner icons comprise a first appearance and a second appearance, wherein the first appearance is associated with a first state in which one of the at least one meal dose administered since the reset time has been classified as a meal type corresponding to the breakfast, lunch, or dinner icon, and the second appearance is associated with a second state in which one of the at least one meal dose administered since the reset time has not been classified as a meal type corresponding to the breakfast, lunch, or dinner icon.

Clause 80. The method of clause 79, wherein the first appearance comprises a shaded appearance.

Clause 81. The method of clause 79, wherein the second appearance comprises an unshaded appearance.

Clause 82. The method of clause 79, wherein the second appearance is brighter than the first presentation.

Clause 83. The method of clause 77, wherein the at least one meal dose administered since the reset time has been classified automatically by one or more processors of a reader device.

Clause 84. The method of clause 77, wherein the at least one meal dose administered since the reset time has been classified by the user.

Clause 85. The method of clause 77, wherein the reset time is determined based on at least one time range associated with at least one meal.

Clause 86. The method of clause 77, wherein the reset time is determined based on a time range associated with dinner dose and a time range associated with breakfast dose.

Clause 87. The method of clause 77, wherein the reset time is midnight.

Clause 88. The method of clause 77, wherein the reset time is a time that is about halfway between an end of a dinner dose time range and a start of a breakfast dose time range.

Clause 89. A system for providing dose guidance to a subject, the system comprising:

an input configured to receive dose data from a medication delivery device;

a display configured to visually present a dose guidance;

one or more processors coupled with the input, the display, and a memory storing instructions, wherein the instructions, when executed by the one or more processors, cause the one or more processors to:
determine if a missed dose alert is active;
in response to a determination that a missed dose alert is not active, display a dose guidance recommendation based on a normal meal dose calculation; and
in response to a determination that a missed dose alert is active, display a dose guidance recommendation based on a late meal dose calculation.

Clause 90. The system of clause 89, wherein the instructions further cause the one or more processors to:

determine if the dose data received from the medication delivery device comprises a dose administered within a period of time of the current time; and

in response to a determination that the dose data received from the medication delivery device comprises a dose administered within a period of time of the current time, display the dose guidance recommendation based on the late meal dose calculation.

Clause 91. The system of clause 90, wherein the instructions cause the one or more processors to:

display the dose guidance recommendation based on the late meal dose calculation in response to the determination that the missed dose alert is active and the determination that the dose data received from the medication delivery device comprises a dose administered within a period of time of the current time.

Clause 92. The system of clause 90, wherein the period of time is about two hours.

Clause 93. The system of clause 89, wherein the normal meal dose calculation is based on a fixed insulin dose associated with a meal and an amount of insulin on board if a current glucose level of the user is below a target glucose value.

Clause 94. The system of clause 89, wherein each of the normal meal dose calculation and the late meal dose calculation are based on a fixed insulin dose associated with a meal, an amount of insulin on board, a correction adjustment, and a trend adjustment, if a current glucose level of the user is above a target glucose value.

Clause 95. The system of clause 94, wherein the normal meal dose calculation is based on a current glucose level determined from scanned glucose data.

Clause 96. The system of clause 94, wherein the late meal dose calculation is based on a current glucose level determined from streaming glucose data.

Clause 97. The system of clause 96, wherein the late meal dose calculation is further based on a trend adjustment determined from streaming glucose data.

Clause 98. The system of clause 94, wherein the late meal dose calculation is based on the amount of insulin on board calculated according to a time of a request for the dose guidance recommendation by the user.

Clause 99. The system of clause 94, further comprising a medication delivery device configured to deliver at least one dose of a drug to a subject.

Clause 100. The system of clause 94, wherein the input comprises wireless communications circuitry.

Clause 101. A method for providing dose guidance, the method comprising:

receiving, by an electronic device, drug dose data of a user from a medication delivery device;

determining if a missed dose alert is active; and

in response to a determination that a missed dose alert is not active, display a dose guidance recommendation based on a normal meal dose calculation; and

in response to a determination that a missed dose alert is active, display a dose guidance recommendation based on a late meal dose calculation.

Clause 102. The method of clause 101, further comprising the step of:

determining if the drug dose data comprises data for doses administered within a period of time;

displaying the dose guidance recommendation based on the late meal dose calculation in response to a determination that the drug dose data does not include any data for doses administered within a period of time.

Clause 103. The method of clause 102, wherein the period of time is about two hours.

Clause 104. The method of clause 101, wherein the normal meal dose calculation is based on a fixed insulin dose associated with a meal and an amount of insulin on board if a current glucose level of the user is below a target glucose value.

Clause 105. The method of clause 101, wherein each of the normal meal dose calculation and the late meal dose calculation are based on a fixed insulin dose associated with a meal, an amount of insulin on board, a correction adjustment, and a trend adjustment, if a current glucose level of the user is above a target glucose value.

Clause 106. The method of clause 105, wherein the normal meal dose calculation is based on a current glucose level determined from scanned glucose data.

Clause 107. The method of clause 105, wherein the late meal dose calculation is based on a current glucose level determined from streaming glucose data.

Clause 108. The method of clause 107, wherein the late meal dose calculation is further based on a trend adjustment determined from streaming glucose data.

Clause 109. The method of clause 105, wherein the late meal dose calculation is based on the amount of insulin on board calculated according to a time of a request for the dose guidance recommendation by the user.

Clause 110. A system for providing alerts to a subject, the system comprising:

an input configured to receive streaming glucose data from a sensor control device;

a display configured to present an alert;

one or more processors coupled with the input, the display, and a memory storing instructions, wherein the instructions, when executed by the one or more processors, cause the one or more processors to:
determine at a current time if a meal dose has been missed in association with a meal having an estimated meal start time by detecting a missed meal dose condition for a consecutive number of minutes;
determine if an insulin dose has not been recorded within about 45 minutes prior to the estimated meal start time; and
in response to a detection of a missed meal dose condition for the consecutive number of minutes and a determination that the insulin dose has not been recorded within about 45 minutes prior to the estimated meal start time, display an alert interface relating to the missed meal dose.

Clause 111. The system of clause 110, wherein the consecutive number of minutes is about 5 minutes.

Clause 112. The system of clause 110, wherein the instructions further cause the one or more processors to:

determine if a meal dose has been recorded within about 2 hours of the current time; and

in response to a determination that the meal dose has been recorded within about 2 hours of the current time, display the alert interface relating to the missed meal dose.

Clause 113. The system of clause 110, wherein the instructions further cause the one or more processors to:

determine if a correction dose alert is asserted; and

in response to a determination that the correction dose alert is not asserted, display the alert interface relating to the missed meal dose.

Clause 114. The system of clause 110, further comprising a sensor control device configured to collect data indicative of an analyte level in a subject, the sensor control device comprising an analyte sensor, wherein at least a portion of the analyte sensor is configured to be in fluid contact with a bodily fluid of the subject.

Clause 115. The system of clause 110, wherein the input comprises wireless communications circuitry.

Clause 116. A method for alerting a user to a missed meal dose, the method comprising:

receiving, by an electronic device, streaming glucose data from a sensor control device;

determining at a current time if a meal dose has been missed in association with a meal having an estimated meal start time by detecting a missed meal dose condition for a consecutive number of minutes;

determining if an insulin dose has not been recorded within about 45 minutes prior to the estimated meal start time; and

in response to a detection of a missed meal dose condition for the consecutive number of minutes and a determination that the insulin dose has not been recorded within about 45 minutes prior to the estimated meal start time, displaying an alert interface relating to the missed meal dose.

Clause 117. The method of clause 116, wherein the consecutive number of minutes is about 5 minutes.

Clause 118. The method of clause 116, further comprising the step of:

if a meal dose has been recorded within about 2 hours of the current time; and

in response to a determination that the meal dose has been recorded within about 2 hours of the current time, displaying the alert interface relating to the missed meal dose.

Clause 119. The method of clause 116, further comprising the steps of:

determining if a correction dose alert is asserted;

in response to a determination that the correction dose alert is not asserted, displaying the alert interface relating to the missed meal dose.

Clause 120. A system for managing alerts, the system comprising:

an input configured to receive streaming glucose data from a sensor control device;

a display configured to present an alert;

one or more processors coupled with the input, the display, and a memory storing instructions, wherein the instructions, when executed by the one or more processors, cause the one or more processors to:
assert a missed meal dose alert;
determine if a missed meal dose condition has been detected for a consecutive number of minutes after the missed meal dose alert has been asserted;
in response to a determination that the missed meal dose condition has not been detected for the consecutive number of minutes after the missed meal dose alert has been asserted, rescind the missed meal dose alert.

Clause 121. The system of clause 120, wherein the consecutive number of minutes is 15 consecutive minutes.

Clause 122. The system of clause 120, wherein the missed meal dose condition comprises a determination that an insulin dose was not administered within a period of time of an estimated meal start time.

Clause 123. The system of clause 120, wherein the input comprises wireless communications circuitry.

Clause 124. A method for rescinding an alert to a missed meal dose, the method comprising:

receiving, by an electronic device, streaming glucose data from a sensor control device;

asserting a missed meal dose alert;

determining if a missed meal dose condition has been detected for a consecutive number of minutes after the missed meal dose alert has been asserted;

in response to a determination that the missed meal dose condition has not been detected for the consecutive number of minutes after the missed meal dose alert has been asserted, rescinding the missed meal dose alert.

Clause 125. The method of clause 124, wherein the consecutive number of minutes is 15 consecutive minutes.

Clause 126. The method of clause 124, wherein the missed meal dose condition comprises determining that an insulin dose was not administered within a period of time of an estimated meal start time.

Clause 127. A system for managing alerts, the system comprising:

an input configured to receive streaming glucose data from a sensor control device and dose data from a medication delivery device;

a display configured to present an alert;

one or more processors coupled with the input, the display, and a memory storing instructions, wherein the instructions, when executed by the one or more processors, cause the one or more processors to:
assert a missed meal dose alert at a current time;
determine if an insulin dose has been recorded within about 2 hours of the current time;
in response to a determination that the insulin dose has been recorded within about 2 hours of the current time, rescind the missed meal dose alert.

Clause 128. The system of clause 127, wherein the input comprises wireless communications circuitry.

Clause 129. A method for rescinding an alert to a missed meal dose, the method comprising:

receiving, by an electronic device, streaming glucose data from a sensor control device;

asserting a missed meal dose alert at a current time;

determining if an insulin dose has been recorded within about 2 hours of the current time;

and in response to a determination that the insulin dose has been recorded within about 2 hours of the current time, rescinding the missed meal dose alert.

Clause 130. A system for managing alerts, the system comprising:

an input configured to receive streaming glucose data from a sensor control device and dose data from a medication delivery device;

a display configured to present an alert;

one or more processors coupled with the input, the display, and a memory storing instructions, wherein the instructions, when executed by the one or more processors, cause the one or more processors to:
assert a missed meal dose alert, wherein the missed meal dose alert relates to a missed meal having an estimated start time;
determine if an insulin dose has been recorded within about 45 minutes of the estimated meal start time; and
in response to a determination that the insulin dose has been recorded within about 45 minutes of the estimated meal start time, rescind the missed meal dose alert.

Clause 131. The system of clause 130, wherein the input comprises wireless communications circuitry

Clause 132. A method for rescinding an alert to a missed meal dose, the method comprising:

receiving, by an electronic device, streaming glucose data from a sensor control device;

asserting a missed meal dose alert, wherein the missed meal dose alert relates to a missed meal having an estimated start time;

determining if an insulin dose has been recorded within about 45 minutes of the estimated meal start time; and

in response to a determination that the insulin dose has been recorded within about 45 minutes of the estimated meal start time, rescinding the missed meal dose alert.

Clause 133. A system for managing alerts, the system comprising:

an input configured to receive streaming glucose data from a sensor control device;

a display configured to present an alert;

one or more processors coupled with the input, the display, and a memory storing instructions, wherein the instructions, when executed by the one or more processors, cause the one or more processors to:
assert a missed meal dose alert at a current time, wherein the missed meal dose alert relates to a missed meal having an estimated start time;
determine if the estimated meal start time is within about 2 hours of the current time; and
in response to a determination that the estimated meal start time did not occur within about 2 hours of the current time, rescind the missed meal dose alert.

Clause 134. The system of clause 133, wherein the input comprises wireless communications circuitry.

Clause 135. A method for rescinding an alert to a missed meal dose, the method comprising:

receiving, by an electronic device, streaming glucose data from a sensor control device;

asserting a missed meal dose alert at a current time, wherein the missed meal dose alert relates to a missed meal having an estimated start time;

determining if the estimated meal start time is within about 2 hours of the current time; and

in response to a determination that the estimated meal start time did not occur within about 2 hours of the current time, rescinding the missed meal dose alert.

Clause 136. A system for providing dose guidance to a subject, the system comprising:

an input configured to receive dose data from an insulin delivery device;

a display configured to visually present a dose guidance;

one or more processors coupled with the input, the display, and a memory storing instructions, wherein the instructions, when executed by the one or more processors, cause the one or more processors to:
determine if a correction dose alert has been asserted at a current time;
determine if an insulin dose has not been administered to the subject within about 2 hours of the current time; and
in response to a determination that the correction dose has been asserted and a determination that the insulin dose has not been administered to the subject within about 2 hours of the current time, display a correction dose guidance.

Clause 137. The system of clause 136, wherein the instructions further cause the one or more processors to:

determine if the correction dose alert has been resolved; and

in response to a determination that the correction dose alert has been resolved, display the correction dose guidance.

Clause 138. The system of clause 136, wherein the input comprises wireless communications circuitry.

Clause 139. A method for recommending correction doses, the method comprising:

receiving, by an electronic device, insulin dose data of a subject from an insulin delivery device;

determining if a correction dose alert has been asserted at a current time;

determining if an insulin dose has not been administered to the subject within about 2 hours of the current time; and

displaying a correction dose guidance,

wherein the correction dose guidance is displayed in response to a determination that the correction dose has been asserted and a determination that the insulin dose has not been administered to the subject within about 2 hours of the current time.

Clause 140. The method of clause 139, further comprising the step of determining if the correction dose alert has been resolved, wherein the correction dose guidance is only displayed if the correction dose alert has been resolved.

Clause 141. A system for providing alerts to a subject, the system comprising:

an input configured to receive streaming glucose data from a sensor control device and dose data from an insulin delivery device;

a display configured to visually present an alert;

one or more processors coupled with the input, the display, and a memory storing instructions, wherein the instructions, when executed by the one or more processors, cause the one or more processors to:
determine at a current time if a correction dose condition has been asserted for a consecutive number of minutes;
determine if an insulin dose has not been recorded within about 2 hours of the current time; and
in response to a determination that the correction dose condition has been asserted for a consecutive number of minutes and a determination that the insulin dose has not been recorded within about 2 hours of the current time, display an alert interface relating to the correction dose alert.

Clause 142. The system of clause 141, wherein the consecutive number of minutes is about 5 minutes.

Clause 143. The system of clause 141, wherein the instructions further cause the one or more processors to:

determine if a missed meal dose alert is asserted;

in response to a determination that the missed dose alert is not asserted, display the alert interface relating to the correction dose alert; and

in response to a determination that the missed dose alert is asserted, not display the alert interface relating to the correction dose alert.

Clause 144. The system of clause 141, wherein the input comprises wireless communications circuitry.

Clause 145. A method for alerting a user regarding a correction dose, the method comprising:

receiving, by an electronic device, streaming glucose data from a sensor control device and insulin dose data of a subject from an insulin delivery device;

determining at a current time if a correction dose condition has been asserted for a consecutive number of minutes;

determining if an insulin dose has not been recorded within about 2 hours of the current time; and

in response to a determination that the correction dose condition has been asserted for a consecutive number of minutes and a determination that the insulin dose has not been recorded within about 2 hours of the current time, displaying an alert interface relating to the correction dose alert.

Clause 146. The method of clause 145, wherein the consecutive number of minutes is about 5 minutes.

Clause 147. The method of clause 145, further comprising the step of determining if a missed meal dose alert is not asserted before displaying the alert interface relating to the correction dose alert.

Clause 148. The method of clause 147, wherein the alert interface relating to the correction dose alert is only displayed in response to a determination that the missed meal dose alert is not asserted.

Clause 149. A system for managing alerts, the system comprising:

an input configured to receive streaming glucose data from a sensor control device;

a display configured to present an alert;

one or more processors coupled with the input, the display, and a memory storing instructions, wherein the instructions, when executed by the one or more processors, cause the one or more processors to:
assert a correction dose alert;
determine if a correction dose condition has been detected for a consecutive number of minutes after the correction dose has been asserted;
in response to a determination that the correction dose condition has not been detected for the consecutive number of minutes after the correction dose has been asserted, rescind the correction dose alert.

Clause 150. The system of clause 149, wherein the consecutive number of minutes is 15 consecutive minutes.

Clause 151. The system of clause 149, wherein the input comprises wireless communications circuitry.

Clause 152. A method for rescinding an alert to a correction dose, the method comprising:

receiving, by an electronic device, streaming glucose data from a sensor control device;

asserting a correction dose alert;

determining if a correction dose condition has been detected for a consecutive number of minutes after the correction dose has been asserted;

in response to a determination that the correction dose condition has not been detected for the consecutive number of minutes after the correction dose has been asserted, rescinding the correction dose alert.

Clause 153. The method of clause 152, wherein the consecutive number of minutes is 15 consecutive minutes.

Clause 154. A system for managing alerts, the system comprising:

an input configured to receive streaming glucose data from a sensor control device;

a display configured to present an alert;

one or more processors coupled with the input, the display, and a memory storing instructions, wherein the instructions, when executed by the one or more processors, cause the one or more processors to:
assert a correction dose alert, wherein the correction dose alert was first asserted at a first time;
determine if a calculation for insulin on board (JOB) has changed since the first time; and
in response to a determination that the calculation for insulin on board (JOB) has changed since the first time, rescind the correction dose alert.

Clause 155. The system of clause 154, wherein the input comprises wireless communications circuitry.

Clause 156. A method for rescinding an alert to a correction dose, the method comprising:

receiving, by an electronic device, streaming glucose data from a sensor control device;

asserting a correction dose alert, wherein the correction dose alert was first asserted at a first time;

determining if a calculation for insulin on board (JOB) has changed since the first time; and

in response to a determination that the calculation for insulin on board (JOB) has changed since the first time, rescind the correction dose alert.

Clause 157. A system for managing alerts, the system comprising:

an input configured to receive streaming glucose data from a sensor control device and dose data from an insulin delivery device;

a display configured to visually present a dose guidance;

one or more processors coupled with the input, the display, and a memory storing instructions, wherein the instructions, when executed by the one or more processors, cause the one or more processors to:
assert a correction dose alert at a current time;
determine if an insulin dose has been recorded within a period of time of the current time; and
in response to a determination that the insulin dose has been recorded within the period of time of the current time, rescind the correction dose alert.

Clause 158. The system of clause 157, wherein the period of time is about 2 hours.

Clause 159. The system of clause 157, wherein the input comprises wireless communications circuitry.

Clause 160. A method for rescinding an alert to a correction dose, the method comprising:

receiving, by an electronic device, streaming glucose data from a sensor control device and insulin dose data of a subject from an insulin delivery device;

asserting a correction dose alert at a current time;

determining if an insulin dose has been recorded within a period of time of the current time; and

rescinding the correction dose alert if it is determined that the insulin dose has been recorded within the period of time of the current time.

Clause 161. The method of clause 160, wherein the period of time is about 2 hours.

Clause 162. A system for classifying doses, the system comprising:

an input configured to receive insulin dose data of a user from a connected insulin delivery device, wherein the insulin dose data comprises a recent dose comprising an insulin amount and a timestamp;

a display configured to visually present a dose guidance;

one or more processors coupled with the input, the display, and a memory storing instructions, wherein the instructions, when executed by the one or more processors, cause the one or more processors to:
provide a dose recommendation guidance for a meal requested by a user at a request time, wherein the meal has a meal type and the dose recommendation guidance comprises a recommended dose amount;
determine if the timestamp of the recent dose is within a period of time of the request time;
determine if the insulin amount of the recent dose is the same as the recommended dose amount of the meal dose guidance recommendation; and
in response to a determination that the timestamp of the recent dose is within a period of time of the request time and a determination that the insulin amount of the recent dose is the same as the recommended dose amount of the meal dose guidance recommendation, classify the recent dose as associated with the meal type of the recent meal.

Clause 163. The system of clause 162, wherein the period of time is less than or equal to about 20 minutes.

Clause 164. The system of clause 162, wherein the meal type is selected from the group consisting of breakfast, lunch, and dinner.

Clause 165. The system of clause 162, wherein the instructions further cause the one or more processors to:

determine if the timestamp of the recent dose is within a determined meal dose time range for the meal type of the recent meal; and

in response to a determination that the timestamp of the recent dose is within the determined meal dose time range for the meal type of the recent meal, classify the recent dose as associated with the meal type of the recent meal.

Clause 166. The system of clause 162, wherein the instructions further cause the one or more processors to:

determine if the recent dose was taken while the user was in a post-meal state; and

in response to a determination that the recent dose was taken while the user was not in the post-meal state, classify the recent dose as associated with the meal type of the recent meal.

Clause 167. The system of clause 166, wherein in the post-meal state, a previous dose administered within about 2 hours of the request time has been associated with the meal type of the recent meal.

Clause 168. The system of clause 162, wherein the input comprises wireless communications circuitry.

Clause 169. A method for classifying doses from a connected insulin delivery device, the method comprising:

providing a dose recommendation guidance for a meal requested by a user at a request time, wherein the meal has a meal type and the dose recommendation guidance comprises a recommended dose amount;

receiving, by an electronic device, insulin dose data of a user from the connected insulin delivery device, wherein the insulin dose data comprises a recent dose comprising an insulin amount and a timestamp;

determining if the timestamp of the recent dose is within a period of time of the request time;

determining if the insulin amount of the recent dose is the same as the recommended dose amount of the meal dose guidance recommendation; and

in response to a determination that the timestamp of the recent dose is within a period of time of the request time and a determination that the insulin amount of the recent dose is the same as the recommended dose amount of the meal dose guidance recommendation, classifying the recent dose as associated with the meal type of the recent meal.

Clause 170. The method of clause 162, wherein the period of time is less than or equal to about 20 minutes.

Clause 171. The method of clause 162, wherein the meal type is selected from the group consisting of breakfast, lunch, and dinner.

Clause 172. The method of clause 162, further comprising the step of determining if the timestamp of the recent dose is within a determined meal dose time range for the meal type of the recent meal.

Clause 173. The method of clause 162, further comprising the step of determining if the recent dose was taken while the user was in a post-meal state.

Clause 174. The method of clause 173, wherein in a post-meal state, a previous dose administered within about 2 hours of the request time has been associated with the meal type of the recent meal.

Clause 175. A system for classifying doses, the system comprising:

an input configured to receive insulin dose data of a user from a connected insulin delivery device, wherein the insulin dose data comprises a recent dose comprising an insulin amount and a timestamp;

a display configured to visually present a dose guidance;

one or more processors coupled with the input, the display, and a memory storing instructions, wherein the instructions, when executed by the one or more processors, cause the one or more processors to:
provide a dose recommendation guidance at a first time;
determine if the timestamp of the recent dose is within a period of time of the first time;
determine if the insulin amount of the recent dose is the same as the recommended dose amount of the dose recommendation guidance; and
classify the recent dose as a meal dose, a correction dose, or as ambiguous.

Clause 176. The system of clause 175, wherein the period of time is less than or equal to about 20 minutes.

Clause 177. The system of clause 175, wherein the dose recommendation guidance was a correction dose recommendation guidance, and wherein the recent dose is classified as a correction dose.

Clause 178. The system of clause 175, wherein the dose recommendation guidance was a dose recommendation guidance for a meal, wherein the meal has a meal type, and wherein the recent dose is classified as a dose for the meal type.

Clause 179. The system of clause 178, wherein the instructions further cause the one or more processors to:

determine if the timestamp of the recent dose is within a determined meal dose time range for the meal type of the recent meal.

Clause 180. The system of clause 178, wherein the instructions further cause the one or more processors to:

determine if the recent dose was taken while the user was in a post-meal state.

Clause 181. The system of clause 175, wherein the recent dose is classified as ambiguous.

Clause 182. The system of clause 181, wherein the instructions further cause the one or more processors to:

prompt the user to classify the recent dose manually.

Clause 183. The system of clause 182, wherein the instructions further cause the one or more processors to:

prompt the user to classify the recent dose manually by prompting the user to select a classification from the group consisting of a breakfast dose, a lunch dose, a dinner dose, and a correction dose.

Clause 184. The system of clause 182, wherein the instructions further cause the one or more processors to:

prompt the user to classify the recent dose manually by prompting the user to select a classification from the group consisting of a snack dose, a priming dose, and a dose not taken.

Clause 185. The system of clause 182, wherein the instructions further cause the one or more processors to:

prompt the user to classify the recent dose manually by prompting the user to select a classification relating to eating more than expected from a previous meal.

Clause 186. The system of clause 181, wherein the recent dose that was classified as ambiguous must be classified as a classification other than ambiguous before providing an additional dose guidance recommendation.

Clause 187. The system of clause 175, wherein the input comprises wireless communications circuitry.

Clause 188. A method for classifying doses from a connected insulin delivery device, the method comprising:

providing a dose recommendation guidance at a first time;

receiving, by an electronic device, insulin dose data of a user from the connected insulin delivery device, wherein the insulin dose data comprises a recent dose comprising an insulin amount and a timestamp;

determining if the timestamp of the recent dose is within a period of time of the first time;

determining if the insulin amount of the recent dose is the same as the recommended dose amount of the dose recommendation guidance; and

classifying the recent dose as a meal dose, a correction dose, or as ambiguous.

Clause 189. The method of clause 188, wherein the period of time is less than or equal to about 20 minutes.

Clause 190. The method of clause 188, wherein the dose recommendation guidance was a correction dose recommendation guidance, and wherein the recent dose is classified as a correction dose.

Clause 191. The method of clause 188, wherein the dose recommendation guidance was a dose recommendation guidance for a meal, wherein the meal has a meal type, and wherein the recent dose is classified as a dose for the meal type.

Clause 192. The method of clause 191, further comprising the steps of determining if the timestamp of the recent dose is within a determined meal dose time range for the meal type of the recent meal.

Clause 193. The method of clause 191, further comprising the step of determining if the recent dose was taken while the user was in a post-meal state.

Clause 194. The method of clause 188, wherein the recent dose is classified as ambiguous.

Clause 195. The method of clause 194, further comprising the step of prompting the user to classify the recent dose manually.

Clause 196. The method of clause 195, wherein the step of prompting the user to classify the recent dose manually comprises prompting the use to select a classification from the group consisting of a breakfast dose, a lunch dose, a dinner dose, and a correction dose.

Clause 197. The method of clause 195, wherein the step of prompting the user to classify the recent dose manually comprises prompting the use to select a classification from the group consisting of a snack dose, a priming dose, and a dose not taken.

Clause 198. The method of clause 195, wherein the step of prompting the user to classify the recent dose manually comprises prompting the use to select a classification relating to eating more than expected from a previous meal.

Clause 199. The method of clause 194, wherein the recent dose that was classified as ambiguous must be classified as a classification other than ambiguous before providing an additional dose guidance recommendation.

Clause 200. An apparatus for providing dose guidance in response to analyte data, the apparatus comprising:

an input configured to receive measured analyte data, meal data, and medication dosing data;

a display configured to visually present information; and

one or more processors coupled with the input, the display, and a memory storing instructions, wherein the instructions, when executed by the one or more processors, cause the apparatus to perform:
receiving time-correlated analyte data of a patient taken over an analysis period into a buffer;
dividing the time-correlated analyte data into discrete time-of-day (TOD) periods;
determining, by executing an algorithm, a recommended fixed dose of the medication for a corresponding one of the TOD periods based on at least one portion of the time-correlated analyte data and a defined dosing strategy of the patient for the analysis period; and
storing an indicator of the recommended fixed dose in a computer memory for output to at least one of a user or a medication dosing device.

Clause 201. The apparatus of clause 200, wherein the memory holds further instructions for determining the recommended fixed dose at least in part by:

classifying each of the medication doses in a medication class, based on the time-correlated data;

grouping each of the doses in one of a set of mealtime groups;

determining a glucose pattern most closely fitting the time-correlated data; and

selecting a glucose pattern indicator, based on the glucose pattern.

Clause 202. The apparatus of clause 200, wherein the memory holds further instructions for classifying doses in classes comprising a fixed basal dose, fixed breakfast dose, fixed lunch dose, fixed dinner dose for corresponding TOD periods.

Clause 203. The apparatus of clause 200, wherein the memory holds further instructions for determining, as a condition for use in determining the recommended fixed dose, that a segment of the time-correlated analyte data is free of any gaps exceeding a defined threshold.

Clause 204. The apparatus of clause 200, wherein the memory holds further instructions for determining, as a condition for use in determining the recommended fixed dose, that a segment of the time-correlated analyte data has an associated initial meal dose.

Clause 205. The apparatus of clause 200, wherein the memory holds further instructions for determining, as a condition for use in determining the recommended fixed dose, that a segment of the time-correlated analyte data is associated with a basal fixed dose within a prior 24-hour period.

Clause 206. The apparatus of clause 200, wherein the memory holds further instructions for clearing data for each TOD period in response to any one or more of: determining the recommended fixed dose, determining a pre-meal correction factor, determining a post-meal correction factor, or determining a manual dose adjustment.

Clause 207. The apparatus of clause 200, wherein the memory holds further instructions for determining a glucose pattern for each TOD period based on associated valid data segments for a set number of prior days, wherein determining the recommended fixed dose is further based on the glucose pattern.

Clause 208. The apparatus of clause 207, wherein the memory holds further instructions for determining, as a condition for determining the recommended fixed dose, that the associated valid data segments are available for the set number of prior days.

Clause 209. The apparatus of clause 207, wherein the memory holds further instructions for determining the glucose pattern is low based on a count of low alarms occurring in each TOD period.

Clause 210. The apparatus of clause 207, wherein the memory holds further instructions for determining the glucose pattern is low based on a count of hypoglycemic instances in each TOD period.

Clause 211. The apparatus of clause 207, wherein the memory holds further instructions for determining the glucose pattern is high based on a count of hyperglycemic instances in each TOD period.

Clause 212. The apparatus of clause 207, wherein the memory holds further instructions for determining a pre-meal correction factor based on the time-correlated analyte data independently of the determining the recommended fixed dose, and if both the pre-meal correction factor and the recommended fixed dose indicate an increase in dose, then maintaining the pre-meal correction factor.

Clause 213. The apparatus of clause 201, wherein the memory holds further instructions for determining a glucose pattern condition based on a count of low alarms, a count of post-meal corrections during each TOD period, and the glucose pattern indicator.

Clause 214. The apparatus of clause 213, wherein the memory holds further instructions for determining that if the count of low alarms exceeds a first threshold, the count of post-meal corrections exceeds a second threshold, and a result of the GPA method indicates a low pattern, then determining the glucose pattern is low.

Clause 215. The apparatus of clause 213, wherein the memory holds further instructions for determining that if the count of low alarms exceeds a first threshold, the count of post-meal corrections exceeds a second threshold, and a result of the GPS method indicates a moderate hypoglycemic risk or high/low pattern, then determining the glucose pattern is high/low.

Clause 216. The apparatus of clause 213, wherein the memory holds further instructions for determining that if the count of low alarms exceeds a first threshold, the count of post-meal corrections exceeds a second threshold, and the glucose pattern indicator indicates no pattern or a high pattern, then determining the glucose pattern is high.

Clause 217. The apparatus of clause 200, wherein the input comprises wireless communications circuitry.

Clause 218. A medication delivery device, comprising:

an input configured to receive a query for dose data for a period of time, wherein the dose data comprises an amount and a time of all doses delivered during the period of time;

one or more processors coupled with the input and a memory storing instructions, wherein the instructions, when executed by the one or more processors, cause the one or more processors to:
store data for doses administered during the period of time to create stored data;
determine if the stored data includes all doses delivered during the period of time; and
in response to a determination that the stored data does not include all doses delivered during the period of time, create an indication of incomplete dose data.

Clause 219. The device of clause 218, wherein the instructions further cause the one or more processors to transmit the indication of incomplete dose data in response to the query for dose data.

Clause 220. The device of clause 218, wherein the indication of incomplete dose data is a counter value.

Clause 221. The device of clause 218, wherein the indication of incomplete dose data is based on a counter value.

Clause 222. The device of clause 218, wherein the indication of incomplete dose data is based on a comparison of a counter value and an estimated counter value.

Clause 223. The device of clause 222, wherein the estimated counter value is calculated based on a prior counter value and an elapsed time since the prior counter value was received.

Clause 224. The device of clause 218, wherein the medication delivery device is a connected insulin pen, and wherein the connected insulin pen is configured to transmit dose data wirelessly.

Clause 225. The device of clause 218, wherein the medication delivery device is an insulin pen and a connected pen cap, wherein the connected pen cap is configured to transmit dose data wirelessly.

Clause 226. The device of clause 218, wherein the indication of incomplete dose data is based on a detection that a pen cap was not attached to an insulin pen for a different period of time.

Clause 227. The device of clause 226, wherein the detection that the pen cap was not attached to the insulin pen for the different period of time comprises a determination that the insulin pen contained a first amount of insulin before the pen cap was not attached and a second amount of insulin after the pen cap was reattached to the insulin pen, wherein the first amount is different than the second amount.

Clause 228. The device of clause 218, wherein the query for dose data from the period of time was sent from an application that provides dose guidance.

Clause 229. The device of clause 218, wherein the input comprises wireless communications circuitry.

Clause 230. A method of transferring data, the method comprising the steps of:

receiving a query for dose data for a period of time, wherein the dose data comprises an amount and a time of all doses delivered during the period of time;

storing data for doses administered during the period of time to create stored data;

determining if the stored data includes all doses delivered during the period of time; and

in response to a determination that the stored data does not include all doses delivered during the period of time, creating an indication of incomplete dose data.

Clause 231. The method of clause 230, further comprising the step of transmitting the indication of incomplete dose data in response to the query for dose data.

Clause 232. The method of clause 230, wherein the indication of incomplete dose data is a counter value.

Clause 233. The method of clause 230, wherein the indication of incomplete dose data is based on a counter value.

Clause 234. The method of clause 230, wherein the indication of incomplete dose data is based on a comparison of a counter value and an estimated counter value.

Clause 235. The method of clause 234, wherein the estimated counter value is calculated based on a prior counter value and an elapsed time since the prior counter value was received.

Clause 236. The method of clause 230, wherein the indication of incomplete dose data is based on a detection that a pen cap was not attached to an insulin pen for a different period of time.

Clause 237. The method of clause 236, wherein the detection that the pen cap was not attached to the insulin pen for the different period of time comprises a determination that the insulin pen contained a first amount of insulin before the pen cap was not attached and a second amount of insulin after the pen cap was reattached to the insulin pen, wherein the first amount is different than the second amount.

Clause 238. The method of clause 230, wherein the query for dose data from the period of time was sent from an application that provides dose guidance.

Clause 239. A system for providing dose guidance to a subject, the system comprising:

an input configured to receive dose data and an indication of incomplete dose data from a medication delivery device, wherein the dose data comprises data related to at least one dose administered during a period of time;

a display configured to visually present a dose guidance;

one or more processors coupled with the input, the display, and a memory storing instructions, wherein the instructions, when executed by the one or more processors, cause the one or more processors to:
query the medication delivery device for the dose data comprising data related to at least one dose administered during the period of time;
determine if the indication of incomplete dose data is received from the medication delivery device;
in response to a determination that the indication of incomplete dose data is received, output a prompt seeking confirmation that the dose data received for the period of time includes dose data for all doses administered during the period of time; and
in response to a determination that the indication of incomplete dose data is not received, calculate a dose guidance.

Clause 240. The system of clause 239, wherein the instructions further cause the one or more processors to:

output the dose guidance on the display.

Clause 241. The system of clause 239, wherein the indication of incomplete dose data is based on a counter value.

Clause 242. The system of clause 239, wherein the indication of incomplete dose data is based on a comparison of a counter value and an estimated counter value.

Clause 243. The system of clause 242, wherein the estimated counter value is calculated based on a prior counter value and an elapsed time since the prior counter value was received.

Clause 244. The system of clause 239, wherein the medication delivery device is a connected insulin pen, wherein the connected insulin pen is configured to transmit dose data wirelessly.

Clause 245. The system of clause 239, wherein the medication delivery device is an insulin pen and a connected pen cap, wherein the connected pen cap is configured to transmit dose data wirelessly.

Clause 246. The system of clause 239, wherein the system further comprises a medication delivery device, and wherein the medication delivery device further comprises:

an input configured to receive the query for the dose data comprising data related to at least one dose administered during the period of time;

one or more processors coupled with the input and a memory storing instructions, wherein the instructions, when executed by the one or more processors, cause the one or more processors to:
store data for doses administered during the period of time to create stored data;
determine if the stored data includes all doses delivered during the period of time;
in response to a determination that the stored data does not include all doses delivered during the period of time, create the indication of incomplete dose data; and
in response to the query for the dose data and to the determination that the stored data does not include all doses delivered during the period of time, transmit the indication of incomplete dose data.

Clause 247. The system of clause 239, wherein the input comprises wireless communications circuitry.

Clause 248. A method for providing dose guidance to a subject, the method comprising the steps of:

receiving dose data and an indication of incomplete dose data from a medication delivery device, wherein the dose data comprises data related to at least one dose administered during a period of time;

querying the medication delivery device for the dose data comprising data related to at least one dose administered during the period of time;

determining if the indication of incomplete dose data is received from the medication delivery device;

in response to a determination that the indication of incomplete dose data is received, outputting a prompt seeking confirmation that the dose data received for the period of time includes dose data for all doses administered during the period of time; and in response to a determination that the indication of incomplete dose data is not received, calculating a dose guidance.

Clause 249. The method of clause 248, further comprising the step of outputting the dose guidance on the display.

Clause 250. The method of clause 248, wherein the indication of incomplete dose data is based on a counter value.

Clause 251. The method of clause 248, wherein the indication of incomplete dose data is based on a comparison of a counter value and an estimated counter value.

Clause 252. The method of clause 251, wherein the estimated counter value is calculated based on a prior counter value and an elapsed time since the prior counter value was received.

Clause 253. The method of clause 248, wherein the medication delivery device is a connected insulin pen, wherein the connected insulin pen is configured to transmit dose data wirelessly.

Clause 254. The method of clause 248, wherein the medication delivery device is an insulin pen and a connected pen cap, wherein the connected pen cap is configured to transmit dose data wirelessly.

Clause 255. A method for recommending a dose for a meal, the method comprising:

prompting a user to input a tag associated with a meal type;

receiving an inputted tag for an instance of the meal type;

associating the inputted tag with an amount of medication administered for the instance of the meal type and a post-prandial analyte data set for the instance of the meal type;

determining whether a threshold number of instances associated with the meal type is met; and

determining a recommended medication dose for the meal type if the threshold number of instances is met.

Clause 256. The method of clause 255, wherein the recommended medication dose for the meal type is based at least in part on the amount of medication administered for the instance of the meal type and the post-prandial analyte data set for the instance of the meal type.

Clause 257. The method of clause 255, wherein the recommended medication dose for the meal type is based at least in part on a plurality of amounts of medication administered for a plurality of instances of the meal type and a plurality of post-prandial analyte data sets for the plurality of instances of the meal type.

Clause 258. The method of clause 257, wherein the instance of the meal type is a first instance of the meal type, and wherein the plurality of instances of the meal type includes the first instance of the meal type.

Clause 259. The method of clause 255, further comprising the step of receiving analyte data from a sensor control device.

Clause 260. The method of clause 259, wherein the recommended medication dose for the meal type is based at least in part on the analyte data received from the sensor control device.

Clause 261. The method of clause 255, further comprising the step of visually outputting to a display the recommended medication dose for the meal type.

Clause 262. The method of clause 255, further comprising the step of prompting the user with an option to track the meal type.

Clause 263. The method of clause 262, wherein the prompting of the user to input the tag associated with the meal type occurs in response to the user selecting the option to track the meal type.

Clause 264. A system for determining a recommended medication dose, the system comprising:

one or more processors coupled with a memory for storing instructions, wherein the instructions, when executed by the one or more processors, cause the one or more processors to:
prompt a user to input a tag associated with a meal type,
receive an inputted tag for an instance of the meal type,
associate the inputted tag with an amount of medication administered for the instance of the meal type and a post-prandial analyte data set for the instance of the meal type,
determine whether a threshold number of instances associated with the meal type is met, and
determine the recommended medication dose for the meal type if the threshold number of instances is met.

Clause 265. The system of clause 264, wherein the recommended medication dose for the meal type is based at least in part on the amount of medication administered for the instance of the meal type and the post-prandial analyte data set for the instance of the meal type.

Clause 266. The system of clause 264, wherein the recommended medication dose for the meal type is based at least in part on a plurality of amounts of medication administered for a plurality of instances of the meal type and a plurality of post-prandial analyte data sets for the plurality of instances of the meal type.

Clause 267. The system of clause 266, wherein the instance of the meal type is a first instance of the meal type, and wherein the plurality of instances of the meal type includes the first instance of the meal type.

Clause 268. The system of clause 264, further comprising wireless communication circuitry configured to receive data indicative of an analyte level from a sensor control device.

Clause 269. The system of clause 268, wherein the recommended medication dose for the meal type is based at least in part on the data indicative of the analyte level received from the sensor control device.

Clause 270. The system of clause 264, wherein the instructions, when executed by the one or more processors, further cause the one or more processors to visually output to a display the recommended medication dose for the meal type.

Clause 271. The system of clause 264, wherein the instructions, when executed by the one or more processors, further cause the one or more processors to prompt the user with an option to track the meal type.

Clause 272. The system of clause 271, wherein the instructions, when executed by the one or more processors, cause the one or more processors to prompt the user to input the tag associated with the meal type only if the user has selected the option to track the meal type.

Clause 273. A system for smart meal tagging, the system comprising:

one or more processors coupled with a memory for storing instructions, wherein the instructions, when executed by the one or more processors, cause the one or more processors to:
prompt a user to input a tag associated with a meal type,
receive an inputted tag for an instance of a first meal type, wherein the first meal type is associated with one or more previously inputted tags,
determine whether a meal type characteristic of the instance of the first meal type exceeds a meal type characteristic threshold, and
associate the inputted tag with a second meal type, wherein the second meal type is different from the first meal type.

Clause 274. The system of clause 273, wherein the meal type characteristic of the instance is based at least in part on a difference between a meal size associated with the one or more previously inputted tags and a meal size of the inputted tag for the instance.

Clause 275. The system of clause 273, wherein the meal type characteristic of the instance is based at least in part on a difference between an amount of medication associated with the one or more previously inputted tags and an amount of medication associated with the inputted tag for the instance.

Clause 276. The system of clause 273, wherein the instructions, when executed by the one or more processors, further cause the one or more processors to prompt the user with an option to create the second tag if the meal type characteristic of the instance of the first meal type exceeds a meal type characteristic threshold.

Clause 277. The system of clause 273, wherein the instructions, when executed by the one or more processors, cause the one or more processors to associate the inputted tag with the second meal type only if the user has selected the option to create the second tag.

Clause 278. The system of clause 273, wherein the instructions, when executed by the one or more processors, further cause the one or more processors to disassociate the inputted tag for the instance from the first meal type if the meal type characteristic of the instance of the first meal type exceeds a meal type characteristic threshold.

Clause 279. The system of clause 278, wherein the instructions, when executed by the one or more processors, cause the one or more processors to disassociate the inputted tag for the instance from the first meal type before the inputted tag is associated with the second meal type.

Clause 280. A method for recommending a dose for a meal, the method comprising:

prompting a user to input a tag associated with a meal type;

receiving a first inputted tag for a first instance of the meal type;

associating the first inputted tag with a first amount of medication administered for the first instance of the meal type;

receiving a second inputted tag for a second instance of the meal type;

associating the second inputted tag with a second amount of medication administered for the second instance of the meal type and a second post-prandial analyte data set for the instance of the meal type; and

in response to a determination that a difference between the second amount of medication and the first amount of medication is greater than a predetermined threshold difference, prompting the user to input a modified tag associated with the meal type.

Clause 281. The method of clause 280, wherein the modified tag comprises a different size of the meal.

Clause 282. The method of clause 280, wherein the predetermined threshold difference is at least about 2 units.

Clause 283. The method of clause 280, wherein the prompting of the user to input the modified tag associated with the meal type occurs in real time.

Clause 284. The method of clause 280, wherein the prompting of the user to input the modified tag associated with the meal type occurs within about 5 minutes or less of receiving the second inputted tag.

Clause 285. The method of clause 280, wherein the prompting of the user to input the modified tag associated with the meal type occurs within about 2 minutes or less of receiving the second inputted tag.

Clause 286. The method of clause 280, further comprising the steps of:

associating the first inputted tag with a first post-prandial analyte data set for the first instance of the meal type; and

associating the second inputted tag with a second post-prandial analyte data set for the second instance of the meal type.

Clause 287. A system for meal tagging, the system comprising:

one or more processors coupled with a memory for storing instructions, wherein the instructions, when executed by the one or more processors, cause the one or more processors to:
prompt a user to input a tag associated with a meal type,
receive a first inputted tag for a first instance of the meal type,
associate the first inputted tag with a first amount of medication administered for the first instance of the meal type,
receive a second inputted tag for a second instance of the meal type;
associate the second inputted tag with a second amount of medication administered for the second instance of the meal type, and
in response to a determination that a difference between the second amount of medication and the first amount of medication is greater than a predetermined threshold difference, prompt the user to input a modified tag associated with the meal type.

Clause 288. The system of clause 287, wherein the modified tag comprises a different size of the meal.

Clause 289. The system of clause 287, wherein the predetermined threshold difference is at least about 2 units.

Clause 290. The system of clause 287, wherein the instructions, when executed by the one or more processors, further cause the one or more processors to prompt the user to input the modified tag associated with the meal type occurs in real time.

Clause 291. The system of clause 287, wherein the instructions, when executed by the one or more processors, further cause the one or more processors to prompt the user to input the modified tag associated with the meal type within about 5 minutes or less of receiving the second inputted tag.

Clause 292. The system of clause 287, wherein the instructions, when executed by the one or more processors, further cause the one or more processors to prompt the user to input the modified tag associated with the meal type within about 2 minutes or less of receiving the second inputted tag.

Clause 293. The system of clause 287, wherein the instructions, when executed by the one or more processors, further cause the one or more processors to associate the first inputted tag with a first post-prandial analyte data set for the first instance of the meal type and associate the second inputted tag with a second post-prandial analyte data set for the second instance of the meal type.

Clause 294. An analyte monitoring system, comprising:

a sensor control device comprising an analyte sensor, wherein at least a portion of the analyte sensor is configured to be in fluid contact with a bodily fluid of a subject; and

a reader device, comprising:
wireless communication circuitry configured to receive analyte levels from the sensor control device; and
one or more processors coupled to a memory, the memory storing instructions that, when executed by the one or more processors, cause the one or more processors to:
   determine a pattern type for at least one time segment of a day based on a hypo risk metric and a hyper risk metric for the at least one time segment of the day; and
   output to a display to a user interface comprising:
      at least one glucose metric determined for a time period based on the analyte levels received from the sensor control device;
      a time in range display comprising a graph of time in ranges comprising a plurality of graph portions, wherein each graph portion of the plurality of graph portions indicates an amount of time that a user's analyte level is within a predefined analyte range associated with each graph portion, wherein the plurality of graph portions comprises at least 4 graph portions;
      a graph comprising a plot of analyte levels of the user across a horizontal representation of a plurality of time segments of a day and an identification of the determined pattern type for the at least one time segment.

Clause 295. The system of clause 294, wherein the at least one glucose metric comprises a glucose average.

Clause 296. The system of clause 294, wherein the at least one glucose metric comprises a glucose management indicator.

Clause 297. The system of clause 294, wherein the instructions further cause the one or more processors to output a display to the user interface comprising a goal value corresponding to the at least one glucose metric.

Clause 298. The system of clause 294, wherein the plurality of graph portions comprises at least 5 graph portions.

Clause 299. The system of clause 294, wherein the plurality of graph portions comprises at least four graph portions selected from a group consisting of: a graph portion below a very low threshold, a graph portion between a very low threshold and a low threshold, a graph portion between a low threshold and a high threshold, a graph portion between a high threshold and a very high threshold, and a graph portion above a very high threshold.

Clause 300. The system of clause 294, wherein the time in range display further comprises a description of the predefined analyte range associated with each graph portion.

Clause 301. The system of clause 294, wherein the time in range display further comprises a value for each graph portion of the plurality of graph portions that relates to the amount of time that the user's analyte level was within the predefined analyte range associated with the graph portion during the time period.

Clause 302. The system of clause 301, wherein the value is a percentage value.

Clause 303. The system of clause 294, wherein the time in range display further comprises a combined value for at least two graph portions of the plurality of graph portions that relates to a sum of the amount of time that the user's analyte level was within each of the predefined analyte ranges associated with at least two graph portions during the time period.

Clause 304. The system of clause 294, wherein the graph of the time in ranges comprises a histogram.

Clause 305. The system of clause 304, wherein each graph portion of the histogram are arranged in a vertical layout, wherein a graph portion below a very low threshold is located below a graph portion between a very low threshold and a low threshold, which is locate below a graph portion between a low threshold and a high threshold, which is located below a graph portion between a high threshold and a very high threshold, which is located below a graph portion above a very high threshold.

Clause 306. The system of clause 294, wherein the identification of the determined pattern type for the at least one time segment comprises at least a partial outline of the time segment on the graph.

Clause 307. The system of clause 306, wherein the identification of the determined pattern type for the at least one time segment further comprises a label of the determined pattern type.

Clause 308. The system of clause 294, wherein the pattern type is at least one of a lows pattern, a highs with some lows pattern, a highs pattern, or no pattern.

Clause 309. The system of clause 294, wherein the graph comprises a plurality of determined pattern types, and wherein an identification of a single pattern type is visibly distinct from other identifications of the plurality of determined pattern types.

Clause 310. The system of clause 294, wherein the instructions further cause the one or more processors to output a display to the user interface an identification of a most important pattern type, wherein the most important pattern type is one of the pattern type determined for the at least one time segment of the day.

Clause 311. The system of clause 310, wherein the identification of the most important pattern type is displayed on the graph.

Clause 312. The system of clause 310, wherein the identification of the determined pattern type for the at least one time segment comprises a plurality of identifications of a determined pattern type for each of the at least one time segment, and wherein the identification of the most important pattern type is visibly distinct from other identifications of the plurality of identifications.

Clause 313. The system of clause 310, wherein the pattern type determined for the at least one time segment of the day comprises a plurality of pattern types for a plurality of time segments of the day.

Clause 314. The system of clause 313, wherein the plurality of pattern types comprises at least two of a lows pattern, a highs with some lows pattern, a highs pattern, or no pattern.

Clause 315. The system of clause 314, wherein if the plurality of pattern types comprises a lows pattern, the identification of the most important pattern type comprises an identification of the lows pattern.

Clause 316. The system of clause 314, wherein if the plurality of pattern types comprises a highs with some lows pattern and does not comprise a lows pattern, the identification of the most important pattern type comprises an identification of the highs with some lows pattern.

Clause 317. The system of clause 314, wherein if the plurality of pattern types comprises a highs pattern and does not comprise a highs with some lows pattern or a lows pattern, the identification of the most important pattern type comprises an identification of the highs pattern.

Clause 318. The system of clause 310, wherein the instructions further cause the one or more processors to output a display to the user interface comprising an identification of at least one time segment of the day that was determined to have the most important pattern type.

Clause 319. The system of clause 310, wherein the display of the identification of the most important pattern type and the identification of the at least one time segment of the day that was determined to have the most important pattern type comprises a tag for the identification of the most important pattern type and at least one tag for the identification of the at least one time segment of the day that was determined to have the most important pattern type.

Clause 320. The system of clause 319, wherein the tag for the identification of the most important pattern type is a different color that the at least one tag for the identification of the at least one time segment of the day that was determined to have the most important pattern type.

Clause 321. The system of clause 294, wherein the instructions further cause the one or more processors to:

determine a variability of at least one time segment of the day;

output a display to the user interface comprising a statement relating to variability if the determined variability is high.

Clause 322. The system of clause 321, wherein the statement relating to variability comprises an identification of behaviors that may contribute to glucose variability.

Clause 323. The system of clause 294, wherein the instructions further cause the one or more processors to output a display to the user interface comprising a statement relating to an excursion below a very low threshold.

Clause 324. The system of clause 323, wherein the very low threshold is between about 50 mg/dL and about 58 mg/dL.

Clause 325. The system of clause 323, wherein the very low threshold is about 54 mg/dL.

Clause 326. The system of clause 294, wherein the instructions further cause the one or more processors to output a display to the user interface comprising statements relating to medication considerations.

Clause 327. The system of clause 326, wherein the statements relating to medication considerations comprise suggestions to adjust a medication.

Clause 328. The system of clause 326, wherein the statements relating to medication considerations comprise suggestions related to medications contributing to low glucose levels.

Clause 329. The system of clause 294, wherein the instructions further cause the one or more processors to output a display to the user interface comprising statements relating to lifestyle considerations.

Clause 330. The system of clause 329, wherein the statements relating to lifestyle considerations comprise statements relating to at least one of missed meals, carbohydrates, activity level, alcohol, and medication.

Clause 331. The system of clause 294, wherein the time period is 14 days.

Clause 332. A method for displaying information related to glucose levels in a subject, comprising the steps of:

receiving analyte levels from a sensor control device;

determining a pattern type for at least one time segment of a day based on a hypo risk metric and a hyper risk metric for the at least one time segment of the day; and

displaying a user interface comprising:
at least one glucose metric determined for a time period based on the analyte levels received from the sensor control device;
a time in range display comprising a graph of time in ranges comprising a plurality of graph portions, wherein each graph portion of the plurality of graph portions indicates an amount of time that a user's analyte level is within a predefined analyte range associated with each graph portion, wherein the plurality of graph portions comprises at least 4 graph portions; and
a graph comprising a plot of analyte levels of the user across a horizontal representation of a plurality of time segments of a day and an identification of the determined pattern type for the at least one time segment.

Clause 333. The method of clause 332, wherein the at least one glucose metric comprises a glucose average.

Clause 334. The method of clause 332, wherein the at least one glucose metric comprises a glucose management indicator.

Clause 335. The method of clause 332, wherein the instructions further cause the one or more processors to output a display to the user interface comprising a goal value corresponding to the at least one glucose metric.

Clause 336. The method of clause 332, wherein the plurality of graph portions comprises at least 5 graph portions.

Clause 337. The method of clause 332, wherein the plurality of graph portions comprises at least four graph portions selected from a group consisting of: a graph portion below a very low threshold, a graph portion between a very low threshold and a low threshold, a graph portion between a low threshold and a high threshold, a graph portion between a high threshold and a very high threshold, and a graph portion above a very high threshold.

Clause 338. The method of clause 332, wherein the time in range display further comprises a description of the predefined analyte range associated with each graph portion.

Clause 339. The method of clause 332, wherein the time in range display further comprises a value for each graph portion of the plurality of graph portions that relates to the amount of time that the user's analyte level was within the predefined analyte range associated with the graph portion during the time period.

Clause 340. The method of clause 339, wherein the value is a percentage value.

Clause 341. The method of clause 332, wherein the time in range display further comprises a combined value for at least two graph portions of the plurality of graph portions that relates to a sum of the amount of time that the user's analyte level was within each of the predefined analyte ranges associated with the at least two graph portions during the time period.

Clause 342. The method of clause 332, wherein the graph of the time in ranges comprises a histogram.

Clause 343. The method of clause 343, wherein each graph portion of the histogram are arranged in a vertical layout, wherein a graph portion below a very low threshold is located below a graph portion between a very low threshold and a low threshold, which is locate below a graph portion between a low threshold and a high threshold, which is located below a graph portion between a high threshold and a very high threshold, which is located below a graph portion above a very high threshold.

Clause 344. The method of clause 332, wherein the identification of the determined pattern type for the at least one time segment comprises at least a partial outline of the time segment on the graph.

Clause 345. The method of clause 344, wherein the identification of the determined pattern type for the at least one time segment further comprises a label of the determined pattern type.

Clause 346. The method of clause 332, wherein the pattern type is at least one of a lows pattern, a highs with some lows pattern, a highs pattern, or no pattern.

Clause 347. The method of clause 332, wherein the graph comprises a plurality of determined pattern types, and wherein an identification of a single pattern type is visibly distinct from other identifications of the plurality of determined pattern types.

Clause 348. The method of clause 332, wherein the instructions further cause the one or more processors to output a display to the user interface an identification of a most important pattern type, wherein the most important pattern type is one of the pattern type determined for the at least one time segment of the day.

Clause 349. The method of clause 348, wherein the identification of the most important pattern type is displayed on the graph.

Clause 350. The method of clause 348, wherein the identification of the determined pattern type for the at least one time segment comprises a plurality of identifications of a determined pattern type for each of the at least one time segment, and wherein the identification of the most important pattern type is visibly distinct from other identifications of the plurality of identifications.

Clause 351. The method of clause 348, wherein the pattern type determined for the at least one time segment of the day comprises a plurality of pattern types for a plurality of time segments of the day.

Clause 352. The method of clause 351, wherein the plurality of pattern types comprises at least two of a lows pattern, a highs with some lows pattern, a highs pattern, or no pattern.

Clause 353. The method of clause 352, wherein if the plurality of pattern types comprises a lows pattern, the identification of the most important pattern type comprises an identification of the lows pattern.

Clause 354. The method of clause 352, wherein if the plurality of pattern types comprises a highs with some lows pattern and does not comprise a lows pattern, the identification of the most important pattern type comprises an identification of the highs with some lows pattern.

Clause 355. The method of clause 352, wherein if the plurality of pattern types comprises a highs pattern and does not comprise a highs with some lows pattern or a lows pattern, the identification of the most important pattern type comprises an identification of the highs pattern.

Clause 356. The method of clause 348, wherein the instructions further cause the one or more processors to output a display to the user interface comprising an identification of at least one time segment of the day that was determined to have the most important pattern type.

Clause 357. The method of clause 348, wherein the display of the identification of the most important pattern type and the identification of the at least one time segment of the day that was determined to have the most important pattern type comprises a tag for the identification of the most important pattern type and at least one tag for the identification of the at least one time segment of the day that was determined to have the most important pattern type.

Clause 358. The method of clause 357, wherein the tag for the identification of the most important pattern type is a different color that the at least one tag for the identification of the at least one time segment of the day that was determined to have the most important pattern type.

Clause 359. The method of clause 332, wherein the instructions further cause the one or more processors to:

determine a variability of at least one time segment of the day;

output a display to the user interface comprising a statement relating to variability if the determined variability is high.

Clause 360. The method of clause 359, wherein the statement relating to variability comprises an identification of behaviors that may contribute to glucose variability.

Clause 361. The method of clause 332, wherein the instructions further cause the one or more processors to output a display to the user interface comprising a statement relating to an excursion below a very low threshold.

Clause 362. The method of clause 361, wherein the very low threshold is between about 50 mg/dL and about 58 mg/dL.

Clause 363. The method of clause 361, wherein the very low threshold is about 54 mg/dL.

Clause 364. The method of clause 332, wherein the instructions further cause the one or more processors to output a display to the user interface comprising statements relating to medication considerations.

Clause 365. The method of clause 364, wherein the statements relating to medication considerations comprise suggestions to adjust a medication.

Clause 366. The method of clause 364, wherein the statements relating to medication considerations comprise suggestions related to medications contributing to low glucose levels.

Clause 367. The method of clause 332, wherein the instructions further cause the one or more processors to output a display to the user interface comprising statements relating to lifestyle considerations.

Clause 368. The method of clause 367, wherein the statements relating to lifestyle considerations comprise statements relating to at least one of missed meals, carbohydrates, activity level, alcohol, and medication.

Clause 369. The method of clause 332, wherein the time period is 14 days.

Clause 370. An apparatus for displaying metrics relating to a subject, the apparatus comprising:
an input configured to receive drug dosing data;
a display configured to visually present information; and
one or more processors coupled with the input, the display, and a memory storing instructions and doses of a medication received by the subject over a period of time and recommended doses of the medication for the subject over the period of time, wherein the instructions, when executed by the one or more processors, cause the apparatus to:
   display a graph plotting a plurality of medication doses taken by the subject at a plurality of times, wherein the graph comprises an x-axis of time and a y-axis of a difference between a dose taken by the subject and a dose recommended for the subject.

Clause 371. The apparatus of clause 370, wherein the plurality of medication doses comprises at least one of basal doses, fixed meal doses, and meal doses with a correction factor.

Clause 372. The apparatus of clause 371, wherein the fixed meal doses comprise at least one of fixed breakfast doses, fixed lunch doses, and fixed dinner doses.

Clause 373. The apparatus of clause 371, wherein the meal doses with a correction factor comprise at least one of breakfast doses with a correction factor, fixed lunch doses with a correction factor, and fixed dinner doses with a correction factor.

Clause 374. The apparatus of clause 370, wherein the difference between the dose taken by the subject and the dose recommended is in units.

Clause 375. The apparatus of clause 370, wherein the input comprises wireless communications circuitry.

Clause 376. An apparatus for displaying metrics relating to a subject, the apparatus comprising:
an input configured to receive measured analyte data and drug dosing data;
a display configured to visually present information; and
one or more processors coupled with the input, the display, and a memory storing instructions and time-correlated data characterizing an analyte of the subject, doses of a medication received by the subject over a period of time, and recommended doses of the medication for the subject over the period of time, wherein the instructions, when executed by the one or more processors, cause the apparatus to:
   display a summary of therapies for the subject comprising dose amounts administered during a time period and analyte metrics determined from the received measured analyte data;
   display a graphic summarizing missed doses during the time period; and
   display a graphic summarizing override doses, wherein override doses comprise doses received by the subject at a time that are a different amount than a recommended dose for the time.

Clause 377. The apparatus of clause 376, wherein the graphic summarizing missed doses comprises a graphic representation of percentages of missed doses for a plurality of dose types.

Clause 378. The apparatus of clause 377, wherein each percentage of the percentages of missed for the plurality of dose types is calculated as a percentage of missed doses of the dose type of a total number of doses of the dose type during a time period.

Clause 379. The apparatus of clause 377, wherein the plurality of dose types comprises at least one of basal doses, breakfast doses, lunch doses, and dinner doses.

Clause 380. The apparatus of clause 376, wherein the graphic summarizing missed doses is a bar graph.

Clause 381. The apparatus of clause 376, wherein the graphic summarizing override doses is a bar graph.

Clause 382. The apparatus of clause 376, wherein the input comprises wireless communications circuitry.

Clause 383. An apparatus for displaying metrics relating to a subject, the apparatus comprising:
an input configured to receive measured analyte data from a plurality of subjects, drug dosing data from a plurality of subjects, and data related to dosing recommendations for the plurality of subjects;
a display configured to visually present information; and
one or more processors coupled with the input, the display, and a memory storing instructions and time-correlated data characterizing an analyte of each of the plurality of subjects, doses of a medication received by each of the plurality of subjects over a period of time, and the data related to dosing recommendations for the plurality of subjects, when executed by the one or more processors, cause the apparatus to:
   display a summary of analyte metrics for the each of the plurality of subjects, wherein the analyte metrics comprise at least two of time in range, time below a low threshold, time above a high threshold, percentage of basal doses taken, and average bolus doses taken per day; and
   display a summary of information related to dosing recommendations, wherein the information related to dosing recommendations comprises an indication of a dosing recommendation for a subject of the plurality of subjects needing approval from a health care provider.

Clause 384. The apparatus of clause 383, wherein the summary of analyte metrics comprises at least three of time in range, time below a low threshold, time above a high threshold, percentage of basal doses taken, and average bolus doses taken per day.

Clause 385. The apparatus of clause 383, wherein the low threshold is about 70 mg/dL.

Clause 386. The apparatus of clause 383, wherein the high threshold is about 180 mg/dL.

Clause 387. The apparatus of clause 383, wherein the indication of the dosing recommendation is an icon.

Clause 388. The apparatus of clause 383, wherein the indication of the dosing recommendation is a statement indicating a number of dosing recommendations needing approval.

Clause 389. The apparatus of clause 383, wherein the input comprises wireless communications circuitry.

Clause 390. An apparatus for displaying treatment information relating to a subject, the apparatus comprising:
an input configured to receive measured analyte data and drug dosing data;
a display configured to visually present information; and
one or more processors coupled with the input, the display, and a memory storing instructions and time-correlated data characterizing an analyte of the subject, doses of a medication received by the subject over a period of time, and meal times of the subject, wherein the instructions, when executed by the one or more processors, cause the apparatus to:
   receive estimated dose parameters and estimated meal dosing time ranges from the subject;
   determine a representative amount of each of a plurality of basal doses and a plurality of meal doses taken by the subject during a period of time based on the drug dosing data;
   determine representative meal dosing time ranges for the subject during the time period based on the drug dosing data;
   determine recommended dose amounts for at least one of a basal dose, a breakfast dose, a lunch dose, and a dinner dose;
   display the estimated dose parameters and estimated meal dosing time ranges received from the subject;
   display the representative amount of each of a plurality of basal doses and a plurality of meal doses and the representative meal dosing time ranges; and
   display the recommended dose amounts for at least one of the basal dose, the breakfast dose, the lunch dose, and the dinner dose.

Clause 391. The apparatus of clause 390, wherein the plurality of meal doses comprises a plurality of breakfast doses, a plurality of lunch doses, and a plurality of dinner doses, and wherein the instructions, when executed by the one or more processors, cause the apparatus to determine an average amount of each of the plurality of basal doses, plurality of breakfast doses, the plurality of lunch doses, and the plurality of dinner doses.

Clause 392. The apparatus of clause 390, wherein the estimated dose parameters comprise estimated amounts for a basal dose, a breakfast dose, a lunch dose, and a dinner dose.

Clause 393. The apparatus of clause 392, wherein the estimated dose parameter further comprises estimated times that the subject takes the basal dose, the breakfast dose, the lunch dose, and the dinner dose.

Clause 394. The apparatus of clause 390, wherein the estimated meal dosing time ranges comprise an estimated dosing start time and an estimated dosing end time for each of breakfast, lunch, and dinner.

Clause 395. The apparatus of clause 390, wherein the representative amount of each of the plurality of basal doses and the plurality of meal doses comprise an average of each of the plurality of basal doses and the plurality of meal doses taken by the subject during a period of time.

Clause 396. The apparatus of clause 390, wherein the representative amount of each of the plurality of basal doses and the plurality of meal doses comprise a mode of each of the plurality of basal doses and the plurality of meal doses taken by the subject during a period of time.

Clause 397. The apparatus of clause 390, wherein the instructions, when executed by the one or more processors, further cause the apparatus to:
determine a pre-meal correction factor and a post-meal correction factor based on the measured analyte data and the drug dosing data; and
display the pre-meal correction factor and the post-meal correction factor.

Clause 398. The apparatus of clause 390, wherein the representative meal dosing time ranges are displayed adjacent to the estimated meal dosing time ranges.

Clause 399. The apparatus of clause 390, wherein the representative amount of each of the plurality of basal doses and the plurality of meal doses is displayed adjacent to the estimated dose parameters.

Clause 400. The apparatus of clause 390, wherein the instructions, when executed by the one or more processors, further cause the apparatus to:
determine a conservative value for at least one of a basal dose, a breakfast dose, a lunch dose, and a dinner dose, wherein the conservative value is lower than a corresponding determined representative amount of each of the plurality of basal doses and a plurality of meal doses; and
display the determined conservative value.

Clause 401. The apparatus of clause 390, wherein the input comprises wireless communications circuitry.

1-401. (canceled)

402. An analyte monitoring system, comprising:
a sensor control device comprising an analyte sensor, wherein at least a portion of the analyte sensor is configured to be in fluid contact with a bodily fluid of a subject; and
a reader device, comprising:
wireless communication circuitry configured to receive analyte levels from the sensor control device; and
one or more processors coupled to a memory, the memory storing instructions that, when executed by the one or more processors, cause the one or more processors to:
determine a pattern type for at least one time segment of a day based on at least one of a hypo risk metric and a hyper risk metric for the at least one time segment of the day; and
output to a display to a user interface comprising:
at least one glucose metric determined for a time period based on the analyte levels received from the sensor control device;
a time in range display comprising a graph of time in ranges comprising a plurality of graph portions, wherein each graph portion of the plurality of graph portions indicates an amount of time that a user's analyte level is within a predefined analyte range associated with each graph portion, wherein the plurality of graph portions comprises at least 4 graph portions;
a graph comprising a plot of analyte levels of the user across a horizontal representation of a plurality of time segments of a day and an identification of the determined pattern type for the at least one time segment.

403. The system of claim 402, wherein the at least one glucose metric comprises a glucose average.

404. The system of claim 402, wherein the at least one glucose metric comprises a glucose management indicator.

405. The system of claim 402, wherein the instructions further cause the one or more processors to output a display to the user interface comprising a goal value corresponding to the at least one glucose metric.

406. The system of claim 402, wherein the plurality of graph portions comprises at least 5 graph portions.

407. The system of claim 402, wherein the plurality of graph portions comprises at least four graph portions selected from a group consisting of: a graph portion below a very low threshold, a graph portion between a very low threshold and a low threshold, a graph portion between a low threshold and a high threshold, a graph portion between a high threshold and a very high threshold, and a graph portion above a very high threshold.

408. The system of claim 402, wherein the time in range display further comprises a description of the predefined analyte range associated with each graph portion.

409. The system of claim 402, wherein the time in range display further comprises a value for each graph portion of the plurality of graph portions that relates to the amount of time that the user's analyte level was within the predefined analyte range associated with the graph portion during the time period.

410. The system of claim 409, wherein the value is a percentage value.

411. The system of claim 402, wherein the time in range display further comprises a combined value for at least two graph portions of the plurality of graph portions that relates to a sum of the amount of time that the user's analyte level was within each of the predefined analyte ranges associated with at least two graph portions during the time period.

412. The system of claim 402, wherein the graph of the time in ranges comprises a histogram.

413. The system of claim 412, wherein each graph portion of the histogram are arranged in a vertical layout, wherein a graph portion below a very low threshold is located below a graph portion between a very low threshold and a low threshold, which is locate below a graph portion between a low threshold and a high threshold, which is located below a graph portion between a high threshold and a very high threshold, which is located below a graph portion above a very high threshold.

414. The system of claim 402, wherein the identification of the determined pattern type for the at least one time segment comprises at least a partial outline of the time segment on the graph.

415. The system of claim 414, wherein the identification of the determined pattern type for the at least one time segment further comprises a label of the determined pattern type.

416. The system of claim 402, wherein the pattern type is at least one of a lows pattern, a highs with some lows pattern, a highs pattern, or no pattern.

417. The system of claim 402, wherein the graph comprises a plurality of determined pattern types, and wherein an identification of a single pattern type is visibly distinct from other identifications of the plurality of determined pattern types.

418. The system of claim 402, wherein the instructions further cause the one or more processors to output a display to the user interface an identification of a most important pattern type, wherein the most important pattern type is one of the pattern type determined for the at least one time segment of the day.

419. The system of claim 418, wherein the identification of the most important pattern type is displayed on the graph.

420. The system of claim 418, wherein the identification of the determined pattern type for the at least one time segment comprises a plurality of identifications of a determined pattern type for each of the at least one time segment, and wherein the identification of the most important pattern type is visibly distinct from other identifications of the plurality of identifications.

421. The system of claim 418, wherein the pattern type determined for the at least one time segment of the day comprises a plurality of pattern types for a plurality of time segments of the day.

422. The system of claim 421, wherein the plurality of pattern types comprises at least two of a lows pattern, a highs with some lows pattern, a highs pattern, or no pattern.

423. The system of claim 422, wherein if the plurality of pattern types comprises a lows pattern, the identification of the most important pattern type comprises an identification of the lows pattern.

424. The system of claim 422, wherein if the plurality of pattern types comprises a highs with some lows pattern and does not comprise a lows pattern, the identification of the most important pattern type comprises an identification of the highs with some lows pattern.

425. The system of claim 422, wherein if the plurality of pattern types comprises a highs pattern and does not comprise a highs with some lows pattern or a lows pattern, the identification of the most important pattern type comprises an identification of the highs pattern.

426. The system of claim 418, wherein the instructions further cause the one or more processors to output a display to the user interface comprising an identification of at least one time segment of the day that was determined to have the most important pattern type.

427. The system of claim 418, wherein the display of the identification of the most important pattern type and the identification of the at least one time segment of the day that was determined to have the most important pattern type comprises a tag for the identification of the most important pattern type and at least one tag for the identification of the at least one time segment of the day that was determined to have the most important pattern type.

428. The system of claim 427, wherein the tag for the identification of the most important pattern type is a different color that the at least one tag for the identification of the at least one time segment of the day that was determined to have the most important pattern type.

429. The system of claim 402, wherein the instructions further cause the one or more processors to:
determine a variability of at least one time segment of the day;
output a display to the user interface comprising a statement relating to variability if the determined variability is high.

430. The system of claim 429, wherein the statement relating to variability comprises an identification of behaviors that may contribute to glucose variability.

431. The system of claim 402, wherein the instructions further cause the one or more processors to output a display to the user interface comprising a statement relating to an excursion below a very low threshold.

432. The system of claim 431, wherein the very low threshold is between about 50 mg/dL and about 58 mg/dL.

433. The system of claim 431, wherein the very low threshold is about 54 mg/dL.

434. The system of claim 402, wherein the instructions further cause the one or more processors to output a display to the user interface comprising statements relating to medication considerations.

435. The system of claim 434, wherein the statements relating to medication considerations comprise suggestions to adjust a medication.

436. The system of claim 434, wherein the statements relating to medication considerations comprise suggestions related to medications contributing to low glucose levels.

437. The system of claim 402, wherein the instructions further cause the one or more processors to output a display to the user interface comprising statements relating to lifestyle considerations.

438. The system of claim 437, wherein the statements relating to lifestyle considerations comprise statements relating to at least one of missed meals, carbohydrates, activity level, alcohol, and medication.

439. The system of claim 402, wherein the time period is 14 days.
